(19) United States
(12) Patent Application Publication
(10) Pub. No.: US 2025/0049036 A1
(43) Pub. Date: Feb. 13, 2025

(54) SUBSTITUTED FUSED BICYCLIC PYRIDINE CARBOXAMIDE COMPOUNDS FOR COMBATING PHYTOPATHOGENIC FUNGI

(71) Applicant: PI Industries Ltd., Rajasthan Udaipur (IN)

(72) Inventors: Arun R. JAGDALE, Maharashtra Pune (IN); Vishal A. MAHAJAN, Pimple Nilakh Pune (IN); Navnath D. RODE, Dist. Maharashtra Pune (IN); Lalit Kumar JENA, Dist-Balasore Odisha (IN); Santosh Kumar YADAV, Dist-Bahraich, Uttar Pradesh (IN); Sukriti SHARMA, Rajasthan Udaipur (IN); Mithil PAREKH, Bilimora Gujrat (IN); Santosh Shridhar AUTKAR, Akola Maharashtra (IN); Alexander G.M. KLAUSENER, Pulheim (DE); Rohit SAXENA, Uttar Pradesh Lucknow (IN)

(21) Appl. No.: 18/720,255

(22) PCT Filed: Dec. 16, 2022
(86) PCT No.: PCT/IN2022/051084

§ 371 Date: Jun. 14, 2024

(30) Foreign Application Priority Data
Dec. 17, 2021 (IN) 202111059075

(51) Int. Cl. A01N 43/90; A01N 43/42; A01P 3/00; C07D 215/54; C07D 221/04; C07D 471/04

(52) U.S. Cl. CPC A01N 43/90; A01N 43/42; A01P 3/00; C07D 215/54; C07D 221/04; C07D 471/04

(57) ABSTRACT

A compound having formula (I) and a process for preparing the compound of formula (I) are provided.

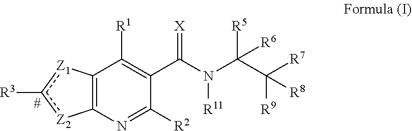

R¹, R², R³, R⁵, R⁶, R⁷, R⁸, R⁹, X, Z₁, and Z₂ are as defined in the detailed description. A composition and a combination containing novel fused bicyclic pyridine carboxamide compounds and method for combating phytopathogenic fungi using the same are also described.

### FIELD OF THE INVENTION

The present invention relates to novel fused bicyclic pyridine carboxamide compounds which are useful in combating phytopathogenic fungi, and to a process for preparing the novel fused bicyclic pyridine carboxamide compounds. The present invention also relates to compositions and to combinations comprising the fused bicyclic pyridine carboxamide compounds and a method for combating phytopathogenic fungi using the same.

### BACKGROUND OF THE INVENTION

Fused bicyclic pyridine carboxamide compounds are described as fungicidal agents in WO2004039783, WO2018172133, WO2017153380, WO2019053010 and WO2021063736. The fused bicyclic pyridine carboxamide compounds reported in the above cited literature have disadvantages in certain aspects, such as that they exhibit a narrow spectrum of efficacy or that they do not have satisfactory fungicidal activity, particularly at low application rates.

Therefore, the need remains for the development of new fungicidal compounds, including such that are belonging to the class of the above cited fused bicyclic pyridine carboxamide compounds, to provide compounds being effective against a broader spectrum of fungi, having lower toxicity, higher selectivity, and being used at lower dosage rate to reduce or avoid unfavorable environmental or toxicological effects whilst still allowing effective and long-lasting control.

Therefore, it is an objective of the present invention to provide compounds having an improved/enhanced activity and/or a broader efficacy spectrum against phytopathogenic fungi.

This objective is achieved by using compounds of the present invention for combating phytopathogenic fungi.

### SUMMARY OF THE INVENTION

The present invention provides a compound of Formula (I),

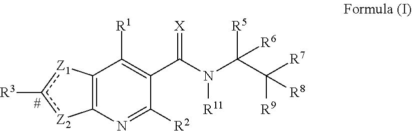

wherein, R¹, R², R³, R⁵, R⁶, R⁷, R⁸, R⁹, R¹¹, X, Z₁, and Z₂ are as defined in the detailed description.

The present invention also relates to a process for preparing a compound of Formula (I).

The compounds of Formula (I) have been found to be advantageous over the compounds reported in the literature in either of improved fungicidal activity, broader spectrum of biological efficacy, lower application rates, more favourable biological, environmental properties, or enhanced plant compatibility.

The present invention further relates to agrochemical compositions comprising a compound of Formula (I) or a compound of Formula (I) in combination with one or more further pesticidally active substance(s) for controlling and/or preventing plant diseases, particularly caused by phytopathogenic fungi.

The present invention still further relates to a method for controlling or preventing infestation of useful plants by phytopathogenic microorganisms, wherein a fungicidally effective amount of a compound of formula (I), a composition or a combination thereof, is applied to the plants, to parts thereof or a locus thereof.

### DETAILED DESCRIPTION OF THE INVENTION

### Definitions

The definitions provided herein for the terminologies used in the present disclosure are for illustrative purpose only and in no manner limit the scope of the present invention disclosed in the present disclosure.

As used herein, the terms “comprises”, “comprising”, “includes”, “including”, “has”, “having”, “contains”, “containing”, “characterized by” or any other variation thereof, are intended to cover a non exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

Further, unless expressly stated to the contrary, “or” refers to an inclusive “or” and not to an exclusive “or”. For example, a condition A “or” B is satisfied by any one of the following: A is true (or present) and B is false (or not present), A is false (or not present) and B is true (or present), and both A and B are true (or present).

Also, the indefinite articles “a” and “an” preceding an element or component of the present invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pests” includes but is not limited to fungi, stramenopiles (oomycetes), bacteria, nematodes, mites, ticks, insects and nematodes of economic importance.

In the context of this disclosure “pest control” means inhibition of invertebrate pest development (including necrosis, retarded growth and/or death), and related expressions are defined analogously.

The term “agronomic” refers to the production of field crops such as for food, feed and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

Nonagronomic applications include protecting an animal from a fungal disease by administering a fungicidally effective (i.e. biologically effective) amount of a compound of the present invention, typically in the form of a composition formulated for veterinary use, to the animal to be protected. As referred to in the present disclosure and claims, the terms “fungicidal” and “fungicidally” refers to observable effects on a fungal pathogen to provide protection of an animal from the disease. Fungicidal effects typically relate to diminishing the occurrence or activity of the target pathogen. Such effects on the pathogens include necrosis, death, retarded growth, or lessened ability to grow on or in the host animal. These effects on pathogens provide control (including prevention, reduction or elimination) of infestation or infection of the animal.

The meaning of various terms used in the description shall now be illustrated.

The term “alkyl”, used either alone or in compound words such as “alkylthio” or “haloalkyl” or —N(alkyl) or alkylcarbonylalkyl includes straight-chain or branched C₁ to C₂₄ alkyl, preferably C₁, to C₁₅ alkyl, more preferably C₁, to C₁₀ alkyl, most preferably C₁, to C₆ alkyl. Non-limiting examples of alkyl include methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl or the different isomers. If the alkyl is at the end of a composite substituent, as, for example, in alkylcycloalkyl, the part of the composite substituent at the start, for example the cycloalkyl, may be mono- or polysubstituted identically or differently and independently by alkyl. The same also applies to composite substituents in which other radicals, for example alkenyl, alkynyl, hydroxy, halogen, carbonyl, carbonyloxy and the like, are at the end.

The term “alkenyl”, used either alone or in compound words includes straight-chain or branched C₂ to C₂₄ alkenes, preferably C₂ to C₁₅ alkenes, more preferably C₂ to C₁₀ alkenes, most preferably C₂ to C₆ alkenes. Non-limiting examples of alkenes include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl and the different isomers. “Alkenyl” also includes polyenes such as 1,2-propadienyl and 2,4-hexadienyl. This definition also applies to alkenyl as a part of a composite substituent, for example haloalkenyl and the like, unless defined specifically elsewhere.

Non-limiting examples of alkynes include ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1, 1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl and the different isomers. This definition also applies to alkynyl as a part of a composite substituent, for example haloalkynyl etc., unless specifically defined elsewhere. The term “alkynyl” can also include moieties comprised of multiple triple bonds such as 2,5-hexadiynyl.

The term “cycloalkyl” means alkyl closed to form a ring. Non-limiting examples include cyclopropyl, cyclopentyl and cyclohexyl. This definition also applies to cycloalkyl as a part of a composite substituent, for example cycloalkylalkyl etc., unless specifically defined elsewhere.

The term “cycloalkenyl” means alkenyl closed to form a ring including monocyclic, partially unsaturated hydrocarbyl groups. Non-limiting examples include cyclopropenyl, cyclopentenyl and cyclohexenyl. This definition also applies to cycloalkenyl as a part of a composite substituent, for example cycloalkenylalkyl etc., unless specifically defined elsewhere.

The term “cycloalkynyl” means alkynyl closed to form a ring including monocyclic, partially unsaturated groups. Non-limiting examples include cyclopropynyl, cyclopentynyl and cyclohexynyl. This definition also applies to cycloalkynyl as a part of a composite substituent, for example cycloalkynylalkyl etc., unless specifically defined elsewhere.

The term “cycloalkoxy”, “cycloalkenyloxy” and the like are defined analogously. Non limiting examples of cycloalkoxy include cyclopropyloxy, cyclopentyloxy and cyclohexyloxy. This definition also applies to cycloalkoxy as a part of a composite substituent, for example cycloalkoxy alkyl etc., unless specifically defined elsewhere.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Non-limiting examples of “haloalkyl” include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1-dichloro-2,2,2-trifluoroethyl, and 1,1,1-trifluoroprop-2-yl. This definition also applies to haloalkyl as a part of a composite substituent, for example haloalkylaminoalkyl etc., unless specifically defined elsewhere.

The terms “haloalkenyl”, “haloalkynyl” are defined analogously except that, instead of alkyl groups, alkenyl and alkynyl groups are present as a part of the substituent.

The term “haloalkoxy” means straight-chain or branched alkoxy groups where some or all of the hydrogen atoms in these groups may be replaced by halogen atoms as specified above. Non-limiting examples of haloalkoxy include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy. This definition also applies to haloalkoxy as a part of a composite substituent, for example haloalkoxyalkyl etc., unless specifically defined elsewhere.

The term “haloalkylthio” means straight-chain or branched alkylthio groups where some or all of the hydrogen atoms in these groups may be replaced by halogen atoms as specified above. Non-limiting examples of haloalkylthio include chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio. This definition also applies to haloalkylthio as a part of a composite substituent, for example haloalkylthioalkyl etc., unless specifically defined elsewhere.

Non-limiting examples of “haloalkylsulfinyl” include CF₃S(O), CCl₃S(O), CF₃CH₂S(O) and CF₃CF₂S(O). Non-limiting examples of “haloalkylsulfonyl” include CF₃S(O)₂, CCl₃S(O)₂, CF₃CH₂S(O)₂ and CF₃CF₂S(O)₂.

The term “hydroxy” means —OH, “cyan” means —CN, “Amino” means —NRR, wherein R can be H or any possible substituent such as alkyl. Carbonyl means —C(═O)—, carbonyloxy means —OC(═O)—, sulfinyl means SO, sulfonyl means S(O)₂.

The term “oxo,” as used herein, alone or in combination, refers to ═O.

The term “alkoxy” used either alone or in compound words includes C₁, to C₂₄ alkoxy, preferably C₁, to C₁₅ alkoxy, more preferably C₁, to C₁₀ alkoxy, most preferably C₁, to C₆ alkoxy. Examples of alkoxy include methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy and the different isomers. This definition also applies to alkoxy as a part of a composite substituent, for example haloalkoxy, alkenylalkoxy, alkynylalkoxy, etc., unless specifically defined elsewhere.

The term “alkenyloxy,” as used herein, refers to an alkenyl group, as defined herein, appended to the parent molecular moiety through an oxy group, as defined herein.

The term “alkynyloxy,” as used herein, refers to an alkynyl group appended to the parent molecular moiety through an oxy group.

The term “alkoxyalkyl” denotes an alkoxy substitution on alkyl. Non-limiting examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkoxyalkoxy” denotes alkoxy substitution on alkoxy.

The term “alkylthio” includes branched or straight-chain alkylthio moieties such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio and the different isomers.

Halocycloalkyl, halocycloalkenyl, alkylcycloalkyl, cycloalkylalkyl, cycloalkoxyalkyl, alkylsulfinylalkyl, alkylsulfonylalkyl, haloalkylcarbonyl, cycloalkylcarbonyl, haloalkoxylalkyl, and the like, are defined analogously to the above examples.

The term “alkylthioalkyl” denotes an alkylthio substitution on alkyl. Non-limiting examples of “alkylthioalkyl” include —CH₂SCH₂, —CH₂SCH₂CH₂, CH₃CH₂SCH₂, CH₃CH₂CH₂CH₂SCH₂ and CH₃CH₂SCH₂CH₂. The term “Alkylthioalkoxy” denotes an alkylthio substitution on alkoxy. The term “cycloalkylalkylamino” denotes a cycloalkyl substitution on alkyl amino.

The terms “alkoxyalkoxyalkyl”, “alkylaminoalkyl”, “dialkylaminoalkyl”, “cycloalkylaminoalkyl”, “cycloalkylaminocarbonyl” and the like, are defined analogously to “alkylthioalkyl” or “cycloalkylalkylamino”.

The term “alkoxycarbonyl” is an alkoxy group bonded to a skeleton via a carbonyl group (—CO—). This definition also applies to alkoxycarbonyl as a part of a composite substituent, for example cycloalkylalkoxycarbonyl and the like, unless specifically defined elsewhere.

The term “alkoxycarbonylalkylamino” denotes an alkoxy carbonyl substitution on alkyl amino. “Alkylcarbonylalkylamino” denotes an alkyl carbonyl substitution on alkyl amino. The terms alkylthioalkoxycarbonyl, cycloalkylalkylaminoalkyl and the like are defined analogously.

Non-limiting examples of “alkylsulfinyl” include methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl and the different isomers. The term “arylsulfinyl” includes Ar—S(O), wherein Ar can be any carbocyle or heterocycle. This definition also applies to alkylsulphinyl as a part of a composite substituent, for example haloalkylsulphinyl etc., unless specifically defined elsewhere.

Non-limiting examples of “alkylsulfonyl” include methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl and the different isomers. The term “arylsulfonyl” includes Ar—S(O)₂, wherein Ar can be any carbocyle or heterocycle. This definition also applies to alkylsulphonyl as a part of a composite substituent, for example alkylsulphonylalkyl etc., unless defined elsewhere.

“Alkylamino”, “dialkylamino”, and the like, are defined analogously to the above examples. The term “carbocycle or carbocyclic” includes an “aromatic carbocyclic ring system” and “non-aromatic carbocylic ring system” or polycyclic or bicyclic (spiro, fused, bridged, nonfused) ring compounds in which the ring may be aromatic or non-aromatic (where aromatic indicates that the Huckel rule is satisfied and non-aromatic indicates that the Huckel rule is not satisfied).

The term “heterocycle or heterocyclic” includes “aromatic heterocycle or heteroaryl ring system” and “non-aromatic heterocycle ring system” or polycyclic or bicyclic (spiro, fused, bridged, nonfused) ring compounds in which the ring may be aromatic or non-aromatic, wherein the heterocycle ring contains at least one heteroatom selected from N, O, S(O)₀₋₂, and or the C ring member of the heterocycle may be replaced by C(═O), C(═S), C(═CR*R*) and C═NR*, * indicates integers. The term “heterocycloxy” refers to an heterocycle-O— group wherein the heterocycle group is as defined before.

The term “non-aromatic heterocycle” or “non-aromatic heterocyclic” means three- to fifteen-membered, preferably three- to twelve-membered, saturated or partially unsaturated heterocycle containing one to four heteroatoms from the group of oxygen, nitrogen and sulphur: mono, bi- or tricyclic heterocycles which contain, in addition to carbon ring members, one to three nitrogen atoms and/or one oxygen or sulphur atom or one or two oxygen and/or sulphur atoms; if the ring contains more than one oxygen atom, they are not directly adjacent; non-limiting examples thietanyl, oxetanyl, oxiranyl, azetidinyl, aziridinyl, 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-1-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, pyrrolinyl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, piperidinyl, 2-piperidinyl, 3-piperidinyl, 4-piperidinyl, pyrazynyl, morpholinyl, thiomorphlinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl, 1,2,4-hexahydrotriazin-3-yl, cycloserines, 2,3,4,5-tetrahydro[1H]azepin-1- or -2- or -3- or -4- or -5- or -6- or -7-yl, 3,4,5,6-tetra-hydro[2H]azepin-2- or -3- or -4- or -5- or -6- or 7-yl, 2,3,4,7-tetrahydro[1H]azepin-1- or -2- or -3- or -4- or -5- or -6- or 7-yl, 2,3,6,7-tetrahydro[1H]azepin-1- or -2- or -3- or -4- or -5- or -6- or -7-yl, hexahydroazepin-1- or -2- or -3- or -4-yl, tetra- and hexahydrooxepinyl such as 2,3,4,5-tetrahydro[1H]oxepin-2- or -3- or -4- or -5- or -6- or -7-yl, 2,3,4,7-tetrahydro[1H]oxepin-2- or -3- or -4- or -5- or -6- or -7-yl, 2,3,6,7-tetrahydro[1H]oxepin-2- or -3- or -4- or -5- or -6- or -7-yl, hexahydroazepin-1- or -2- or -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl. This definition also applies to a heterocyclyl as a part of a composite substituent, for example heterocyclylalkyl etc., unless specifically defined elsewhere.

The term “heteroaryl” or “aromatic heterocyclic” means 5 or 6-membered, fully unsaturated monocyclic ring systems containing one to four heteroatoms from the group of oxygen, nitrogen and sulphur, if the ring contains more than one oxygen atom, they are not directly adjacent; 5-membered heteroaryl containing one to four nitrogen atoms or one to three nitrogen atoms and one sulphur or oxygen atom; 5-membered heteroaryl groups which, in addition to carbon atoms, may contain one to four nitrogen atoms or one to three nitrogen atoms and one sulphur or oxygen atom as ring members, non-limiting examples furyl, thienyl, pyrrolyl, isoxazolyl, isothiazolyl, pyrazolyl, oxazolyl, thiazolyl, imidazolyl, 1,2,4-oxadiazolyl, 1,2,4-thiadiazolyl, 1,2,4-triazolyl, 1,3,4-oxadiazolyl, 1,3,4-thiadiazolyl, 1,3,4-triazolyl, tetrazolyl; nitrogen-bonded 5-membered heteroaryl containing one to four nitrogen atoms, or benzofused nitrogen-bonded 5-membered heteroaryl containing one to three nitrogen atoms: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, non-limiting examples 1-pyrrolyl, 1-pyrazolyl, 1,2,4-triazol-1-yl, 1-imidazolyl, 1,2,3-triazol-1-yl and 1,3,4-triazol-1-yl.

6-membered heteroaryl which contains one to four nitrogen atoms: 6-membered heteroaryl groups which, in addition to carbon atoms, may contain, respectively, one to three and one to four nitrogen atoms as ring members, non-limiting examples 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl; benzofused 5-membered heteroaryl containing one to three nitrogen atoms or one nitrogen atom and one oxygen or sulphur atom: non-limiting examples indol-1-yl, indol-2-yl, indol-3-yl, indol-4-yl, indol-5-yl, indol-6-yl, indol-7-yl, benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4yl, 1-benzothiophen-5-yl, 1-benzothiophen-6-yl, 1-benzothiophen-7-yl, 1,3-benzothiazol-2-yl, 1,3-benzothiazol-4yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl; benzofused 6-membered heteroaryl which contains one to three nitrogen atoms: non-limiting examples quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

An “aralkyl” or “arylalkyl” is an aryl group connected, as a substituent, via an alkylene group. The alkylene and aryl group of an aralkyl may be substituted. Examples include but are not limited to benzyl, substituted benzyl, 2-phenylethyl, 3-phenylpropyl, and naphthylalkyl. In some cases, the alkylene group is a lower alkylene group.

The term “aryloxy” as used herein refers to phenoxy, 1-naphthyloxy, 2-naphthyloxy and the like.

The term “heteroaryloxy” as used herein, alone or in combination, refers to a heteroaryl as defined herein linked to an oxygen atom having its free valence bond from the oxygen atom e.g. pyrrolyloxy, imidazolyloxy, pyrazolyloxy, triazolyloxy, pyrazinyloxy, pyrimidinyloxy, pyridazinyloxy, isothiazolyloxy, isoxazolyloxy, oxazolyloxy, oxadiazolyloxy, thiadiazolyloxy, quinolinyloxy, isoquinolinyloxy, quinazolinyloxy, quinoxalinyloxy, indolyloxy, benzimidazolyloxy, benzofuranyloxy, pteridinyloxy and purinyloxy and the like.

Non-limiting examples of “alkylcarbonyl” include C(O)CH₃, C(O)CH₂CH₂CH₃ and C(═O)CH(CH₃)₂. Non-limiting examples of “alkoxycarbonyl” include CH₃OC(═O), CH₃CH₂OC(═O), CH₃CH₂CH₂OC(═O), (CH₃)₂CHOC(═O) and the different butoxy- or pentoxycarbonyl isomers. Non-limiting examples of “alkylaminocarbonyl” include CH₃NHC(O), CH₃CH₂NHC(O), CH₃CH₂CH₂NHC(═O), (CH₃)₂CHNHC(═O) and the different butylamino- or pentylaminocarbonyl isomers. Non-limiting examples of “dialkylaminocarbonyl” include (CH₃)₂NC(O), (CH₃CH₂)₂NC(O), CH₃CH₂(CH₃)NC(═O), CH₃CH₂CH₂(CH₃)NC(═O) and (CH₃)₂CHN(CH₃)C(═O). Non-limiting examples of “alkoxyalkylcarbonyl” include CH₃OCH₂C(O), CH₃OCH₂CH₂C(O), CH₃CH₂OCH₂C(═O), CH₃CH₂CH₂CH₂OCH₂C(═O) and CH₃CH₂OCH₂CH₂C(═O). Non-limiting examples of “alkylthioalkylcarbonyl” include CH₃SCH₂C(═O), CH₃SCH₂CH₂C(═O), CH₃CH₂SCH₂C(═O), CH₃CH₂CH₂CH₂SCH₂C(═O) and CH₃CH₂SCH₂CH₂C(═O). The term haloalkylsufonylaminocarbonyl, alkylsulfonylaminocarbonyl, alkylthioalkoxycarbonyl, alkoxycarbonylalkyl amino and the like are defined analogously

Non-limiting examples of “alkylaminoalkylcarbonyl” include CH₃NHCH₂C(O), CH₃NHCH₂CH₂C(O), CH₃CH₂NHCH₂C(O), CH₃CH₂CH₂CH₂NHCH₂C(═O) and CH₃CH₂NHCH₂CH₂C(═O).

The term “amide” means A-R′C═ONR″-B, wherein R′ and R″ indicate substituents and A and B indicate any group.

The term “thioamide” means A-R′C═SNR″-B, wherein R′ and R″ indicate substituents and A and B indicate any group.

The total number of carbon atoms in a substituent group is indicated by the “Cᵢ-Cⱼ” prefix where i and j are numbers from 1 to 21. For example, C₁-C₃ alkylsulfonyl designates methylsulfonyl through propylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂. In the above recitations, when a compound of Formula (I) is comprised of one or more heterocyclic rings, all substituents are attached to these rings through any available carbon or nitrogen by the replacement of a hydrogen on said carbon or nitrogen.

When a compound is substituted with a substituent bearing a subscript that indicates the number of said substituents can exceed 1, said substituents (when they exceed 1) are independently selected from the group of defined substituents. Further, when the subscript m in (R)ₘ indicates an integer ranging from for example 0 to 4, then the number of substituents may be selected from the integers between 0 and 4 inclusive. Similarly, the subscript 1-2 in (R⁴) indicates an integer ranging from for example 1 to 2, then the number of substituents may be selected from the integers between 1 and 2 inclusive.

When a group contains a substituent which can be hydrogen, then, when this substituent is taken as hydrogen, it is recognized that said group is being un-substituted.

Optionally substituted groups may be mono- or poly substituted, where the substituents in the case of polysubstitutions may be the same or different.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments herein.

The description of the specific embodiments will so fully reveal the general nature of the embodiments herein so that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all these matters form a part of the prior art base or were common general knowledge in the field relevant to the disclosure as it existed anywhere before the priority date of this application.

The numerical values mentioned in the description and the description/claims though might form a critical part of the present invention, any deviation from such numerical values shall still fall within the scope of the present invention if that deviation follows the same scientific principle as that of the present invention disclosed in the present invention.

The compound of the present invention may, if appropriate, be present as mixtures of different possible isomeric forms, especially of stereoisomers, for example E and Z, threo and erythro, and also optical isomers, but if appropriate also of tautomers. Both the E and the Z isomers, but also the threo and erythro isomers, and the optical isomers, and any desired mixtures of these isomers and the possible tautomeric forms are disclosed and claimed.

The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula (I) can exhibit beneficial effects (e.g., suitability for preparation of useful formulations, improved biological performance) relative to another polymorph or a mixture of polymorphs of the same compound represented by Formula (I). The preparation and isolation of a particular polymorph of a compound represented by Formula (I) can be achieved by methods known to those skilled in the art including, for example, crystallization using selected solvents and temperatures.

The term “pest” for the purpose of the present disclosure includes but is not limited to fungi, stramenopiles (oomycetes) and bacteria.

The term “plant” is understood here to mean all plants and plant populations, such as desired and undesired wild plants or crop plants (including naturally occurring crop plants). Crop plants may be plants which can be obtained by conventional breeding and optimization methods or by biotechnological and genetic engineering methods or combinations of these methods, including the transgenic plants and including the plant cultivars which are protectable and non-protectable by plant breeders' rights.

For the purpose of the present disclosure the term “plant” includes a living organism of the kind exemplified by trees, shrubs, herbs, grasses, ferns, and mosses, typically growing in a site, absorbing water and required substances through its roots, and synthesizing nutrients in its leaves by photosynthesis.

Examples of “plant” for the purpose of the present invention include but are not limited to agricultural crops such as wheat, rye, barley, triticale, oats or rice; beet, e.g. sugar beet or fodder beet; fruits and fruit trees, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit and citrus trees, such as oranges, lemons, grapefruits or mandarins; any horticultural plants, vegetables, such as spinach, lettuce, asparagus, cabbages, carrots, onions, tomatoes, potatoes, cucurbits or paprika; lauraceous plants, such as avocados, cinnamon or camphor; cucurbitaceae; oleaginous plants; energy and raw material plants, such as cereals, corn, soybean, other leguminous plants, rape, sugar cane or oil palm; tobacco; nuts; coffee; tea; cacao; bananas; peppers; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants.

Preferably, the plant for the purpose of the present invention includes but is not limited to cereals, corn, rice, soybean and other leguminous plants, fruits and fruit trees, grapes, nuts and nut trees, citrus and citrus trees, any horticultural plants, cucurbitaceae, oleaginous plants, tobacco, coffee, tea, cacao, sugar beet, sugar cane, cotton, potato, tomato, onions, peppers and vegetables, ornamentals, any floricultural plants and other plants for the use of humans and animals.

The term “plant parts” is understood to mean all parts and organs of plants above and below the ground. For the purpose of the present disclosure the term plant parts include but is not limited to cuttings, leaves, twigs, tubers, flowers, seeds, branches, roots including taproots, lateral roots, root hairs, root apex, root cap, rhizomes, slips, shoots, fruits, fruit bodies, bark, stem, buds, auxiliary buds, meristems, nodes and internodes.

The term “locus thereof” includes soil, surroundings of plant or plant parts and equipment or tools used before, during or after sowing/planting a plant or a plant part.

The term “pesticidally active substance” and “additional active ingredient” can be used interchangeably.

Application of the compounds of the present disclosure or the compound of the present disclosure in a composition optionally comprising other compatible compounds to a plant or a plant material or locus thereof include application by a technique known to a person skilled in the art which include but is not limited to spraying, coating, dipping, fumigating, impregnating, injecting and dusting.

The term “applied” means adhered to a plant or plant part either physically or chemically including impregnation.

Accordingly, the present invention provides a compound of formula (I),

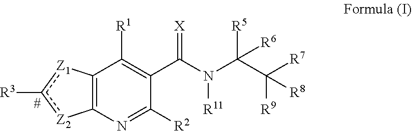

wherein,
“═” indicates presence or absence of double bond;
X is O or S;
Z¹ is C(R⁴)₁₋₂, CHR⁴—*CHR⁴, O—*CHR⁴, O, S. or N(R¹⁰)₀₋₁; wherein carbon atom marked with “*” indicates the point of attachment to carbon atom marked with “#”;
Z² is C(R⁴)₁₋₂ or NR¹⁰;
provided that
   a) when Z¹ is S and Z² is CR⁴ then R⁵ and R⁶ will not form a ring;
   b) when Z¹ is NH, Z² is NH and R³ is oxo then R⁵ and R⁶ will not form a ring;
   c) when Z¹ is N, and Z² is N(CH₃) then R⁵ and R⁶ will not form a ring;
R¹ is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy may optionally be substituted with one or more groups R^{1a};
R^{1a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R² is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy may optionally be substituted with one or more groups of R^{2a};
R^{2a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R³ is selected from the group consisting of hydrogen, halogen, oxo, cyan, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl may optionally be substituted with one or more groups of R^{3a};
R^{3a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁴ is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl may optionally be substituted with one or more groups of R^{4a};
R^{4a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁵ and R⁶ are each independently selected from the group consisting of hydrogen, cyano, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylcarbonyl, C₁-C₆-alkylthio, C₃-C₆-cycloalkyl, C₃-C₆-cycloalkyl-C₁-C₆-alkyl and C₆-C₁₀-aryl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylcarbonyl, C₁-C₆-alkylthio, C₃-C₆-cycloalkyl, C₃-C₆-cycloalkyl-C₁-C₆-alkyl and C₆-C₁₀-aryl may optionally be substituted with one or more groups of R^{5a}; or
R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₁₀-cycloalkyl and C₁-C₆-alkoxy-C₁-C₆-alkyl;
R⁷ and R⁸ are each independently selected from the group consisting of hydrogen, cyano, halogen, hydroxy, C₁-C₆-alkyl, C₁-C₆-haloalkyl, C₁-C₆-alkoxy, C₂-C₆-alkenyloxy, C₂-C₆-alkynyloxy, C₁-C₆-alkylcarbonyl and C₃-C₁₀-cycloalkyl; wherein C₁-C₆-alkyl, C₁-C₆-haloalkyl, C₁-C₆-alkoxy, C₂-C₆-alkenyloxy, C₂-C₆-alkynyloxy, C₁-C₆-alkylcarbonyl and C₃-C₁₀-cycloalkyl may optionally be substituted with one or more groups of R^{7a}; or
R⁷ and R⁸ together with the atoms to which they are attached may form a 3 to 6 membered non-aromatic or aromatic carbocyclic ring, or non-aromatic or aromatic heterocyclic ring; wherein one or more heteroatoms of said ring are selected from N, O or S(═O)₀₋₂ and one or more C atom/s of the ring may optionally be replaced by C(═O) or C(═S); the ring may optionally be substituted with one or more identical or different groups of R^{7a}; provided that when R⁷ and R⁸ form an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₁₀-cycloalkyl and C₁-C₆-alkoxy-C₁-C₆-alkyl;
R⁹ is selected from the group consisting of hydrogen, halogen, C₃-C₁₀-cycloalkyl, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contain 1, 2 or 3 heteroatoms selected from N, O and S; and wherein in each case one or two CH₂ groups of the carbo- or heterocycle may be replaced by a group independently selected from C(═O) and C(═S); wherein C₃-C₁₀-cycloalkyl, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy may optionally be substituted with one or more groups of R^{9a}; or
R⁵ and R⁹ together with the atoms to which they are attached may form a 3 to 12 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{9a};
R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₁₀-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy;
R′ is selected from the group consisting of C₁-C₆-alkyl, C₂-C₆-alkenyl, C₂-C₆-alkynyl, wherein each group of R′ may optionally be substituted with groups selected from halogen, cyano, C₁-C₆-alkoxy, C₃-C₁₀-cycloalkyl, C₃-C₁₀-cycloalkyloxy, C₆-C₁₀-aryl, C₇-C₁₄-aralkyl and C₆-C₁₀-aryloxy;
R″ is selected from hydrogen, C₁-C₆-alkyl, C₂-C₆-alkenyl, C₂-C₆-alkynyl, C₃-C₁₀-cycloalkyl and C₃-C₁₀-cycloalkenyl;
R¹⁰ is selected from the group consisting of hydrogen, C₁-C₆-alkyl, C₁-C₆-haloalkyl, C₃-C₁₀-cycloalkyl and C₃-C₁₀-cycloalkyl-C₁-C₆-alkyl;
R¹¹ is selected from the group consisting of hydrogen, C₁-C₆-alkyl and C₁-C₆-alkoxy; each group of R¹ to R¹¹, R^{1a} to R^{5a}, R^{7a} or R^{9a} may optionally be substituted with one or more groups selected from cyano, halogen, C₁-C₆-alkyl, C₂-C₆-alkenyl, C₂-C₆-alkynyl, C₃-C₁₀-cycloalkyl, C₃-C₁₀-cycloalkenyl and C₆-C₁₀-aryl;
and N-oxides, metal complexes, stereoisomers, polymorphs, or the agriculturally acceptable salts thereof;
with the proviso that, following compounds are excluded from the definition of compounds of formula (I),
2-methyl-N-(2,4,4-trimethylpentan-2-yl)-6,7-dihydro-5H-cyclopenta[b]pyridine-3-carboxamide,
N-(tert-butyl)-2-methyl-6,7-dihydro-5H-cyclopenta[b]pyridine-3-carboxamide
N-(tert-butyl)-2-methyl-5,6,7,8-tetrahydroquinoline-3-carboxamide,
2-methyl-N-(2,4,4-trimethylpentan-2-yl)-5,6,7,8-tetrahydroquinoline-3-carboxamide,
N-(1-(2-chlorophenoxy)-2-cyanopropan-2-yl)-4-methyl-3,4-dihydro-2H-pyrido[3,2-b][1,4]oxazine-7-carboxamide,
N-(2-cyano-1-(5-cyano-2-(trifluoromethyl)phenoxy)propan-2-yl)-4-methyl-3,4-dihydro-2H-pyrido[3,2-b][1,4]oxazine-7-carboxamide,
N-(2-(1,2,4-oxadiazol-3-yl)propan-2-yl)-3-methyl-3H-imidazo[4,5-b]pyridine-6-carboxamide,
N-(2-cyclohexylpropan-2-yl)-3-methyl-3H-imidazo[4,5-b]pyridine-6-carboxamide,
3-methyl-N-(1,1,1-trifluoro-2-methylbutan-2-yl)-3H-imidazo[4,5-b]pyridine-6-carboxamide,
N-(2-cyclopropyl-1,1,1-trifluoropropan-2-yl)-3-methyl-3H-imidazo[4,5-b]pyridine-6-carboxamide,
3-methyl-N-(4,4,4-trifluoro-2-methylbutan-2-yl)-3H-imidazo[4,5-b]pyridine-6-carboxamide,
N-(4-(azepan-1-yl)-2-methylbutan-2-yl)-3-methyl-3H-imidazo[4,5-b]pyridine-6-arboxamide,
N-(2-cyclohexyl-1-hydroxypropan-2-yl)-3-methyl-3H-imidazo[4,5-b]pyridine-6-arboxamide,
N-(2-cyclopropyl-1-hydroxypropan-2-yl)-3-methyl-3H-imidazo[4,5-b]pyridine-6-arboxamide,
N-(1-hydroxy-2-methylbutan-2-yl)-3-methyl-3H-imidazo[4,5-b]pyridine-6-arboxamide,
N-(tert-butyl)-1H-imidazo[4,5-b]pyridine-6-carboxamide,
N-(1-(azepan-1-yl)-2-methylpropan-2-yl)-N-methyl-3H-imidazo[4,5-b]pyridine-6-carboxamide,
N-(1-(azepan-1-yl)-2-methylpropan-2-yl)-N-methyl-2-oxo-2,3-dihydro-1H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(2-methyl-4-(4-methylpiperazin-1-yl)butan-2-yl)-2-oxo-2,3-dihydro-1H-imidazo[4,5-b]pyridine-6-carboxamide,
N-(2-(3,5-dimethylisoxazol-4-yl)propan-2-yl)-3-methyl-3H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(1-(3-methoxyphenyl)-2-methylpropan-2-yl)-3-methyl-3H-imidazo[4,5-b]pyridine-6-carboxamide,
N-(2-(tetrahydro-2H-pyran-4-yl)propan-2-yl)thieno[3,2-b]pyridine-6-carboxamide,
N-(2-methyl-1-morpholinopropan-2-yl)thieno[3,2-b]pyridine-6-carboxamide and N-(2-(1-methyl-1H-1,2,3-triazol-4-yl)propan-2-yl)thieno[3,2-b]pyridine-6-carboxamide.

In one embodiment, the compound of Formula (I) is represented by a compound of Formula (I-A);

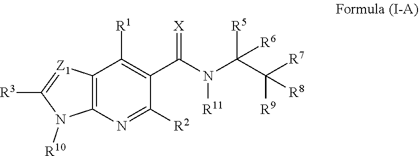

wherein, X, Z₁, R¹, R², R³, R⁵, R⁶, R⁷, R⁸, R⁹, R¹⁰ and R¹¹ are as defined in the detailed description above. In another embodiment, the compound of Formula (I) is represented by a compound of Formula (I-B);

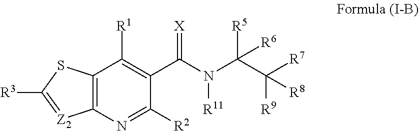

wherein, X, Z₂, R¹, R², R³, R⁵, R⁶, R⁷, R⁸, R⁹ and R¹⁰ are as defined in the detailed description above.

In yet another embodiment, the compound of Formula (I) is represented by a compound of Formula (I-C);

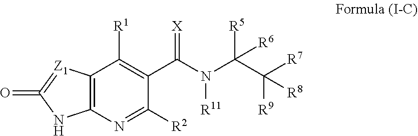

wherein, X, Z₁, R¹, R², R³, R⁵, R⁶, R⁷, R⁸, R⁹ and R¹¹ are as defined in the detailed description above.

In yet another embodiment, the compound of Formula (I) is represented by a compound of Formula (I-D);

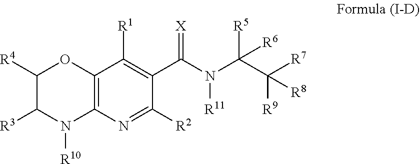

wherein, X, R¹, R², R³, R⁴, R⁵, R⁶, R⁷, R⁸, R⁹, R¹⁰ and R¹¹ are as defined in the detailed description above.

In yet another embodiment, the compound of Formula (I) is represented by a compound of Formula (I-E);

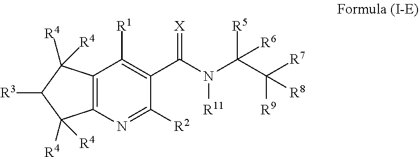

wherein, X, R¹, R², R³, R⁴, R⁵, R⁶, R⁷, R⁸, R⁹ and R¹⁰ are as defined in the detailed description above.

In yet another embodiment, the compound of Formula (I) is represented by a compound of Formula (I-F);

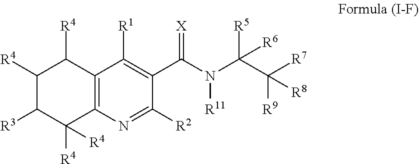

wherein, X, R¹, R², R³, R⁴, R⁵, R⁶, R⁷, R⁸, R⁹ and R¹¹ are as defined in the detailed description above.

In yet another embodiment, the compound of Formula (I) is represented by a compound of Formula (I-G);

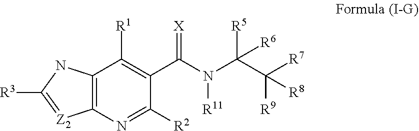

wherein, X, Z₂, R¹, R², R³, R⁵, R⁶, R⁷, R⁸, R⁹ and R¹¹ are as defined in the detailed description above. In another embodiment, the compound of Formula (I)

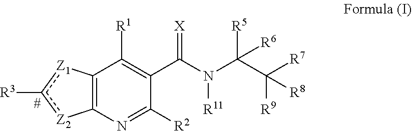

wherein,
“═” indicates presence or absence of double bond;
X is O or S;
Z¹ is C(R⁴)₁₋₂, CHR⁴—*CHR⁴, O—*CHR⁴, O, S. or N(R¹⁰)₀₋₁; wherein the carbon atom marked with “*” indicates the point of attachment to the carbon atom marked with “#”;
Z² is C(R⁴)₁₋₂ or NR¹⁰;
provided that
   a) when Z¹ is S and Z² is CR⁴ then R⁵ and R⁶ will not form a ring;
   b) when Z¹ is NH, Z² is NH and R³ is oxo then R⁵ and R⁶ will not form a ring;
   c) when Z¹ is N, and Z² is N(CH₃) then R⁵ and R⁶ will not form a ring;
R¹ is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₆-cycloalkyl and phenoxy; wherein each group of R¹ may optionally be substituted with one or more groups of R^{1a};
R^{1a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R² is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₆-cycloalkyl and phenoxy; wherein each group of R² may optionally be substituted with one or more groups of R^{2a};
R^{2a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R³ is selected from the group consisting of hydrogen, halogen, oxo, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₆-cycloalkyl; wherein each group of R³ may optionally be substituted with one or more groups of R^{3a};
R^{3a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁴ is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₆-cycloalkyl; wherein each group of R⁴ may optionally be substituted with one or more groups of R^{4a};
R^{4a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁵ and R⁶ are each independently selected from the group consisting of hydrogen, cyano, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylcarbonyl, C₁-C₆-alkylthio, C₃-C₆-cycloalkyl, C₃-C₆-cycloalkyl-C₁-C₆-alkyl and C₆-C₁₀-aryl; wherein each group of R⁵ and R⁶ may optionally be substituted with one or more groups of R^{5a}; or
R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl and C₁-C₄-alkoxy-C₁-C₄-alkyl;
R⁷ and R⁸ are each independently selected from the group consisting of hydrogen, cyano, halogen, hydroxy, C₁-C₆-alkyl, C₁-C₆-haloalkyl, C₁-C₆-alkoxy, C₂-C₆-alkenyloxy, C₂-C₆-alkynyloxy, C₁-C₆-alkylcarbonyl and C₃-C₅-cycloalkyl; wherein each group of R⁷ and R⁸ may optionally be substituted with one or more groups of R^{7a}; or
R⁷ and R⁸ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O) or C(═S); the ring may optionally be substituted with one or more identical or different groups of R^{7a}; provided that when R⁷ and R⁸ forms an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl and C₁-C₄-alkoxy-C₁-C₄-alkyl;
R⁹ is selected from the group consisting of hydrogen, halogen, C₃-C₁₀-cycloalkyl, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contains 1, 2 or 3 heteroatoms selected from N, O and S; and wherein in each case one or two CH₂ groups of the carbo- or heterocycle may be replaced by a group independently selected from C(O) and C(═S); wherein each group of R⁹ may optionally be substituted with one or more groups of R^{9a}; or
R⁵ and R⁹ together with the atoms to which they are attached may form a 3 to 12 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{9a};
R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy;
R′ is selected from the group consisting of C₁-C₆-alkyl, C₂-C₆-alkenyl, C₂-C₆-alkynyl, wherein each group of R′ may optionally be substituted with groups selected from halogen, cyano, C₁-C₆-alkoxy, C₃-C₆-cycloalkyl, C₃-C₆-cycloalkyloxy, C₆-C₁₀-aryl, C₇-C₁₄-aralkyl and C₆-C₁₀-aryloxy;
R″ is selected from hydrogen, C₁-C₆-alkyl, C₂-C₆-alkenyl, C₂-C₆-alkynyl, C₃-C₆-cycloalkyl and C₃-C₆-cycloalkenyl;
R¹⁰ is selected from the group consisting of hydrogen, C₁-C₄-alkyl, C₁-C₄-haloalkyl, C₃-C₅-cycloalkyl and C₃-C₅-cycloalkyl-C₁-C₃-alkyl;
R¹¹ is selected from the group consisting of hydrogen, C₁-C₄-alkyl and C₁-C₄-alkoxy;
each group of R¹ to R¹¹, R^{1a} to R^{5a}, R^{7a} or R^{9a} may optionally be substituted with one or more groups selected from cyano, halogen, C₁-C₆-alkyl, C₂-C₆-alkenyl, C₂-C₆-alkynyl, C₃-C₆-cycloalkyl, C₃-C₆-cycloalkenyl and C₆-C₁₀-aryl;
and N-oxides, metal complexes, stereoisomers, polymorphs, or the agriculturally acceptable salts thereof;
with the proviso that, following compounds are excluded from the definition of compounds of formula (I),
2-methyl-N-(2,4,4-trimethylpentan-2-yl)-6,7-dihydro-5H-cyclopenta[b]pyridine-3-carboxamide,
N-(tert-butyl)-2-methyl-6,7-dihydro-5H-cyclopenta[b]pyridine-3-carboxamide
N-(tert-butyl)-2-methyl-5,6,7,8-tetrahydroquinoline-3-carboxamide,
2-methyl-N-(2,4,4-trimethylpentan-2-yl)-5,6,7,8-tetrahydroquinoline-3-carboxamide,
N-(1-(2-chlorophenoxy)-2-cyanopropan-2-yl)-4-methyl-3,4-dihydro-2H-pyrido[3,2-b][1,4]oxazine-7-carboxamide,
N-(2-cyano-1-(5-cyano-2-(trifluoromethyl)phenoxy)propan-2-yl)-4-methyl-3,4-dihydro-2H-pyrido[3,2-b][1,4]oxazine-7-carboxamide,
N-(2-(1,2,4-oxadiazol-3-yl)propan-2-yl)-3-methyl-3H-imidazo[4,5-b]pyridine-6-carboxamide,
N-(2-cyclohexylpropan-2-yl)-3-methyl-3H-imidazo[4,5-b]pyridine-6-carboxamide,
3-methyl-N-(1,1,1-trifluoro-2-methylbutan-2-yl)-3H-imidazo[4,5-b]pyridine-6-carboxamide,
N-(2-cyclopropyl-1,1,1-trifluoropropan-2-yl)-3-methyl-3H-imidazo[4,5-b]pyridine-6-carboxamide,
3-methyl-N-(4,4,4-trifluoro-2-methylbutan-2-yl)-3H-imidazo[4,5-b]pyridine-6-carboxamide,
N-(4-(azepan-1-yl)-2-methylbutan-2-yl)-3-methyl-3H-imidazo[4,5-b]pyridine-6-carboxamide,
N-(2-cyclohexyl-1-hydroxypropan-2-yl)-3-methyl-3H-imidazo[4,5-b]pyridine-6-carboxamide,
N-(2-cyclopropyl-1-hydroxypropan-2-yl)-3-methyl-3H-imidazo[4,5-b]pyridine-6-carboxamide,
N-(1-hydroxy-2-methylbutan-2-yl)-3-methyl-3H-imidazo[4,5-b]pyridine-6-carboxamide,
N-(tert-butyl)-1H-imidazo[4,5-b]pyridine-6-carboxamide,
N-(1-(azepan-1-yl)-2-methylpropan-2-yl)-N-methyl-3H-imidazo[4,5-b]pyridine-6-carboxamide,
N-(1-(azepan-1-yl)-2-methylpropan-2-yl)-N-methyl-2-oxo-2,3-dihydro-1H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(2-methyl-4-(4-methylpiperazin-1-yl)butan-2-yl)-2-oxo-2,3-dihydro-1H-imidazo[4,5-b]pyridine-6-carboxamide,
N-(2-(3,5-dimethylisoxazol-4-yl)propan-2-yl)-3-methyl-3H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(1-(3-methoxyphenyl)-2-methylpropan-2-yl)-3-methyl-3H-imidazo[4,5-b]pyridine-6-carboxamide,
N-(2-(tetrahydro-2H-pyran-4-yl)propan-2-yl)thieno[3,2-b]pyridine-6-carboxamide,
N-(2-methyl-1-morpholinopropan-2-yl)thieno[3,2-b]pyridine-6-carboxamide and
N-(2-(1-methyl-1H-1,2,3-triazol-4-yl)propan-2-yl)thieno[3,2-b]pyridine-6-carboxamide.

In one preferred embodiment, the present invention provides a compound of Formula (I), wherein X is O and Z, R¹, R², R³, R⁴, R⁵, R⁶, R⁷, R⁸, R⁹ and R¹¹ are as defined in the detailed description above.

In another preferred embodiment, the present invention provides a compound of Formula (I), wherein, Z¹ is CH(R⁴); Z² is NR¹⁰ and X, R¹, R², R³, R⁴, R⁵, R⁶, R⁷, R⁸, R⁹ and R¹¹ are as defined in the detailed description above.

In yet another preferred embodiment, the present invention provides a compound of Formula (I), wherein Z¹ is S; Z² is CHR⁴ and X, R¹, R², R³, R⁴, R⁵, R⁶, R⁷, R⁸, R⁹ and R¹¹ are as defined in the detailed description above.

In yet another preferred embodiment, the present invention provides a compound of Formula (I), wherein Z¹ is CHR⁴—*CHR⁴; wherein the carbon atom marked with “*” indicates the point of attachment to the carbon atom marked with “#”; Z² is CHR⁴ and X, R¹, R², R³, R⁴, R⁵, R⁶, R⁷, R⁸, R⁹ and R¹¹ areas defined in the detailed description above.

In yet another preferred embodiment, the present invention provides a compound of Formula (I), wherein Z¹ is CHR⁴; Z² is CHR⁴ and X, R¹, R², R³, R⁴, R⁵, R⁶, R⁷, R⁸, R⁹ and R¹¹ are as defined in the detailed description above.

In yet another preferred embodiment, the present invention provides a compound of Formula (I), wherein Z¹ is O—*CHR⁴; wherein the carbon atom marked with “*” indicates the point of attachment to the carbon atom marked with “#”; Z² is NR¹⁰ and X, R¹, R², R³, R⁴, R⁵, R⁶, R⁷, R⁸, R⁹ and R¹¹ are as defined in the detailed description above.

In yet another preferred embodiment, the present invention provides a compound of Formula (I), wherein Z¹ is NR¹⁰; Z² is CHR⁴ and X, R¹, R², R³, R⁴, R⁵, R⁶, R⁷, R⁸, R⁹ and R¹¹ are as defined in the detailed description above.

In yet another preferred embodiment, the present invention provides a compound of Formula (I), wherein Z¹ is O; Z² is NR¹⁰; R³ is oxo and X, R¹, R², R³, R⁴, R⁵, R⁶, R⁷, R⁸, R⁹ and R¹¹ are as defined in the detailed description above.

In one embodiment, the present invention provides a compound of Formula (I), wherein X is O;
R¹ is selected from the group consisting of hydrogen, halogen, cyano, hydroxy, C₁-C₆-alkyl, C₁-C₆-haloalkyl and C₁-C₆-alkoxy;
R² is selected from the group consisting of hydrogen, halogen, cyano, hydroxy, C₁-C₆-alkyl, C₁-C₆-haloalkyl, C₁-C₆-alkoxy, C₁-C₆-alkylthio and C₃-C₆-cycloalkyl;
R³ is selected from the group consisting of hydrogen, halogen, oxo, cyano, C₁-C₆-alkyl, C₁-C₆-haloalkyl, C₁-C₆-alkoxy, C₁-C₆-alkylthio and C₃-C₆-cycloalkyl;
R⁴ is selected from the group consisting of hydrogen, halogen, cyano, C₁-C₆-alkyl, C₁-C₆-haloalkyl, C₁-C₆-alkoxy, C₁-C₆-alkylthio and C₃-C₆-cycloalkyl;
R⁵ and R⁶ are each independently selected from the group consisting of hydrogen, cyano, halogen, C₁-C₆-alkyl, C₂-C₆-alkenyl, C₁-C₆-alkoxy, C₁-C₆-alkylcarbonyl, C₁-C₆-alkylthio, C₃-C₆-cycloalkyl and C₃-C₆-cycloalkyl-C₁-C₆-alkyl; wherein each group of R⁵ and R⁶ may optionally be substituted with one or more groups of R^{5a}; or
R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino, C₁-C₆-alkyl;
R⁷ and R⁸ are each independently selected from the group consisting of hydrogen, cyano, halogen, hydroxy, C₁-C₆-alkyl, C₁-C₆-haloalkyl and C₁-C₆-alkoxy, wherein each group of R⁷ and R⁸ may optionally be substituted with one or more groups of R^{7a}; or
R⁷ and R⁸ together with the atoms to which they are attached may form a 3 to 6 membered non-aromatic or aromatic carbocyclic ring, or non-aromatic or aromatic heterocyclic ring; wherein one or more heteroatoms of said ring are selected from N, O or S(═O)₀₋₂ and one or more C atom/s of the ring may optionally be replaced by C(═O) or C(═S); the ring may optionally be substituted with one or more identical or different groups of R^{7a}; provided that when R⁷ and R⁸ forms an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl and C₁-C₄-alkoxy-C₁-C₄-alkyl;
R⁹ is selected from the group consisting of hydrogen, halogen, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contains 1, 2 or 3 heteroatoms selected from N, O and S; and wherein in each case one or two CH₂ groups of the carbo- or heterocycle may be replaced by a group independently selected from C(═O) and C(═S); wherein C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy may optionally be substituted with one or more groups of R^{9a}; or
R⁵ and R⁹ together with the atoms to which they are attached may form a 3 to 12 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{9a};
R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy;
R′ is selected from the group consisting of C₁-C₆-alkyl, C₂-C₆-alkenyl, C₂-C₆-alkynyl, wherein each group of R′ may optionally be substituted with groups selected from halogen, cyano, C₁-C₆-alkoxy, C₃-C₆-cycloakyl, C₃-C₆-cycloalkyloxy, C₆-C₁₀-aryl, C₇-C₁₄-aralkyl and C₆-C₁₀-aryloxy;
R″ is selected from hydrogen, C₁-C₆-alkyl, C₂-C₆-alkenyl, C₂-C₆-alkynyl, C₃-C₆-cycloalkyl and C₃-C₆-cycloalkenyl;
R¹⁰ is selected from the group consisting of hydrogen, C₁-C₄-alkyl, C₁-C₄-haloalkyl, C₃-C₅-cycloalkyl and C₃-C₅-cycloalkyl-C₁-C₃-alkyl; and
R¹¹ is selected from the group consisting of hydrogen, C₁-C₄-alkyl and C₁-C₄-alkoxy; and/or N-oxides, metal complexes, stereoisomers, polymorphs, or the agriculturally acceptable salts thereof.

In one preferred embodiment, the present invention provides a compound of Formula (I), wherein R¹ is selected from the group consisting of hydrogen, halogen, cyano, hydroxy, C₁-C₆-alkyl, C₁-C₆-haloalkyl and C₁-C₆-alkoxy.

In another preferred embodiment, the present invention provides a compound of Formula (I), wherein R² is selected from the group consisting of hydrogen, halogen, cyano, hydroxy, C₁-C₆-alkyl, C₁-C₆-haloalkyl, C₁-C₆-alkoxy, C₁-C₆-alkylthio and C₃-C₆-cycloalkyl.

In yet another preferred embodiment, the present invention provides a compound of Formula (I), wherein R³ is selected from the group consisting of hydrogen, halogen, oxo, cyano, C₁-C₆-alkyl, C₁-C₆-haloalkyl, C₁-C₆-alkoxy, C₁-C₆-alkylthio and C₃-C₆-cycloalkyl.

In yet another preferred embodiment, the present invention provides a compound of Formula (I), wherein R⁴ is selected from the group consisting of hydrogen, halogen, cyano, C₁-C₆-alkyl, C₁-C₆-haloalkyl, C₁-C₆-alkoxy, C₁-C₆-alkylthio and C₃-C₆-cycloalkyl.

In yet another preferred embodiment, the present invention provides a compound of Formula (I), wherein R⁵ and R⁶ are each independently selected from the group consisting of hydrogen, cyano, halogen, C₁-C₆-alkyl, C₂-C₆-alkenyl, C₁-C₆-alkoxy, C₁-C₆-alkylcarbonyl, C₁-C₆-alkylthio, C₃-C₆-cycloalkyl and C₃-C₆-cycloalkyl-C₁-C₆-alkyl; wherein each group of R⁵ and R⁶ may optionally be substituted with one or more groups of R^{5a};
R^{5a} is selected from the group consisting of halogen, cyano, nitro, hydroxyl, thiol, amino and C₁-C₆-alkyl.

In yet another preferred embodiment, the present invention provides a compound of Formula (I), wherein R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino and C₁-C₆-alkyl.

In yet another preferred embodiment, the present invention provides a compound of Formula (I), wherein R⁷ and R⁸ are each independently selected from the group consisting of hydrogen, cyano, halogen, hydroxy, C₁-C₆-alkyl, C₁-C₆-haloalkyl and C₁-C₆-alkoxy.

In yet another preferred embodiment, the present invention provides a compound of Formula (I), wherein R⁷ and R⁸ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O) or C(═S); the ring may optionally be substituted with one or more identical or different groups of R^{7a}; provided that when R⁷ and R⁸ forms an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl and C₁-C₄-alkoxy-C₁-C₄-alkyl.

In yet another preferred embodiment, the present invention provides a compound of Formula (I), wherein R⁹ is selected from the group consisting of hydrogen, halogen, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contains 1, 2 or 3 heteroatoms selected from N, O and S; and wherein in each case one or two CH₂ groups of the carbo- or heterocycle may be replaced by a group independently selected from C(═O) and C(═S); wherein each group of R⁹ may optionally be substituted with one or more groups of R^{9a}.

In yet another preferred embodiment, the present invention provides a compound of Formula (I), wherein R⁵ and R⁹ together with the atoms to which they are attached may form a 3 to 12 membered non-aromatic carbocyclic ring, non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{9a}.

In yet another preferred embodiment, the present invention provides a compound of Formula (I), wherein R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy.

In a more preferred embodiment, the present invention provides a compound of Formula (I) selected from 1-methyl-N-(2-methyl-1-phenylpropan-2-yl)-1H-pyrrolo[2,3-b]pyridine-5-carboxamide, N-(2,4-dimethyl-1-phenylpentan-2-yl)-1-methyl-1H-pyrrolo[2,3-b]pyridine-5-carboxamide, N-(3-(2-fluorophenyl)-2-methylbutan-2-yl)-1-methyl-1H-pyrrolo[2,3-b]pyridine-5-carboxamide, N-(2,3-dimethyl-3-phenylbutan-2-yl)-1-methyl-1H-pyrrolo[2,3-b]pyridine-5-carboxamide, 1-methyl-N-(2-methyl-1-phenylbutan-2-yl)-1H-pyrrolo[2,3-b]pyridine-5-carboxamide, N-(3-(2-chlorophenyl)-2-methylbutan-2-yl)-1-methyl-1H-pyrrolo[2,3-b]pyridine-5-carboxamide, 1-methyl-N-(2-methyl-3-phenylbutan-2-yl)-1H-pyrrolo[2,3-b]pyridine-5-carboxamide, N-(1-benzylcyclopentyl)-1-methyl-1H-pyrrolo[2,3-b]pyridine-5-carboxamide, N-(1-benzylcyclohexyl)-1-methyl-1H-pyrrolo[2,3-b]pyridine-S-carboxamide, N-(1-(3-chlorobenzyl)cyclohexyl)-1-methyl-1H-pyrrolo[2,3-b]pyridine-5-carboxamide, N-(1-(2-fluorophenyl)-2-methylpropan-2-yl)-1-methyl-1H-pyrrolo[2,3-b]pyridine-5-carboxamide, 1-methyl-N-(2-methyl-1-(3-(trifluoromethyl)phenyl)propan-2-yl)-1H-pyrrolo[2,3-b]pyridine-5-carboxamide, N-(1-(3,5-difluorophenyl)-2-methylpropan-2-yl)-1-methyl-1H-pyrrolo[2,3-b]pyridine-5-carboxamide, N-(1-(4-(tert-butyl)phenyl)-2-methylpropan-2-yl)-1-methyl-1H-pyrrolo[2,3-b]pyridine-5-carboxamide, N-(1-(2-chlorophenyl)-2-methylpropan-2-yl)-1-methyl-1H-pyrrolo[2,3-b]pyridine-5-carboxamide, N-(1-(4-isopropylphenyl)-2-methylpropan-2-yl)-1-methyl-1H-pyrrolo[2,3-b]pyridine-5-carboxamide, 1-methyl-N-(2-methyl-1-(4-(trifluoromethoxy)phenyl)propan-2-yl)-1H-pyrrolo[2,3-b]pyridine-5-carboxamide, N-(1-(2,4-difluorophenyl)-2-methylpropan-2-yl)-1-methyl-1H-pyrrolo[2,3-b]pyridine-5-carboxamide, N-(1-(3-fluorophenyl)-2-methylpropan-2-yl)-1-methyl-1H-pyrrolo[2,3-b]pyridine-5-carboxamide, N-(1-(3-chlorophenyl)-2-methylpropan-2-yl)-1-methyl-1H-pyrrolo[2,3-b]pyridine-5-carboxamide, N-(1-(3,4-difluorophenyl)-2-methylpropan-2-yl)-1-methyl-1H-pyrrolo[2,3-b]pyridine-5-carboxamide, N-(1-(3,4-dichlorophenyl)-2-methylpropan-2-yl)-1-methyl-1H-pyrrolo[2,3-b]pyridine-5-carboxamide, N-(4,4,4-trifluoro-2-methyl-1-phenylbutan-2-yl)thieno[3,2-b]pyridine-6-carboxamide, N-(1-(2-bromophenyl)-2-methylpropan-2-yl)-1-methyl-1H-pyrrolo[2,3-b]pyridine-5-carboxamide, 1-methyl-N-(2-methyl-1-(m-tolyl)propan-2-yl)-1H-pyrrolo[2,3-b]pyridine-5-carboxamide, N-(2,4-dimethyl-1-phenylpentan-2-yl)-1H-pyrrolo[2,3-b]pyridine-5-carboxamide, N-(2,4-dimethyl-1-phenylpentan-2-yl)thieno[3,2-b]pyridine-6-carboxamide, N-(2-methyl-1-phenylpropan-2-yl)thieno[3,2-b]pyridine-6-carboxamide, 1,6-dimethyl-N-(2-methyl-1-phenylpropan-2-yl)-1H-pyrrolo[2,3-b]pyridine-5-carboxamide, N-(2,4-dimethyl-1-phenylpentan-2-yl)-1,6-dimethyl-1H-pyrrolo[2,3-b]pyridine-5-carboxamide, N-(3-(2-fluorophenyl)-2-methylbutan-2-yl)-1,6-dimethyl-1H-pyrrolo[2,3-b]pyridine-5-carboxamide, N-(1-(2-fluorophenyl)-2,4-dimethylpentan-2-yl)-1-methyl-1H-pyrrolo[2,3-b]pyridine-5-carboxamide, N-(2,3-dimethyl-3-phenylbutan-2-yl)-1,6-dimethyl-1H-pyrrolo[2,3-b]pyridine-5-carboxamide, N-(1-(2-fluorophenyl)-2-methylpropan-2-yl)-1,6-dimethyl-1H-pyrrolo[2,3-b]pyridine-5-carboxamide, N-(1-benzylcyclopropyl)-1-methyl-1H-pyrrolo[2,3-b]pyridine-5-carboxamide, N-(3-(2-chlorophenyl)-2-methylbutan-2-yl)-1,6-dimethyl-1H-pyrrolo[2,3-b]pyridine-5-carboxamide, N-(1-(4-fluorophenyl)-2-methylpropan-2-yl)-1H-pyrrolo[2,3-b]pyridine-5-carboxamide, N-(1-(4-chloro-3-fluorophenyl)-2-methylpropan-2-yl)-1-methyl-1H-pyrrolo[2,3-b]pyridine-5-carboxamide, N-(1-(4-chlorophenyl)-2-methylpropan-2-yl)-1-methyl-1H-pyrrolo[2,3-b]pyridine-5-carboxamide, N-(2,4-dimethyl-1-phenylpentan-2-yl)-5,6,7,8-tetrahydroquinoline-3-carboxamide, N-(2-methyl-1-phenylpropan-2-yl)-5,6,7,8-tetrahydroquinoline-3-carboxamide, 3-bromo-1-methyl-N-(2-methyl-1-phenylpropan-2-yl)-1H-pyrrolo[2,3-b]pyridine-5-carboxamide, N-(1-(4-chloro-2-fluorophenyl)-2-methylpropan-2-yl)-1-methyl-1H-pyrrolo[2,3-b]pyridine-5-carboxamide, N-(1-(3-bromophenyl)-2-methylpropan-2-yl)-1-methyl-1H-pyrrolo[2,3-b]pyridine-5-carboxamide, N-(1-(2-methoxyphenyl)-2-methylpropan-2-yl)-1-methyl-1H-pyrrolo[2,3-b]pyridine-5-carboxamide, 1-methyl-N-(2-methyl-1-(p-tolyl)propan-2-yl)-1H-pyrrolo[2,3-b]pyridine-5-carboxamide, 3-bromo-N-(2,4-dimethyl-1-phenylpentan-2-yl)-1-methyl-1H-pyrrolo[2,3-b]pyridine-5-carboxamide, 3-chloro-N-(2,4-dimethyl-1-phenylpentan-2-yl)-1-methyl-1H-pyrrolo[2,3-b]pyridine-5-carboxamide, 3-chloro-1-methyl-N-(2-methyl-1-phenylpropan-2-yl)-1H-pyrrolo[2,3-b]pyridine-5-carboxamide, 3-cyclopropyl-1-methyl-N-(2-methyl-1-phenylpropan-2-yl)-1H-pyrrolo[2,3-b]pyridine-5-carboxamide, 3-cyclopropyl-N-(2,4-dimethyl-1-phenylpentan-2-yl)-1-methyl-1H-pyrrolo[2,3-b]pyridine-5-carboxamide, 3-chloro-N,1-dimethyl-N-(2-methyl-1-phenylpropan-2-yl)-1H-pyrrolo[2,3-b]pyridine-5-carboxamide, 3-cyclopropyl-N, I-dimethyl-N-(2-methyl-1-phenylpropan-2-yl)-1H-pyrrolo[2,3-b]pyridine-5-carboxamide, 3-bromo-N-(1-(2-fluorophenyl)-2-methylpropan-2-yl)-1-methyl-1H-pyrrolo[2,3-b]pyridine-5-carboxamide, 3-chloro-N-(1-(2-fluorophenyl)-2-methylpropan-2-yl)-1-methyl-1H-pyrrolo[2,3-b]pyridine-5-carboxamide, 3-cyclopropyl-N-(1-(2-fluorophenyl)-2-methylpropan-2-yl)-1-methyl-1H-pyrrolo[2,3-b]pyridine-5-carboxamide, N-(1-(4-bromophenyl)-2-methylpropan-2-yl)-1-methyl-1H-pyrrolo[2,3-b]pyridine-5-carboxamide, N-(2,4-dimethyl-1-phenylpentan-2-yl)-6,7-dihydro-5H-cyclopenta[b]pyridine-3-carboxamide, 3-bromo-N-(3-(2-fluorophenyl)-2-methylbutan-2-yl)-1-methyl-1H-pyrrolo[2,3-b]pyridine-5-carboxamide, 3-chloro-N-(3-(2-fluorophenyl)-2-methylbutan-2-yl)-1-methyl-1H-pyrrolo[2,3-b]pyridine-5-carboxamide, 3-cyclopropyl-N-(3-(2-fluorophenyl)-2-methylbutan-2-yl)-1-methyl-1H-pyrrolo[2,3-b]pyridine-5-carboxamide, N-(1-(4-chlorophenyl)-2-methylpropan-2-yl)-3-fluoro-1-methyl-1H-pyrrolo[2,3-b]pyridine-5-carboxamide, N-(2-methyl-1-phenylpropan-2-yl)-6,7-dihydro-5H-cyclopenta[b]pyridine-3-carboxamide, 3-fluoro-N-(1-(2-fluorophenyl)-2-methylpropan-2-yl)-1-methyl-1H-pyrrolo[2,3-b]pyridine-5-carboxamide, N-(1-(4-chloro-3-fluorophenyl)-2-methylpropan-2-yl)-3-fluoro-1-methyl-1H-pyrrolo[2,3-b]pyridine-5-carboxamide, 3-fluoro-1-methyl-N-(2-methyl-1-(p-tolyl)propan-2-yl)-1H-pyrrolo[2,3-b]pyridine-5-carboxamide, N-(3-(2-chlorophenyl)-2-methylbutan-2-yl)-3-fluoro-1-methyl-1H-pyrrolo[2,3-b]pyridine-5-carboxamide, 3-fluoro-N-(1-(2-methoxyphenyl)-2-methylpropan-2-yl)-1-methyl-1H-pyrrolo[2,3-b]pyridine-5-carboxamide, N-(2,4-dimethyl-1-phenylpentan-2-yl)-1H-pyrrolo[3,2-b]pyridine-6-carboxamide, N-(1-(4-fluorophenyl)-2-methylpropan-2-yl)-1-methyl-1H-pyrrolo[2,3-b]pyridine-5-carboxamide, 1-methyl-N-(4-phenylbutan-2-yl)-1H-pyrrolo[2,3-b]pyridine-5-carboxamide, N-(2,3-dihydro-1H-inden-2-yl)-1-methyl-1H-pyrrolo[2,3-b]pyridine-5-carboxamide, 1-methyl-N-(2-methyl-1-(naphthalen-2-yl)propan-2-yl)-1H-pyrrolo[2,3-b]pyridine-5-carboxamide, 1-methyl-N-(2-methyl-2,3-dihydro-1H-inden-2-yl)-1H-pyrrolo[2,3-b]pyridine-5-carboxamide, 1-methyl-N-((1-phenylcyclopropyl)methyl)-1H-pyrrolo[2,3-b]pyridine-5-carboxamide, N-(2,3-dimethyl-4-phenylbutan-2-yl)-1-methyl-1H-pyrrolo[2,3-b]pyridine-5-carboxamide, N-(2,4-dimethyl-1-phenylpentan-2-yl)-4-methyl-3,4-dihydro-2H-pyrido[3,2-b][1,4]oxazine-7-carboxamide, 4-methyl-N-(2-methyl-1-phenylpropan-2-yl)-3,4-dihydro-2H-pyrido[3,2-b][1,4]oxazine-7-carboxamide, 3-chloro-N-(1-(2-chlorophenyl)-2-methylpropan-2-yl)-1-methyl-1H-pyrrolo[2,3-b]pyridine-5-carboxamide, 3-bromo-N-(1-(2-chlorophenyl)-2-methylpropan-2-yl)-1-methyl-1H-pyrrolo[2,3-b]pyridine-5-carboxamide, N-(1-(2-chlorophenyl)-2-methylpropan-2-yl)-3-cyclopropyl-1-methyl-1H-pyrrolo[2,3-b]pyridine-5-carboxamide, 1-methyl-N-(2-methyl-1-phenylpropan-2-yl)-1H-pyrrolo[3,2-b]pyridine-6-carboxamide, 3-chloro-1-methyl-N-(2-methyl-3-phenylbutan-2-yl)-1H-pyrrolo[2,3-b]pyridine-5-carboxamide, 6-methoxy-N-(2-methyl-1-phenylpropan-2-yl)-1H-pyrrolo[2,3-b]pyridine-5-carboxamide, N-(2,4-dimethyl-1-phenylpentan-2-yl)-6-methoxy-1H-pyrrolo[2,3-b]pyridine-5-carboxamide, 1-methyl-N-(2-methyl-1-(4-(methylthio)phenyl)propyl)-1H-pyrrolo[2,3-b]pyridine-5-carboxamide, 6-methoxy-1-methyl-N-(2-methyl-1-phenylpropan-2-yl)-1H-pyrrolo[2,3-b]pyridine-5-carboxamide, N-(2,4-dimethyl-1-phenylpentan-2-yl)-6-methoxy-1-methyl-1H-pyrrolo[2,3-b]pyridine-5-carboxamide, 6-methyl-N-(2-methyl-1-phenylpropan-2-yl)-1H-pyrrolo[2,3-b]pyridine-5-carboxamide, N-(1-(4-fluorophenyl)-2-methylpropan-2-yl)-6-methyl-1H-pyrrolo[2,3-b]pyridine-5-carboxamide, N-(2-methyl-1-phenylpropan-2-yl)-1H-pyrrolo[3,2-b]pyridine-6-carboxamide, N-(2,4-dimethyl-1-phenylpentan-2-yl)-6-methyl-1H-pyrrolo[2,3-b]pyridine-5-carboxamide, N-(1-benzylcyclopropyl)-6-methyl-1H-pyrrolo[2,3-b]pyridine-5-carboxamide, N-(2,4-dimethyl-1-phenylpentan-2-yl)-2-oxo-2,3-dihydrooxazolo[4,5-b]pyridine-6-carboxamide, 1,2-dimethyl-N-(2-methyl-1-phenylpropan-2-yl)-1H-pyrrolo[2,3-b]pyridine-5-carboxamide, N-(2,3-dimethyl-3-phenylbutan-2-yl)-1,2-dimethyl-1H-pyrrolo[2,3-b]pyridine-5-carboxamide, N-(1-(4-chlorophenyl)-2-methylpropan-2-yl)-1,2-dimethyl-1H-pyrrolo[2,3-b]pyridine-5-carboxamide, 1,2-dimethyl-N-(2-methyl-1-(p-tolyl)propan-2-yl)-1H-pyrrolo[2,3-b]pyridine-5-carboxamide, N-(1-(2-fluorophenyl)-2-methylpropan-2-yl)-1,2-dimethyl-1H-pyrrolo[2,3-b]pyridine-5-carboxamide, N-(3-hydroxy-3-methyl-1-(m-tolyl)butan-2-yl)-1H-pyrrolo[2,3-b]pyridine-5-carboxamide, N-(3-hydroxy-2-methyl-4-phenylbutan-2-yl)thieno[3,2-b]pyridine-6-carboxamide, N-(2-cyanopropan-2-yl)thieno[3,2-b]pyridine-6-carboxamide, N-(tert-butyl)thieno[3,2-b]pyridine-6-carboxamide, N-(1-(trifluoromethyl)cyclopropyl)thieno[3,2-b]pyridine-6-carboxamide, N-(1,1,1-trifluoro-2-methylpropan-2-yl)thieno[3,2-b]pyridine-6-carboxamide, N-(1,1,1-trifluoro-2-methylbutan-2-yl)thieno[3,2-b]pyridine-6-carboxamide, N-(1-hydroxy-2-methylpropan-2-yl)thieno[3,2-b]pyridine-6-carboxamide, N-(3-hydroxy-3-methyl-1-(m-tolyl)butan-2-yl)thieno[3,2-b]pyridine-6-carboxamide, N-(3-hydroxy-2-methyl-4-phenylbutan-2-yl)-1-methyl-1H-pyrrolo[2,3-b]pyridine-5-carboxamide, N-(1-hydroxy-2-methyl-1-phenylpropan-2-yl)-1-methyl-1H-pyrrolo[2,3-b]pyridine-5-carboxamide, N-(1-hydroxy-2-methyl-1-phenylpropan-2-yl)thieno[3,2-b]pyridine-6-carboxamide, N-(1-(4-fluoro-3-methoxyphenyl)-1-hydroxy-2-methylpropan-2-yl)thieno[3,2-b]pyridine-6-carboxamide, N-(2-ethyl-2-hydroxy-1-phenylbutyl)-1-methyl-1H-pyrrolo[2,3-b]pyridine-5-carboxamide, N-(1-(4-fluorophenoxy)-2,4-dimethylpent-4-en-2-yl)-1-methyl-1H-pyrrolo[2,3-b]pyridine-5-carboxamide, N-(1-(4-fluorophenoxy)-2,4-dimethylpent-4-en-2-yl)thieno[3,2-b]pyridine-6-carboxamide, N-(1-(4-fluoro-3-methoxyphenyl)-1-hydroxy-2-methylpropan-2-yl)-1-methyl-1H-pyrrolo[2,3-b]pyridine-5-carboxamide, 2-methyl-2-(thieno[3,2-b]pyridine-6-carboxamido)propyl 4-fluoro-3-methoxybenzoate, 2-methyl-2-(1-methyl-1H-pyrrolo[2,3-b]pyridine-5-carboxamido)propyl 4-fluoro-2-methylbenzoate, N-(2-ethyl-2-hydroxy-1-phenylbutyl)thieno[3,2-b]pyridine-6-carboxamide, N-(1-(2-fluorophenyl)-2,4-dimethylpent-4-en-2-yl)-1-methyl-1H-pyrrolo[2,3-b]pyridine-5-carboxamide, N-(1-(4-fluorophenoxy)-2,4-dimethylpent-4-en-2-yl)-6-methyl-1H-pyrrolo[2,3-b]pyridine-5-carboxamide, N-(1-(2-fluorophenyl)-2,4-dimethylpent-4-en-2-yl)thieno[3,2-b]pyridine-6-carboxamide, N-(1-(4-fluoro-2-methylphenyl)-1-hydroxy-2-methylpropan-2-yl)-1-methyl-1H-pyrrolo[2,3-b]pyridine-5-carboxamide, N-(2-ethyl-1-(4-fluorophenyl)-2-hydroxybutyl)-1-methyl-1H-pyrrolo[2,3-b]pyridine-5-carboxamide, N-(2-ethyl-1-(4-fluorophenyl)-2-hydroxybutyl)thieno[3,2-b]pyridine-6-carboxamide, N-(1-(5-fluoro-2-methylphenyl)-1-hydroxy-2-methylpropan-2-yl)thieno[3,2-b]pyridine-6-carboxamide, N-(1-(2-fluorophenyl)-2-methylpent-4-en-2-yl)thieno[3,2-b]pyridine-6-carboxamide, 3-bromo-N-(1-(5-fluoro-2-methylphenyl)-1-hydroxy-2-methylpropan-2-yl)-1-methyl-1H-pyrrolo[2,3-b]pyridine-5-carboxamide, N-(1-(4-fluorophenyl)-2-hydroxy-2-methylpropyl)thieno[3,2-b]pyridine-6-carboxamide, 3-chloro-N-(2,3-dihydro-1H-inden-2-yl)-1-methyl-1H-pyrrolo[2,3-b]pyridine-5-carboxamide, 3-chloro-N-(2-ethyl-2-hydroxy-1-phenylbutyl)-1-methyl-1H-pyrrolo[2,3-b]pyridine-5-carboxamide, N-(1-(2-fluorophenyl)-2,4-dimethylpent-4-en-2-yl)-1H-pyrrolo[2,3-b]pyridine-5-carboxamide, 3-chloro-N-(1-(3,5-dichlorophenyl)cyclopropyl)-1-methyl-1H-pyrrolo[2,3-b]pyridine-5-carboxamide, (S)—N-(1,1-bis(3,5-dichlorophenyl)-1-hydroxypropan-2-yl)-1-methyl-1H-pyrrolo[2,3-b]pyridine-5-carboxamide, 3-bromo-N-(1-(2-fluorophenyl)-2,4-dimethylpent-4-en-2-yl)-1-methyl-1H-pyrrolo[2,3-b]pyridine-5-carboxamide, 3-chloro-N-(1-(3-methoxyphenyl)cyclopropyl)-1-methyl-1H-pyrrolo[2,3-b]pyridine-5-carboxamide, N-(1-(2-fluorophenyl)-2,4-dimethylpent-4-en-2-yl)-1H-pyrrolo[3,2-b]pyridine-6-carboxamide, 2-(6,7-dihydro-5H-cyclopenta[b]pyridine-3-carboxamido)-2-methylpropyl 3-fluorobenzoate, 2-methyl-2-(1H-pyrrolo[2,3-b]pyridine-5-carboxamido)propyl 3-fluorobenzoate, 2-(6,7-dihydro-5H-cyclopenta[b]pyridine-3-carboxamido)-2-methylpropyl 2-(trifluoromethyl)benzoate, 2-(6,7-dihydro-5H-cyclopenta[b]pyridine-3-carboxamido)-2-methylpropyl 2-methylbenzoate, 2-methyl-2-(5,6,7,8-tetrahydroquinoline-3-carboxamido)propyl 2-(trifluoromethyl)benzoate, 2-methyl-2-(5,6,7,8-tetrahydroquinoline-3-carboxamido)propyl 2-methylbenzoate, 2-methyl-2-(5,6,7,8-tetrahydroquinoline-3-carboxamido)propyl 3-fluorobenzoate, 2-(3-chloro-1-methyl-1H-pyrrolo[2,3-b]pyridine-5-carboxamido)-2-methylpropyl 3-fluorobenzoate, N-(1-(2-fluorophenoxy)-2,4-dimethylpent-4-en-2-yl)-1-methyl-1H-pyrrolo[2,3-b]pyridine-5-carboxamide, 2-methyl-2-(1H-pyrrolo[3,2-b]pyridine-6-carboxamido)propyl 3-fluorobenzoate, 2-methyl-2-(1H-pyrrolo[2,3-b]pyridine-5-carboxamido)propyl 2-(trifluoromethyl)benzoate, N-(1-(2-fluorophenoxy)-2,4-dimethylpent-4-en-2-yl)thieno[3,2-b]pyridine-6-carboxamide, N-(1-(2-fluorophenoxy)-2,4-dimethylpent-4-en-2-yl)-6,7-dihydro-5H-cyclopenta[b]pyridine-3-carboxamide, 2-methyl-2-(thieno[3,2-b]pyridine-6-carboxamido)propyl 2-(trifluoromethyl)benzoate, 2-methyl-2-(1H-pyrrolo[2,3-b]pyridine-6-carboxamido)propyl 2-(trifluoromethyl)benzoate, 2-methyl-2-(thieno[3,2-b]pyridine-6-carboxamido)propyl 2-methylbenzoate, N-(1-(4-fluoro-3-methoxyphenyl)-1-hydroxy-2-methylpropan-2-yl)-6,7-dihydro-5H-cyclopenta[b]pyridine-3-carboxamide, 2-methyl-2-(1H-pyrrolo[2,3-b]pyridine-5-carboxamido)propyl 2-methylbenzoate, 2-methyl-2-(1-methyl-1H-pyrrolo[2,3-b]pyridine-5-carboxamido)propyl 2-(trifluoromethyl)benzoate, 2-methyl-2-(1-methyl-1H-pyrrolo[2,3-b]pyridine-5-carboxamido)propyl 3-fluorobenzoate, N-(1-(4-fluorophenyl)-2-hydroxy-2-methylpropyl)-1H-pyrrolo[2,3-b]pyridine-5-carboxamide, N-(1-(2-fluorophenoxy)-2,4-dimethylpent-4-en-2-yl)-1H-pyrrolo[2,3-b]pyridine-5-carboxamide, N-(1-(4-fluoro-2-methylphenyl)-1-hydroxy-2-methylpropan-2-yl)-6,7-dihydro-5H-cyclopenta[b]pyridine-3-carboxamide, N-(1-(4-fluoro-2-methylphenyl)-1-hydroxy-2-methylpropan-2-yl)thieno[3,2-b]pyridine-6-carboxamide, N-(1-(2-fluorophenoxy)-2,4-dimethylpent-4-en-2-yl)-1H-pyrrolo[3,2-b]pyridine-6-carboxamide, 3-bromo-N-(1-(2-fluorophenoxy)-2,4-dimethylpent-4-en-2-yl)-1-methyl-1H-pyrrolo[2,3-b]pyridine-5-carboxamide, N-(1-(2-fluorophenoxy)-2,4-dimethylpent-4-en-2-yl)-6-methyl-1H-pyrrolo[2,3-b]pyridine-5-carboxamide, 2-(3-bromo-1-methyl-1H-pyrrolo[2,3-b]pyridine-5-carboxamido)-2-methylpropyl 3-fluorobenzoate, 3-chloro-N-(1-(2-fluorophenoxy)-2,4-dimethylpent-4-en-2-yl)-1-methyl-1H-pyrrolo[2,3-b]pyridine-5-carboxamide, 2-(3-bromo-1-methyl-1H-pyrrolo[2,3-b]pyridine-5-carboxamido)-2-methylpropyl 2-(trifluoromethyl)benzoate, N-(1-(4-fluorophenyl)-2,4-dimethylpent-4-en-2-yl)-1-methyl-1H-pyrrolo[2,3-b]pyridine-5-carboxamide, N-(1-(4-fluorophenyl)-2,4-dimethylpent-2-n-2-yl)thieno[3,2-b]pyridine-6-carboxamide, 2-(3-bromo-1-methyl-1H-pyrrolo[2,3-b]pyridine-5-carboxamido)-2-methylpropyl 2-methylbenzoate, N-(1-(5-fluoro-2-methylphenyl)-1-hydroxy-2-methylpropan-2-yl)-6,7-dihydro-5H-cyclopenta[b]pyridine-3-carboxamide, N-(1-((2-fluorobenzyl)oxy)-2-methylpropan-2-yl)-1-methyl-1H-pyrrolo[2,3-b]pyridine-5-carboxamide, N-(1-((2-cyanobenzyl)oxy)-2-methylpropan-2-yl)-6,7-dihydro-5H-cyclopenta[b]pyridine-3-carboxamide, N-(1-((2-fluorobenzyl)oxy)-2-methylpropan-2-yl)thieno[3,2-b]pyridine-6-carboxamide, N-(1-((2-fluorobenzyl)oxy)-2-methylpropan-2-yl)-6,7-dihydro-5H-cyclopenta[b]pyridine-3-carboxamide, N-(1-((2-chlorobenzyl)oxy)-2-methylpropan-2-yl)-6,7-dihydro-5H-cyclopenta[b]pyridine-3-carboxamide, N-(1-((2-chlorobenzyl)oxy)-2-methylpropan-2-yl)thieno[3,2-b]pyridine-6-carboxamide, N-(1-((2-cyanobenzyl)oxy)-2-methylpropan-2-yl)thieno[3,2-b]pyridine-6-carboxamide, 3-bromo-N-(1-((2-fluorobenzyl)oxy)-2-methylpropan-2-yl)-1-methyl-1H-pyrrolo[2,3-b]pyridine-5-carboxamide, 3-bromo-N-(1-((2-chlorobenzyl)oxy)-2-methylpropan-2-yl)-1-methyl-1H-pyrrolo[2,3-b]pyridine-5-carboxamide, 3-bromo-N-(1-((2-cyanobenzyl)oxy)-2-methylpropan-2-yl)-1-methyl-1H-pyrrolo[2,3-b]pyridine-5-carboxamide, N-(1-((2-chloro-5-fluorobenzyl)oxy)-2-methylpropan-2-yl)-6,7-dihydro-5H-cyclopenta[b]pyridine-3-carboxamide, N-(1-((2-chloro-5-fluorobenzyl)oxy)-2-methylpropan-2-yl)thieno[3,2-b]pyridine-6-carboxamide, N-(1-((2,4-dimethylbenzyl)oxy)-2-methylpropan-2-yl)-6,7-dihydro-5H-cyclopenta[b]pyridine-3-carboxamide, N-(1-((2,4-dimethylbenzyl)oxy)-2-methylpropan-2-yl)thieno[3,2-b]pyridine-6-carboxamide, 3-bromo-N-(1-((2,4-dimethylbenzyl)oxy)-2-methylpropan-2-yl)-1-methyl-1H-pyrrolo[2,3-b]pyridine-5-carboxamide, N-(2-methyl-1-(pyridin-2-yloxy)propan-2-yl)-6,7-dihydro-5H-cyclopenta[b]pyridine-3-carboxamide, 3-bromo-1-methyl-N-(2-methyl-1-(pyridin-2-yloxy)propan-2-yl)-1H-pyrrolo[2,3-b]pyridine-5-carboxamide, N-(1-((2-chloropyridin-4-yl)oxy)-2-methylpropan-2-yl)-6,7-dihydro-5H-cyclopenta[b]pyridine-3-carboxamide, N-(1-((2-chloropyridin-4-yl)oxy)-2-methylpropan-2-yl)thieno[3,2-b]pyridine-6-carboxamide, 3-bromo-N-(1-((2-chloropyridin-4-yl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)thieno[3,2-b]pyridine-6-carboxamide, 3-chloro-N-(1-((2-chlorobenzyl)oxy)-2-methylpropan-2-yl)-1-methyl-1H-pyrrolo[2,3-b]pyridine-5-carboxamide, 3-chloro-N-(1-((2-cyanobenzyl)oxy)-2-methylpropan-2-yl)-1-methyl-1H-pyrrolo[2,3-b]pyridine-5-carboxamide, 2-chloro-N-(2-methyl-1-phenylpropan-2-yl)-6,7-dihydro-5H-cyclopenta[b]pyridine-3-carboxamide, 2-chloro-N-(2,4-dimethyl-1-phenylpentan-2-yl)-6,7-dihydro-5H-cyclopenta[b]pyridine-3-carboxamide, N-(2-methyl-1-(pyridin-2-yloxy)propan-2-yl)thieno[3,2-b]pyridine-6-carboxamide, 3-chloro-N-(1-((2-fluorobenzyl)oxy)-2-methylpropan-2-yl)-1-methyl-1H-pyrrolo[2,3-b]pyridine-5-carboxamide, 3-chloro-N-(1-((2-chloro-5-fluorobenzyl)oxy)-2-methylpropan-2-yl)-1-methyl-1H-pyrrolo[2,3-b]pyridine-5-carboxamide, 3-bromo-N-(1-((5-fluoropyridin-2-yl)oxy)-2-methylpropan-2-yl)-1-methyl-1H-pyrrolo[2,3-b]pyridine-5-carboxamide, 2-methoxy-N-(2-methyl-1-phenylpropan-2-yl)-6,7-dihydro-5H-cyclopenta[b]pyridine-3-carboxamide, 3-chloro-N-(1-((2,4-dimethylbenzyl)oxy)-2-methylpropan-2-yl)-1-methyl-1H-pyrrolo[2,3-b]pyridine-5-carboxamide, 3-chloro-1-methyl-N-(2-methyl-1-(pyridin-2-yloxy)propan-2-yl)-1H-pyrrolo[2,3-b]pyridine-5-carboxamide, 3-chloro-N-(1-((2-chloropyridin-4-yl)oxy)-2-methylpropan-2-yl)-1-methyl-1H-pyrrolo[2,3-b]pyridine-5-carboxamide, 3-chloro-N-(1-((5-fluoropyridin-2-yl)oxy)-2-methylpropan-2-yl)-1-methyl-1H-pyrrolo[2,3-b]pyridine-5-carboxamide, 3-chloro-N-(1-((5-cyanopyridin-2-yl)oxy)-2-methylpropan-2-yl)-1-methyl-1H-pyrrolo[2,3-b]pyridine-5-carboxamide, N-(1-((S-cyanopyridin-2-yl)oxy)-2-methylpropan-2-yl)-6,7-dihydro-5H-cyclopenta[b]pyridine-3-carboxamide, N-(1-((5-cyanopyridin-2-yl)oxy)-2-methylpropan-2-yl)thieno[3,2-b]pyridine-6-carboxamide, 3-bromo-N-(1-((5-cyanopyridin-2-yl)oxy)-2-methylpropan-2-yl)-1-methyl-1H-pyrrolo[2,3-b]pyridine-5-carboxamide, N-(1-((2-chloro-5-fluorobenzyl)oxy)-2-methylpropan-2-yl)-1-methyl-1H-pyrrolo[2,3-b]pyridine-5-carboxamide, N-(1-((2,5-dimethylbenzyl)oxy)-2-methylpropan-2-yl)-1-methyl-1H-pyrrolo[2,3-b]pyridine-5-carboxamide, 1-methyl-N-(2-methyl-1-(pyridin-2-yloxy)propan-2-yl)-1H-pyrrolo[2,3-b]pyridine-5-carboxamide, N-(1-((2-chloropyridin-4-yl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)-1-methyl-1H-pyrrolo[2,3-b]pyridine-5-carboxamide, N-(1-((S-cyanopyridin-2-yl)oxy)-2-methylpropan-2-yl)-1-methyl-1H-pyrrolo[2,3-b]pyridine-5-carboxamide, 3-bromo-N-(1-((2-chloro-5-fluorobenzyl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)-6,7-dihydro-5H-cyclopenta[b]pyridine-3-carboxamide, N-(1-((2-cyanobenzyl)oxy)-2-methylpropan-2-yl)-2-methoxy-6,7-dihydro-5H-cyclopenta[b]pyridine-3-carboxamide, 2-chloro-N-(1-((2-cyanobenzyl)oxy)-2-methylpropan-2-yl)-6,7-dihydro-5H-cyclopenta[b]pyridine-3-carboxamide, N-(1-((2,4-dimethylbenzyl)oxy)-2-methylpropan-2-yl)-2-methoxy-6,7-dihydro-5H-cyclopenta[b]pyridine-3-carboxamide, 2-chloro-N-(1-((2,4-dimethylbenzyl)oxy)-2-methylpropan-2-yl)-6,7-dihydro-5H-cyclopenta[b]pyridine-3-carboxamide, N-(2,4-dimethyl-1-phenylpentan-2-yl)-2-methoxy-6,7-dihydro-5H-cyclopenta[b]pyridine-3-carboxamide, N-(1-(2-fluorophenyl)propan-2-yl)-N-methoxy-1-methyl-1H-pyrrolo[2,3-b]pyridine-5-carboxamide, 2-(2-methoxy-6,7-dihydro-5H-cyclopenta[b]pyridine-3-carboxamido)-2-methylpropyl 3-fluorobenzoate, 2-methoxy-N-(2-methyl-1-(pyridin-2-yloxy)propan-2-yl)-6,7-dihydro-5H-cyclopenta[b]pyridine-3-carboxamide, 2-chloro-N-(2-methyl-1-(pyridin-2-yloxy)propan-2-yl)-6,7-dihydro-5H-cyclopenta[b]pyridine-3-carboxamide, N-(1-((2-chloropyridin-4-yl)oxy)-2-methylpropan-2-yl)-2-methoxy-6,7-dihydro-5H-cyclopenta[b]pyridine-3-carboxamide, 2-chloro-N-(1-((2-chloropyridin-4-yl)oxy)-2-methylpropan-2-yl)-6,7-dihydro-5H-cyclopenta[b]pyridine-3-carboxamide, N-(1-benzylcyclopropyl)-2-methoxy-6,7-dihydro-5H-cyclopenta[b]pyridine-3-carboxamide, N-(1-benzylcyclopropyl)-2-chloro-6,7-dihydro-5H-cyclopenta[b]pyridine-3-carboxamide, 2-methoxy-N-((1-phenylcyclopropyl)methyl)-6,7-dihydro-5H-cyclopenta[b]pyridine-3-carboxamide, 2-chloro-N-((1-phenylcyclopropyl)methyl)-6,7-dihydro-5H-cyclopenta[b]pyridine-3-carboxamide, 2-methoxy-N-(4-phenylbutan-2-yl)-6,7-dihydro-5H-cyclopenta[b]pyridine-3-carboxamide, 2-chloro-N-(4-phenylbutan-2-yl)-6,7-dihydro-5H-cyclopenta[b]pyridine-3-carboxamide, 2-chloro-N-(1-phenylcyclopropyl)-6,7-dihydro-5H-cyclopenta[b]pyridine-3-carboxamide, 2-methoxy-N-(1-phenylcyclopropyl)-6,7-dihydro-5H-cyclopenta[b]pyridine-3-carboxamide, N-(1-(2-fluorophenyl)propan-2-yl)-N-methoxy-6,7-dihydro-5H-cyclopenta[b]pyridine-3-carboxamide, 2-chloro-N-(1-(2-fluorophenyl)propan-2-yl)-N-methoxy-6,7-dihydro-5H-cyclopenta[b]pyridine-3-carboxamide, N-(1-(2-fluorophenyl)propan-2-yl)-N-methoxy-1H-pyrrolo[2,3-b]pyridine-5-carboxamide, 2-chloro-N-(1-(4-fluorophenyl)-2,4-dimethylpent-4-en-2-yl)-6,7-dihydro-5H-cyclopenta[b]pyridine-3-carboxamide, 2-chloro-N-(1-(2-fluorophenoxy)-2,4-dimethylpent-4-en-2-yl)-6,7-dihydro-5H-cyclopenta[b]pyridine-3-carboxamide, 2-(2-chloro-6,7-dihydro-5H-cyclopenta[b]pyridine-3-carboxamido)-2-methylpropyl 3-fluorobenzoate, 2-methoxy-N-(4,4,4-trifluoro-2-methyl-1-phenylbutan-2-yl)-6,7-dihydro-5H-cyclopenta[b]pyridine-3-carboxamide, 2-chloro-N-(2-methyl-1-phenylpropan-2-yl)-5,6,7,8-tetrahydroquinoline-3-carboxamide, 2-chloro-N-(2,4-dimethyl-1-phenylpentan-2-yl)-5,6,7,8-tetrahydroquinoline-3-carboxamide, 2-chloro-N-(1-((2,4-dimethylbenzyl)oxy)-2-methylpropan-2-yl)-5,6,7,8-tetrahydroquinoline-3-carboxamide, N-(2,4-dimethyl-1-phenylpentan-2-yl)-2-methyl-1H-pyrrolo[2,3-b]pyridine-5-carboxamide, N-(2,4-dimethyl-1-phenylpentan-2-yl)-1,2-dimethyl-1H-pyrrolo[2,3-b]pyridine-5-carboxamide, N-(1-benzylcyclopropyl)-1,2-dimethyl-1H-pyrrolo[2,3-b]pyridine-5-carboxamide, 2-chloro-N-(1-((2-chlorobenzyl)oxy)-2-methylpropan-2-yl)-5,6,7,8-tetrahydroquinoline-3-carboxamide, 2-chloro-N-(1-((2-fluorobenzyl)oxy)-2-methylpropan-2-yl)-5,6,7,8-tetrahydroquinoline-3-carboxamide, 2-chloro-N-(1-((2-cyanobenzyl)oxy)-2-methylpropan-2-yl)-5,6,7,8-tetrahydroquinoline-3-carboxamide, 2-chloro-N-(1-(4-fluorophenyl)-2,4-dimethylpent-4-en-2-yl)-5,6,7,8-tetrahydroquinoline-3-carboxamide, 2-chloro-N-(1-(2-fluorophenoxy)-2,4-dimethylpent-4-en-2-yl)-5,6,7,8-tetrahydroquinoline-3-carboxamide, N-(1-((2-chlorobenzyl)oxy)-2-methylpropan-2-yl)-2-methoxy-6,7-dihydro-5H-cyclopenta[b]pyridine-3-carboxamide, N-(1-((2-fluorobenzyl)oxy)-2-methylpropan-2-yl)-2-methoxy-6,7-dihydro-5H-cyclopenta[b]pyridine-3-carboxamide, N-(1-(4-fluorophenyl)-2,4-dimethylpent-4-en-2-yl)-2-methoxy-6,7-dihydro-5H-cyclopenta[b]pyridine-3-carboxamide, N-(1-(2-fluorophenoxy)-2,4-dimethylpent-4-en-2-yl)-2-methoxy-6,7-dihydro-5H-cyclopenta[b]pyridine-3-carboxamide, N-(1-((2-chloro-5-fluorobenzyl)oxy)-2-methylpropan-2-yl)-2-methoxy-6,7-dihydro-5H-cyclopenta[b]pyridine-3-carboxamide, N-(1-(4-fluorophenyl)-2-methylpropan-2-yl)-1,2-dimethyl-1H-pyrrolo[2,3-b]pyridine-5-carboxamide, 1,2-dimethyl-N-(2-phenylpropyl)-1H-pyrrolo[2,3-b]pyridine-5-carboxamide, 1,2-dimethyl-N-((1-phenylcyclopropyl)methyl)-1H-pyrrolo[2,3-b]pyridine-5-carboxamide, 2-chloro-N-methoxy-N-(1-phenylpropan-2-yl)-6,7-dihydro-5H-cyclopenta[b]pyridine-3-carboxamide, 4-methyl-N-(1-phenylcyclopropyl)-3,4-dihydro-2H-pyrido[3,2-b][1,4]oxazine-7-carboxamide, N-(1-(4-fluorophenyl)cyclopropyl)-4-methyl-3,4-dihydro-2H-pyrido[3,2-b][1,4]oxazine-7-carboxamide, 4-methyl-N-((1-phenylcyclopropyl)methyl)-3,4-dihydro-2H-pyrido[3,2-b][1,4]oxazine-7-carboxamide, N-(1-benzylcyclopropyl)-4-methyl-3,4-dihydro-2H-pyrido[3,2-b][1,4]oxazine-7-carboxamide, 2-chloro-N-(2-methyl-1-(pyridin-2-yloxy)propan-2-yl)-5,6,7,8-tetrahydroquinoline-3-carboxamide, 2-chloro-N-(1-((2-chloropyridin-4-yl)oxy)-2-methylpropan-2-yl)-5,6,7,8-tetrahydroquinoline-3-carboxamide, 2-chloro-N-(4,4,4-trifluoro-2-methyl-1-phenylbutan-2-yl)-6,7-dihydro-5H-cyclopenta[b]pyridine-3-carboxamide, 2-chloro-N-(1-((2-chloro-5-fluorobenzyl)oxy)-2-methylpropan-2-yl)-5,6,7,8-tetrahydroquinoline-3-carboxamide, 2-methoxy-N-(2-methyl-1-(pyridin-2-yloxy)propan-2-yl)-5,6,7,8-tetrahydroquinoline-3-carboxamide, 2-methoxy-N-(2-methyl-1-phenylpropan-2-yl)-5,6,7,8-tetrahydroquinoline-3-carboxamide, N-(2,4-dimethyl-1-phenylpentan-2-yl)-2-methoxy-5,6,7,8-tetrahydroquinoline-3-carboxamide, 2-methoxy-N-(1-phenylcyclopropyl)-5,6,7,8-tetrahydroquinoline-3-carboxamide, 2-methoxy-N-((1-phenylcyclopropyl)methyl)-5,6,7,8-tetrahydroquinoline-3-carboxamide, 2-methoxy-N-(4-phenylbutan-2-yl)-5,6,7,8-tetrahydroquinoline-3-carboxamide, N-(1-(4-fluorophenyl)-2-methylpropan-2-yl)-2-methoxy-5,6,7,8-tetrahydroquinoline-3-carboxamide, 2-chloro-N-(4-phenylbutan-2-yl)-5,6,7,8-tetrahydroquinoline-3-carboxamide, 2-chloro-N-(1-(4-fluorophenyl)-2-methylpropan-2-yl)-5,6,7,8-tetrahydroquinoline-3-carboxamide, N-(1-(4-fluorophenyl)-2,4-dimethylpent-4-en-2-yl)-5,6,7,8-tetrahydroquinoline-3-carboxamide, N-(1-(2-fluorophenoxy)-2,4-dimethylpent-4-en-2-yl)-5,6,7,8-tetrahydroquinoline-3-carboxamide, N-(1-(4-fluorophenyl)cyclopropyl)-2-methoxy-5,6,7,8-tetrahydroquinoline-3-carboxamide, N-(4,4,4-trifluoro-2-methyl-1-phenylbutan-2-yl)-6,7-dihydro-5H-cyclopenta[b]pyridine-3-carboxamide, 2-chloro-N-(4,4,4-trifluoro-2-methyl-1-phenylbutan-2-yl)-5,6,7,8-tetrahydroquinoline-3-carboxamide, 2-methoxy-N-(4,4,4-trifluoro-2-methyl-1-phenylbutan-2-yl)-5,6,7,8-tetrahydroquinoline-3-carboxamide, 1-methyl-N-(4,4,4-trifluoro-2-methyl-1-phenylbutan-2-yl)-1H-pyrrolo[2,3-b]pyridine-5-carboxamide, 3-chloro-1-methyl-N-(4,4,4-trifluoro-2-methyl-1-phenylbutan-2-yl)-1H-pyrrolo[2,3-b]pyridine-5-carboxamide, 3-chloro-6-methoxy-1-methyl-N-(4,4,4-trifluoro-2-methyl-1-phenylbutan-2-yl)-1H-pyrrolo[2,3-b]pyridine-5-carboxamide, 6,6-difluoro-N-(4,4,4-trifluoro-2-methyl-1-phenylbutan-2-yl)-5,6,7,8-tetrahydroquinoline-3-carboxamide, N-(2,4-dimethyl-1-phenylpentan-2-yl)-6,6-difluoro-5,6,7,8-tetrahydroquinoline-3-carboxamide, 4-chloro-N-(4,4,4-trifluoro-2-methyl-1-phenylbutan-2-yl)-5,6,7,8-tetrahydroquinoline-3-carboxamide, 4-chloro-N-(2,4-dimethyl-1-phenylpentan-2-yl)-6,7-dihydro-5H-cyclopenta[b]pyridine-3-carboxamide, 4-chloro-N-(2,4-dimethyl-1-phenylpent-2-n-2-yl)-6,7-dihydro-5H-cyclopenta[b]pyridine-3-carboxamide, 4-chloro-N-(2,4-dimethyl-1-phenylpentan-2-yl)-5,6,7,8-tetrahydroquinoline-3-carboxamide, N-(2,4-dimethyl-1-phenylpentan-2-yl)-4-methoxy-5,6,7,8-tetrahydroquinoline-3-carboxamide, N-(2,4-dimethyl-1-phenylpentan-2-yl)-4-methoxy-6,7-dihydro-5H-cyclopenta[b]pyridine-3-carboxamide, N-(2,4-dimethyl-1-phenylpent-4-en-2-yl)-4-methoxy-6,7-dihydro-5H-cyclopenta[b]pyridine-3-carboxamide, 4-chloro-N-(1-(2-fluorophenyl)-2-methylpropan-2-yl)-6,7-dihydro-5H-cyclopenta[b]pyridine-3-carboxamide, 4-chloro-N-(1-(4-fluorophenyl)-2-methylpropan-2-yl)-6,7-dihydro-5H-cyclopenta[b]pyridine-3-carboxamide, N-(1-(2-fluorophenyl)-2-methylpropan-2-yl)-4-methoxy-6,7-dihydro-5H-cyclopenta[b]pyridine-3-carboxamide, or N-(1-(4-fluorophenyl)-2-methylpropan-2-yl)-4-methoxy-6,7-dihydro-5H-cyclopenta[b]pyridine-3-carboxamide.

The agriculturally acceptable salts of the compounds of Formula (I) encompass especially the salts of those cations or the acid addition salts of those acids whose cations and anions, respectively, have no adverse effect on the fungicidal action of the compounds of Formula (I). Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

The salts obtainable in this way likewise have fungicidal properties.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound of Formula (I) with an acid of the corresponding anion, preferably of hydrochloric acid, hydrobromic acid, sulfuric acid, phosphoric acid, or nitric acid.

The compounds of the present invention may be present either in pure form or as mixtures of different possible isomeric forms such as stereoisomers e.g. a racemate, individual stereoisomers, or constitutional isomers or as an optically active form. The various stereoisomers include enantiomers, diastereomers, chiral isomers, atropisomers, conformers, rotamers, tautomers, optical isomers, polymorphs, and geometric isomers. Any desired mixtures of these isomers fall within the scope of the claims of the present disclosure. One skilled in the art will appreciate that one stereoisomer may be more active and/or may exhibit beneficial effects when enriched relative to the other isomer(s) or when separated from the other isomer(s). Additionally, the person skilled in the art knows processes or methods or technologies to separate, enrich, and/or to selectively prepare said isomers.

The compounds of Formula (I) can be present in different crystal modifications whose biological activity may differ. They also form part of the subject matter of the present invention. The compounds of Formula (I) can be present in atropisomers arising from restricted rotation about a single bond of asymmetric groups. They also form part of the subject matter of the present invention.

The compound selected from Formula (I) (including all stereoisomers, N-oxides, and salts thereof) may typically exist in more than one form. The compounds of Formula (I) thus include all crystalline and non-crystalline forms of the compound that Formula (I) represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types).

The following schemes illustrate approaches for preparing compounds of Formula (I). The following descriptions and examples are provided for illustrative purposes only and should not be construed as limiting in terms of substituents or substitution patterns. Further, the mentioned reagents, solvents and reaction conditions are intended for the purpose of exemplification only and should not be construed as limiting.

In one embodiment, the present invention provides a process for the synthesis of compounds of formula (I).

The compound of Formula (I) can be prepared by any of the following methods. The definitions of R¹, R², R³, R⁴, R⁵, R⁶, R⁷, R⁸, R⁹, R¹¹, X, Z₁, and Z₂ in the compounds of Formula (I) and compounds of Formula 1-8 are as defined above in the detailed description of the invention unless otherwise stated specifically.

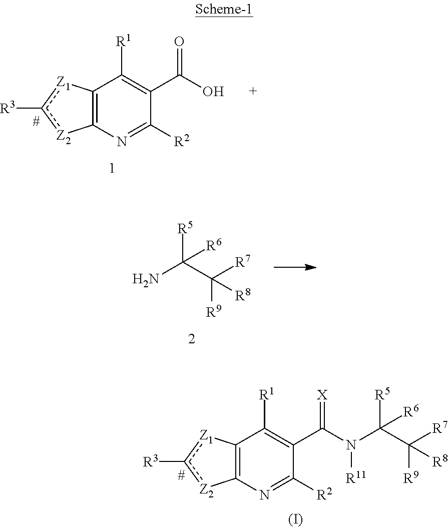

The compounds of Formula (I) wherein X is O and R¹¹ is H, can be prepared by a coupling reaction of compounds of Formula (1) and compounds of Formula (2) in the presence of a suitable coupling reagent known in the literature (Comprehensive Organic Synthesis (2nd Edition) (2014), 6, 427-478; Synthesis, 2019, 51, 2261-2277). The suitable coupling reagent is selected from 1-(3-dimethylaminopropyl)-3-ethylcarbodiimide hydrochloride, N,N′-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate, 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

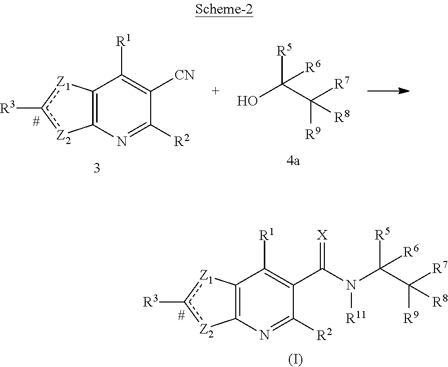

The compounds of Formula (I) wherein X is O and R¹¹ is H, can be prepared by a Brønsted acid catalyzed Ritter reaction of compounds of Formula (3) with a suitable secondary or tertiary alcohol of Formula (4a) in the presence of a suitable Brønsted acid such as sulfuric acid by following the analogous procedure as described in WO2019154665 (scheme 2).

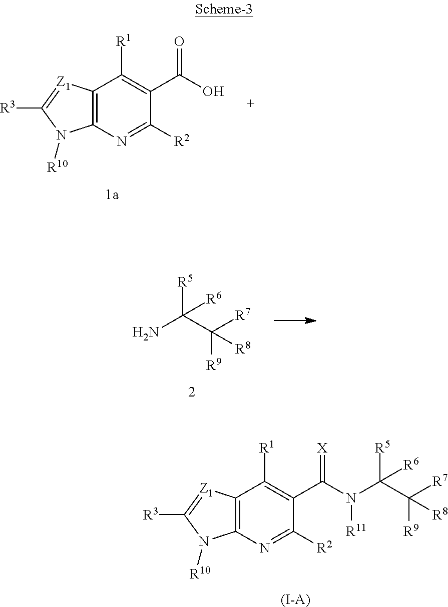

The compounds of Formula (I-A) wherein X is O and R¹¹ is H, can be obtained by the coupling of a suitable acid of formula (1a) with a suitable amine of Formula (2) in the presence of a suitable coupling reagent known in the literature (Comprehensive Organic Synthesis (2nd Edition) (2014), 6, 427-478; Synthesis 2019, 51, 2261-2277). The suitable coupling reagent is selected from 1-(3-dimethylaminopropyl)-3-ethylcarbodiimide hydrochloride, N,N′-dicyclohexylcarbodiimide, benzotriazol-1-yloxytris(dimethylamino)phosphonium hexafluorophosphate, I-[bis(dimethylamino)methylene]-1H-1,2,3-triazolo[4,5-b]pyridinium 3-oxide hexafluorophosphate, 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

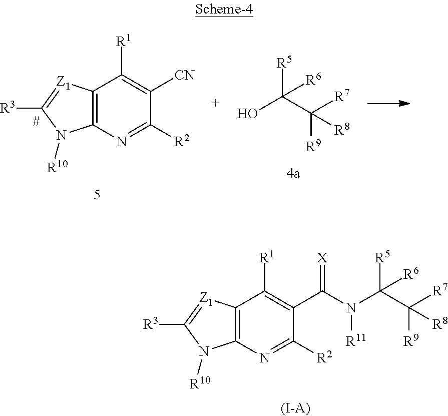

The compounds of Formula (I-A) wherein X is O and R¹¹ is H, can be obtained by a Brønsted acid catalyzed Ritter reaction of compounds of Formula (5) with a suitable secondary or tertiary alcohol of Formula (4a) in the presence of a suitable acid such as sulfuric acid by following the analogous procedure as described in WO2019154665 (scheme 4).

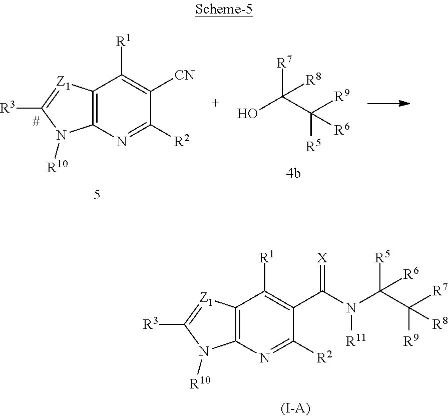

The compounds of Formula (I-A) wherein X is O and R¹¹ is H, can be obtained by reacting a secondary or tertiary alcohol of Formula (5) with compounds of Formula (4b) in a Ritter reaction as shown in scheme 5 (Synthesis, 2021, 53, 146-160).

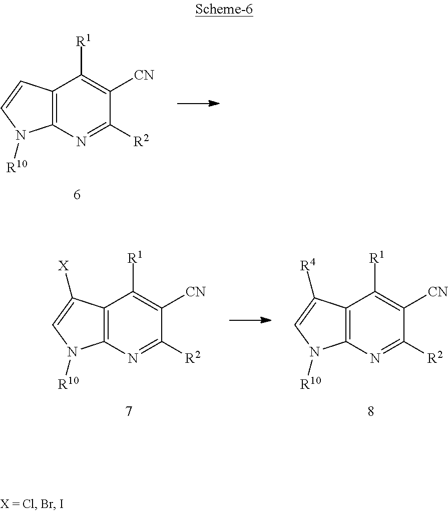

The compounds of Formula (7) can be obtained by the halogenation of pyrrolo-pyridine-nitrile of Formula (6) with a suitable reagent such as selectfluor, N-chlorosuccinimide, N-bromosuccinimide, N-iodosuccinimide, and other halogenating reagents known in the literature (WO2020012424). The compounds of Formula (7) can be converted to compounds of Formula (8) wherein Z₁, is CR⁴ by Suzuki coupling reaction with suitable alkyl-boronates. This transformation can also be carried out with various coupling reagents known in WO2014004064 (scheme 6).

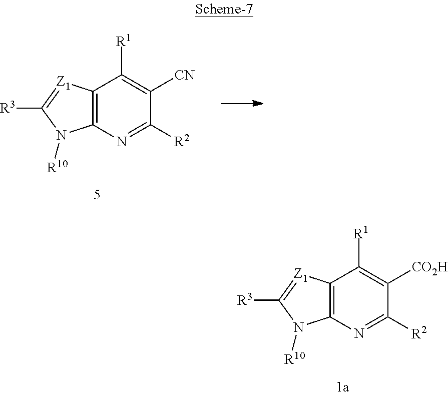

The substituted pyrrolo-pyridine-nitrile of Formula (1a) wherein Z₁ is S, can be obtained by the hydrolysis of compounds of Formula (5) in the presence of a suitable reagent such as a basic reagent selected from sodium hydroxide, potassium hydroxide, and the like or a suitable acid selected from concentrated hydrochloric acid, sulfuric acid and the like. The hydrolysis of nitrile of Formula (5) can also be achieved by methods known in the literature (WO2009155017) as shown in scheme 7.

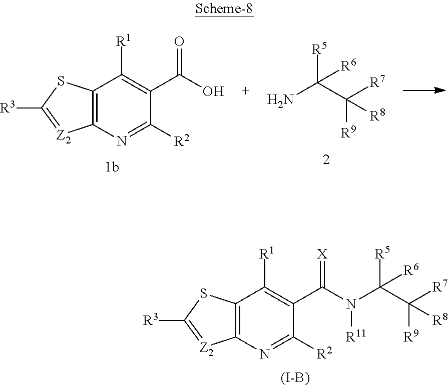

The compounds of Formula (I-B) wherein X is O and R¹¹ is H, can be obtained by the coupling of a suitable acid of formula (1b) with a suitable amine of Formula (2) in the presence of a suitable coupling reagent known in the literature (Comprehensive Organic Synthesis (2nd Edition) (2014), 6, 427-478; Synthesis; 2019, 51, 2261-2277). The suitable coupling reagent is selected from 1-(3-dimethylaminopropyl)-3-ethylcarbodiimide hydrochloride, N,N′-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate, 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

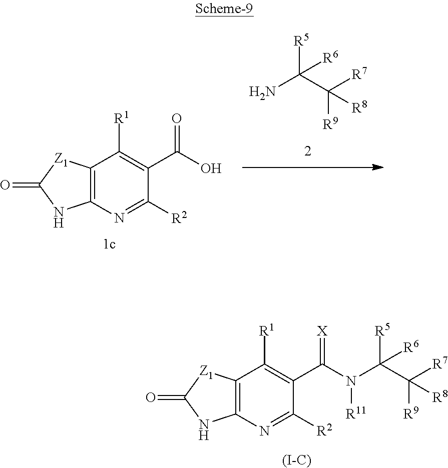

The compounds of Formula (I-C) wherein X is O and R¹¹ is H, can be obtained by the coupling of a suitable acid of Formula (1c) with a suitable amine of Formula (2) in the presence of a suitable coupling reagent known in the literature (Comprehensive Organic Synthesis (2nd Edition) (2014), 6, 427-478; Synthesis; 2019, 51, 2261-2277). The suitable coupling reagent is selected from 1-(3-dimethylaminopropyl)-3-ethylcarbodiimide hydrochloride, N,N′-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate and 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

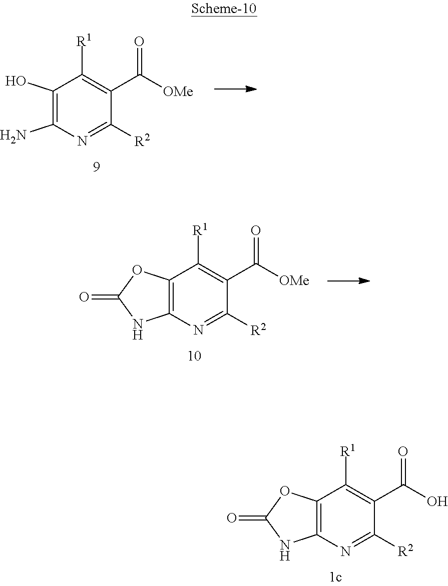

The heteroaryl acid of Formula (1c) wherein Z₁ is O, can be obtained by the reaction of a heteroaryl ester of Formula (9) with a suitable reagent such as 1,1′-carbonyldiimidazole, ethyl chloroformate, triphosgene, and the like, used for the synthesis of carbamates from amino alcohols as shown in scheme 10 (CN111943885). Hydrolysis of the compounds of Formula (10) to obtain the acid of Formula (1c) can be achieved with suitable reagents such as lithium hydroxide, sodium hydroxide, potassium hydroxide, sodium carbonate, potassium carbonate and other regents known in the literature.

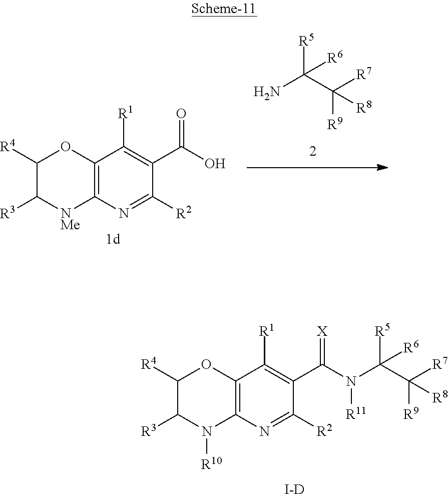

The compounds of Formula (I-D) wherein X is O and R¹¹ is H, can be obtained by the coupling of a suitable acid of Formula (1d) with a suitable amine of Formula (2) in the presence of a suitable coupling reagent known in the literature (Comprehensive Organic Synthesis (2nd Edition) (2014), 6, 427-478; Synthesis, 2019, 51, 2261-2277). The suitable coupling reagent is selected from 1-(3-dimethylaminopropyl)-3-ethylcarbodiimide hydrochloride, N,N′-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate and 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

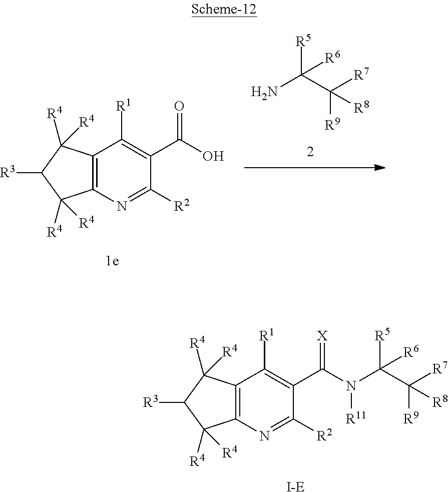

The compounds of Formula (I-E) wherein X is O and R¹¹ is H, can be obtained by the coupling of a suitable acid of Formula (1e) with a suitable amine of Formula (2) in the presence of a suitable coupling reagent known in the literature, (Comprehensive Organic Synthesis (2nd Edition) (2014), 6, 427-478; Synthesis, 2019, 51, 2261-2277), for example 1-(3-dimethylaminopropyl)-3-ethylcarbodiimide hydrochloride, NM-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate and 3-[bis(dimethylamino)methyliumyl]-3H-benzotriazol-1-oxide hexafluorophosphate and other coupling reagents that are known for the synthesis of amide (Chem. Soc. Rev., 2009, 38, 606-631).

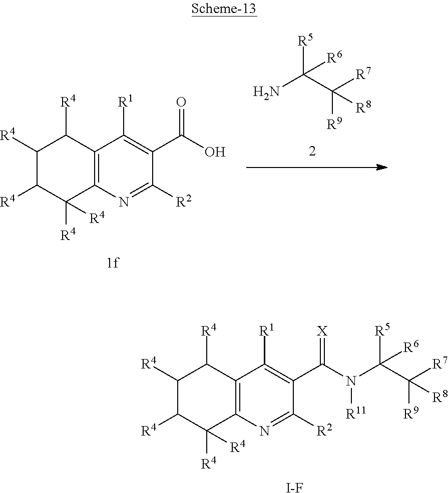

The compounds of Formula (I-F) wherein X is O and R¹¹ is H, can be obtained by the coupling of a suitable acid of Formula (1f) with a suitable amine of Formula (2) in the presence of a suitable coupling reagent known in the literature, (Comprehensive Organic Synthesis (2nd Edition) (2014), 6, 427-478; Synthesis, 2019, 51, 2261-2277), for example 1-(3-dimethylaminopropyl)-3-ethylcarbodiimide hydrochloride, NM-dicyclohexylcarbodiimide, benzotriazol-1-yloxytris(dimethylamino)phosphonium hexafluorophosphate, 1-[bis(dimethylamino)methylene]-1H-1,2,3-triazolo[4,5-b]pyridinium 3-oxide hexafluorophosphate and 3-[bis(dimethylamino)methyliumyl]-3H-benzotriazol-1-oxide hexafluorophosphate and other coupling reagents that are known for amide synthesis (Chem. Soc. Rev., 2009, 38, 606-631).

In one embodiment, the present invention provides an agrochemical composition comprising a compound of Formula (I), agriculturally acceptable salts, metal complexes, constitutional isomers, stereo-isomers, diastereoisomers, enantiomers, chiral isomers, atropisomers, conformers, rotamers, tautomers, optical isomers, polymorphs, geometric isomers, or N-oxides thereof, optionally with one or more additional active ingredient(s), and optionally together with an auxiliary such as an inert carrier or any other essential ingredient(s) such as surfactants, additives, solid diluents and liquid diluents.

An agrochemical composition comprises a fungicidally effective amount of a compound of Formula (I). The term “effective amount” denotes an amount of the composition or of the compound of Formula (I), which is sufficient for controlling harmful fungi on cultivated plants or in the protection of materials, and which does not result in a substantial damage to the treated plants. Such an amount can vary in a broad range and is dependent on various factors, such as the fungal species to be controlled, the treated cultivated plant or material, the climatic conditions and the specific compound of Formula (I) being used.

The term “fungicidally effective amount” means the quantity of such a compound or of a combination of such compounds that is capable of producing an effect on the growth of fungi. Controlling or modifying effects include all kind of deviation from natural development, such as killing, retardation and the like, and prevention includes barrier or other defensive formation in or on a plant to prevent fungal infection.

The compounds of the present disclosure may be applied by any of a variety of known techniques, either as the compounds themselves or as formulations comprising these compounds. For example, the compounds may be applied to the roots or foliage of plants for the control of various fungi, without damaging the commercial value of the plants. The materials may be applied in the form of any of the generally used formulation types, for example, as solutions, dusts, wettable powders, flowable concentrate, or emulsifiable concentrates.

Preferably, the compounds of the present disclosure are applied in the form of a formulation, comprising one or more of the compounds of Formula (I) together with a phytologically acceptable carrier. Concentrated formulations may be dispersed in water or other liquids for application, or formulations may be dust-like or granular, which may then be applied without further treatment. The formulations can be prepared according to procedures that are conventional in the agricultural chemical art.

The present disclosure contemplates all vehicles by which one or more of the compounds may be formulated for delivery and use as a fungicide. Typically, formulations are applied as aqueous suspensions or emulsions. Such suspensions or emulsions may be produced from water-soluble, water-suspendable, or emulsifiable formulations which are solids, usually known as wettable powders; or liquids, usually known as emulsifiable concentrates, aqueous suspensions, or suspension concentrates. As will be readily appreciated, any material to which these compounds may be added, may be used, provided it will yield the desired utility without significant interference with the activity of these compounds, as antifungal agents.

The term “plant health” is to be understood to denote a condition of the plant and/or its products which is determined by several indicators alone, or in combination with each other such as yield (e.g. increased biomass and/or increased content of valuable ingredients), plant vigor (e.g. improved plant growth and/or greener leaves (“greening effect”)), quality (e.g. improved content or composition of certain ingredients) and tolerance to abiotic and/or biotic stress. The above identified indicators for the health condition of a plant may be interdependent or may result from each other.

The compounds of Formula (I) are employed as such or in the form of a composition for treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials, or rooms by the fungi.

Plant propagation material may be treated with a compound of Formula (I), the combination and the composition thereof protectively either at or before planting or transplanting.

In another embodiment, the present invention provides a composition comprising at least one compound of Formula (I), and seed. The amount of the compound of Formula (I) in the composition ranges from 0.1 g ai (gram per active ingredient) to 1 kg ai (kilogram per active ingredient) per 100 kg of seeds.

The compound of Formula (I), their oxides, metal complexes, isomers, polymorphs or the agriculturally acceptable salts thereof can be converted into customary types of agrochemical compositions, e.g. into solutions, emulsions, suspensions, dusts, powders, pastes, granules, pressings, capsules, and mixtures thereof. Examples for such composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further composition types are defined in the “Catalogue of pesticide Formulation types and international coding system”, Technical Monograph No. 2, 6^{th} Ed. May 2008, CropLife International.

The mentioned compositions are prepared in a known manner, such as described by Mollet and Grubemann, “Formulation Technology”, Wiley VCH, Weinheim, 2001; or Knowles, “New Developments in Crop Protection Product Formulation”, Agrow Reports DS243, T & F Informs, London, 2005.

Examples for suitable auxiliaries for formulations and/or agrochemicals compositions according to the invention are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers in this context are for instance water and organic solvents, such as mineral oil fractions of medium to high boiling point, e.g. kerosene, diesel oil, oils of vegetable or animal origin; aliphatic, cyclic and aromatic hydrocarbons, e.g. toluene, paraffin, tetrahydronaphthalene, alkylated naphthalenes; alcohols, e.g. ethanol, propanol, butanol, benzyl alcohol, cyclohexanol; glycols; dimethyl sulfoxide; ketones, e.g. cyclohexanone; esters, e.g. lactates, carbonates, fatty acid esters, gamma-butyrolactone; fatty acids; phosphonates; amines; amides, e.g. N-methyl pyrrolidone, fatty acid dimethyl amides; and mixtures thereof. Suitable solid carriers or fillers are for instance mineral earths, e.g. silicates, silica gels, talc, kaolins, limestone, lime, chalk, clays, dolomite, diatomaceous earth, bentonite, calcium sulphate, magnesium sulphate, magnesium oxide; polysaccharides, e.g. cellulose, starch; fertilizers, e.g. ammonium sulphate, ammonium phosphate, ammonium nitrate, ureas; products of vegetable origin, e.g. cereal meal, tree bark meal, wood meal, nutshell meal, and mixtures thereof.

Suitable surfactants are surface-active compounds, for instance such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: “Emulsifiers & Detergents”, McCutcheon's Directories, Glen Rock, USA, 2008 (International Ed. or North American Ed.).

Suitable anionic surfactants are for instance alkali, alkaline earth or ammonium salts of sulfonates, sulphates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulphates are sulphates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are for instance alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinyl pyrrolidone, vinyl alcohols, or vinyl acetate.

Suitable cationic surfactants are for instance quaternary surfactants, for example quaternary ammonium compounds with one or two hydrophobic groups, or salts of long-chain primary amines. Suitable amphoteric surfactants are alkylbetains and imidazolines. Suitable block polymers are for instance block polymers of the A-B or A-B-A type comprising blocks of polyethylene oxide and polypropylene oxide, or of the A-B-C type comprising alkanol, polyethylene oxide and polypropylene oxide. Suitable polyelectrolytes are for instance polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound of Formula (I) on the target. Examples are surfactants, mineral or vegetable oils, and other auxiliaries. Further examples are listed by Knowles, “Adjuvants and Additives”, Agrow Reports DS256, T & F Informs UK, 2006, chapter 5.

Suitable thickeners are for instance polysaccharides (e.g. xanthan gum, carboxymethyl cellulose), inorganic clays (organically modified or unmodified), polycarboxylates, and silicates.

Suitable bactericides are for instance bronopol and isothiazolinone derivatives such as alkylisothiazolinones and benzisothiazolinones.

Suitable anti-freezing agents are for instance ethylene glycol, propylene glycol, urea and glycerin.

Suitable anti-foaming agents are for instance silicones, long chain alcohols, and salts of fatty acids.

Suitable colorants (e.g. in red, blue, or green) are for instance pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are for instance polyvinyl pyrrolidones, polyvinyl acetates, polyvinyl alcohols, polyacrylates, biological or synthetic waxes, and cellulose ethers.

### Examples for Composition Types and their Preparation are

i) Water-soluble concentrates (SL, LS)
10-60 wt % of a compound of Formula (I) and 5-15 wt % wetting agent (e.g. alcohol alkoxylates) are dissolved in water and/or in a water-soluble solvent (e.g. alcohols) ad 100 wt %. The active substance dissolves upon dilution with water.
ii) Dispersible concentrates (DC)
5-25 wt % of a compound of Formula (I) and 1-10 wt % dispersant (e.g. polyvinyl pyrrolidone) are dissolved in an organic solvent (e.g. cyclohexanone) ad 100 wt %. Dilution with water gives a dispersion of the active substance.
iii) Emulsifiable concentrates (EC)
15-70 wt % of a compound of Formula (I) and 5-10 wt % emulsifiers (e.g. calcium dodecylbenzenesulfonate and castor oil ethoxylate) are dissolved in a water-insoluble organic solvent (e.g. aromatic hydrocarbon) ad 100 wt %. Dilution with water gives an emulsion of the active substance.
iv) Emulsions (EW, EO, ES)
5-40 wt % of a compound of Formula (I) and 1-10 wt % emulsifiers (e.g. calcium dodecylbenzenesulfonate and castor oil ethoxylate) are dissolved in 20-40 wt % of a water-insoluble organic solvent (e.g. aromatic hydrocarbon). This mixture is introduced into water ad 100 wt % by means of an emulsifying machine and made into a homogeneous emulsion. Dilution with water gives an emulsion of the active substance.
v) Suspensions (SC, OD, FS)

In an agitated ball mill, 20-60 wt % of a compound of Formula (I) are comminuted with the addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % of binder (e.g. polyvinyl alcohol) is added.
vi) Water-dispersible granules and water-soluble granules (WG, SG)
50-80 wt % of a compound of Formula (I) are ground finely with the addition of dispersants and wetting agents (e.g. sodium lignosulfonate and alcohol ethoxylate) ad 100 wt % and prepared as water-dispersible or water-soluble granules by means of technical appliances (e.g. extrusion, spray tower, fluidized bed). Dilution with water gives a stable dispersion or solution of the active substance.
vii) Water-dispersible powders and water-soluble powders (WP, SP, WS) 50-80 wt % of a compound of formula (I) are ground in a rotor-stator mill with the addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.
viii) Gel (GW, GF)

In an agitated ball mill, 5-25 wt % of a compound of Formula (I) are comminuted with the addition of 3-10 wt % dispersants (e.g. sodium lignosulfonate), 1-5 wt % thickener (e.g. carboxymethyl cellulose) and water ad 100 wt % to give a fine suspension of the active substance. Dilution with water gives a stable suspension of the active substance.
ix) Microemulsion (ME)
5-20 wt % of a compound of Formula (I) are added to 5-30 wt % of an organic solvent blend (e.g. fatty acid dimethyl amide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion of the active substance.
x) Microcapsules (CS)

An oil phase comprising 5-50 wt % of a compound of Formula (I), 0-40 wt % of a water insoluble organic solvent (e.g. aromatic hydrocarbon), 2-15 wt % of acrylic monomers (e.g. methylmethacrylate, methacrylic acid and a di- or triacrylate) are dispersed into an aqueous solution of a protective colloid (e.g. polyvinyl alcohol). Radical polymerization results in the formation of poly(meth)acrylate microcapsules. Alternatively, an oil phase comprising 5-50 wt % of a compound of Formula (I) according to the invention, 0-40 wt % of a water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of polyurea microcapsules comprising the active substance. The monomers amount to 1-10 wt %. The wt % relates to the total CS composition.
xi) Dustable powders (DP, DS)
1-10 wt % of a compound of Formula (I) are ground finely and mixed intimately with a solid carrier (e.g. finely divided kaolin) ad 100 wt %.
xii) Granules (GR, FG)
0.5-30 wt % of a compound of Formula (I) are ground finely and associated with a solid carrier (e.g. silicate) ad 100 wt %. Granulation is achieved by extrusion, spray-drying or fluidized bed.
xiii) Ultra-low volume liquids (UL)
1-50 wt % of a compound of Formula (I) are dissolved in an organic solvent (e.g. aromatic hydrocarbon) ad 100 wt %.

The compositions types i) to xiii) may optionally comprise further auxiliaries, such as 0.1-1 wt % of bactericides, 5-15 wt % of anti-freezing agents, 0.1-1 wt % of anti-foaming agents, and 0.1-1 wt % of colorants.

The agrochemical compositions generally comprise between 0.01 and 95%, preferably between 0.1 and 90%, and in particular between 0.5 and 75%, by weight of active ingredient (ai). The active ingredients (ai) are employed in a purity of from 90% to 100%, preferably from 95% to 100% (according to the NMR spectrum).

For the purposes of the treatment of plant propagation materials, particularly seeds, solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations from 0.01 to 60% by weight, preferably from 0.1 to 40%, in the ready-to-use preparations.

When employed in plant protection, the amounts of active substances applied are, depending on the kind of effect desired, from 0.001 to 2 kg per ha, preferably from 0.005 to 2 kg per ha, more preferably from 0.05 to 1.0 kg per ha, and in particular from 0.1 to 0.5 kg per ha.

In the treatment of plant propagation materials such as seeds, e.g. by dusting, coating or drenching seed, the amounts of active substance generally required are ranging from 0.1 to 1000 g, preferably from 1 to 1000 g, more preferably from 1 to 100 g and most preferably from 5 to 100 g, per 100 kg of plant propagation material (preferably seeds).

When used in the protection of materials or stored products, the amount of active substance applied depends on the kind of application area and on the desired effect. Amounts customarily applied in the protection of materials are ranging from 0.001 g to 2 kg, preferably from 0.005 g to 1 kg, of active substance per cubic meter of treated material.

Various types of oils, welters, adjuvants, fertilizer, or micronutrients, and further pesticides (e.g. herbicides, insecticides, fungicides, growth regulators, safeners, biopesticides) may be added to the active substances or the compositions comprising them, as a premix or, if appropriate, not until immediately prior to use, as a tank mix. These agents can be mixed with the composition according to the invention in a weight ratio of 1:100 to 100:1, preferably 1:20 to 20:1.

A pesticide is generally a chemical or biological agent (such as pesticidally active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease.

The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense against certain pests; safeners that reduce unwanted herbicidal action of pesticides on crop plants; and plant growth promoters that affect the plant physiology e.g. to increase plant growth, biomass, yield or any other quality parameter of the harvestable goods of a crop plant.

The user applies the composition according to the invention usually from a pr dosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 30 to 400 liters, of the ready-to-use spray liquor are applied per hectare of agricultural useful area.

In one embodiment, the present invention provides a combination comprising the compound of Formula (I) and at least one further pesticidally active substance selected from the group consisting of fungicides, insecticides, nematicides, acaricides, biopesticides, herbicides, safeners, plant growth regulators, antibiotics, fertilizers and nutrients.

The compounds of Formula (I), the combination and the composition thereof comprising them in the use as fungicides with other fungicides may result in an expansion of the fungicidal spectrum of activity being obtained or in a prevention of fungicide resistance development. Furthermore, in many cases, extraordinary and unexpected effects are obtained.

The present invention also relates to the combination comprising at least one compound of Formula (I) and at least one further pesticidally active substance selected from the group of fungicides, insecticides, nematicides, acaricides, biopesticides, herbicides, safeners, plant growth regulators, antibiotics, fertilizers and nutrients. The pesticidally active substances reported in WO2015185485 pages 36-43 and WO2017093019 pages 42-56 can be used in conjunction with the compound of Formula (I).

The active substances referred to as component 2, their preparation and their activity e.g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP141317; EP152031; EP226917; EP243970; EP256503; EP428941; EP532022; EP1028125; EP1035122; EP1201648; EP1122244, JP2002316902; DE19650197; DE10021412; DE102005009458; U.S. Pat. Nos. 3,296,272; 3,325,503; WO9846608; WO9914187; WO9924413; WO9927783; WO0029404; WO0046148; WO0065913; WO0154501; WO0156358; WO0222583; WO0240431; WO0310149; WO0311853; WO0314103; WO0316286; WO0353145; WO0361388; WO0366609; WO0374491; WO0449804; WO0483193; WO05120234; WO05123689; WO05123690; WO0563721; WO0587772; WO0587773; WO0615866; WO0687325; WO0687343; WO0782098; WO0790624; WO11028657; WO2012168188; WO2007006670; WO201177514; WO13047749; WO10069882; WO13047441; WO0316303; WO0990181; WO13007767; WO1310862; WO13127704; WO13024009; WO13024010; WO13047441; WO13162072; WO13092224 and WO11135833.

The present invention furthermore relates to agrochemical mixtures comprising at least one compound of formula (I) (component 1) and at least one further active substance useful for plant protection.

By applying the compound of formula (I) together with at least one pesticidally active compound an additional effect can be obtained.

This can be obtained by applying the compound of formula (I) and at least one further pesticidally active substance simultaneously, either jointly (e.g. as tank-mix) or separately, or in succession, wherein the time interval between the individual applications is selected to ensure that the active substance applied first still occurs at the site of action in a sufficient amount at the time of application of the further pesticidally active substance(s). The order of application is not essential for the working of the present invention.

When applying the compound of Formula (I) and a pesticidally active substance sequentially the time between both applications may vary e.g. between 2 hours to 7 days. Also, a broader range is possible ranging from 0.25 hour to 30 days, preferably from 0.5 hour to 14 days, particularly from 1 hour to 7 days or from 1.5 hours to 5 days, even more preferred from 2 hours to 1 day. In the binary mixtures and the composition according to the invention the weight ratio of the component 1) and the component 2) generally depends on the properties of the active components used, usually it is in the range of 1:1000 to 1000:1, often in the range of 1:100 to 100:1, regularly in the range of 1:50 to 50:1, preferably in the range of 1:20 to 20:1, more preferably in the range of 1:10 to 10:1, even more preferably in the range of 1:4 to 4:1 and in particular in the range of 1:2 to 2:1.

According to a further embodiment of the binary mixtures and the composition thereof, the weight ratio of the component 1) and the component 2) usually is in the range of 1000:1 to 1:1000, often in the range of 100:1 to 1:100, regularly in the range of 50:1 to 1:50, preferably in the range of 20:1 to 1:20, more preferably in the range of 10:1 to 1:10, even more preferably in the range of 4:1 to 1:4 and in particular in the range of 2:1 to 1:2.

In the ternary mixtures, i.e. the composition according to the invention comprising the (component 1 and component 2) and a compound III (component 3), the weight ratio of component 1) and component 2) depend of the properties of the active substances being used. Usually it is in the range of 1:100 to 100:1, regularly in the range of 1:50 to 50:1, preferably in the range of 1:20 to 20:1, more preferably in the range of 1:10 to 10:1 and in particular in the range of 1:4 to 4:1, and the weight ratio of component 1) and component 3) usually is in the range of 1:100 to 100:1, regularly in the range of 1:50 to 50:1, preferably in the range of 1:20 to 20:1, more preferably in the range of 1:10 to 10:1 and in particular in the range of 1:4 to 4:1.

Any further active components are, if desired, are added in a ratio of 20:1 to 1:20 to the component 1). These ratios are also suitable for inventive mixtures applied by seed treatment.

According to one embodiment, individual components of the composition according to the invention such as parts of a kit or parts of a binary or ternary mixture may be mixed by the user himself in a spray tank or any other kind of vessel used for applications (e.g. seed treater drums, seed pelleting machinery, knapsack sprayer). Further auxiliaries may be added, if appropriate.

Consequently, one embodiment of the invention is a kit for preparing a usable pesticidal composition, comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Application of the compounds of Formula (I), the combinations and the compositions thereof can be carried out before or during sowing. Methods for applying the compound of formula (I), the combination and the composition thereof, respectively, are application onto plant propagation material, especially seeds, including dressing, coating, pelleting, dusting, and soaking as well as in-furrow application methods.

Preferably, the compound of Formula (I), the combinations and the compositions thereof, respectively, are applied on to the plant propagation material by a method such that germination is not induced, e.g. by seed dressing, pelleting, coating and dusting.

It is also possible to use the compounds of Formula (I) as fungicides. The term “fungicide” as used herein means a compound that controls, modifies, or prevents the growth of phytopathogenic fungi.

The compounds of Formula (I), the combinations and the compositions thereof, respectively, may be used for improving the health of a plant. The invention also relates to a method for improving plant health by treating a plant, its propagation material and/or the locus where the plant is growing or is to grow with an effective amount of a compound of Formula (I) and/or a composition thereof, respectively.

In one embodiment, the present invention provides a method for controlling or preventing infestation of plants by phytopathogenic microorganisms in agricultural crops and/or horticultural crops, wherein an effective amount of at least one compound of Formula (I) or the combination or the composition is applied to the plants, to parts thereof or to a locus thereof.

In another embodiment, the present invention provides a method for controlling or preventing infestation of plants by phytopathogenic microorganisms in agricultural crops and/or horticultural crops wherein an effective amount of at least one compound of Formula (I) or the combination or the composition thereof, is applied to the seeds of plants.

The compounds of Formula (I) and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically active and they can be used in crop protection as foliar fungicides, fungicides for seed dressing and soil fungicides. Moreover, they are suitable for controlling harmful fungi, which inter alia occur in wood or roots of plants.

The compounds of Formula (I) and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e.g. conifers; and on the plant propagation material, such as seeds, and the crop material of these plants. Particularly, the compound of Formula (I) and the composition according to the invention are important in the control of phytopathogenic fungi on soybeans and on the plant propagation material, such as seeds, and the crop material of soybeans.

Preferably, the compounds of Formula (I) and compositions thereof, respectively are used for controlling a multitude of fungi on field crops, such as potatoes, sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative or reproductive parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts, twigs, flowers, and other parts of plants, including seedlings and young plants, which are to be transplanted after germination or after emergence from soil.

These young plants may also be protected before transplantation by a total or partial treatment by immersion or pouring.

Preferably, a treatment of plant propagation materials with the compound of formula (I), the combination and or the composition thereof, respectively, is used for controlling a multitude of fungi on cereals, such as wheat, rye, barley and oats; on rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein). Genetically modified plants are plants, which genetic material has been so modified by recombinant DNA techniques that otherwise cannot readily be obtained by cross breeding under natural circumstances, mutations or natural recombination. Typically, one or more genes have been integrated into the genetic material of a genetically modified plant to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties. Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i.e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e.g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1 185; and references quoted therein. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox, or ExpressSun® sunflowers (DuPont, USA) being tolerant to sulfonyl ureas, e.g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants capable to synthesize one or more insecticidal proteins, especially those known from the bacterial genus (Bacillus), by use of recombinant DNA techniques are within the scope of the present invention. The Bacillus are particularly from Bacillus thuringiensis, such as δ-endotoxins, e.g. Cry1A(b), Cry1A(c), Cry1F, Cry1F(a2), Cry11A(b), Cry111A, Cry111B(b1) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO02/015701). Further examples of such toxins or genetically modified plants capable of synthesizing such toxins are disclosed, e.g., in EP374753, WO93/007278, WO95/34656, EP427529, EP451878, WO03/18810 and WO03/52073. The methods for producing such genetically modified plants are generally known to the person skilled in the art and are described, e.g. in the publications mentioned above. These insecticidal proteins contained in the genetically modified plants impart to the plants, producing these proteins, tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e.g., described in the publications mentioned above, and some of which are commercially available such as YieldGard® (corn cultivars producing the Cry1Ab toxin), YieldGard® Plus (corn cultivars producing Cry1Ab and Cry3Bb 1 toxins), Starlink® (corn cultivars producing the Cry9c toxin), Herculex® RW (corn cultivars producing Cry34Ab1, Cry35Ab1 and the enzyme phosphinothricin N-acetyltransferase [PAT]); NuCOTN® 33B (cotton cultivars producing the Cry1Ac toxin), Bollgard® I (cotton cultivars producing the Cry1 Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e.g. Agrisure® CB) and Bt176 from Syngenta Seeds SAS, France, (corn cultivars producing the Cry1Ab toxin and PAT enyzme), MIR604 from Syngenta Seeds SAS, France (corn cultivars producing a modified version of the Cry3A toxin, c.f. WO03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants capable to synthesize one or more proteins to increase the resistance or tolerance of those plants to bacterial, viral or fungal pathogens by the use of recombinant DNA techniques are also within the scope of the present invention. Examples of such proteins are the so-called “pathogenesis-related proteins” (PR proteins, see, e.g. EP392225), plant disease resistance genes (e.g. potato cultivars, which express resistance genes acting against Phytophthora infestans derived from the Mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g. in the publications mentioned above.

Furthermore, plants capable to synthesize one or more proteins, by the use of recombinant DNA techniques, to increase the productivity (e.g. biomass production, grain yield, starch content, oil content or protein content), tolerance to drought, salinity or other growth-limiting environmental factors or tolerance to pests and fungal, bacterial or viral pathogens of those plants are within the scope of the present invention.

Furthermore, plants that contain a modified quantity of substances of content or new substances of content, by the use of recombinant DNA techniques, to improve human or animal nutrition, e.g. oil crops that produce health-promoting long-chain omega-3 fatty acids or unsaturated omega-9 fatty acids (e.g. Nexera® rape, DOW Agro Sciences, Canada) are also within the scope of the present invention.

Furthermore, plants that contain a modified quantity of substances of content or new substances of content, by the use of recombinant DNA techniques, to improve raw material production, e.g. potatoes that produce increased amounts of amylopectin (e.g. Amflora® potato, BASF SE, Germany) are also within the scope of the present invention.

The compounds of Formula (I) may be, for example, effective against fungi and fungal vectors of disease as well as phytopathogenic bacteria and viruses. These fungi and fungal vectors of disease as well as phytopathogenic bacteria and viruses are for example:
Absidia corymbifera, Alternaria spp, Aphanomyces spp, Ascochyta spp, Aspergillus spp. including A. flavus, A. fumigatus, A. nidulans, A. niger, A. terms, Aureobasidium spp. including A. pullulans, Blastomyces dermatitidis, Blumeria graminis, Bremia lactucae, Botryosphaeria spp. including B. dothidea, B. obtusa, Botrytis spp. including B. cinerea, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, Cercosporidium personatum, Cladosporium spp, Claviceps purpurea, Coccidioides immitis, Cochliobolus spp, Colletotrichum spp. including C. musae, Cryptococcus neoformans, Colletotrichum capsici, Diaporthe spp, Didymella spp, Drechslera spp, Elsinoe spp, Epidermophyton spp, Erwinia amylovora, Erysiphe spp. including E. cichoracearum, Eutypa lata, Fusarium spp. including F. culmorum, F. graminearum, F. langsethiae, F. moniliforme, F. oxysporum, F. proliferatum, F. subglutinans, F. solani, Gaeumannomyces graminis, Gibberella fujikuroi, Gloeodes pomigena, Gloeosporium musarum, Glomerella cingulate, Guignardia bidwellii, Gymnosporangium juniperi-virginianae, Helminthosporium spp, Hemileia spp, Histoplasma spp. including H. capsulatum, Laetisaria fuciformis, Leptographium lindbergi, Leveillula taurica, Lophodermium seditiosum, Fusarium culmorum, Gibberella zeae (Fusarium graminearum), Mycosphaerella arachidis (Cercospora arachidicola), Pyrenophora teres, Blumeria graminisf sp. tritici (Erysiphe graminis f. sp. tritici), Microdochium nivale, Microsporum spp, Monilinia spp, Mucor spp, Mycosphaerella spp. including M. graminicola, M. pomi, Oncobasidium theobromaeon, Ophiostoma piceae, Paracoccidioides spp, Penicillium spp. including P. digitatum, P. italicum, Petriellidium spp, Peronosclerospora spp. Including P. maydis, P. philippinensis and P. sorghi, Peronospora spp, Parastagonospora nodorum, Phaeosphaeria nodorum, Phakopsora pachyrhizi, Phellinus igniarus, Phialophora spp, Phoma spp, Phomopsis viticola, Phytophthora spp. including P. infestans, Plasmopara spp. including P. halstedii, P. viticola, Pleospora spp., Podosphaera spp. including P. leucotricha, Polymyxa graminis, Polymyxa betae, Pseudocercosporella herpotrichoides, Pseudomonas spp, Pseudoperonospora spp. including P. cubensis, P. humuli, Pseudopeziza tracheiphila, Puccinia spp. including P. hordei, P. recondita, P. striiformis, P. triticina, Pyrenopeziza spp, Pyrenophora spp, Pyricularia spp. including P. oryzae, Pythium spp. including P. ultimum, Ramularia spp, Rhizoctonia spp, Rhizomucor pusillus, Rhizopus arrhizus, Rhynchosporium spp, Scedosporium spp. including S. apiospermum and S. prolificans, Schizothyrium pomi, Sclerotinia spp, Sclerotium spp, Septoria spp, including S. nodorum, S. tritici, Septoria lycopersici, Sphaerotheca macularis, Sphaerotheca fusca (Sphaerotheca fuliginea), Sporothorix spp, Stagonospora nodorum, Stemphylium spp., Stereum hirsutum, Thanatephorus cucumeris, Thielaviopsis basicola, Tilletia spp, Trichoderma spp., including T. harzianum, T. pseudokoningii, T. viride, Trichophyton spp, Typhula spp, Uncinula necator, Urocystis spp, Ustilago spp, Venturia spp. including V. inaequalis, Verticillium spp., and Xanthomonas spp, Ustilaginales such as Ustilaginoidea virens, Ustilago nuda, Ustilago tritici, Ustilago zeae, Cerotelium fici, Chrysomyxa arctostaphyli, Coleosporium ipomoeae, Hemileia vastatrix, Puccinia arachidis, Puccinia cacabata, Puccinia graminis, Puccinia sorghi, Puccinia striiformis f.sp. Hordei, Puccinia striiformis f.sp. Secalis, Pucciniastrum coryli, or Uredinales such as Cronartium ribicola, Gymnosporangium juniperi-virginianae, Melampsora medusae, Phragmidium mucronatum, Physopella ampelosidis, Tranzschelia discolor and Uromyces viciae-fabae, Cryptococcus spp., Exobasidium vexans, Marasmiellus inoderma, Mycena spp., Sphacelotheca reiliana, Typhula ishikariensis, Urocystis agropyri, Itersonilia perplexans, Corticium invisum, Waitea circinata, Rhizoctonia solani, Thanetephorus cucurmeris, Entyloma dahliae, Entylomella microspora, Neovossia moliniae and Tilletia caries, Blastocladiomycetes, such as Physoderma maydis, Mucoromycetes, such as Choanephora cucurbitarum.

Non-limiting examples of pathogens of fungal diseases which can be treated in accordance with the invention include the diseases caused by rust disease pathogens, for example Gymnosporangium species, for example Gymnosporangium sabinae; Hemileia species, for example Hemileia vastatrix, Phakopsora species, for example Phakopsora pachyrhizi or Phakopsora meibomiae; Puccinia species, for example Puccinia recondita, Puccinia graminis oder Puccinia striiformis; Uromyces species, for example Uromyces appendiculatus.

In particular, Cronartium ribicola (White pine blister rust); Gymnosporangium juniperi-virginianae (Cedar-apple rust); Hemileia vastatrix (Coffee rust); Phakopsora meibomiae and P. pachyrhizi (Soybean rust); Puccinia coronata (Crown Rust of Oats and Ryegrass); Puccinia graminis (Stem rust of wheat and Kentucky bluegrass, or black rust of cereals); Puccinia hemerocallidis (Daylily rust); Puccinia persistens subsp. triticina (wheat rust or ‘brown or red rust’); Puccinia sorghi (rust in corn); Puccinia striiformis (‘Yellow rust’ in cereals); Uromyces appendiculatus (rust of beans); Uromyces phaseoli (Bean rust); Puccinia melanocephala (‘Brown rust’ in sugarcane); Puccinia kuehnii (‘Orange rust’ in sugarcane).

The present invention further relates to the use of the compound of Formula (I), the combination or the composition thereof for controlling or preventing against phytopathogenic fungi such as Septoria spp., powdery mildews, Botrytis spp., anthracnose, Alternaria spp., scab and Monilinia spp. of agricultural crops and or horticultural crops.

The present invention further relates to the use of the compound of Formula (I), the combination or the composition thereof for controlling or preventing against phytopathogenic fungi such as Septoria spp., powdery mildews, Botrytis spp., leaf spot diseases like anthracnose, Alternaria spp., apple scab, Cercospora spp., Corynespora ssp. and Monilinia spp. in cereals, soybeans, grapevines, fruits, nuts and vegetables.

Plants which can be treated in accordance with the invention include the following: cotton, flax, grapevine, fruits, vegetables, such as Rosaceae sp (for example pome fruits such as apples, pears, apricots, cherries, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Vitaceae sp. (for example grapes); Solanaceae sp. (for example tomatoes, peppers), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leek, onion), Papilionaceae sp. (for example peas); major crop plants, such as Poaceae/Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak choi, kohlrabi, radishes, and oilseed rape, mustard, horseradish and cress), Fabacae sp. (for example bean, peanuts), Papilionaceae sp. (for example soya bean), Solanaceae sp. (for example potatoes), Chenopodiaceae sp. (for example sugar beet, fodder beet, swiss chard, beetroot); Malvaceae (for example cotton); useful plants and ornamental plants for gardens and wooded areas; and genetically modified varieties of each of these plants.

More preference is given to controlling the following diseases of soya beans: Fungal diseases on leaves, stems, pods and seeds caused, for example, by Altemaria leaf spot (Altemaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectiia crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti, Fusarium culmorum), net blotch (Pyrenophora teres), powdery mildew (Blumeria graminisf sp. Tritici), leaf spot (Cercospora arachidicola), fusarium head blight (Gibberella zeae), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidennatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The present invention also relates to the use of the compound of Formula (I), the combination or the composition thereof for controlling or preventing the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye and Phakopsoraceae spp. on various plants, in particular Phakopsora pachyrhizi and P. meibomiae (soybean rust) on soybeans, Hemileia vastatrix (Coffee rust), Uromyces appendiculatus, Uromyces fabae and Uromyces phaseoli (rust of beans).

The compound, the combination and the composition of the present invention can be used for controlling or preventing plant diseases. The compound of Formula (I), the combination and/or the composition thereof, respectively, are particularly suitable for controlling the following plant diseases: Albugo spp. (white rust) on ornamentals, vegetables (e.g. A. Candida) and sunflowers (e.g. A. tragopogonis); Altemaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (C. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping oil) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chiamydospora (earlier Phaeoacremonium chlamydosporum), Phaeoacremonium aleophilum and/or Botryosphaeria obtusa, Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (L. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-of) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), soybeans, potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or .rotbrenner′, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, P. kuehnii (orange rust) on sugar cane and P. asparagi on asparagus; Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-of) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria]nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

In one embodiment, the compounds of Formula (I) have broad ranges of activity against fungal pathogens. Exemplary pathogens may include, but are not limited to, the causing agent of wheat leaf blotch (Zymoseptoria tritici), wheat brown rust (Puccinia triticina), wheat stripe rust (Puccinia striiformis), scab of apple (Venturia inaequalis), powdery mildew of grapevine (Uncinula necator), barley scald (Rhynchosporium secalis), blast of rice (Pyricularia oryzae), rust of soybean (Phakopsora pachyrhizi), glume blotch of wheat (Leptosphaeria nodorum), powdery mildew of wheat (Blumeria graminis f. sp. tritici), powdery mildew of barley (Blumeria graminis f. sp. hordei), powdery mildew of cucurbits (Erysiphe cichoracearum), anthracnose of cucurbits (Colletotrichum lagenarium), leaf spot of beet (Cercospora beticola), early blight of tomato (Alternaria solani), and spot blotch of barley (Cochliobolus sativus), gray mold (Botrytis cinerea), common root rot (Fusarium culmorum), white mold (Sclerotinia sclerotiorum), pink snow mold (Microdochium nivale), fusarium head blight (Fusarium graminearum), Take-all (Gaeumannomyces graminis), Wheat leaf rust (Puccinia triticina), early leaf spot (Mycosphaerella arachidis), net blotch (Pyrenophora teres).

The exact amount of the active material to be applied is dependent not only on the specific active material being applied, but also on the particular action desired, the fungal species to be controlled, and the stage of growth thereof, as well as the parts of the plant or other products to be contacted with the compound. Thus, all the compounds, and formulations containing the same, may not be equally effective at similar concentrations or against the same fungal species.

The compound of Formula (I), the combination and the composition thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fibre or fabrics, against the infestation and destruction by harmful microorganisms, such as fungi and bacteria.

As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Pora spp., Serpula spp. and Tyromyces spp., Deuteromycetes such as Aspergillus spp., Cladosporium spp., Penicillium spp., Trichoderma spp., Altemaria spp., Paecilomyces spp. and Zygomycetes such as Mucor spp., and in addition in the protection of stored products and harvest the following yeast fungi are worthy of note: Candida spp. and Saccharomyces cerevisae.

In one embodiment, the present invention provides a method for controlling or preventing phytopathogenic fungi. The method comprises treating the fungi or the materials, plants, plant parts, locus thereof, soil or seeds to be protected against fungal attack, with an effective amount of at least one compound of Formula (I) or a combination or a composition comprising at least one compound of Formula (I).

The method of treatment according to the invention can also be used in the field of protecting stored products or harvest against the attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in their processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combination according to the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The treatment of the plants and plant parts with the compounds of the Formula (I) is carried out directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, furthermore as a powder for dry seed treatment, a solution for liquid seed treatment, a water-soluble powder for slurry treatment, by incrusting, by coating with one or more coats, etc. It is furthermore possible to apply the compounds of the formula (I) by the ultra-low volume method or to inject the application form or the compound of the formula (I) itself into the soil. A preferred direct treatment of the plants is foliar application, i.e. the compounds of the formula (I) are applied to the foliage, where treatment frequency and the application rate should be adjusted according to the level of infestation with the disease in question.

In the case of systemically active compounds, the compounds of the Formula (I) also access the plants via the root system. The plants are then treated by the action of the compounds of the Formula (I) on the habitat of the plant. This may be done, for example, by drenching, or by mixing into the soil or the nutrient solution, i.e. the locus of the plant (e.g. soil or hydroponic systems) is impregnated with a liquid form of the compounds of the Formula (I), or by soil application, i.e. the compounds of the Formula (I) according to the invention are introduced in solid form (e.g. in the form of granules) into the locus of the plants, or by drip application (often also referred to as “chemigation”), i.e. the liquid application of the compounds of the Formula (I) according to the invention from surface or sub-surface driplines over a certain period of time together with varying amounts of water at defined locations in the vicinity of the plants. In the case of paddy rice crops, this can also be done by metering the compound of the Formula (I) in a solid application form (for example as granules) into a flooded paddy field.

The compounds of the invention can be used in combination with models e.g. embedded in computer programs for site specific crop management, satellite farming, precision farming or precision agriculture. Such models support the site specific management of agricultural sites with data from various sources such as soils, weather, crops (e.g. type, growth stage, plant health), weeds (e.g. type, growth stage), diseases, pests, nutrients, water, moisture, biomass, satellite data, yield etc. with the purpose to optimize profitability, sustainability and protection of the environment. In particular, such models can help to optimize agronomical decisions, control the precision of pesticide applications and record the work performed.

As an example, the compounds of the invention can be applied to a crop plant according to an appropriate dose regime if a model models the development of a pest and calculates that a threshold has been reached for which it is recommendable to apply the compound of the invention to the crop plant Commercially available systems which include agronomic models are e.g. FieldScripts™ from The Climate Corporation, Xarvio™ from BASF, AGLogic™ from John Deere, etc.

The compounds of the invention can also be used in combination with smart spraying equipment such as e.g. spot spraying or precision spraying equipment attached to or housed within a farm vehicle such as a tractor, robot, helicopter, airplane, unmanned aerial vehicle (UAV) such as a drone, etc. Such an equipment usually includes input sensors (such as e.g. a camera) and a processing unit configured to analyze the input data and configured to provide a decision based on the analysis of the input data to apply the compound of the invention to the crop plants (respectively the weeds) in a specific and precise manner. The use of such smart spraying equipment usually also requires positions systems (e.g. UPS receivers) to localize recorded data and to guide or to control farm vehicles; geographic information systems (GIS) to represent the information on intelligible maps, and appropriate farm vehicles to perform the required farm action such as the spraying.

Any range or desired value given herein may be extended or altered without losing the effects sought, as is apparent to the skilled person for an understanding of the teachings herein.

The invention disclosed in the present disclosure shall now be elaborated with the help of non-limiting examples.

### CHEMISTRY EXAMPLES

### Example 1: Preparation of 3-cyclopropyl-1-methyl-1H-pyrrolo[2,3-b]pyridine-5-carbonitrile

### a) Step 1: Preparation of 5-bromo-1-methyl-1H-pyrrolo[2,3-b]pyridine

To a stirred solution of 5-bromo-1H-pyrrolo[2,3-b]pyridine (600 mg, 3.05 mmol) in dimethylformamide (20 mL), sodium hydride (183 mg, 4.57 mmol, 60% dispersion in mineral oil) was added at 0° C. and the resulting reaction mixture was stirred at 0° C. for 20 min. To this reaction mixture, methyl iodide (0.19 mL, 3.05 mmol) was added and the reaction mixture was further stirred at 25° C. for 3 h. After the completion of the reaction, the reaction mixture was quenched with saturated ammonium chloride (20 mL) and extracted with ethyl acetate (3×20 mL). The organic layer was washed with brine solution (20 mL), dried over anhydrous sodium sulphate, concentrated and purified by column chromatography to obtain 5-bromo-1-methyl-1H-pyrrolo[2,3-b]pyridine (400 mg, 1.9 mmol, 62% yield). ¹H-NMR (400 MHz, DMSO-d6) δ 8.32-8.42 (m, 1H), 8.21 (d, J=2.2 Hz, 1H), 7.59 (t, J=3.7 Hz, 1H), 6.46 (d, J=3.4 Hz, 1H), 3.64-3.82 (m, 3H).

### b) Step 2: Preparation of 1-methyl-1H-pyrrolo[2,3-b]pyridine-5-carbonitrile

5-bromo-1-methyl-1H-pyrrolo[2,3-b]pyridine (1.0 g, 4.74 mmol), zinc cyanide (1.11 g, 9.48 mmol), tris(dibenzylideneacetone)dipalladium(0) (0.22 g, 0.24 mmol) and 2-dicyclohexylphosphino-2′,4′,6′-tri-iso-propyl-1,1′-biphenyl (0.12 g, 0.24 mmol) were taken in dimethylformamide (10 mL)/water (0.5 mL). The resulting reaction mixture was heated in a microwave at 150° C. for 1 h. After the completion of the reaction, the reaction mixture was quenched with water (20 mL) and extracted with ethyl acetate (3×30 mL). The organic layer was washed with brine, dried over anhydrous sodium sulphate, concentrated and purified by column chromatography on silica gel to obtain 1-methyl-1H-pyrrolo[2,3-b]pyridine-5-carbonitrile (500 mg, 3.18 mmol, 67% yield). ¹H-NMR (400 MHz, DMSO-d6) δ 8.64 (d, J=2.0 Hz, 1H), 8.51 (d, J=2.0 Hz, 1H), 7.71-7.75 (m, 1H), 6.63 (d, J=3.7 Hz, 1H), 3.85 (s, 3H); ESI MS (m/z) 158.10 (MH)⁺.

### c) Step 3: Preparation of 3-bromo-1-methyl-1H-pyrrolo[2,3-b]pyridine-5-carbonitrile

To a stirred solution of 1-methyl-1H-pyrrolo[2,3-b]pyridine-5-carbonitrile (5.0 g, 31.8 mmol) in dichloromethane (50 mL), N-bromosuccinimide (6.23 g, 35.0 mmol) was added at 25° C. under a nitrogen atmosphere and the resulting reaction mixture was stirred for 1 h. After the completion of the reaction, the reaction mixture was added to water (100 mL), extracted with ethyl acetate (3×100 mL), dried over anhydrous sodium sulphate, concentrated and purified by column chromatography to obtain 3-bromo-1-methyl-1H-pyrrolo[2,3-b]pyridine-5-carbonitrile (6.9 g, 29.2 mmol, 92% yield). ¹H-NMR (400 MHz, CDCl₃) δ 8.59-8.55 (m, 1H), 8.14 (d, J=2.0 Hz, 1H), 7.37 (s, 1H), 3.90 (s, 3H).

### d) Step 4: Preparation of 3-cyclopropyl-1-methyl-1H-pyrrolo[2,3-b]pyridine-5-carbonitrile

To a stirred solution of 3-bromo-1-methyl-1H-pyrrolo[2,3-b]pyridine-5-carbonitrile (2.5 g, 10.59 mmol) in toluene (25 mL), sodium carbonate (3.37 g, 31.8 mmol) was added at 25° C. and nitrogen was purged for 20 min. The resulting reaction mixture was stirred at 100° C. for 5 h. After the completion of the reaction, the reaction mixture was added to water (100 mL) and ethyl acetate (100 mL) and filtered. The organic layer was separated, dried over anhydrous sodium sulphate, concentrated and purified by column chromatography to obtain 3-cyclopropyl-1-methyl-1H-pyrrolo[2,3-b]pyridine-5-carbonitrile (1.7 g, 8.62 mmol, 81% yield). ¹H-NMR (400 MHz, CDCl₃) δ 8.54 (d, J=2.0 Hz, 1H), 8.25 (d, J=2.0 Hz, 1H), 7.00 (d, J=0.7 Hz, 1H), 3.84 (s, 3H), 1.91-1.87 (m, 1H), 0.96-0.91 (m, 2H), 0.64-0.61 (m, 2H).

### Example 2: Preparation of 3-chloro-1-methyl-1H-pyrrolo[2,3-b]pyridine-5-carbonitrile

To a stirred solution of 1-methyl-1H-pyrrolo[2,3-b]pyridine-5-carbonitrile (2.5 g, 15.9 mmol) in dichloromethane (50 mL), N-chlorosuccinimide (2.13 g, 15.9 mmol) was added at 25° C. under nitrogen atmosphere and the reaction mixture was stirred at 25° C. for 6 h. After the completion of the reaction, the reaction mixture was poured into water (100 mL). The crude product was extracted with ethyl acetate (3×100 mL), dried over anhydrous sodium sulphate, concentrated and purified by column chromatography to obtain 3-chloro-1-methyl-1H-pyrrolo[2,3-b]pyridine-5-carbonitrile (2.5 g, 13.0 mmol, 82% yield). ¹H-NMR (400 MHz, CDCl₃) δ 8.59 (d, J=2.0 Hz, 1H), 8.21 (d, J=2.0 Hz, 1H), 7.31 (s, 1H), 3.91 (s, 3H); ESI MS (m/z) 191.95 (MH)⁺.

### Example 3: Preparation of 1-methyl-1H-pyrrolo[2,3-b]pyridine-5-carboxylic acid

To a stirred solution of 1-methyl-1H-pyrrolo[2,3-b]pyridine-5-carbonitrile (0.5 g, 3.18 mmol) in acetic acid (10 mL), sulfuric acid (1.696 mL, 31.8 mmol) was slowly added at 25° C. The resulting reaction mixture was stirred at 100° C. for 4 h. After the completion of the reaction, acetic acid was evaporated from the reaction mixture to obtain a crude solid product. The obtained crude product was washed with hexane (20 mL) to obtain 1-methyl-1H-pyrrolo[2,3-b]pyridine-5-carboxylic acid (0.55 g, 3.12 mmol, 98% yield). ¹H-NMR (400 MHz, DMSO-d6) δ 8.80 (d, J=2.0 Hz, 1H), 8.53 (d, J=2.0 Hz, 1H), 7.64 (d, J=3.4 Hz, 1H), 6.62 (d, J=3.4 Hz, 1H), 3.85 (s, 3H); ESI MS (m/z) 176.95.

### Example 4: Preparation of 2-oxo-2,3-dihydrooxazolo[4,5-b]pyridine-6-carboxylic acid

### a) Step 1: Preparation of methyl 2-oxo-2,3-dihydrooxazolo[4,5-b]pyridine-6-carboxylate

To a stirred solution of methyl 6-amino-5-hydroxynicotinate (1 g, 5.95 mmol) in dimethylformamide (10 mL), 1,1-carbonyldiimidazole (1.45 g, 8.92 mmol) was added under an inert atmosphere at 25° C. and the resulting reaction mixture was heated at 80° C. for 2 h. After the completion of the reaction, the reaction mixture was cooled and dimethylformamide was evaporated under reduced pressure. The residue was acidified with 1 N hydrochloric acid to obtain a brown precipitate. The solid material was filtered, washed with water (20 mL) and dried under reduced pressure to obtain methyl 2-oxo-2,3-dihydrooxazolo[4,5-b]pyridine-6-carboxylate (1.1 g, 5.67 mmol, 95% yield). ¹H-NMR (400 MHz, CDCl₃) δ 8.86 (d, J=1.7 Hz, 1H), 8.03 (d, J=1.7 Hz, 1H), 3.97 (s, 3H).

### b) Step 2: Preparation of 2-oxo-2,3-dihydrooxazolo[4,5-b]pyridine-6-carboxylic acid

To a stirred solution of methyl 2-oxo-2,3-dihydrooxazolo[4,5-b]pyridine-6-carboxylate (1.1 g, 5.67 mmol) in methanol (20 mL) and water (2 mL), lithium hydroxide (0.41 g, 17 mmol) was added at 25° C. and the resulting reaction mixture was stirred at 75° C. for 2 h. After the completion of the reaction, the reaction mixture was cooled and methanol was evaporated under reduced pressure to obtain residue. The obtained residue was then neutralized with 10 N hydrochloric acid to obtain a precipitate. The solid material was filtered, washed with water (20 mL) and dried under reduced pressure to obtain 2-oxo-2,3-dihydrooxazolo[4,5-b]pyridine-6-carboxylic acid (0.88 g, 4.86 mmol, 86% yield) as off white solid. ¹H-NMR (400 MHz, DMSO-d6) δ 13.15-12.90 (bs, 2H), 8.61 (d, J=1.7 Hz, 1H), 7.96 (d, J=1.7 Hz, 1H).

### Example 5: Preparation 2-methoxy-6,7-dihydro-5H-cyclopenta[b]pyridine-3-carboxylic acid

### a) Step 1: Preparation of 2-chloro-6,7-dihydro-5H-cyclopenta[b]pyridine-3-carboxylic acid

To a stirred solution of phosphorus oxychloride (42.9 mL, 460 mmol), 2-oxo-2,5,6,7-tetrahydro-1H-cyclopenta[b]pyridine-3-carboxylic acid (5.50 g, 30.7 mmol) was added at 25° C. The resulting reaction mixture was stirred at 110° C. for 5 h. After the completion of the reaction, the reaction mixture was evaporated under reduced pressure and diluted with water (50 mL). The aqueous layer was extracted with ethyl acetate (3×200 mL). The organic layer was dried over anhydrous sodium sulphate, concentrated and purified by column chromatography to obtain 2-chloro-6,7-dihydro-5H-cyclopenta[b]pyridine-3-carboxylic acid (2.180 g, 11.03 mmol, 36% yield). 1H-NMR (400 MHz, CHLOROFORM-D) δ 8.14 (s, 1H), 3.09 (t, J=7.8 Hz, 2H), 3.03 (d, J=7.6 Hz, OH), 2.99 (t, J=7.3 Hz, 2H), 2.27-2.14 (m, 2H); MS (m/z) 197.85 (MH)⁺.

### b) Step 2: Preparation of 2-methoxy-6,7-dihydro-5H-cyclopenta[b]pyridine-3-carboxylic acid

To a stirred solution of 2-chloro-6,7-dihydro-5H-cyclopenta[b]pyridine-3-carboxylic acid (3.0 g, 15.18 mmol) in methanol (5 mL), sodium methoxide solution (30% in methanol) (14.09 mL, 76 mmol) was added at 25° C. The resulting reaction mixture was stirred at 80° C. for 24 h. After completion of the reaction, the reaction mixture was evaporated under reduced pressure and diluted with water (10 mL), the aqueous layer was adjusted to pH 3 with concentrated hydrochloric acid to obtain precipitate. The precipitated aqueous layer was extracted with ethyl acetate (3×200 mL). The organic layer was dried over anhydrous sodium sulphate, concentrated and purified by column chromatography to obtain 2-methoxy-6,7-dihydro-5H-cyclopenta[b]pyridine-3-carboxylic acid (2.45 g, 12.68 mmol, 84% yield). ¹H-NMR (400 MHz, CHLOROFORM-D) δ 8.24 (s, 1H), 4.17 (s, 3H), 3.00-2.86 (m, 4H), 2.27-2.11 (m, 2H); ESI MS (m/z) 193.9 (MH)⁺.

### Example 6: Preparation 2-methoxy-5,6,7,8-tetrahydroquinoline-3-carboxylic acid

### a) Step 1: Preparation of Sodium (E)-(2-oxocyclohexylidene)methanolate

To a stirred suspension of sodium ethoxide (27.0 g, 397 mmol) in diethyl ether (200 mL), cyclohexanone (31.6 mL, 306 mmol) and ethyl formate (24.59 mL, 306 mmol) were added under nitrogen atmosphere at 0° C. The resulting reaction mixture was stirred at 10-15° C. for 24 h. After completion of the reaction, the precipitated product was filtered, washed with hexane (2×20 mL) and dried under reduced pressure to obtain sodium (E)-(2-oxocyclohexylidene)methanolate (32.84 g, 222 mmol, 73% yield). ¹H-NMR (400 MHz, D₂O) δ 8.95 (s, 1H), 8.32 (s, 1H), 2.10-2.03 (m, 4H), 1.60-1.52 (m, 2H), 1.51-1.43 (m, 2H).

### b) Step 2: Preparation of 2-hydroxy-5,6,7,8-tetrahydroquinoline-3-carbonitrile

To a stirred solution of sodium (E)-(2-oxocyclohexylidene)methanolate (30 g, 203 mmol) in water (200 mL), 2-cyanoacetamide (17.88 g, 213 mmol) and piperidine acetate (30 mL) (prepared using 6.81 mL of acetic acid, 11.52 mL of water and 11.77 mL of piperidine) were added at 25° C. The resulting reaction mixture was stirred at 100° C. for 12 h. After completion of the reaction, the reaction mixture was cooled to 25° C. and acetic acid (20 mL) was added followed by stirring for additional 1 h at 25° C. The resulting reaction mixture was cooled to 0° C. to obtain precipitate. The obtained precipitate was filtered, washed with water and dried to obtain 2-hydroxy-5,6,7,8-tetrahydroquinoline-3-carbonitrile (21.49 g, 123 mmol, 61% yield). ¹H-NMR (400 MHz, DMSO-d6) δ 7.87 (s, 1H), 2.54 (t, J=5.9 Hz, 3H), 2.41 (t, J=5.9 Hz, 2H), 1.77-1.61 (m, 4H); ESI MS (m/z) 174.95 (MH)⁺.

### c) Step 3: Preparation of 2-hydroxy-5,6,7,8-tetrahydroquinoline-3-carboxylic acid

A suspension of 2-hydroxy-5,6,7,8-tetrahydroquinoline-3-carbonitrile (10 g, 57.4 mmol) in concentrated hydrochloric acid (60 mL, 37%) was stirred at 100-110° C. for 12 h. After completion of the reaction, the reaction mixture was evaporated under reduced pressure and dried to obtain 2-hydroxy-5,6,7,8-tetrahydroquinoline-3-carboxylic acid (10.05 g, 52.0 mmol, 91% yield). ¹H-NMR (400 MHz, DMSO-d6) δ14.91 (s, 1H), 13.05 (s, 1H), 8.11 (s, 1H), 2.65 (t, J=6.0 Hz, 2H), 2.55 (t, J=5.9 Hz, 2H), 1.75-1.64 (m, 4H); ESI MS (m/z) 193.90 (MH)⁺.
d) Step 4: Preparation of 2-chloro-5,6,7,8-tetrahydroquinoline-3-carboxylic acid

To a stirred solution of phosphorus oxychloride (28.9 mL, 311 mmol), 2-hydroxy-5,6,7,8-tetrahydroquinoline-3-carboxylic acid (3.0 g, 15.53 mmol) was added at 25° C. The resulting reaction mixture was stirred at 110° C. for 5 h. After completion of the reaction, the reaction mixture was evaporated under reduced pressure and diluted with water (50 mL). The aqueous layer was extracted with ethyl acetate (3×200 mL). The organic layer was dried over anhydrous sodium sulphate, concentrated to obtain 2-chloro-5,6,7,8-tetrahydroquinoline-3-carboxylic acid (1.97 g, 9.30 mmol, 60% yield). ¹H-NMR (400 MHz, CHLOROFORM-D) δ 8.02 (s, 1H), 2.95 (t, J=6.4 Hz, 2H), 2.79 (t, J=6.3 Hz, 2H), 1.94-1.80 (m, 4H); ESI MS (m/z) 211.85 (MH)⁺.
e) Step 5: Preparation of 2-methoxy-5,6,7,8-tetrahydroquinoline-3-carboxylic acid

To a stirred solution of 2-chloro-5,6,7,8-tetrahydroquinoline-3-carboxylic acid (1.0 g, 4.72 mmol) in methanol (5 mL), sodium methoxide solution (30% in methanol) (8.77 mL, 47.2 mmol) was added at 25° C. The resulting reaction mixture was stirred at 60° C. for 48 h. After completion of the reaction, the reaction mixture was evaporated under reduced pressure and diluted with water (10 mL), the aqueous layer was acidified with 1 N HCl (10 mL) and extracted with ethyl acetate (2×150 mL). The organic layer was dried over anhydrous sodium sulphate, concentrated under reduced pressure to obtain 2-methoxy-5,6,7,8-tetrahydroquinoline-3-carboxylic acid (740 mg, 3.6 mmol, 74% yield). ¹H-NMR (400 MHz, CHLOROFORM-D) δ 8.11 (s, 1H), 4.13 (s, 3H), 2.86-2.72 (m, 4H), 1.88-1.80 (m, 4H); ESI MS (m/z) 207.95 (MH)⁺.

### Example 7: Preparation of 2,4-dimethyl-1-phenylpentan-2-amine

### a) Step 1: Preparation of 2,4-dimethyl-1-phenylpentan-2-ol

To a stirred solution of 4-methylpentan-2-one (5 g, 49.9 mmol) in tetrahydrofuran (40 mL), benzylmagnesium chloride (37.4 mL, 74.9 mmol) was added dropwise at 0° C. and the resulting reaction mixture was allowed to warm to 25° C. and stirred at 25° C. for 3 h. After completion of the reaction, the reaction mixture was quenched with 2 N hydrochloric acid (50 mL) and extracted with ethyl acetate (3×100 mL). The organic layer was dried over anhydrous sodium sulphate, concentrated and purified by column chromatography to obtain 2,4-dimethyl-1-phenylpentan-2-ol (4 g, 42% yield). ¹H-NMR (400 MHz, CDCl₃) δ 7.29-7.33 (m, 2H), 7.27 (d, J=1.5 Hz, 1H), 7.20-7.25 (m, 3H), 2.80 (d, J=13.4 Hz, 1H), 2.71 (d, J=13.1 Hz, 1H), 1.85-1.95 (m, 1H), 1.36-1.48 (m, 2H), 1.16 (s, 3H), 0.90-1.00 (m, 6H).

### b) Step 2: Preparation of 2-chloro-N-(2,4-dimethyl-1-phenylpentan-2-yl)acetamide

To a stirred solution of 2-chloroacetonitrile (0.99 mL, 15.60 mmol) and 2,4-dimethyl-1-phenylpentan-2-ol (2 g, 10.40 mmol) in acetic acid (6 mL), sulfuric acid (2.0 ml, 37.5 mmol) was added dropwise at 0° C. The resulting reaction mixture was stirred at 25° C. for 16 h. After completion of the reaction, the reaction mixture was neutralized with saturated sodium bicarbonate solution (20 mL) and extracted with ethyl acetate (3×20 mL). The organic layer was washed with brine (20 mL), dried over anhydrous sodium sulphate, concentrated and purified by column chromatography to obtain 2-chloro-N-(2,4-dimethyl-1-phenylpentan-2-yl)acetamide (1.7 g, 6.35 mmol, 61% yield). ESI MS (m/z) 267.80 (MH)⁺.

### c) Step 3: Preparation of 2,4-dimethyl-1-phenylpentan-2-amine

To a stirred solution of 2-chloro-N-(2,4-dimethyl-1-phenylpentan-2-yl)acetamide (1.7 g, 6.35 mmol) and thiourea (0.58 g, 7.62 mmol) in ethanol (20 mL), acetic acid (3 mL, 52.4 mmol) was added at 25° C. The resulting reaction mixture was stirred at 80° C. for 36 h. After completion of the reaction, the reaction mixture was diluted with 1 N hydrochloric acid (20 mL) and extracted with ethyl acetate (2×20 mL). The aqueous layer was neutralized with 2 N sodium hydroxide and extracted with hexane (2×100 mL). The combined organic layers were concentrated under reduced pressure to obtain 2,4-dimethyl-1-phenylpentan-2-amine (0.7 g, 3.66 mmol, 58% yield). ESI MS (m/z) 191.80 (MH)⁺.

### Example 8: Preparation of 3-amino-3-methyl-1-phenylbutan-2-ol

### a) Step 1: Preparation of tort-butyl (1-hydroxy-2-methylpropan-2-yl)carbamate

To a solution of 2-amino-2-methylpropan-1-01 (4 g, 44.9 mmol) in dichloromethane (50 mL), di-tert-butyl dicarbonate (10.42 mL, 44.9 mmol) and triethyl amine (6.25 mL, 44.9 mmol) were added at 25° C. and the resulting reaction mixture was stirred for 2.5 h. After completion of the reaction, the reaction mixture was concentrated under reduced pressure and diluted with a 10% citric acid aqueous solution. The aqueous layer was extracted with ethyl acetate (3×50 mL). The organic layer was dried over anhydrous sodium sulphate, concentrated and purified by column chromatography to obtain tert-butyl (1-hydroxy-2-methylpropan-2-yl)carbamate (8 g, 42.3 mmol, 94% yield). ¹H-NMR (400 MHz, DMSO-d6) δ 6.09 (s, 1H), 4.68 (t, J=5.8 Hz, 1H), 3.28 (d, J=5.8 Hz, 2H), 1.35 (s, 9H), 1.12 (s, 6H).

### b) Step 2: Preparation of tort-butyl (2-methyl-1-oxopropan-2-yl)carbamate

To a solution of tert-butyl (1-hydroxy-2-methylpropan-2-yl)carbamate (10 g, 52.8 mmol)) in dichloromethane (50 mL), Dess-Martin periodinane (28.0 g, 66.0 mmol)) was added at 0° C. and the resulting reaction mixture was stirred at 25° C. for 16 h. After completion of the reaction, the reaction mixture was filtered and the filtrate was washed with saturated sodium thiosulphate solution (50 mL) and 10% sodium bicarbonate solution (100 mL). The organic layer was dried over anhydrous sodium sulphate, concentrated and purified by column chromatography to obtain tert-butyl (2-methyl-1-oxopropan-2-yl)carbamate as a white solid tert-butyl (2-methyl-1-oxopropan-2-yl)carbamate (8 g, 42.7 mmol, 81% yield). ¹H-NMR (400 MHz, DMSO-d6) δ 9.28 (s, 1H), 7.44 (s, 1H), 1.37 (s, 9H), 1.11 (s, 6H).

### c) Step 3: Preparation of tort-butyl (3-hydroxy-2-methyl-4-phenylbutan-2-yl)carbamate

To a stirred solution of tert-butyl (2-methyl-1-oxopropan-2-yl)carbamate (4.2 g, 22.43 mmol) in tetrahydrofuran (30 mL), benzyl magnesium chloride (2 M. solution in tetrahydrofuran, 33.6 mL, 67.3 mmol) was added dropwise at −78° C. and the resulting reaction mixture was stirred at the same temperature for 2 h. After completion of the reaction, the reaction mixture was quenched with water (50 mL) and the aqueous layer was extracted with ethyl acetate (3×50 mL). The organic layer was dried over anhydrous sodium sulphate, concentrated and purified by column chromatography to obtain tert-butyl (3-hydroxy-2-methyl-4-phenylbutan-2-yl)carbamate (2.1 g, 7.5 mmol, 34% yield). ¹H-NMR (400 MHz, DMSO-d6) δ 7.28-7.23 (m, 2H), 7.20-7.09 (m, 3H), 6.38 (s, 1H), 4.66 (d, J=7.1 Hz, 1H), 3.87-3.80 (m, 1H), 2.79 (d, J=13.0 Hz, 1H), 2.35-2.29 (m, 1H), 1.38 (s, 9H), 1.24 (s, 3H), 1.14 (s, 3H); ESI MS (m/z) 280.1 (MH)⁺.

### d) Step 4: Preparation of 3-amino-3-methyl-1-phenylbutan-2-ol

To a stirred solution of tert-butyl (3-hydroxy-2-methyl-4-phenylbutan-2-yl)carbamate (2.0 g, 7.16 mmol) in dichloromethane (20 mL), trifluoroacetic acid (5.52 mL, 71.6 mmol) was added dropwise at 0° C. and the resulting reaction mixture was allowed to warm to 25° C. and stirred for 1 h. After completion of the reaction, the reaction mixture was evaporated under reduced pressure and neutralized with sat. sodium carbonate (10 mL), the aqueous layer was extracted with dichloromethane (3×25 mL). The organic layer was dried over anhydrous sodium sulphate, concentrated and purified by column chromatography to obtain 3-amino-3-methyl-1-phenylbutan-2-ol (1.2 g, 6.7 mmol, 94% yield). ¹H-NMR (400 MHz, CHLOROFORM-D) δ 7.33-7.26 (m, 3H), 7.25-7.20 (m, 2H), 3.47 (dd, J=10.5, 2.4 Hz, 1H), 2.87 (dd, J=13.7, 2.2 Hz, 1H), 2.53 (dd, J=13.7, 10.5 Hz, 1H), 1.20 (s, 3H), 1.15 (s, 3H); ESI MS (m/z) 180.1 (MH)⁺.

### Example 9: Preparation of 2-amino-2-methyl-1-phenylbutan-2-ol

### a) Step 1: Preparation of tert-butyl (1-hydroxy-2-methyl-1-phenylpropan-2-yl)carbamate

To a stirred solution of tert-butyl (2-methyl-1-oxopropan-2-yl)carbamate (3.5 g, 18.7 mmol) in tetrahydrofuran (30 mL), phenyl magnesium bromide (18.7 mL, 2 M. solution in tetrahydrofuran, 37.4 mmol) was added under inert atmosphere at −78° C. and the resulting reaction mixture was stirred at the same temperature for 2 h. After completion of the reaction, the reaction mixture was quenched with water (50 mL) and the aqueous layer was extracted with ethyl acetate (3×50 mL). The organic layer was dried over anhydrous sodium sulphate, concentrated and purified by column chromatography to obtain tert-butyl (1-hydroxy-2-methyl-1-phenylpropan-2-yl)carbamate (2.7 g, 10.2 mmol, 54% yield). ¹H-NMR (400 MHz, CHLOROFORM-D) δ 7.32-7.27 (m, 5H), 5.07-5.28 (1H), 4.71 (s, 1H), 4.51 (s, 1H), 1.47 (s, 9H), 1.42 (s, 3H), 1.18 (s, 3H); ESI MS (m/z) 266.05 (MH)⁺.

### b) Step 2: Preparation of 2-amino-2-methyl-1-phenylpropan-1-ol

To a stirred solution of tert-butyl (1-hydroxy-2-methyl-1-phenylpropan-2-yl)carbamate (2.5 g, 9.42 mmol) in dichloromethane (10 mL), trifluoroacetic acid (3.63 mL, 47.1 mmol) was added dropwise at 0° C. and the resulting reaction mixture was allowed to warm to 25° C. and stirred for 1 h. After completion of the reaction, the reaction mixture was evaporated under reduced pressure and neutralized with saturated sodium carbonate (20 mL), the aqueous layer was extracted with dichloromethane (3×25 mL). The organic layer was dried over anhydrous sodium sulphate, concentrated and purified by column chromatography to obtain 2-amino-2-methyl-1-phenylpropan-1-01 (1.2 g, 7.26 mmol, 77% yield). ¹H-NMR (400 MHz, CHLOROFORM-D) δ 7.34-7.28 (m, 5H), 4.50 (s, 1H), 4.12 (q, J=7.2 Hz, 1H), 1.19 (s, 3H), 1.02 (s, 3H).

### Example 10: Preparation 2-amino-2-methylpropyl 2-methylbenzoate

### a) Step 1: Preparation of 2-((tort-butoxycarbonyl)amino)-2-methylpropyl 2-methylbenzoate

To a stirred solution of tert-butyl (1-hydroxy-2-methylpropan-2-yl)carbamate (5.2 g, 27.5 mmol) and N, N-diisopropylethylamine (7.20 mL, 41.2 mmol) in dichloromethane (50 mL), 2-methylbenzoyl chloride (4.25 g, 27.5 mmol) was added dropwise at 0° C. and the resulting mixture was stirred at 25° C. for 3 h. After completion of the reaction, the reaction mixture was evaporated under reduced pressure and diluted with water (20 mL), the aqueous layer was extracted with ethyl acetate (3×25 mL). The organic layer was dried over anhydrous sodium sulphate, concentrated to obtain 2-((tert-butoxycarbonyl)amino)-2-methylpropyl 2-methylbenzoate (8 g, 26.0 mmol, 95% yield). 1H-NMR (400 MHz, CHLOROFORM-D) δ 7.91 (dd, J=8.2, 1.3 Hz, 1H), 7.43-7.39 (m, 1H), 7.35-7.30 (m, 1H), 7.20-7.15 (m, 1H), 4.64 (s, 1H), 4.36 (s, 2H), 2.60 (s, 3H), 1.38 (s, 6H).

### b) Step 2: Preparation of 2-amino-2-methylpropyl 2-methylbenzoate

To a stirred solution of 2-((tert-butoxycarbonyl)amino)-2-methylpropyl 2-methylbenzoate (8 g, 26.0 mmol) in dichloromethane (50 mL), trifluoroacetic acid (10.03 mL, 130 mmol) was added dropwise at 0° C. and the resulting reaction mixture was allowed to warm to 25° C. and stirred for 1 h. After completion of the reaction, the reaction mixture was evaporated under reduced pressure and neutralized with saturated sodium carbonate (20 mL), the aqueous layer was extracted with dichloromethane (3×25 mL). The organic layer was dried over anhydrous sodium sulphate, concentrated and purified by column chromatography to obtain 2-amino-2-methylpropyl 2-methylbenzoate (3.6 g, 17.37 mmol, 67% yield). ¹H-NMR (400 MHz, CHLOROFORM-D) δ 8.00 (dd, J=7.7, 1.3 Hz, 1H), 7.38 (td, J=7.5, 1.3 Hz, 1H), 7.24-7.17 (m, 2H), 4.38-5.16 (2H), 4.23 (s, 2H), 2.55 (s, 3H), 1.38 (s, 6H); ESI MS (m/z) 208.10 (MH)⁺.

### Example 11: Preparation of 1-((2-fluorobenzyl)oxy)-2-methylpropan-2-amine

To a stirred suspension of sodium hydride (1.346 g, 33.7 mmol, 60% dispersion in mineral oil) in tetrahydrofuran (10 mL), a solution of 2-amino-2-methylpropan-1-ol (1 g, 11.2 mmol) in tetrahydrofuran (5 mL) was added under nitrogen atmosphere at 25° C. and stirred for 1 h. To this reaction mixture, 1-(bromomethyl)-2-fluorobenzene (1.767 mL, 14.58 mmol) was added at 0° C. and the resulting reaction mixture was further stirred for 2 h. After completion of the reaction, the reaction mixture was quenched with saturated ammonium chloride solution (50 mL) and the aqueous layer was extracted with ethyl acetate (3×50 mL). The organic layer was dried over anhydrous sodium sulphate, concentrated and purified by column chromatography to obtain 1-((2-fluorobenzyl)oxy)-2-methylpropan-2-amine (0.67 g, 3.40 mmol, 30% yield). 1H-NMR (400 MHz, CHLOROFORM-D) δ 7.41 (td, J=7.5, 1.7 Hz, 1H), 7.26-7.22 (m, 1H), 7.11 (td, J=7.5, 1.1 Hz, 1H), 7.04-6.99 (m, 1H), 4.59 (s, 2H), 3.22 (s, 2H), 1.09 (s, 6H); ESI MS (m/z) 197.95 (MH)⁺.

### Example 12: Preparation of 2-methyl-1-(pyridin-2-yloxy)propan-2-amine

To a stirred suspension of sodium hydride (0.897 g, 22.4 mmol, 60% dispersion in mineral oil) in tetrahydrofuran (10 mL), a solution of 2-amino-2-methylpropan-1-01 (1 g, 11.22 mmol) in tetrahydrofuran (10 mL) was added under nitrogen atmosphere at 25° C. and stirred for 1 h. To this reaction mixture, 2-fluoropyridine (1.307 g, 13.46 mmol) was added at 0° C. and the resulting reaction mixture was further stirred for 3 h. After completion of the reaction, the reaction mixture was quenched with saturated ammonium chloride solution (50 mL) and the aqueous layer was extracted with ethyl acetate (3×50 mL). The organic layer was dried over anhydrous sodium sulphate, concentrated and purified by column chromatography to obtain 2-methyl-1-(pyridin-2-yloxy)propan-2-amine (1.552 g, 9.3 mmol, 83% yield). ¹H-NMR (400 MHz, CHLOROFORM-D) δ 8.13 (dq, J=5.0, 1.0 Hz, 1H), 7.57 (ddd, J=8.7, 6.7, 1.6 Hz, 1H), 6.86 (ddd, J=7.1, 5.1, 1.0 Hz, 1H), 6.77 (dt, J=8.4, 0.9 Hz, 1H), 4.05 (s, 2H), 1.28-1.18 (m, 7H); ESI MS (m/z)=167.1 (MH)⁺.

### Example 13: Preparation 1-amino-1-(4-fluorophenyl)-2-methylpropan-2-ol

To a stirred solution of methyl 2-amino-2-(4-fluorophenyl)acetate (5.0 g, 27.3 mmol) in tetrahydrofuran (50 mL), methyl magnesium bromide (78 mL, 1.4 M. solution in tetrahydrofuran, 39 mL, 109 mmol) was added under inert atmosphere at −78° C. and the resulting reaction mixture was stirred at the same temperature for 2 h. After completion of the reaction, the reaction mixture was quenched with water (50 mL) and the aqueous layer was extracted with ethyl acetate (3×50 mL). The organic layer was dried over anhydrous sodium sulphate, concentrated and purified by column chromatography to obtain 1-amino-1-(4-fluorophenyl)-2-methylpropan-2-ol(4.0 g, 27.0 mmol, 80% yield). ESI MS (m/z)=184.0 (MH)⁺.

### Example 14: Preparation N-(1-(2-fluorophenyl)propan-2-yl)-O-methyl hydroxylamine

### a) Step 1: Preparation of 1-(2-fluorophenyl)propan-2-one O-methyl oxime

To a stirred solution of 1-(2-fluorophenyl)propan-2-one (5 g, 32.9 mmol) in ethanol (20 mL), O-methyl hydroxylamine hydrochloride (4.12 g, 49.3 mmol) and sodium acetate (4.04 g, 49.3 mmol) were added at 25° C. and the resulting reaction mixture was stirred for 2 h. After completion of the reaction, the reaction mixture was evaporated under reduced pressure to obtain 1-(2-fluorophenyl)propan-2-one O-methyl oxime. 1H-NMR (400 MHz, CHLOROFORM-D) δ 7.23-7.16 (m, 2H), 7.09-7.00 (m, 2H), 3.87 (s, 3H), 3.52 (s, 2H), 1.76 (s, 3H); ESI MS (m/z)=181.95 (MH)⁺.

### b) Step 2: Preparation of N-(1-(2-fluorophenyl)propan-2-yl)-O-methyl hydroxylamine

To a stirred solution of 1-(2-fluorophenyl)propan-2-one O-methyl oxime (1 g, 5.52 mmol) in acetic acid (5 mL), sodium cyanoborohydride (0.694 g, 11.04 mmol) was added slowly at 20° C. and the resulting reaction mixture was stirred for 30 min. After completion of the reaction, the reaction mixture was quenched with water (30 mL) and saturated solution of sodium bicarbonate, the aqueous layer was extracted with ethyl acetate (3×50 mL). The organic layer was dried over anhydrous sodium sulphate, concentrated and purified by column chromatography to obtain N-(1-(2-fluorophenyl)propan-2-yl)-O-methyl hydroxylamine (0.8 g, 4.37 mmol, 79% yield). ¹H-NMR (400 MHz, CHLOROFORM-D) δ 7.22-7.13 (m, 2H), 7.08-6.99 (m, 2H), 4.50 (s, 1H), 3.56 (s, 3H), 3.33-3.24 (m, 1H), 2.92-2.87 (m, 1H), 2.65 (qd, J=6.8, 1.1 Hz, 1H), 1.07 (dd, J=6.5, 0.6 Hz, 3H); ESI MS (m/z)=184.0 (MH)⁺.

### Example 15: Preparation 1-(4-fluorophenoxy)-2,4-dimethylpent-4-en-2-amine

### a) Step 1: Preparation of 1-(4-fluorophenoxy)propan-2-one

To a stirred solution of 4-fluorophenol (10 g, 89 mmol) in acetone (150 ml), potassium carbonate (14.79 g, 107 mmol), potassium iodide (1.481 g, 8.92 mmol) and 1-chloropropan-2-one (8.54 mL, 107 mmol) were added under inert atmosphere at 25° C. The resulting reaction mixture was stirred at 50° C. for 6 h. After completion of the reaction, the reaction mixture was quenched with water (50 mL) and the aqueous layer was extracted with ethyl acetate (3×100 mL). The organic layer was dried over anhydrous sodium sulphate, concentrated to obtain 1-(4-fluorophenoxy)propan-2-one (15 g, 89 mmol, 100% yield). ¹H-NMR (400 MHz, CHLOROFORM-D) δ 7.01-6.95 (m, 2H), 6.86-6.81 (m, 2H), 4.51 (s, 2H), 2.27 (s, 3H).

### b) Step 2: Preparation of N-(1-(4-fluorophenoxy)propan-2-ylidene)-2-methylpropane-2-sulfinamide

To a stirred solution of 1-(4-fluorophenoxy)propan-2-one (10 g, 59.5 mmol) in tetrahydrofuran (100 mL), titanium(IV) isopropoxide (22.65 mL, 77 mmol) and 2-methylpropane-2-sulfinamide (8.65 g, 71.4 mmol) were added under inert atmosphere at 25° C. The resulting reaction mixture was stirred at 60° C. for 6 h. After completion of the reaction, the reaction mixture was quenched with saturated solution of sodium bicarbonate (100 mL) and the aqueous layer was extracted with ethyl acetate (3×100 mL). The organic layer was dried over anhydrous sodium sulphate, concentrated and purified by column chromatography to obtain N-(1-(4-fluorophenoxy)propan-2-ylidene)-2-methylpropane-2-sulfinamide (5.6 g, 20.6 mmol, 48% yield). 1H-NMR (400 MHz, CHLOROFORM-D) δ 7.01-6.94 (m, 3H), 6.88-6.81 (m, 3H), 4.61 (d, J=1.7 Hz, 2H), 2.40 (s, 3H), 1.21 (d, J=17.4 Hz, 10H)); ESI MS (m/z)=272.05 (MH)⁺.

### c) Step 3: Preparation of N-(-1-(4-fluorophenoxy)-2,4-dimethylpent-4-en-2-yl)-2-methylpropane-2-sulfinamide

To a stirred suspension of magnesium (0.448 g, 18.43 mmol) and iodine (100 mg) in tetrahydrofuran (20 mL), 3-chloro-2-methylprop-1-ene (1.092 mL, 11.1 mmol) was added at 65° C. and stirred for 1 h. The reaction mixture was cooled to −78° C. and a solution ofN-(1-(4-fluorophenoxy)propan-2-ylidene)-2-methylpropane-2-sulfinamide (2.5 g, 9.21 mmol) in tetrahydrofuran (5 mL) was added under inert atmosphere. The resulting reaction mixture was further stirred at 25° C. for 8 h. After completion of the reaction, the reaction mixture was quenched with saturated ammonium chloride (20 mL) and the aqueous layer was extracted with ethyl acetate (3×25 mL). The organic layer was dried over anhydrous sodium sulphate, concentrated and purified by column chromatography to obtain N-1-(4-fluorophenoxy)-2,4-dimethylpent-4-en-2-yl)-2-methylpropane-2-sulfinamide (1 g, 3.1 mmol, 40% yield). ESI MS (m/z)=328.05 (MH)⁺.

### d) Step 4: Preparation of 1-(4-fluorophenoxy)-2,4-dimethylpent-4-en-2-amine

To a stirred solution of N-(-1-(4-fluorophenoxy)-2,4-dimethylpent-4-en-2-yl)-2-methylpropane-2-sulfinamide (500 mg, 1.527 mmol) in methanol (10 mL), hydrochloric acid (15 mmol, 2.5 mL, 4 N solution in dioxane) was added at 0° C. and the resulting reaction mixture was stirred for 4 h. After completion of the reaction, the reaction mixture was evaporated under reduced pressure to obtain 1-(4-fluorophenoxy)-2,4-dimethylpent-4-en-2-amine (341 mg, 1.53 mmol, 100% yield). ESI MS (m/z)=224.05 (MH)⁺.

### Example 16: Preparation 1-(4-fluorophenyl)-2,4-dimethylpent-4-en-2-amine

### a) Step 1: Preparation of N-(1-(4-fluorophenyl)propan-2-ylidene)-2-methylpropane-2-sulfinamide

To a stirred solution of 1-(4-fluorophenoxy)propan-2-one (10 g, 59.5 mmol) in tetrahydrofuran (100 mL), titanium(IV) isopropoxide (22.65 mL, 77 mmol) and 2-methylpropane-2-sulfinamide (8.65 g, 71.4 mmol) were added under inert atmosphere at 25° C. The resulting reaction mixture was stirred at 60° C. for 6 h. After completion of the reaction, the reaction mixture was quenched with saturated solution of sodium bicarbonate (100 mL) and the aqueous layer was extracted with ethyl acetate (3×100 mL). The organic layer was dried over anhydrous sodium sulphate, concentrated and purified by column chromatography to obtain N-(1-(4-fluorophenyl)propan-2-ylidene)-2-methylpropane-2-sulfinamide (13.4 g, 52.5 mmol, 80% yield).

### b) Step 2: Preparation of N-(1-(4-fluorophenyl)-2,4-dimethylpent-4-en-2-yl)-2-methylpropane-2-sulfinamide

To a stirred suspension of magnesium (3.71 g, 153 mmol) and iodine (100 mg) in tetrahydrofuran (150 mL), 3-chloro-2-methylprop-1-ene (15.08 mL, 153 mmol) was added at 65° C. and the resulting reaction mixture was stirred for 1 h. The reaction mixture was cooled to −78° C. and a solution of N-(1-(4-fluorophenyl)propan-2-ylidene)-2-methylpropane-2-sulfinamide (13 g, 50.9 mmol) in tetrahydrofuran (20 mL) was added under inert atmosphere. The resulting reaction mixture was further stirred at 25° C. for 8 h. After completion of the reaction, the reaction mixture was quenched with saturated ammonium chloride (20 mL) and the aqueous layer was extracted with ethyl acetate (3×25 mL). The organic layer was dried over anhydrous sodium sulphate, concentrated and purified by column chromatography to obtain N-(1-(4-fluorophenyl)-2,4-dimethylpent-4-en-2-yl)-2-methylpropane-2-sulfinamide (8 g, 25.7 mmol, 50% yield). ESI MS (m/z)=311.6 (MH)⁺.

### c) Step 3: Preparation of 1-(4-fluorophenyl)-2,4-dimethylpent-4-en-2-amine

To a stirred solution of N-(1-(4-fluorophenyl)-2,4-dimethylpent-4-en-2-yl)-2-methylpropane-2-sulfinamide (10 g, 32.1 mmol) in methanol (30 mL), hydrochloric acid (321 mmol, 80 mL, 4 N solution in dioxane) was added at 0° C. and the resulting reaction mixture was stirred for 4 h. After completion of the reaction, the reaction mixture was evaporated under reduced pressure to obtain 1-(4-fluorophenyl)-2,4-dimethylpent-4-en-2-amine (6.5 g, 31.4 mmol, 98% yield). ESI MS (m/z)=207.65 (MH)⁺.

### Example 17: Preparation of 1-methyl-N-(2-methyl-1-phenylpropan-2-yl)-1H-pyrrolo[2,3-b]pyridine-5-carboxamide (compound no. 1

To a stirred solution of 1-methyl-1H-pyrrolo[2,3-b]pyridine-5-carbonitrile (200 mg, 1.27 mmol) and 2-methyl-1-phenylpropan-2-ol (382 mg, 2.54 mmol) in acetic acid (6 mL), sulfuric acid (0.8 mL) was added dropwise at 0° C. The resulting reaction mixture was stirred at 25° C. for 8 h. After completion of the reaction, the reaction mixture was neutralized with saturated sodium bicarbonate solution (50 mL) and extracted with ethyl acetate (3×25 mL). The organic layer was washed with brine (20 mL), dried over anhydrous sodium sulphate, concentrated and purified using reverse phase HPLC to afford 1-methyl-N-(2-methyl-1-phenylpropan-2-yl)-1H-pyrrolo[2,3-b]pyridine-5-carboxamide (250 mg, 0.813 mmol, 64% yield). ¹H-NMR (400 MHz, DMSO-d6) δ 8.66 (d, J=2.1 Hz, 1H), 8.33 (d, J=2.1 Hz, 1H), 7.62 (s, 1H), 7.58 (d, J=3.4 Hz, 1H), 7.24-7.20 (m, 2H), 7.18-7.12 (m, 3H), 6.54 (d, J=3.4 Hz, 1H), 3.83 (s, 3H), 3.14 (s, 2H), 1.35 (s, 6H); ESI MS (m/z) 308 (MH)⁺.

### Example 18: Preparation of N-(1-(4-fluorophenyl)-2-methylpropan-2-yl)-1-methyl-1H-pyrrolo[2,3-b]pyridine-5-carboxamide (compound no.74

To a stirred solution of 1-methyl-1H-pyrrolo[2,3-b]pyridine-5-carboxylic acid (329 mg, 1.87 mmol) in dichloromethane (10 mL), hydroxybenzotriazole (286 mg, 1.87 mmol) and 1-(3-dimethylaminopropyl)-3-ethylcarbodiimide hydrochloride (358 mg, 1.87 mmol) were added. After 10 min, to this reaction mixture, triethylamine (0.87 mL, 6.23 mmol) and 1-(4-fluorophenyl)-2-methylpropan-2-amine (250 mg, 1.45 mmol) were added under inert atmosphere at 25° C. and the resulting reaction mixture was stirred at 25° C. for 16 h. After completion of the reaction, dichloromethane was evaporated and the reaction mixture was quenched with water (10 mL). The reaction mixture was extracted with ethyl acetate (2×20 mL) and washed with brine (20 mL). The organic layer was dried over anhydrous sodium sulphate and evaporated to obtain a crude product, which was purified by column chromatography using suitable eluent to obtain pure product N-(1-(4-fluorophenyl)-2-methylpropan-2-yl)-1-methyl-1H-pyrrolo[2,3-b]pyridine-5-carboxamide (120 mg, 0.37 mmol, 30% yield). ¹H-NMR (400 MHz, CHLOROFORM-D) δ 8.58 (d, J=2.0 Hz, 1H), 8.16 (d, J=2.0 Hz, 1H), 7.28-7.20 (m, 5H), 6.49 (d, J=3.7 Hz, 1H), 3.90 (s, 3H), 3.76 (q, J=7.3 Hz, 1H), 1.58 (s, 3H), 1.39-1.36 (m, 6H); ESI MS (m/z) 326.05 (MH)⁺.

The following compounds in Table 1 were prepared by using analogous procedures as described in above examples 17 and 18.

TABLE 1

| Compd | | |
|---|---|---|
| No. | IUPAC Name | Analytical Data |
| 1 | 1-methyl-N-(2-methyl- | ¹H-NMR (400 MHz, DMSO-d6) δ 8.66 (d, J = 2.1 Hz, 1H), 8.33 |
| | 1-phenylpropan-2-yl)- | (d, J = 2.1 Hz, 1H), 7.62 (s, 1H), 7.58 (d, J = 3.4 Hz, 1H), 7.24- |
| | 1H-pyrrolo[2,3-b] | 7.20 (m, 2H), 7.18-7.12 (m, 3H), 6.54 (d, J = 3.4 Hz, 1H), 3.83 |
| | pyridine-5- | (s, 3H), 3.14 (s, 2H), 1.35 (s, 6H); ESI MS (m/z) 308 (MH)⁺ |
| | carboxamide | |
| 2 | N-(2,4-dimethyl-1- | ¹H-NMR (400 MHz, DMSO-d6) δ 8.65 (d, J = 2.0 Hz, 1H), 8.32 |
| | phenylpentan-2-yl)-1- | (d, J = 2.0 Hz, 1H), 7.60 (d, J = 3.4 Hz, 1H), 7.50 (s, 1H), 7.25- |
| | methyl-1H- | 7.21 (m, 2H), 7.18-7.14 (m, 3H), 6.56 (d, J = 3.4 Hz, 1H), 3.85 |
| | pyrrolo[2,3-b]pyridine- | (s, 3H), 3.61 (d, J = 13.0 Hz, 1H), 2.69 (d, J = 12.7 Hz, 1H), 2.34 |
| | 5-carboxamide | (q, J = 6.9 Hz, 1H), 1.86-1.80 (m, 1H), 1.42 (dd, J = 14.1, 5.0 Hz, |
| | | 1H), 1.21 (d, J = 19.3 Hz, 3H), 0.93 (dd, J = 11.5, 6.6 Hz, 6H); |
| | | ESI MS (m/z) 350 (MH)⁺ |
| 3 | N-(3-(2-fluorophenyl)- | ¹H-NMR (400 MHz, DMSO-d6) δ 8.66 (d, J = 2.2 Hz, 1H), 8.35 |
| | 2-methylbutan-2-yl)-1- | (d, J = 2.0 Hz, 1H), 7.73 (s, 1H), 7.58 (d, J = 3.7 Hz, 1H), 7.38 |
| | methyl-1H- | (td, J = 7.7, 1.7 Hz, 1H), 7.27-7.21 (m, 1H), 7.16-7.07 (m, 2H), |
| | pyrrolo[2,3-b]pyridine- | 6.55 (d, J = 3.7 Hz, 1H), 4.26 (q, J = 7.3 Hz, 1H), 3.84 (s, 3H), |
| | 5-carboxamide | 1.33 (s, 3H), 1.27-1.21 (m, 6H) ; ESI MS (m/z) 340.05 (MH)⁺ |
| 5 | N-(2,3-dimethyl-3- | ¹H-NMR (400 MHz, DMSO-d6) δ 8.56 (d, J = 2.0 Hz, 1H), 8.25 |
| | phenylbutan-2-yl)-1- | (d, J = 2.2 Hz, 1H), 7.61-7.55 (m, 1H), 7.48 (dd, J = 8.4, 1.1 Hz, |
| | methyl-1H- | 2H), 7.33-7.28 (m, 2H), 7.26-7.19 (m, 1H), 7.06 (s, 1H), 6.55 (d, |
| | pyrrolo[2,3-b]pyridine- | J = 3.7 Hz, 1H), 3.84 (d, J = 5.9 Hz, 3H), 1.46 (s, 6H), 1.37 (s, |
| | 5-carboxamide | 6H); ESI MS (m/z) 336.05 (MH)⁺ |
| 6 | 1-methyl-N-(2-methyl- | ¹H-NMR (400 MHz, DMSO-d6) δ 8.65 (d, J = 2.1 Hz, 1H), 8.32 |
| | 1-phenylbutan-2-yl)- | (d, J = 2.1 Hz, 1H), 7.58 (d, J = 3.4 Hz, 1H), 7.44 (s, 1H), 7.29- |
| | 1H-pyrrolo[2,3-b] | 7.19 (m, 2H), 7.17-7.12 (m, 3H), 6.54 (d, J = 3.4 Hz, 1H), 3.83 |
| | pyridine-5- | (s, 3H), 3.39 (d, J = 13.1 Hz, 1H), 2.85 (d, J = 12.8 Hz, 1H), 2.18 |
| | carboxamide | (dt, J = 21.1, 7.5 Hz, 1H), 1.57-1.47 (m, 1H), 1.18 (s, 3H), 0.85 |
| | | (s, 3H); ESI MS (m/z) 322 (MH)⁺ |
| 7 | N-(3-(2-chlorophenyl)- | ¹H-NMR (400 MHz, DMSO-d6) δ 8.68 (d, J = 2.2 Hz, 1H), 8.38 |
| | 2-methylbutan-2-yl)-1- | (d, J = 2.2 Hz, 1H), 7.76 (s, 1H), 7.58 (d, J = 3.7 Hz, 1H), 7.47 |
| | methyl-1H- | (dd, J = 7.8, 1.5 Hz, 1H), 7.40 (dd, J = 7.9, 1.3 Hz, 1H), 7.30 (td, |
| | pyrrolo[2,3-b]pyridine- | J = 7.6, 1.3 Hz, 1H), 7.22 (td, J = 7.6, 1.7 Hz, 1H), 6.56 (d, J = |
| | 5-carboxamide | 3.4 Hz, 1H), 4.50 (q, J = 7.2 Hz, 1H), 3.84 (s, 3H), 1.32 (s, 6H), |
| | | 1.23 (d, J = 7.3 Hz, 3H); ESI MS (m/z) 356.05 (MH)⁺ |
| 8 | 1-methyl-N-(2-methyl- | ¹H-NMR (400 MHz, DMSO-d6) δ 8.64 (d, J = 2.0 Hz, 1H), 8.32 |
| | 3-phenylbutan-2-yl)- | (d, J = 2.0 Hz, 1H), 7.63-7.59 (m, 2H), 7.28-7.17 (m, 5H), 6.56 |
| | 1H-pyrrolo[2,3-b] | (d, J = 3.4 Hz, 1H), 4.02-3.89 (m, 1H), 3.85 (s, 3H), 1.42 (d, J = |
| | pyridine-5- | 11.5 Hz, 3H), 1.28-1.23 (m, 6H); ESI MS (m/z) 321.95 (MH)⁺ |
| | carboxamide | |
| 10 | N-(1- | ¹H-NMR (400 MHz, DMSO-d6) δ 8.65 (d, J = 2.0 Hz, 1H), 8.31 |
| | benzylcyclopentyl)-1- | (d, J = 2.0 Hz, 1H), 7.66 (s, 1H), 7.57 (d, J = 3.4 Hz, 1H), 7.21- |
| | methyl-1H- | 7.18 (m, 2H), 7.15-7.09 (m, 3H), 6.54 (d, J = 3.4 Hz, 1H), 3.83 |
| | pyrrolo[2,3-b]pyridine- | (s, 3H), 3.18 (s, 2H), 2.09-2.06 (m, 2H), 1.73-1.65 (m, 6H); ESI |
| | 5-carboxamide | MS (m/z) 334.15 (MH)⁺ |
| 11 | N-(1- | ¹H-NMR (400 MHz, DMSO-d6) δ 8.66 (d, J = 1.8 Hz, 1H), 8.32 |
| | benzylcyclohexyl)-1- | (d, J = 1.8 Hz, 1H), 7.58 (d, J = 3.4 Hz, 1H), 7.33 (s, 1H), 7.21- |
| | methyl-1H- | 7.17 (m, 2H), 7.15-7.08 (m, 3H), 6.54 (d, J = 3.4 Hz, 1H), 3.83 |
| | pyrrolo[2,3-b]pyridine- | (s, 3H), 3.10 (s, 2H), 2.23 (d, J = 12.8 Hz, 2H), 1.58-1.49 (m, |
| | 5-carboxamide | 5H), 1.39-1.32 (m, 2H) 1.23 (m, 1H); ESI MS (m/z) 347.75 |
| | | (MH)⁺ |
| 12 | N-(1-(3- | ¹H-NMR (400 MHz, DMSO-d6) δ 8.64 (d, J = 2.1 Hz, 1H), 8.30 |
| | chlorobenzyl) | (d, J = 2.0 Hz, 1H), 7.58 (d, J = 3.4 Hz, 1H), 7.38 (s, 1H), 7.25- |
| | cyclohexyl)-1- | 7.19 (m, 2H), 7.13 (d, J = 1.5 Hz, 1H), 7.06 (dt, J = 6.9, 1.6 Hz, |
| | methyl-1H- | 1H), 6.55 (d, J = 3.4 Hz, 1H), 3.83 (s, 3H), 3.11 (s, 2H), 2.22 (d, |
| | pyrrolo[2,3-b]pyridine- | J = 12.8 Hz, 2H), 1.59-1.16 (m, 8H); ESI MS (m/z) 382 (MH)⁺ |
| | 5-carboxamide | |
| 13 | N-(1-(2-fluorophenyl)- | ¹H-NMR (400 MHz, DMSO-d6) δ 8.67 (d, J = 2.0 Hz, 1H), 8.34 |
| | 2-methylpropan-2-yl)- | (d, J = 2.0 Hz, 1H), 7.71 (s, 1H), 7.58 (d, J = 3.4 Hz, 1H), 7.23 |
| | 1-methyl-1H- | (d, J = 7.8 Hz, 1H), 7.17-7.10 (m, 2H), 7.04 (t, J = 7.5 Hz, 1H), |
| | pyrrolo[2,3-b]pyridine- | 6.55 (d, J = 3.4 Hz, 1H), 3.84 (s, 3H), 3.20 (s, 2H), 1.36 (s, 6H); |
| | 5-carboxamide | ESI MS (m/z) 325.90 (MH)⁺ |
| 14 | 1-methyl-N-(2-methyl- | ¹H-NMR (400 MHz, DMSO-d6) δ 8.63 (d, J = 2.0 Hz, 1H), 8.28 |
| | 1-(3- | (d, J = 2.2 Hz, 1H), 7.65 (s, 1H), 7.57 (d, J = 3.4 Hz, 1H), 7.53 |
| | (trifluoromethyl)phenyl) | (d, J = 7.8 Hz, 1H), 7.49-7.43 (m, 3H), 6.53 (d, J = 3.7 Hz, 1H), |
| | propan-2-yl)-1H- | 3.83 (s, 3H), 3.26 (s, 2H), 1.36 (s, 6H); ESI MS (m/z) 376.10 |
| | pyrrolo[2,3-b]pyridine- | (MH)⁺ |
| | 5-carboxamide | |
| 15 | N-(1-(3,5- | ¹H-NMR (400 MHz, DMSO-d6) δ 8.65 (d, J = 2.0 Hz, 1H), 8.32 |
| | difluorophenyl)-2- | (d, J = 2.2 Hz, 1H), 7.71 (s, 1H), 7.59 (d, J = 3.4 Hz, 1H), 7.03 |
| | methylpropan-2-yl)-1- | (tt, J = 9.5, 2.4 Hz, 1H), 6.84-6.80 (m, 2H), 6.55 (d, J = 3.4 Hz, |
| | methyl-1H- | 1H), 3.84 (s, 3H), 3.19 (s, 2H), 1.36 (s, 6H); ESI MS (m/z) 344.05 |
| | pyrrolo[2,3-b]pyridine- | (MH)⁺ |
| | 5-carboxamide | |
| 16 | N-(1-(4-(tert- | ¹H-NMR (400 MHz, DMSO-d6) δ 8.68 (d, J = 2.0 Hz, 1H), 8.34 |
| | butyl)phenyl)-2- | (d, J = 2.2 Hz, 1H), 7.64 (s, 1H), 7.58 (d, J = 3.4 Hz, 1H), 7.23 |
| | methylpropan-2-yl)-1- | (d, J = 8.3 Hz, 2H), 7.05 (d, J = 8.3 Hz, 2H), 6.54 (d, J = 3.4 Hz, |
| | methyl-1H- | 1H), 3.83 (s, 3H), 3.10 (s, 2H), 1.35 (s, 6H), 1.22 (s, 9H); ESI MS |
| | pyrrolo[2,3-b]pyridine- | (m/z) 364.10 (MH)⁺ |
| | 5-carboxamide | |
| 17 | N-(1-(2-chlorophenyl)- | ¹H-NMR (400 MHz, DMSO-d6) δ 8.69 (d, J = 2.2 Hz, 1H), 8.37 |
| | 2-methylpropan-2-yl)- | (d, J = 2.2 Hz, 1H), 7.76 (s, 1H), 7.59 (d, J = 3.7 Hz, 1H), 7.43- |
| | 1-methyl-1H- | 7.40 (m, 1H), 7.23-7.18 (m, 3H), 6.55 (d, J = 3.4 Hz, 1H), 3.84 |
| | pyrrolo[2,3-b]pyridine- | (s, 3H), 3.35 (s, 2H), 1.39 (s, 6H); ESI MS (m/z) 342.05 (MH)⁺ |
| | 5-carboxamide | |
| 18 | N-(1-(4- | ¹H-NMR (400 MHz, DMSO-d6) δ 8.67 (d, J = 1.8 Hz, 1H), 8.33 |
| | isopropylphenyl)-2- | (d, J = 2.1 Hz, 1H), 7.63 (s, 1H), 7.58 (d, J = 3.4 Hz, 1H), 7.06 |
| | methylpropan-2-yl)-1- | (dd, J = 17.1, 7.9 Hz, 4H), 6.54 (d, J = 3.7 Hz, 1H), 3.83 (s, 3H), |
| | methyl-1H- | 3.10 (s, 2H), 2.83-2.76 (m, 1H), 1.38 (s, 6H), 1.15 (dd, J = 15.3, |
| | pyrrolo[2,3-b]pyridine- | 7.0 Hz, 6H); ESI MS (m/z) 350.30 (MH)⁺ |
| | 5-carboxamide | |
| 19 | 1-methyl-N-(2-methyl- | ¹H-NMR (400 MHz, DMSO-d6) δ 8.66 (d, J = 2.0 Hz, 1H), 8.32 |
| | 1-(4- | (d, J = 2.0 Hz, 1H), 7.66 (s, 1H), 7.58 (d, J = 3.7 Hz, 1H), 7.25- |
| | (trifluoromethoxy)phe | 7.20 (m, 4H), 6.54 (d, J = 3.4 Hz, 1H), 3.83 (s, 3H), 3.19 (s, 2H), |
| | nyl)propan-2-yl)-1H- | 1.35 (s, 6H); ESI MS (m/z) 392 (MH)⁺ |
| | pyrrolo[2,3-b]pyridine- | |
| | 5-carboxamide | |
| 20 | N-(1-(2,4- | ¹H-NMR (400 MHz, DMSO-d6) δ 8.67 (d, J = 2.0 Hz, 1H), 8.34 |
| | difluorophenyl)-2- | (d, J = 2.2 Hz, 1H), 7.70 (s, 1H), 7.58 (d, J = 3.4 Hz, 1H), 7.21- |
| | methylpropan-2-yl)-1- | 7.13 (m, 2H), 6.95 (td, J = 8.4, 2.1 Hz, 1H), 6.55 (d, J = 3.4 Hz, |
| | methyl-1H- | 1H), 3.83 (s, 3H), 3.18 (s, 2H), 1.35 (s, 6H); ESI MS (m/z) 343.70 |
| | pyrrolo[2,3-b]pyridine- | (MH)⁺ |
| | 5-carboxamide | |
| 21 | N-(1-(3-fluorophenyl)- | ¹H-NMR (400 MHz, DMSO-d6) δ 8.66 (d, J = 2.0 Hz, 1H), 8.32 |
| | 2-methylpropan-2-yl)- | (d, J = 2.0 Hz, 1H), 7.68 (s, 1H), 7.58 (d, J = 3.4 Hz, 1H), 7.26 |
| | 1-methyl-1H- | (q, J = 7.3 Hz, 1H), 7.02-6.90 (m, 3H), 6.55 (d, J = 3.4 Hz, 1H), |
| | pyrrolo[2,3-b]pyridine- | 3.83 (s, 3H), 3.17 (s, 2H), 1.35 (s, 6H); ESI MS (m/z) 326.10 |
| | 5-carboxamide | (MH)⁺ |
| 22 | N-(1-(3-chlorophenyl)- | ¹H-NMR (400 MHz, DMSO-d6) δ 8.65 (d, J = 2.0 Hz, 1H), 8.31 |
| | 2-methylpropan-2-yl)- | (d, J = 2.2 Hz, 1H), 7.67 (s, 1H), 7.58 (d, J = 3.4 Hz, 1H), 7.28- |
| | 1-methyl-1H- | 7.21 (m, 2H), 7.17 (d, J = 1.7 Hz, 1H), 7.09 (dt, J = 6.8, 1.8 Hz, |
| | pyrrolo[2,3-b]pyridine- | 1H), 6.54 (d, J = 3.4 Hz, 1H), 3.83 (s, 3H), 3.16 (s, 2H), 1.35 (s, |
| | 5-carboxamide | 6H); ESI MS (m/z) 342.05 (MH)⁺ |
| 23 | N-(1-(3,4- | ¹H-NMR (400 MHz, DMSO-d6) δ 8.66 (d, J = 2.0 Hz, 1H), 8.33 |
| | difluorophenyl)-2- | (d, J = 2.2 Hz, 1H), 7.66 (s, 1H), 7.59 (d, J = 3.4 Hz, 1H), 7.32- |
| | methylpropan-2-yl)-1- | 7.25 (m, 1H), 7.12 (ddd, J = 12.0, 8.0, 2.0 Hz, 1H), 6.97-6.93 (m, |
| | methyl-1H- | 1H), 6.55 (d, J = 3.4 Hz, 1H), 3.85 (s, 3H), 3.15 (s, 2H), 1.34 (s, |
| | pyrrolo[2,3-b]pyridine- | 6H); ESI MS (m/z) 344.05 (MH)⁺ |
| | 5-carboxamide | |
| 24 | N-(1-(3,4- | ¹H-NMR (400 MHz, DMSO-d6) δ 8.65 (d, J = 2.2 Hz, 1H), 8.31 |
| | dichlorophenyl)-2- | (d, J = 2.0 Hz, 1H), 7.67 (s, 1H), 7.58 (d, J = 3.4 Hz, 1H), 7.49 |
| | methylpropan-2-yl)-1- | (d, J = 8.3 Hz, 1H), 7.34 (d, J = 2.0 Hz, 1H), 7.11 (dd, J = 8.3, 2.0 |
| | methyl-1H- | Hz, 1H), 6.55 (d, J = 3.4 Hz, 1H), 3.83 (s, 3H), 3.16 (s, 2H), 1.35 |
| | pyrrolo[2,3-b]pyridine- | (s, 6H); ESI MS (m/z) 376 (MH)⁺ |
| | 5-carboxamide | |
| 25 | N-(4,4,4-trifluoro-2- | ¹H-NMR (400 MHz, DMSO-d6) δ 8.93 (d, J = 2.0 Hz, 1H), 8.76 |
| | methyl-1-phenylbutan- | (q, J = 0.9 Hz, 1H), 8.31 (d, J = 5.4 Hz, 1H), 8.09 (s, 1H), 7.63 |
| | 2-yl)thieno[3,2-b] | (dd, J = 5.4, 0.7 Hz, 1H), 7.28-7.18 (m, 3H), 7.15-7.13 (m, 2H), |
| | pyridine-6- | 3.64 (d, J = 13.0 Hz, 1H), 3.51 (dd, J = 15.3, 12.3 Hz, 1H), 2.80 |
| | carboxamide | (d, J = 13.2 Hz, 1H), 2.65 (td, J = 11.6, 3.8 Hz, 1H), 1.31 (s, 3H); |
| | | ESI MS (m/z) 379.05 (MH)⁺ |
| 26 | N-(1-(2-bromophenyl)- | ¹H-NMR (400 MHz, CHLOROFORM-D) δ 8.70 (d, J = 2.0 Hz, |
| | 2-methylpropan-2-yl)- | 1H), 8.38 (d, J = 2.2 Hz, 1H), 7.79 (s, 1H), 7.60-7.58 (m, 2H), |
| | 1-methyl-1H- | 7.22-7.18 (m, 2H), 7.14-7.10 (m, 1H), 6.56 (d, J = 3.4 Hz, 1H), |
| | pyrrolo[2,3-b]pyridine- | 3.84 (s, 3H), 3.37 (s, 2H), 1.39 (s, 6H); ESI MS (m/z) 387.75 |
| | 5-carboxamide | (MH)⁺ |
| 27 | 1-methyl-N-(2-methyl- | ¹H-NMR(400 MHz, ACETONITRILE-D3) δ 8.60 (d, J = 2.0 Hz, |
| | 1-(m-tolyl)propan-2- | 1H), 8.21 (d, J = 2.0 Hz, 1H), 7.37 (d, J = 3.4 Hz, 1H), 7.13-7.09 |
| | yl)-1H-pyrrolo[2,3-b] | (m, 1H), 7.01-6.96 (m, 3H), 6.51 (d, J = 3.7 Hz, 1H), 6.30 (d, J = |
| | pyridine-5- | 14.2 Hz, 1H), 3.84 (s, 3H), 3.13 (s, 2H), 2.19 (s, 3H), 1.39 (s, |
| | carboxamide | 6H); ESI MS (m/z) 322.25 (MH)⁺ |
| 28 | N-(2,4-dimethyl-1- | ¹H-NMR (400 MHz, ACETONITRILE-D3) δ 9.82 (s, 1H), 8.56 |
| | phenylpentan-2-yl)- | (d, J = 2.0 Hz, 1H), 8.22 (dd, J = 2.1, 0.6 Hz, 1H), 7.42 (dd, J = |
| | 1H-pyrrolo[2,3-b] | 3.4, 2.4 Hz, 1H), 7.24-7.14 (m, 5H), 6.53 (q, J = 1.8 Hz, 1H), 6.19 |
| | pyridine-5- | (s, 1H), 3.53 (d, J = 13.0 Hz, 1H), 2.81 (d, J = 13.0 Hz, 1H), 2.26 |
| | carboxamide | (dd, J = 14.2, 6.6 Hz, 1H), 1.90-1.82 (m, 1H), 1.51 (dd, J = 14.1, |
| | | 5.0 Hz, 1H), 1.25 (s, 3H), 0.95 (dd, J = 7.7, 6.7 Hz, 6H); ESI MS |
| | | (m/z) 336.10 (MH)⁺ |
| 29 | N-(2,4-dimethyl-1- | ¹H-NMR (400 MHz, CHLOROFORM-D) δ 8.88 (s, 1H), 8.57 (s, |
| | phenylpentan-2- | 1H), 7.92 (d, J = 5.6 Hz, 1H), 7.62 (d, J = 5.4 Hz, 1H), 7.27-7.16 |
| | yl)thieno[3,2-b] | (m, 5H), 5.75 (s, 1H), 3.45 (d, J = 13.4 Hz, 1H), 2.96 (d, J = 13.2 |
| | pyridine-6- | Hz, 1H), 2.17 (q, J = 7.1 Hz, 1H), 1.92-1.84 (m, 1H), 1.64 (dd, J = |
| | carboxamide | 14.3, 5.0 Hz, 1H), 1.41 (s, 3H), 1.01 (dd, J = 6.7, 2.8 Hz, 6H); |
| | | ESI MS (m/z) 353.00 (MH)⁺ |
| 30 | N-(2-methyl-1- | ¹H-NMR (400 MHz, CHLOROFORM-D) δ 8.89 (d, J = 1.7 Hz, |
| | phenylpropan-2- | 1H), 8.58 (q, J = 0.9 Hz, 1H), 7.91 (d, J = 5.4 Hz, 1H), 7.61 (dd, |
| | yl)thieno[3,2-b] | J = 5.6, 0.7 Hz, 1H), 7.29-7.17 (m, 5H), 5.91 (s, 1H), 3.19 (s, 2H), |
| | pyridine-6- | 1.53 (d, J = 16.4 Hz, 6H); ESI MS (m/z) 311.05 (MH)⁺ |
| | carboxamide | |
| 31 | 1,6-dimethyl-N-(2- | ¹H-NMR (400 MHz, DMSO-d6) δ 7.75 (s, 1H), 7.65 (s, 1H), 7.44 |
| | methyl-1- | (d, J = 3.4 Hz, 1H), 7.31-7.28 (m, 2H), 7.23-7.19 (m, 3H), 6.42 |
| | phenylpropan-2-yl)- | (d, J = 3.4 Hz, 1H), 3.78 (s, 3H), 3.13 (s, 2H), 2.61 (s, 3H), 1.32 |
| | 1H-pyrrolo[2,3-b] | (s, 6H); ESI MS (m/z) 322.10 (MH)⁺ |
| | pyridine-5- | |
| | carboxamide | |
| 34 | N-(2,4-dimethyl-1- | ¹H-NMR(400 MHz, DMSO-d6) δ 7.70 (s, 1H), 7.52 (s, 1H), 7.44 |
| | phenylpentan-2-yl)- | (d, J = 3.4 Hz, 1H), 7.30-7.26 (m, 2H), 7.22-7.19 (m, 3H), 6.42 |
| | 1,6-dimethyl-1H- | (d, J = 3.7 Hz, 1H), 3.77 (s, 3H), 3.52 (d, J = 12.7 Hz, 1H), 2.73- |
| | pyrrolo[2,3-b]pyridine- | 2.70 (m, 1H), 2.65 (s, 3H), 2.32-2.27 (m, 1H), 1.86-1.78 (m, 1H), |
| | 5-carboxamide | 1.40 (dd, J = 13.9, 5.6 Hz, 1H), 1.15 (s, 3H), 0.96 (q, J = 3.3 Hz, |
| | | 6H); ESI MS (m/z) 364.45 (MH)⁺ |
| 35 | N-(3-(2-fluorophenyl)- | ¹H-NMR (400 MHz, DMSO-d6) δ 7.80 (d, J = 3.1 Hz, 2H), 7.44 |
| | 2-methylbutan-2-yl)- | (d, J = 3.4 Hz, 1H), 7.41-7.37 (m, 1H), 7.27-7.23 (m, 1H), 7.17- |
| | 1,6-dimethyl-1H- | 7.10 (m, 2H), 6.44 (d, J = 3.4 Hz, 1H), 4.25 (q, J = 7.2 Hz, 1H), |
| | pyrrolo[2,3-b]pyridine- | 3.78 (s, 3H), 2.59 (s, 3H), 1.28 (s, 9H); ESI MS (m/z) 354.10 |
| | 5-carboxamide | (MH)⁺ |
| 36 | N-(1-(2-fluorophenyl)- | ¹H-NMR (400 MHz, DMSO-d6) δ 8.65 (d, J = 2.0 Hz, 1H), 8.31 |
| | 2,4-dimethylpentan-2- | (d, J = 2.0 Hz, 1H), 7.59-7.57 (m, 2H), 7.25-7.19 (m, 1H), 7.16- |
| | yl)-1-methyl-1H- | 7.10 (m, 2H), 7.02 (td, J = 7.3, 1.1 Hz, 1H), 6.55 (d, J = 3.4 Hz, |
| | pyrrolo[2,3-b]pyridine- | 1H), 3.83 (s, 3H), 3.51 (d, J = 13.4 Hz, 1H), 2.88 (d, J = 13.0 Hz, |
| | 5-carboxamide | 1H), 2.39 (q, J = 6.9 Hz, 1H), 1.84-1.74 (m, 1H), 1.41 (dd, J = |
| | | 13.9, 4.9 Hz, 1H), 1.16 (s, 3H), 0.91 (dd, J = 11.6, 6.7 Hz, 6H); |
| | | ESI MS (m/z) 368.10 (MH)⁺ |
| 37 | N-(2,3-dimethyl-3- | ¹H-NMR(400 MHz, DMSO-d6) δ 7.77 (s, 1H), 7.47-7.44 (m, |
| | phenylbutan-2-yl)-1,6- | 3H), 7.31-7.27 (m, 3H), 7.19 (t, J = 7.2 Hz, 1H), 6.43 (d, J = 3.4 |
| | dimethyl-1H- | Hz, 1H), 3.78 (s, 3H), 2.55 (s, 3H), 1.46 (s, 6H), 1.32 (s, 6H); ESI |
| | pyrrolo[2,3-b]pyridine- | MS (m/z) 350.15 (MH)⁺ |
| | 5-carboxamide | |
| 38 | N-(1-(2-fluorophenyl)- | ¹H-NMR (400 MHz, DMSO-d6) δ 7.80 (s, 1H), 7.76 (s, 1H), 7.44 |
| | 2-methylpropan-2-yl)- | (d, J = 3.4 Hz, 1H), 7.32-7.25 (m, 2H), 7.18-7.13 (m, 2H), 6.43 |
| | 1,6-dimethyl-1H- | (d, J = 3.4 Hz, 1H), 3.78 (s, 3H), 3.20 (s, 2H), 2.62 (s, 3H), 1.33 |
| | pyrrolo[2,3-b]pyridine- | (s, 6H); ESI MS (m/z) 340.10 (MH)⁺ |
| | 5-carboxamide | |
| 39 | N-(1- | ¹H-NMR (400 MHz, DMSO-d6) δ 8.66 (d, J = 2.2 Hz, 1H), 8.50 |
| | benzylcyclopropyl)-1- | (s, 1H), 8.33 (d, J = 2.2 Hz, 1H), 7.57 (d, J = 3.4 Hz, 1H), 7.27- |
| | methyl-1H- | 7.23 (m, 2H), 7.19-7.16 (m, 3H), 6.53 (d, J = 3.4 Hz, 1H), 3.82 |
| | pyrrolo[2,3-b]pyridine- | (s, 3H), 2.97 (s, 2H), 0.79-0.78 (m, 4H); ESI MS (m/z) 306.05 |
| | 5-carboxamide | (MH)⁺ |
| 40 | N-(3-(2-chlorophenyl)- | ¹H-NMR (400 MHz, DMSO-d6) δ 7.85 (s, 1H), 7.83 (s, 1H), 7.47 |
| | 2-methylbutan-2-yl)- | (dd, J = 7.8, 1.5 Hz, 1H), 7.45 (d, J = 3.4 Hz, 1H), 7.42 (dd, J = |
| | 1,6-dimethyl-1H- | 7.8, 1.5 Hz, 1H), 7.30 (td, J = 7.5, 1.4 Hz, 1H), 7.22 (td, J = 7.6, |
| | pyrrolo[2,3-b]pyridine- | 1.5 Hz, 1H), 6.45 (d, J = 3.4 Hz, 1H), 4.48 (q, J = 7.2 Hz, 1H), |
| | 5-carboxamide | 3.79 (s, 3H), 2.59 (s, 3H), 1.33 (s, 3H), 1.28 (d, J = 7.3 Hz, 3H), |
| | | 1.23 (d, J = 9.0 Hz, 3H); ESI MS (m/z) 370.10 (MH)⁺ |
| 41 | N-(1-(4-fluorophenyl)- | ¹H-NMR (400 MHz, DMSO-d6) δ 11.85 (s, 1H), 8.61 (d, J = 2.0 |
| | 2-methylpropan-2-yl)- | Hz, 1H), 8.32 (d, J = 1.5 Hz, 1H), 7.60 (s, 1H), 7.53 (dd, J = 3.3, |
| | 1H-pyrrolo[2,3-b] | 2.6 Hz, 1H), 7.16-7.13 (m, 2H), 7.07-7.02 (m, 2H), 6.52 (q, J = |
| | pyridine-5- | 1.8 Hz, 1H), 3.13 (s, 2H), 1.33 (s, 6H); ESI MS (m/z) 312.05 |
| | carboxamide | (MH)⁺ |
| 42 | N-(1-(4-chloro-3- | ¹H-NMR (400 MHz, CHLOROFORM-D) δ 8.64 (d, J = 2.0 Hz, |
| | fluorophenyl)-2- | 1H), 8.22 (d, J = 2.2 Hz, 1H), 7.25-7.21 (m, 2H), 7.00-6.96 (m, |
| | methylpropan-2-yl)-1- | 1H), 6.89 (dd, J = 8.2, 1.3 Hz, 1H), 6.52 (d, J = 3.4 Hz, 1H), 5.67 |
| | methyl-1H- | (s, 1H), 3.91 (s, 3H), 3.22 (s, 2H), 1.44 (s, 6H); ESI MS (m/z) |
| | pyrrolo[2,3-b]pyridine- | 360.05 (MH)⁺ |
| | 5-carboxamide | |
| 43 | N-(1-(4-chlorophenyl)- | ¹H-NMR (400 MHz, CHLOROFORM-D) δ 8.63 (d, J = 2.0 Hz, |
| | 2-methylpropan-2-yl)- | 1H), 8.21 (d, J = 2.2 Hz, 1H), 7.25 (d, J = 3.4 Hz, 1H), 7.20 (dt, |
| | 1-methyl-1H- | J = 8.9, 2.2 Hz, 2H), 7.10 (dt, J = 8.7, 2.2 Hz, 2H), 6.52 (d, J = |
| | pyrrolo[2,3-b]pyridine- | 3.4 Hz, 1H), 5.66 (s, 1H), 3.91 (s, 3H), 3.19 (s, 2H), 1.46 (s, 6H); |
| | 5-carboxamide | ESI MS (m/z) 342.0 (MH)⁺ |
| 44 | N-(2,4-dimethyl-1- | ¹H-NMR (400 MHz, CHLOROFORM-D) δ 8.51 (d, J = 2.2 Hz, |
| | phenylpentan-2-yl)- | 1H), 7.67 (t, J = 1.1 Hz, 1H), 7.25-7.18 (m, 3H), 7.15-7.13 (m, |
| | 5,6,7,8- | 2H), 5.54 (s, 1H), 3.39 (d, J = 13.2 Hz, 1H), 2.95-2.91 (m, 3H), |
| | tetrahydroquinoline-3- | 2.80 (t, J = 6.4 Hz, 2H), 2.12-2.07 (m, 1H), 1.93-1.79 (m, 5H), |
| | carboxamide | 1.60 (dd, J = 14.4, 4.9 Hz, 1H), 1.36 (s, 3H), 0.98 (dd, J = 6.6, 5.9 |
| | | Hz, 6H); ESI MS (m/z) 351.20 (MH)⁺ |
| 45 | N-(2-methyl-1- | ¹H-NMR (400 MHz, CHLOROFORM-D) δ 8.52 (d, J = 2.2 Hz, |
| | phenylpropan-2-yl)- | 1H), 7.67 (t, J = 1.1 Hz, 1H), 7.29-7.27 (m, 1H), 7.25-7.20 (m, |
| | 5,6,7,8- | 2H), 7.17-7.15 (m, 2H), 5.68 (s, 1H), 3.14 (s, 2H), 2.93 (t, J = 6.5 |
| | tetrahydroquinoline-3- | Hz, 2H), 2.79 (t, J = 6.2 Hz, 2H), 1.93-1.87 (m, 2H), 1.84-1.78 |
| | carboxamide | (m, 2H), 1.47 (s, 6H); ESI MS (m/z) 309.40 (MH)⁺ |
| 46 | 3-bromo-1-methyl-N- | ¹H-NMR (400 MHz, DMSO-d6) δ 8.75 (d, J = 2.0 Hz, 1H), 8.24 |
| | (2-methyl-1- | (d, J = 2.0 Hz, 1H), 7.84 (s, 1H), 7.82 (s, 1H), 7.24-7.12 (m, 5H), |
| | phenylpropan-2-yl)- | 3.84 (s, 3H), 3.14 (s, 2H), 1.34 (d, J = 14.7 Hz, 6H); ESI MS |
| | 1H-pyrrolo[2,3-b] | (m/z) 387.65 (MH)⁺ |
| | pyridine-5- | |
| | carboxamide | |
| 47 | N-(1-(4-chloro-2- | ¹H-NMR (400 MHz, CHLOROFORM-D) δ 8.66 (d, J = 2.0 Hz, |
| | fluorophenyl)-2- | 1H), 8.24 (d, J = 2.2 Hz, 1H), 7.25 (d, J = 3.4 Hz, 1H), 7.13-7.06 |
| | methylpropan-2-yl)-1- | (m, 2H), 6.99 (dd, J = 8.4, 1.8 Hz, 1H), 6.52 (d, J = 3.4 Hz, 1H), |
| | methyl-1H- | 5.82 (s, 1H), 3.91 (s, 3H), 3.24 (d, J = 1.2 Hz, 2H), 1.49 (d, J = |
| | pyrrolo[2,3-b]pyridine- | 0.6 Hz, 6H); ESI MS (m/z) 361.05 (MH)⁺ |
| | 5-carboxamide | |
| 48 | N-(1-(3-bromophenyl)- | ¹H-NMR (400 MHz, CHLOROFORM-D) δ 8.64 (d, J = 2.0 Hz, |
| | 2-methylpropan-2-yl)- | 1H), 8.20 (d, J = 2.2 Hz, 1H), 7.35-7.31 (m, 2H), 7.23 (d, J = 3.4 |
| | 1-methyl-1H- | Hz, 1H), 7.10-7.09 (m, 2H), 6.51 (d, J = 3.4 Hz, 1H), 5.68 (s, 1H), |
| | pyrrolo[2,3-b]pyridine- | 3.90 (s, 3H), 3.19 (s, 2H), 1.47 (s, 6H); ESI MS (m/z) 387.25 |
| | 5-carboxamide | (MH)⁺ |
| 49 | N-(1-(2- | ¹H-NMR (400 MHz, CHLOROFORM-D) δ 8.63 (d, J = 2.0 Hz, |
| | methoxyphenyl)-2- | 1H), 8.21 (d, J = 2.0 Hz, 1H), 7.33 (s, 1H), 7.25-7.23 (m, 1H), |
| | methylpropan-2-yl)-1- | 7.22 (d, J = 3.4 Hz, 1H), 7.18 (dd, J = 7.7, 1.6 Hz, 1H), 6.96-6.93 |
| | methyl-1H- | (m, 2H), 6.49 (d, J = 3.7 Hz, 1H), 3.91 (s, 6H), 2.98 (s, 2H), 1.59 |
| | pyrrolo[2,3-b]pyridine- | (s, 6H); ESI MS (m/z) 338.10 (MH)⁺ |
| | 5-carboxamide | |
| 50 | 1-methyl-N-(2-methyl- | ¹H-NMR (400 MHz, CHLOROFORM-D) δ 8.64 (d, J = 2.2 Hz, |
| | 1-(p-tolyl)propan-2- | 1H), 8.22 (d, J = 2.2 Hz, 1H), 7.23 (d, J = 3.4 Hz, 1H), 7.09-7.05 |
| | yl)-1H-pyrrolo[2,3-b] | (m, 4H), 6.49 (dd, J = 16.4, 3.4 Hz, 1H), 5.74 (s, 1H), 3.90 (s, |
| | pyridine-5- | 3H), 3.13 (s, 2H), 2.31 (s, 3H), 1.49 (s, 6H); ESI MS (m/z) 322.15 |
| | carboxamide | (MH)⁺ |
| 51 | 3-bromo-N-(2,4- | ¹H-NMR (400 MHz, CHLOROFORM-D) δ 8.67 (d, J = 2.2 Hz, |
| | dimethyl-1- | 1H), 8.10 (d, J = 2.2 Hz, 1H), 7.27 (t, J = 2.2 Hz, 1H), 7.25-7.17 |
| | phenylpentan-2-yl)-1- | (m, 5H), 5.63 (s, 1H), 3.89 (s, 3H), 3.44 (d, J = 13.3 Hz, 1H), 2.97 |
| | methyl-1H- | (d, J = 13.3 Hz, 1H), 2.17-2.12 (m, 1H), 1.94-1.85 (m, 1H), 1.64 |
| | pyrrolo[2,3-b]pyridine- | (dd, J = 14.3, 5.0 Hz, 1H), 1.41 (s, 3H), 1.02 (s, 3H), 1.00 (s, 3H); |
| | 5-carboxamide | ESI MS (m/z) 429.95 (MH)⁺ |
| 52 | 3-chloro-N-(2,4- | ¹H-NMR (400 MHz, CHLOROFORM-D) δ 8.67 (d, J = 2.0 Hz, |
| | dimethyl-1- | 1H), 8.16 (d, J = 2.0 Hz, 1H), 7.27-7.16 (m, 6H), 5.64 (s, 1H), |
| | phenylpentan-2-yl)-1- | 3.87 (s, 3H), 3.44 (d, J = 13.8 Hz, 1H), 2.96 (d, J = 13.8 Hz, 1H), |
| | methyl-1H- | 2.15 (q, J = 7.1 Hz, 1H), 1.92-1.86 (m, 1H), 1.63 (dd, J = 13.8, |
| | pyrrolo[2,3-b]pyridine- | 4.9 Hz, 1H), 1.40 (s, 3H), 1.0 (d, J = 6.7 Hz, 6H); ESI MS (m/z) |
| | 5-carboxamide | 384.30 (MH)⁺ |
| 53 | 3-chloro-1-methyl-N- | ¹H-NMR (400 MHz, CHLOROFORM-D) δ 8.70 (d, J = 2.0 Hz, |
| | (2-methyl-1- | 1H), 8.16 (d, J = 2.2 Hz, 1H), 7.28 (dt, J = 6.5, 1.8 Hz, 1H), 7.25- |
| | phenylpropan-2-yl)- | 7.18 (m, 5H), 5.77 (s, 1H), 3.88 (s, 3H), 3.19 (s, 2H), 1.50 (s, 6H); |
| | 1H-pyrrolo[2,3-b] | ESI MS (m/z) 342.35 (MH)⁺ |
| | pyridine-5- | |
| | carboxamide | |
| 54 | 3-cyclopropyl-1- | ¹H-NMR (400 MHz, CHLOROFORM-D) δ 8.57 (d, J = 2.0 Hz, |
| | methyl-N-(2-methyl-1- | 1H), 8.32 (d, J = 2.0 Hz, 1H), 7.29-7.16 (m, 5H), 6.91 (d, J = 1.0 |
| | phenylpropan-2-yl)- | Hz, 1H), 5.77 (s, 1H), 3.82 (s, 3H), 3.19 (s, 2H), 1.92-1.88 (m, |
| | 1H-pyrrolo[2,3-b] | 1H), 1.50 (s, 6H), 0.93-0.88 (m, 2H), 0.64-0.61 (m, 2H); ESI MS |
| | pyridine-5- | (m/z) 348.10 (MH)⁺ |
| | carboxamide | |
| 55 | 3-cyclopropyl-N-(2,4- | ¹H-NMR (400 MHz, CHLOROFORM-D) δ 8.55 (d, J = 2.2 Hz, |
| | dimethyl-1- | 1H), 8.33 (d, J = 2.0 Hz, 1H), 7.25-7.17 (m, 5H), 6.91 (s, 1H), |
| | phenylpentan-2-yl)-1- | 5.64 (s, 1H), 3.83 (s, 3H), 3.43 (d, J = 13.4 Hz, 1H), 2.99 (d, J = |
| | methyl-1H- | 13.4 Hz, 1H), 1.94-1.87 (m, 2H), 1.64 (dd, J = 14.2, 4.9 Hz, 2H), |
| | pyrrolo[2,3-b]pyridine- | 1.42 (d, J = 5.9 Hz, 3H), 1.02-0.97 (m, 6H), 0.93-0.88 (m, 2H), |
| | 5-carboxamide | 0.65-0.61 (m, 2H); ESI MS (m/z) 390.15 (MH)+ |
| 56 | 3-chloro-N,1-dimethyl- | ¹H-NMR (400 MHz, DMSO-d6) δ 8.34 (d, J = 1.7 Hz, 1H), 7.86 |
| | N-(2-methyl-1- | (d, J = 2.2 Hz, 1H), 7.83 (s, 1H), 7.31-7.27 (m, 2H), 7.24-7.20 |
| | phenylpropan-2-yl)- | (m, 3H), 3.82 (s, 3H), 3.28 (s, 2H), 2.52 (s, 3H), 1.47 (s, 6H); ESI |
| | 1H-pyrrolo[2,3-b] | MS (m/z) 355.05 (MH)⁺ |
| | pyridine-5- | |
| | carboxamide | |
| 57 | 3-cyclopropyl-N,1- | ¹H-NMR (400 MHz, CHLOROFORM-D) δ 8.34 (d, J = 2.0 Hz, |
| | dimethyl-N-(2-methyl- | 1H), 8.07 (d, J = 2.0 Hz, 1H), 7.29 -7.20 (m, 5H), 6.91 (s, 1H), |
| | 1-phenylpropan-2-yl)- | 3.82 (s, 3H), 3.39 (s, 2H), 2.63 (s, 3H), 1.98-1.99 (S, 1H), 1.56 |
| | 1H-pyrrolo[2,3-b] | (s, 6H), 0.93-0.88 (m, 2H), 0.65-0.61 (m, 2H); ESI MS (m/z) |
| | pyridine-5- | 362.15 (MH)⁺ |
| | carboxamide | |
| 58 | 3-bromo-N-(1-(2- | ¹H-NMR (400 MHz, CHLOROFORM-D) δ 8.71 (d, J = 2.0 Hz, |
| | fluorophenyl)-2- | 1H), 8.14 (d, J = 2.2 Hz, 1H), 7.27 (s, 1H), 7.24-7.16 (m, 2H), |
| | methylpropan-2-yl)-1- | 7.09-7.01 (m, 2H), 6.02 (s, 1H), 3.90 (s, 3H), 3.22 (d, J = 1.0 Hz, |
| | methyl-1H- | 2H), 1.50 (s, 6H); ESI MS (m/z) 405.95 (MH)⁺ |
| | pyrrolo[2,3-b]pyridine- | |
| | 5-carboxamide | |
| 59 | 3-chloro-N-(1-(2- | ¹H-NMR (400 MHz, CHLOROFORM-D) δ 8.73 (d, J = 2.0 Hz, |
| | fluorophenyl)-2- | 1H), 8.20 (d, J = 2.0 Hz, 1H), 7.24-7.16 (m, 3H), 7.08-7.01 (m, |
| | methylpropan-2-yl)-1- | 2H), 6.00 (s, 1H), 3.88 (s, 3H), 3.22 (d, J = 1.2 Hz, 2H), 1.51 (s, |
| | methyl-1H- | 6H); ESI MS (m/z) 360.25 (MH)⁺ |
| | pyrrolo[2,3-b]pyridine- | |
| | 5-carboxamide | |
| 60 | 3-cyclopropyl-N-(1-(2- | ¹H-NMR (400 MHz, CHLOROFORM-D) δ 8.61 (d, J = 2.2 Hz, |
| | fluorophenyl)-2- | 1H), 8.36 (d, J = 2.2 Hz, 1H), 7.23-7.18 (m, 2H), 7.08-7.00 (m, |
| | methylpropan-2-yl)-1- | 2H), 6.92 (d, J = 0.7 Hz, 1H), 6.01 (s, 1H), 3.82 (s, 3H), 3.22 (d, |
| | methyl-1H- | J = 1.2 Hz, 2H), 1.95-1.88 (m, 1H), 1.54-1.52 (s, 6H), 0.93-0.89 |
| | pyrrolo[2,3-b]pyridine- | (m, 2H), 0.65-0.61 (m, 2H); ESI MS (m/z) 366.40 (MH)⁺ |
| | 5-carboxamide | |
| 61 | N-(1-(4-bromophenyl)- | ¹H-NMR(400 MHz, CHLOROFORM-D) δ 8.63 (d, J = 2.2 Hz, |
| | 2-methylpropan-2-yl)- | 1H), 8.21 (d, J = 2.0 Hz, 1H), 7.35 (dt, J = 8.8, 2.3 Hz, 2H), 7.24 |
| | 1-methyl-1H- | (d, J = 3.4 Hz, 1H), 7.06-7.02 (m, 2H), 6.51 (d, J = 3.4 Hz, 1H), |
| | pyrrolo[2,3-b]pyridine- | 5.67 (s, 1H), 3.91 (s, 3H), 3.18 (s, 2H), 1.46 (s, 6H); ESI MS |
| | 5-carboxamide | (m/z) 387.95 (MH)⁺ |
| 62 | N-(2,4-dimethyl-1- | ¹H-NMR (400 MHz, CHLOROFORM-D) δ 8.51 (t, J = 1.0 Hz, |
| | phenylpentan-2-yl)- | 1H), 7.79 (t, J = 1.0 Hz, 1H), 7.25-7.18 (m, 3H), 7.16-7.14 (m, |
| | 6,7-dihydro-5H- | 2H), 5.53 (s, 1H), 3.41 (d, J = 13.4 Hz, 1H), 3.05-2.91 (m, 5H), |
| | cyclopenta[b]pyridine- | 2.20-2.09 (m, 3H), 1.89-1.83 (m, 1H), 1.60 (dd, J = 14.3, 5.0 Hz, |
| | 3-carboxamide | 1H), 1.36 (s, 3H), 0.98 (dd, J = 6.6, 5.4 Hz, 6H); ESI MS (m/z) |
| | | 337.40 (MH)⁺ |
| 63 | 3-bromo-N-(3-(2- | ¹H-NMR (400 MHz, DMSO-d6) δ 8.73 (d, J = 2.0 Hz, 1H), 8.24 |
| | fluorophenyl)-2- | (d, J = 2.0 Hz, 1H), 7.91 (s, 1H), 7.85 (s, 1H), 7.40-7.36 (m, 1H), |
| | methylbutan-2-yl)-1- | 7.27-7.22 (m, 1H), 7.16-7.07 (m, 2H), 4.27 (q, J = 7.3 Hz, 1H), |
| | methyl-1H- | 3.84 (s, 3H), 1.34 (d, J = 14.9 Hz, 3H), 1.27-1.24 (m, 6H); ESI |
| | pyrrolo[2,3-b]pyridine- | MS (m/z) 420.00 (MH)⁺ |
| | 5-carboxamide | |
| 64 | 3-chloro-N-(3-(2- | ¹H-NMR (400 MHz, DMSO-d6) δ 8.74 (d, J = 2.0 Hz, 1H), 8.34 |
| | fluorophenyl)-2- | (d, J = 2.3 Hz, 1H), 7.89 (s, 1H), 7.82 (s, 1H), 7.38 (td, J = 7.6, |
| | methylbutan-2-yl)-1- | 1.7 Hz, 1H), 7.27-7.22 (m, 1H), 7.16-7.07 (m, 2H), 4.27 (q, J = |
| | methyl-1H- | 7.3 Hz, 1H), 3.83 (s, 3H), 1.36 (s, 3H), 1.27-1.24 (m, 6H); ESI |
| | pyrrolo[2,3-b]pyridine- | MS (m/z) 374.05 (MH)⁺ |
| | 5-carboxamide | |
| 65 | 3-cyclopropyl-N-(3-(2- | ¹H-NMR (400 MHz, CHLOROFORM-D) δ 8.59 (d, J = 2.0 Hz, |
| | fluorophenyl)-2- | 1H), 8.34 (d, J = 2.2 Hz, 1H), 7.32 (td, J = 7.6, 1.7 Hz, 1H), 7.25- |
| | methylbutan-2-yl)-1- | 7.19 (m, 1H), 7.14-7.04 (m, 2H), 6.92 (d, J = 0.7 Hz, 1H), 6.13 |
| | methyl-1H- | (s, 1H), 3.82 (m, 4H), 1.94-1.89 (m, 1H), 1.57 (s, 3H), 1.50 (s, |
| | pyrrolo[2,3-b]pyridine- | 3H), 1.41 (d, J = 7.2 Hz, 3H), 0.94-0.88 (m, 2H), 0.66-0.62 (m, |
| | 5-carboxamide | 2H); ESI MS (m/z) 380.40 (MH)⁺ |
| 66 | N-(1-(4-chlorophenyl)- | ¹H-NMR (400 MHz, CHLOROFORM-D) δ 8.67 (d, J = 2.0 Hz, |
| | 2-methylpropan-2-yl)- | 1H), 8.21 (q, J = 1.0 Hz, 1H), 7.21 (dt, J = 8.7, 2.3 Hz, 2H), 7.10 |
| | 3-fluoro-1-methyl-1H- | (dt, J = 8.9, 2.2 Hz, 2H), 7.01 (d, J = 2.3 Hz, 1H), 5.66 (s, 1H), |
| | pyrrolo[2,3-b]pyridine- | 3.85 (s, 3H), 3.19 (s, 2H), 1.47 (s, 6H); ESI MS (m/z) 360.00 |
| | 5-carboxamide | (MH)⁺ |
| 67 | N-(2-methyl-1- | ¹H-NMR (400 MHz, CHLOROFORM-D) δ 8.52 (t, J = 1.1 Hz, |
| | phenylpropan-2-yl)- | 1H), 7.81 (t, J = 1.0 Hz, 1H), 7.29-7.27 (m, 1H), 7.25-7.20 (m, |
| | 6,7-dihydro-5H- | 2H), 7.17-7.15 (m, 2H), 5.66 (s, 1H), 3.15 (s, 2H), 3.00 (dt, J = |
| | cyclopenta[b]pyridine- | 28.3, 7.6 Hz, 4H), 2.20-2.12 (m, 2H), 1.47 (s, 6H); ESI MS (m/z) |
| | 3-carboxamide | 295.30 (MH)⁺ |
| 68 | 3-fluoro-N-(1-(2- | ¹H-NMR (400 MHz, CHLOROFORM-D) δ 8.70 (d, J = 2.2 Hz, |
| | fluorophenyl)-2- | 1H), 8.24 (q, J = 1.0 Hz, 1H), 7.23-7.16 (m, 2H), 7.08-7.00 (m, |
| | methylpropan-2-yl)-1- | 3H), 5.96 (s, 1H), 3.85 (s, 3H), 3.22 (s, 2H), 1.50 (s, 6H); ESI MS |
| | methyl-1H- | (m/z) 343.50 (MH)⁺ |
| | pyrrolo[2,3-b]pyridine- | |
| | 5-carboxamide | |
| 69 | N-(1-(4-chloro-3- | ¹H-NMR (400 MHz, CHLOROFORM-D) δ 8.67 (d, J = 2.0 Hz, |
| | fluorophenyl)-2- | 1H), 8.22 (q, J = 1.0 Hz, 1H), 7.23 (d, J = 8.1 Hz, 1H), 7.01 (d, J = |
| | methylpropan-2-yl)-3- | 2.4 Hz, 1H), 7.00-6.95 (m, 1H), 6.89 (dd, J = 8.2, 1.3 Hz, 1H), |
| | fluoro-1-methyl-1H- | 5.68 (s, 1H), 3.85 (s, 3H), 3.22 (s, 2H), 1.47 (s, 6H); ESI MS |
| | pyrrolo[2,3-b]pyridine- | (m/z) 377.35 (MH)⁺ |
| | 5-carboxamide | |
| 70 | 3-fluoro-1-methyl-N- | ¹H-NMR (400 MHz, CHLOROFORM-D) δ 8.67 (d, J = 2.2 Hz, |
| | (2-methyl-1-(p- | 1H), 8.19 (d, J = 1.0 Hz, 1H), 7.07 (s, 4H), 7.00 (d, J = 2.7 Hz, |
| | tolyl)propan-2-yl)-1H- | 1H), 5.75 (s, 1H), 3.85 (s, 3H), 3.13 (s, 2H), 2.30 (s, 3H), 1.49 (s, |
| | pyrrolo[2,3-b]pyridine- | 6H); ESI MS (m/z) 340.00 (MH)⁺ |
| | 5-carboxamide | |
| 71 | N-(3-(2-chlorophenyl)- | ¹H-NMR (400 MHz, CHLOROFORM-D) δ 8.68 (d, J = 2.3 Hz, |
| | 2-methylbutan-2-yl)-3- | 1H), 8.22 (dd, J = 2.1, 0.6 Hz, 1H), 7.40 (qd, J = 7.7, 1.5 Hz, 2H), |
| | fluoro-1-methyl-1H- | 7.28 (d, J = 7.3 Hz, 1H), 7.18 (td, J = 7.6, 1.7 Hz, 1H), 6.99 (d, J = |
| | pyrrolo[2,3-b]pyridine- | 2.7 Hz, 1H), 6.11 (s, 1H), 4.13 (q, J = 7.2 Hz, 1H), 3.85 (s, 3H), |
| | 5-carboxamide | 1.57 (s, 3H), 1.55 (s, 3H), 1.37 (d, J = 7.1 Hz, 3H); ESI MS (m/z) |
| | | 374.15 (MH)⁺ |
| 72 | 3-fluoro-N-(1-(2- | ¹H-NMR (400 MHz, CHLOROFORM-D) δ 8.72 (d, J = 2.0 Hz, |
| | methoxyphenyl)-2- | 1H), 8.17-8.16 (m, 1H), 7.46 (s, 1H), 7.29-7.27 (m, 1H), 7.18 (dd, |
| | methylpropan-2-yl)-1- | J = 7.5, 1.6 Hz, 1H), 6.99-6.95 (m, 3H), 3.95 (s, 3H), 3.85 (s, 3H), |
| | methyl-1H- | 2.96 (s, 2H), 1.59 (s, 6H); ESI MS (m/z) 355.90 (MH)⁺ |
| | pyrrolo[2,3-b]pyridine- | |
| | 5-carboxamide | |
| 73 | N-(2,4-dimethyl-1- | ¹H-NMR (400 MHz, DMSO-d6) δ 11.53 (s, 1H), 8.68 (d, J = 2.0 |
| | phenylpentan-2-yl)- | Hz, 1H), 8.07 (q, J = 1.0 Hz, 1H), 7.77 (t, J = 2.9 Hz, 1H), 7.54 |
| | 1H-pyrrolo[3,2- | (s, 1H), 7.23-7.12 (m, 5H), 6.60-6.59 (m, 1H), 3.60 (d, J = 13.0 |
| | b]pyridine-6- | Hz, 1H), 2.66 (d, J = 12.7 Hz, 1H), 2.33 (q, J = 6.9 Hz, 1H), 1.82- |
| | carboxamide | 1.78 (m, 1H), 1.43-1.35 (m, 1H), 1.17 (s, 3H), 0.96-0.89 (m, 6H); |
| | | ESI MS (m/z) 336.35 (MH)⁺ |
| 74 | N-(1-(4-fluorophenyl)- | ¹H-NMR (400 MHz, CHLOROFORM-D) δ 8.58 (d, J = 2.0 Hz, |
| | 2-methylpropan-2-yl)- | 1H), 8.16 (d, J = 2.0 Hz, 1H), 7.28-7.20 (m, 5H), 6.49 (d, J = 3.7 |
| | 1-methyl-1H- | Hz, 1H), 3.90 (s, 3H), 3.76 (q, J = 7.3 Hz, 1H), 1.58 (s, 3H), 1.39- |
| | pyrrolo[2,3-b]pyridine- | 1.36 (m, 6H); ESI MS (m/z) 326.05 (MH)⁺ |
| | 5-carboxamide | |
| 75 | 1-methyl-N-(4- | ¹H-NMR (400 MHz, CHLOROFORM-D) δ 8.69 (d, J = 2.2 Hz, |
| | phenylbutan-2-yl)-1H- | 1H), 8.21 (d, J = 2.2 Hz, 1H), 7.30-7.28 (m, 1H), 7.26-7.16 (m, |
| | pyrrolo[2,3-b]pyridine- | 5H), 6.51 (d, J = 3.7 Hz, 1H), 5.95 (d, J = 8.1 Hz, 1H), 4.32 (dt, |
| | 5-carboxamide | J = 14.8, 6.7 Hz, 1H), 3.91 (s, 3H), 2.74 (dd, J = 15.5, 7.5 Hz, |
| | | 2H), 1.97-1.88 (m, 2H), 1.32 (d, J = 6.5 Hz, 3H); ESI MS (m/z) |
| | | 308.05 (MH)⁺ |
| 76 | N-(2,3-dihydro-1H- | ¹H-NMR (400 MHz, CHLOROFORM-D) δ 8.73 (d, J = 2.0 Hz, |
| | inden-2-yl)-1-methyl- | 1H), 8.35 (d, J = 2.0 Hz, 1H), 7.28-7.27 (m, 2H), 7.25 (d, J = 3.7 |
| | 1H-pyrrolo[2,3-b] | Hz, 1H), 7.22-7.18 (m, 2H), 6.54 (d, J = 3.4 Hz, 1H), 6.42 (s, 1H), |
| | pyridine-5- | 5.02-4.97 (m, 1H), 3.93 (s, 3H), 3.45 (dd, J = 16.1, 7.1 Hz, 2H), |
| | carboxamide | 3.01-2.95 (m, 2H); ESI MS (m/z) 292 (MH)⁺ |
| 77 | 1-methyl-N-(2-methyl- | ¹H-NMR (400 MHz, CHLOROFORM-D) δ 8.67 (d, J = 2.2 Hz, |
| | 1-(naphthalen-2- | 1H), 8.23 (d, J = 2.0 Hz, 1H), 7.79-7.76 (m, 1H), 7.75-7.70 (m, |
| | yl)propan-2-yl)-1H- | 2H), 7.64 (s, 1H), 7.44-7.39 (m, 2H), 7.33 (dd, J = 8.3, 1.7 Hz, |
| | pyrrolo[2,3-b]pyridine- | 1H), 7.24 (d, J = 3.4 Hz, 1H), 6.50 (d, J = 3.4 Hz, 1H), 5.76 (s, |
| | 5-carboxamide | 1H), 3.93 (s, 3H), 3.37 (s, 2H), 1.54 (s, 6H); ESI MS (m/z) 358 |
| | | (MH)⁺ |
| 78 | 1-methyl-N-(2-methyl- | ¹H-NMR (400 MHz, CHLOROFORM-D) δ 8.70 (d, J = 2.0 Hz, |
| | 2,3-dihydro-1H-inden- | 1H), 8.29 (d, J = 2.0 Hz, 1H), 7.22 (dt, J = 8.9, 3.6 Hz, 3H), 7.19- |
| | 2-yl)-1H-pyrrolo[2,3-b] | 7.14 (m, 2H), 6.52 (d, J = 3.4 Hz, 1H), 6.31 (s, 1H), 3.91 (s, 3H), |
| | pyridine-5- | 3.51 (d, J = 15.6 Hz, 2H), 3.16 (d, J = 15.9 Hz, 2H), 1.70 (s, 3H); |
| | carboxamide | ESI MS (m/z) 306.05 (MH)⁺ |
| 79 | 1-methyl-N-((1- | ¹H-NMR (400 MHz, CHLOROFORM-D) δ 8.62 (d, J = 2.0 Hz, |
| | phenylcyclopropyl) | 1H), 8.26 (d, J = 2.0 Hz, 1H), 7.39-7.32 (m, 4H), 7.25-7.22 (m, |
| | methyl)-1H-pyrrolo | 2H), 6.51 (d, J = 3.4 Hz, 1H), 6.13 (s, 1H), 3.90 (s, 3H), 3.68 (d, |
| | [2,3-b]pyridine-5- | J = 5.6 Hz, 2H), 1.02-0.93 (m, 4H); ESI MS (m/z) 305.95 (MH)⁺ |
| | carboxamide | |
| 80 | N-(2,3-dimethyl-4- | ¹H-NMR (400 MHz, CHLOROFORM-D) δ 8.70 (d, J = 2.0 Hz, |
| | phenylbutan-2-yl)-1- | 1H), 8.22 (d, J = 2.0 Hz, 1H), 7.27-7.23 (m, 3H), 7.21-7.13 (m, |
| | methyl-1H- | 3H), 6.52 (d, J = 3.4 Hz, 1H), 5.94 (s, 1H), 3.92 (s, 3H), 3.01 (dd, |
| | pyrrolo[2,3-b]pyridine- | J = 13.1, 3.3 Hz, 1H), 2.78-2.69 (m, 1H), 2.22 (dd, J = 13.1, 10.9 |
| | 5-carboxamide | Hz, 1H), 1.52 (d, J = 10.8 Hz, 6H), 0.86 (d, J = 6.8 Hz, 3H); ESI |
| | | MS (m/z) 336.05 (MH)⁺ |
| 81 | N-(2,4-dimethyl-1- | ¹H-NMR (400 MHz, CHLOROFORM-D) δ 8.02 (d, J = 2.0 Hz, |
| | phenylpentan-2-yl)-4- | 1H), 7.25-7.12 (m, 6H), 5.39 (s, 1H), 4.23 (t, J = 4.6 Hz, 2H), |
| | methyl-3,4-dihydro- | 3.49 (t, J = 4.5 Hz, 2H), 3.36 (d, J = 13.2 Hz, 1H), 3.16 (s, 3H), |
| | 2H-pyrido[3,2-b] | 2.94 (d, J = 13.4 Hz, 1H), 2.03 (q, J = 7.1 Hz, 1H), 1.87-1.81 (m, |
| | [1,4]oxazine-7- | 1H), 1.55 (dd, J = 14.2, 4.9 Hz, 1H), 1.33 (s, 3H), 0.96 (q, J = 3.3 |
| | carboxamide | Hz, 6H); ESI MS (m/z) 367.95 (MH)⁺ |
| 82 | 4-methyl-N-(2-methyl- | ¹H-NMR (400 MHz, CHLOROFORM-D) δ 8.03 (d, J = 2.0 Hz, |
| | 1-phenylpropan-2-yl)- | 1H), 7.26-7.18 (m, 4H), 7.16-7.14 (m, 2H), 5.51 (s, 1H), 4.22 (t, |
| | 3,4-dihydro-2H- | J = 4.5 Hz, 2H), 3.49 (t, J = 4.6 Hz, 2H), 3.16 (s, 3H), 3.13 (s, |
| | pyrido[3,2-b] | 2H), 1.43 (s, 6H); ESI MS (m/z) 326.10 (MH)⁺ |
| | [1,4]oxazine-7- | |
| | carboxamide | |
| 83 | 3-chloro-N-(1-(2- | ¹H-NMR (400 MHz, CHLOROFORM-D) δ 8.75 (d, J = 2.2 Hz, |
| | chlorophenyl)-2- | 1H), 8.21 (dd, J = 10.8, 2.0 Hz, 1H), 7.40 (dd, J = 7.5, 1.8 Hz, |
| | methylpropan-2-yl)-1- | 1H), 7.24-7.21 (m, 2H), 7.20-7.12 (m, 2H), 6.04 (s, 1H), 3.89 (s, |
| | methyl-1H- | 3H), 3.38 (d, J = 1.7 Hz, 2H), 1.56 (s, 6H); ESI MS (m/z) 375.95 |
| | pyrrolo[2,3-b]pyridine- | (MH)⁺ |
| | 5-carboxamide | |
| 84 | 3-bromo-N-(1-(2- | ¹H-NMR (400 MHz, CHLOROFORM-D) δ 8.74 (d, J = 2.0 Hz, |
| | chlorophenyl)-2- | 1H), 8.16 (d, J = 2.2 Hz, 1H), 7.40 (dd, J = 7.5, 1.8 Hz, 1H), 7.28 |
| | methylpropan-2-yl)-1- | (s, 1H), 7.24 (dd, J = 7.1, 2.4 Hz, 1H), 7.19-7.12 (m, 2H), 6.06 |
| | methyl-1H- | (s, 1H), 3.90 (s, 3H), 3.38 (s, 2H), 1.57 (s, 6H); ESI MS (m/z) |
| | pyrrolo[2,3-b]pyridine- | 421.90 (MH)⁺ |
| | 5-carboxamide | |
| 85 | N-(1-(2-chlorophenyl)- | ¹H-NMR (400 MHz, CHLOROFORM-D) δ 8.63 (d, J = 2.2 Hz, |
| | 2-methylpropan-2-yl)- | 1H), 8.37 (d, J = 2.0 Hz, 1H), 7.39 (dd, J = 7.5, 1.8 Hz, 1H), 7.24 |
| | 3-cyclopropyl-1- | (d, J = 2.4 Hz, 1H), 7.19-7.12 (m, 2H), 6.92 (s, 1H), 6.04 (s, 1H), |
| | methyl-1H- | 3.83 (s, 3H), 3.38 (s, 2H), 1.94-1.90 (m, 1H), 1.57 (s, 6H), 0.94- |
| | pyrrolo[2,3-b]pyridine- | 0.89 (m, 2H), 0.65-0.61 (m, 2H); ESI MS (m/z) 382.10 (MH)⁺ |
| | 5-carboxamide | |
| 86 | 1-methyl-N-(2-methyl- | ¹H-NMR (400 MHz, CHLOROFORM-D) δ 8.58 (d, J = 1.5 Hz, |
| | 1-phenylpropan-2-yl)- | 1H), 8.16 (d, J = 0.9 Hz, 1H), 7.41 (d, J = 3.2 Hz, 1H), 7.28 (s, |
| | 1H-pyrrolo[3,2-b] | 1H), 7.24-7.18 (m, 4H), 6.71 (d, J = 3.2 Hz, 1H), 5.88 (s, 1H), |
| | pyridine-6- | 3.86 (s, 3H), 3.19 (s, 2H), 1.51 (s, 6H); ESI MS (m/z) 307.95 |
| | carboxamide | (MH)⁺ |
| 87 | 3-chloro-1-methyl-N- | ¹H-NMR (400 MHz, CHLOROFORM-D) δ 8.71 (d, J = 1.8 Hz, |
| | (2-methyl-3- | 1H), 8.14 (d, J = 2.1 Hz, 1H), 7.37-7.28 (m, 6H), 5.86 (s, 1H), |
| | phenylbutan-2-yl)-1H- | 3.94 (s, 3H), 3.79 (q, J = 7.2 Hz, 1H), 1.66 (s, 3H), 1.45-1.43 (m, |
| | pyrrolo[2,3-b]pyridine- | 6H); ESI MS (m/z) 356.05 (MH)⁺ |
| | 5-carboxamide | |
| 88 | 6-methoxy-N-(2- | ¹H-NMR (400 MHz, CHLOROFORM-D) δ 8.82 (d, J = 0.5 Hz, |
| | methyl-1- | 1H), 8.46 (s, 1H), 7.71 (s, 1H), 7.25-7.17 (m, 5H), 7.13 (dd, J = |
| | phenylpropan-2-yl)- | 3.5, 2.3 Hz, 1H), 6.53 (q, J = 1.9 Hz, 1H), 3.96 (s, 3H), 3.19 (s, |
| | 1H-pyrrolo[2,3-b] | 2H), 1.47 (s, 6H); ESI MS (m/z) 324.20 (MH)⁺ |
| | pyridine-5- | |
| | carboxamide | |
| 89 | N-(2,4-dimethyl-1- | ¹H-NMR (400 MHz, CHLOROFORM-D) δ 8.83 (s, 1H), 8.47 (s, |
| | phenylpentan-2-yl)-6- | 1H), 7.61 (s, 1H), 7.23-7.13 (m, 6H), 6.53 (q, J = 1.9 Hz, 1H), |
| | methoxy-1H- | 3.94 (s, 3H), 3.37 (d, J = 13.2 Hz, 1H), 3.06 (d, J = 13.2 Hz, 1H), |
| | pyrrolo[2,3-b]pyridine- | 2.05-1.99 (m, 1H), 1.90-1.84 (m, 1H), 1.57-1.54 (m, 1H), 1.40 (s, |
| | 5-carboxamide | 3H), 0.97 (dd, J = 6.6, 5.4 Hz, 6H); ESI MS (m/z) 366.30 (MH)⁺ |
| 90 | 1-methyl-N-(2-methyl- | ¹H-NMR (400 MHz, CHLOROFORM-D) δ 8.76 (d, J = 2.1 Hz, |
| | 1-(4- | 1H), 8.32 (d, J = 2.1 Hz, 1H), 7.25-7.22 (m, 5H), 6.53-6.51 (m, |
| | (methylthio)phenyl)pro | 1H), 6.38 (d, J = 8.9 Hz, 1H), 4.96 (t, J = 8.3 Hz, 1H), 3.91 (s, |
| | pyl)-1H-pyrrolo[2,3-b] | 3H), 2.48 (s, 3H), 2.16 (td, J = 13.9, 7.0 Hz, 1H), 1.06 (d, J = 6.7 |
| | pyridine-5- | Hz, 3H), 0.92 (d, J = 6.7 Hz, 3H); ESI MS (m/z) 353.85 (MH)⁺ |
| | carboxamide | |
| 91 | 6-methoxy-1-methyl- | ¹H-NMR (400 MHz, CHLOROFORM-D) δ 8.78 (s, 1H), 7.75 (s, |
| | N-(2-methyl-1- | 1H), 7.23-7.16 (m, 5H), 7.02 (t, J = 1.8 Hz, 1H), 6.47 (d, J = 3.4 |
| | phenylpropan-2-yl)- | Hz, 1H), 4.00 (s, 3H), 3.80 (s, 3H), 3.19 (s, 2H), 1.46 (s, 6H); ESI |
| | 1H-pyrrolo[2,3-b] | MS (m/z) 338.25 (MH)⁺ |
| | pyridine-5- | |
| | carboxamide | |
| 92 | N-(2,4-dimethyl-1- | ¹H-NMR (400 MHz, CHLOROFORM-D) δ 8.79 (s, 1H), 7.66 (s, |
| | phenylpentan-2-yl)-6- | 1H), 7.22-7.15 (m, 5H), 7.02 (d, J = 3.4 Hz, 1H), 6.48 (d, J = 3.4 |
| | methoxy-1-methyl-1H- | Hz, 1H), 3.99 (s, 3H), 3.80 (s, 3H), 3.36 (d, J = 13.2 Hz, 1H), |
| | pyrrolo[2,3-b]pyridine- | 3.06 (d, J = 13.2 Hz, 1H), 2.05-1.99 (m, 1H), 1.90-1.84 (m, 1H), |
| | 5-carboxamide | 1.58-1.53 (m, 1H), 1.40 (s, 3H), 0.97 (dd, J = 6.6, 5.4 Hz, 6H); |
| | | ESI MS (m/z) 380.10 (MH)⁺ |
| 93 | 6-methyl-N-(2-methyl- | ¹H-NMR (400 MHz, DMSO-d6) δ 11.53 (s, 1H), 7.74 (s, 1H), |
| | 1-phenylpropan-2-yl)- | 7.63 (s, 1H), 7.39 (dd, J = 3.4, 2.4 Hz, 1H), 7.31-7.28 (m, 2H), |
| | 1H-pyrrolo[2,3-b] | 7.23-7.19 (m, 3H), 6.40 (q, J = 1.8 Hz, 1H), 3.13 (s, 2H), 2.58 (s, |
| | pyridine-5- | 3H), 1.32 (s, 6H); ESI MS (m/z) 308.25 (MH)⁺ |
| | carboxamide | |
| 94 | N-(1-(4-fluorophenyl)- | ¹H-NMR (400 MHz, DMSO-d6) δ 11.53 (s, 1H), 7.74 (s, 1H), |
| | 2-methylpropan-2-yl)- | 7.63 (s, 1H), 7.39 (dd, J = 3.3, 2.6 Hz, 1H), 7.26-7.22 (m, 2H), |
| | 6-methyl-1H- | 7.14-7.10 (m, 2H), 6.41 (q, J = 1.8 Hz, 1H), 3.13 (s, 2H), 2.57 (s, |
| | pyrrolo[2,3-b]pyridine- | 3H), 1.31 (s, 6H); ESI MS (m/z) 326 (MH)⁺ |
| | 5-carboxamide | |
| 95 | N-(2-methyl-1- | ¹H-NMR (400 MHz, DMSO-d6) δ 11.53 (s, 1H), 8.71 (d, J = 2.0 |
| | phenylpropan-2-yl)- | Hz, 1H), 8.10 (q, J = 0.9 Hz, 1H), 7.77 (t, J = 2.9 Hz, 1H), 7.67 |
| | 1H-pyrrolo[3,2-b] | (s, 1H), 7.24-7.12 (m, 5H), 6.60-6.58 (m, 1H), 3.14 (s, 2H), 1.35 |
| | pyridine-6- | (s, 6H); ESI MS (m/z) 294.0 (MH)⁺ |
| | carboxamide | |
| 96 | N-(2,4-dimethyl-1- | ¹H-NMR(400 MHz, DMSO-d6) δ 11.54 (s, 1H), 7.70 (s, 1H), |
| | phenylpentan-2-yl)-6- | 7.51 (s, 1H), 7.39 (t, J = 2.9 Hz, 1H), 7.30-7.27 (m, 2H), 7.21 (t, |
| | methyl-1H- | J = 6.5 Hz, 3H), 6.40 (q, J = 1.8 Hz, 1H), 3.53 (d, J = 13.0 Hz, |
| | pyrrolo[2,3-b]pyridine- | 1H), 2.71 (d, J = 12.7 Hz, 1H), 2.60 (s, 3H), 2.30 (dd, J = 13.9, |
| | 5-carboxamide | 5.6 Hz, 1H), 1.86-1.80 (m, 1H), 1.40 (dd, J = 13.9, 5.4 Hz, 1H), |
| | | 1.13 (s, 3H), 0.96 (dd, J = 6.6, 3.9 Hz, 6H); ESI MS (m/z) 350.10 |
| | | (MH)⁺ |
| 97 | N-(1- | ¹H-NMR (400 MHz, DMSO-d6) δ 11.53 (s, 1H), 8.28 (s, 1H), |
| | benzylcyclopropyl)-6- | 7.66 (s, 1H), 7.37 (dd, J = 3.3, 2.6 Hz, 1H), 7.32-7.25 (m, 4H), |
| | methyl-1H- | 7.23-7.18 (m, 1H), 6.36 (q, J = 1.8 Hz, 1H), 2.99 (s, 2H), 2.48 (s, |
| | pyrrolo[2,3-b]pyridine- | 3H), 0.82-0.74 (m, 4H); ESI MS (m/z) 306.25 (MH)⁺ |
| | 5-carboxamide | |
| 98 | N-(2,4-dimethyl-1- | ¹H-NMR (400 MHz, DMSO-d6) δ 12.63 (s, 1H), 8.42 (d, J = 1.7 |
| | phenylpentan-2-yl)-2- | Hz, 1H), 7.88 (d, J = 1.7 Hz, 1H), 7.51 (s, 1H), 7.25-7.20 (m, 2H), |
| | oxo-2,3- | 7.17-7.14 (m, 1H), 7.11-7.07 (m, 2H), 3.52 (d, J = 13.0 Hz, 1H), |
| | dihydrooxazolo[4,5-b] | 2.67 (d, J = 13.0 Hz, 1H), 2.27 (q, J = 6.9 Hz, 1H), 1.81-1.75 (m, |
| | pyridine-6- | 1H), 1.39 (dd, J = 13.9, 5.1 Hz, 1H), 1.18-1.12 (m, 3H), 0.91 (d, |
| | carboxamide | J = 6.6 Hz, 3H), 0.88 (d, J = 6.6 Hz, 3H); ESI MS (m/z) 354.25 |
| | | (MH)⁺ |
| 99 | 1,2-dimethyl-N-(2- | ¹H-NMR (400 MHz, DMSO-d6) δ 8.56 (d, J = 2.2 Hz, 1H), 8.19 |
| | methyl-1- | (d, J = 2.2 Hz, 1H), 7.57 (s, 1H), 7.23-7.12 (m, 5H), 6.31 (d, J = |
| | phenylpropan-2-yl)- | 1.0 Hz, 1H), 3.73 (s, 3H), 3.13 (s, 2H), 2.45 (s, 3H), 1.34 (s, 6H); |
| | 1H-pyrrolo[2,3-b] | ESI MS (m/z) 322.30 (MH)⁺ |
| | pyridine-5- | |
| | carboxamide | |
| 100 | N-(2,3-dimethyl-3- | ¹H-NMR (400 MHz, DMSO-d6) δ 8.47 (d, J = 2.0 Hz, 1H), 8.11 |
| | phenylbutan-2-yl)-1,2- | (d, J = 2.0 Hz, 1H), 7.49-7.47 (m, 2H), 7.32-7.15 (m, 3H), 7.00 |
| | dimethyl-1H- | (s, 1H), 6.32 (d, J = 1.0 Hz, 1H), 3.73 (s, 3H), 2.45 (dd, J = 5.5, |
| | pyrrolo[2,3-b]pyridine- | 4.8 Hz, 3H), 1.45 (s, 6H), 1.36 (s, 6H); ESI MS (m/z) 350.45 |
| | 5-carboxamide | (MH)⁺ |
| 101 | N-(1-(4-chlorophenyl)- | ¹H-NMR (400 MHz, DMSO-d6) δ 8.56 (d, J = 1.8 Hz, 1H), 8.19 |
| | 2-methylpropan-2-yl)- | (d, J = 2.1 Hz, 1H), 7.58 (s, 1H), 7.27 (dt, J = 8.9, 2.2 Hz, 2H), |
| | 1,2-dimethyl-1H- | 7.15-7.12 (m, 2H), 6.32 (d, J = 1.2 Hz, 1H), 3.73 (s, 3H), 3.13 (s, |
| | pyrrolo[2,3-b]pyridine- | 2H), 2.45 (s, 3H), 1.33 (s, 6H); ESI MS (m/z) 356 (MH)⁺ |
| | 5-carboxamide | |
| 102 | 1,2-dimethyl-N-(2- | ¹H-NMR (400 MHz, DMSO-d6) δ 8.56 (d, J = 2.2 Hz, 1H), 8.19 |
| | methyl-1-(p-tolyl) | (d, J = 2.2 Hz, 1H), 7.53 (s, 1H), 7.01 (s, 4H), 6.31 (d, J = 1.2 Hz, |
| | propan-2-yl)-1H- | 1H), 3.73 (s, 3H), 3.08 (s, 2H), 2.45 (s, 3H), 2.21 (s, 3H), 1.33 (s, |
| | pyrrolo[2,3-b]pyridine- | 6H); ESI MS (m/z) 336.50 (MH)⁺ |
| | 5-carboxamide | |
| 103 | N-(1-(2-fluorophenyl)- | ¹H-NMR (400 MHz, DMSO-d6) δ 8.58 (d, J = 1.8 Hz, 1H), 8.21 |
| | 2-methylpropan-2-yl)- | (d, J = 2.1 Hz, 1H), 7.66 (s, 1H), 7.26-7.20 (m, 1H), 7.17-7.10 |
| | 1,2-dimethyl-1H- | (m, 2H), 7.03 (td, J = 7.5, 1.1 Hz, 1H), 6.32 (d, J = 0.9 Hz, 1H), |
| | pyrrolo[2,3-b]pyridine- | 3.73 (s, 3H), 3.20 (s, 2H), 2.45 (s, 3H), 1.34 (s, 6H); ESI MS |
| | 5-carboxamide | (m/z) 340.00 (MH)⁺ |
| 104 | N-(3-hydroxy-3- | ¹H-NMR(400 MHz, CHLOROFORM-D) δ 9.59 (s, 1H), 8.46 (d, |
| | methyl-1-(m- | J = 1.5 Hz, 1H), 8.12 (d, J = 2.0 Hz, 1H), 7.37-7.36 (m, 1H), 7.17 |
| | tolyl)butan-2-yl)-1H- | (t, J = 7.5 Hz, 1H), 7.06-7.00 (m, 4H), 6.53 (q, J = 1.7 Hz, 1H), |
| | pyrrolo[2,3-b]pyridine- | 6.25 (d, J = 8.6 Hz, 1H), 4.25-4.19 (m, 1H), 3.21 (dd, J = 14.2, |
| | 5-carboxamide | 3.9 Hz, 1H), 2.82 (dd, J = 14.2, 11.0 Hz, 1H), 2.29 (s, 3H), 1.40 |
| | | (s, 3H), 1.38 (s, 3H); ESI MS (m/z) 338.10 (MH)⁺ |
| 105 | N-(3-hydroxy-2- | ¹H-NMR (400 MHz, CHLOROFORM-D) δ 9.00 (d, J = 2.2 Hz, |
| | methyl-4-phenylbutan- | 1H), 8.61-8.60 (m, 1H), 7.92 (d, J = 5.6 Hz, 1H), 7.62 (dd, J = |
| | 2-yl)thieno[3,2-b] | 5.6, 0.7 Hz, 1H), 7.34-7.28 (m, 4H), 7.25-7.21 (m, 1H), 6.46 (s, |
| | pyridine-6- | 1H), 3.95-3.90 (m, 1H), 3.52 (d, J = 5.9 Hz, 1H), 3.01 (dd, J = |
| | carboxamide | 13.7, 2.4 Hz, 1H), 2.63 (dd, J = 13.7, 10.0 Hz, 1H), 1.57 (s,, 3H), |
| | | 1.56 (s,, 3H); ESI MS (m/z) 341.05 (MH)⁺ |
| 106 | N-(2-cyanopropan-2- | ¹H-NMR (400 MHz, DMSO-d6) δ 9.07 (d, J = 2.0 Hz, 1H), 8.99 |
| | yl)thieno[3,2-b] | (s, 1H), 8.94 (q, J = 1.0 Hz, 1H), 8.35 (t, J = 2.8 Hz, 1H), 7.65 |
| | pyridine-6- | (dd, J = 5.6, 0.7 Hz, 1H), 1.72 (s, 6H); ESI MS (m/z) 246.0 (MH)⁺ |
| | carboxamide | |
| 107 | N-(tert- | ¹H-NMR (400 MHz, DMSO-d6) δ 9.01 (d, J = 2.0 Hz, 1H), 8.84 |
| | butyl)thieno[3,2-b] | (q, J = 1.0 Hz, 1H), 8.28 (d, J = 5.6 Hz, 1H), 8.04 (s, 1H), 7.61 |
| | pyridine-6- | (dd, J = 5.6, 0.7 Hz, 1H), 1.40 (s, 9H); ESI MS (m/z) 235.0 (MH)⁺ |
| | carboxamide | |
| 108 | N-(1-(trifluoromethyl) | ¹H-NMR (400 MHz, DMSO-d6) δ 9.39 (s, 1H), 9.06 (d, J = 2.0 |
| | cyclopropyl)thieno | Hz, 1H), 8.90 (q, J = 0.9 Hz, 1H), 8.34 (d, J = 5.4 Hz, 1H), 7.64 |
| | [3,2-b]pyridine-6- | (dd, J = 5.5, 0.9 Hz, 1H), 1.37-1.29 (m, 2H), 1.22-1.16 (m, 2H); |
| | carboxamide | ESI MS (m/z) 286.9 (MH)⁺ |
| 109 | N-(1,1,1-trifluoro-2- | ¹H-NMR (400 MHz, CHLOROFORM-D) δ 8.98 (d, J = 2.1 Hz, |
| | methylpropan-2- | 1H), 8.57 (q, J = 1.0 Hz, 1H), 7.92 (d, J = 5.5 Hz, 1H), 7.60 (dd, |
| | yl)thieno[3,2-b] | J = 5.5, 0.6 Hz, 1H), 6.17 (s, 1H), 1.76 (s, 6H); ESI MS (m/z) |
| | pyridine-6- | 289.0 (MH)⁺ |
| | carboxamide | |
| 110 | N-(1,1,1-trifluoro-2- | ¹H-NMR (400 MHz, CHLOROFORM-D) δ 9.06 (d, J = 1.8 Hz, |
| | methylbutan-2- | 1H), 8.67 (q, J = 0.9 Hz, 1H), 7.95 (q, J = 2.8 Hz, 1H), 7.64 (dd, |
| | yl)thieno[3,2-b] | J = 5.7, 0.8 Hz, 1H), 6.29 (d, J = 10.1 Hz, 1H), 4.88-4.78 (m, 1H), |
| | pyridine-6- | 2.35-2.26 (m, 1H), 1.10 (t, J = 7.3 Hz, 6H); ESI MS (m/z) 303.0 |
| | carboxamide | (MH)⁺ |
| 111 | N-(1-hydroxy-2- | ¹H-NMR (400 MHz, CHLOROFORM-D) δ 8.99 (d, J = 2.1 Hz, |
| | methylpropan-2- | 1H), 8.62 (d, J = 1.5 Hz, 1H), 7.93 (d, J = 5.5 Hz, 1H), 7.62 (d, J = |
| | yl)thieno[3,2-b] | 5.5 Hz, 1H), 6.30 (s, 1H), 4.21 (s, 1H), 3.75 (s, 2H), 1.47 (s, |
| | pyridine-6- | 6H); ESI MS (m/z) 251.0 (MH)⁺ |
| | carboxamide | |
| 114 | N-(3-hydroxy-3- | ¹H-NMR (400 MHz, DMSO-d6) δ 8.91 (d, J = 2.0 Hz, 1H), 8.75 |
| | methyl-1-(m- | (d, J = 2.5 Hz, 1H), 8.28 (d, J = 2.5 Hz, 1H), 8.22 (d, J = 9.3 Hz, |
| | tolyl)butan-2- | 1H), 7.61 (dd, J = 5.6, 0.7 Hz, 1H), 7.08-7.00 (m, 3H), 6.91-6.88 |
| | yl)thieno[3,2-b] | (m, 1H), 4.60 (t, J = 16.9 Hz, 1H), 4.18-4.08 (m, 1H), 3.10 (dd, J = |
| | pyridine-6- | 13.8, 2.3 Hz, 1H), 2.68 (dd, J = 13.7, 11.7 Hz, 1H), 2.18 (s, |
| | carboxamide | 3H), 1.21 (s, 3H), 1.16 (s, 3H); ESI MS (m/z) 354.85 (MH)⁺ |
| 116 | N-(3-hydroxy-2- | ¹H-NMR (400 MHz, DMSO-d6) δ 8.70 (t, J = 2.6 Hz, 1H), 8.40- |
| | methyl-4-phenylbutan- | 8.39 (m, 1H), 7.77 (s, 1H), 7.59 (d, J = 3.7 Hz, 1H), 7.26-7.20 |
| | 2-yl)-1-methyl-1H- | (m, 4H), 7.16-7.12 (m, 1H), 6.56 (d, J = 3.4 Hz, 1H), 5.02 (d, J = |
| | pyrrolo[2,3-b]pyridine- | 7.3 Hz, 1H), 4.12-4.07 (m, 1H), 3.84 (s, 3H), 2.87 (d, J = 12.5 |
| | 5-carboxamide | Hz, 1H), 2.47-2.41 (m, 1H), 1.43 (d, J = 7.9 Hz, 3H), 1.38 (s, 3H); |
| | | ESI MS (m/z) 338.10 (MH)⁺ |
| 117 | N-(1-hydroxy-2- | ¹H-NMR (400 MHz, CHLOROFORM-D) δ 8.67 (d, J = 2.1 Hz, |
| | methyl-1- | 1H), 8.27 (d, J = 2.1 Hz, 1H), 7.36-7.33 (m, 2H), 7.29 (dd, J = |
| | phenylpropan-2-yl)-1- | 8.4, 1.7 Hz, 2H), 7.25-7.21 (m, 2H), 6.54 (t, J = 3.5 Hz, 1H), 5.93 |
| | methyl-1H- | (d, J = 40.0 Hz, 1H), 4.80 (s, 1H), 3.90 (s, 3H), 1.64 (s, 3H), 1.39 |
| | pyrrolo[2,3-b]pyridine- | (s, 3H); ESI MS (m/z) 324.0 (MH)⁺ |
| | 5-carboxamide | |
| 118 | N-(1-hydroxy-2- | ¹H-NMR (400 MHz, DMSO-d6) δ 8.96 (d, J = 1.8 Hz, 1H), 8.79 |
| | methyl-1- | (d, J = 1.5 Hz, 1H), 8.29 (d, J = 5.8 Hz, 1H), 7.85 (s, 1H), 7.62 |
| | phenylpropan-2- | (d, J = 5.5 Hz, 1H), 7.33-7.18 (m, 5H), 5.68 (d, J = 5.2 Hz, 1H), |
| | yl)thieno[3,2-b] | 5.26 (d, J = 4.9 Hz, 1H), 1.45 (s, 3H), 1.22 (s, 3H); ESI MS (m/z) |
| | pyridine-6- | 327.0 (MH)⁺ |
| | carboxamide | |
| 119 | N-(1-(4-fluoro-3- | ¹H-NMR (400 MHz, DMSO-d6) δ 8.97 (d, J = 1.8 Hz, 1H), 8.81- |
| | methoxyphenyl)-1- | 7.79 (m, 1H), 8.29 (d, J = 5.5 Hz, 1H), 7.84 (s, 1H), 7.62 (dd, J = |
| | hydroxy-2- | 5.5, 0.6 Hz, 1H), 7.09 (dd, J = 11.6, 8.3 Hz, 1H), 7.02 (dd, J = |
| | methylpropan-2- | 8.6, 2.1 Hz, 1H), 6.87 (qd, J = 4.2, 1.9 Hz, 1H), 5.74 (d, J = 5.2 |
| | yl)thieno[3,2-b] | Hz, 1H), 5.24 (d, J = 4.9 Hz, 1H), 3.66 (s, 3H), , 1.48 (s, 3H), |
| | pyridine-6- | 1.21 (s, 3H) ESI MS (m/z) 375.15 (MH)⁺ |
| | carboxamide | |
| 120 | N-(2-ethyl-2-hydroxy- | ¹H-NMR (400 MHz, CHLOROFORM-D) δ 8.81 (d, J = 2.0 Hz, |
| | 1-phenylbutyl)-1- | 1H), 8.33 (d, J = 2.0 Hz, 1H), 7.45-7.42 (m, 2H), 7.36-7.32 (m, |
| | methyl-1H- | 2H), 7.30-7.28 (m, 2H), 7.24 (d, J = 3.7 Hz, 1H), 6.52 (d, J = 3.4 |
| | pyrrolo[2,3-b]pyridine- | Hz, 1H), 5.18 (d, J = 9.0 Hz, 1H), 3.91 (s, 3H), 1.81-1.71 (m, 2H), |
| | 5-carboxamide | 1.40-1.29 (m, 1H), 1.25-1.12 (m, 1H), 0.96 (t, J = 7.5 Hz, 3H), |
| | | 0.87-0.80 (t, J = 7.5 Hz, 3H); ESI MS (m/z) 352.15 (MH)⁺ |
| 121 | N-(1-(4- | ¹H-NMR (400 MHz, DMSO-d6) δ 8.67 (d, J = 2.0 Hz, 1H), 8.37 |
| | fluorophenoxy)-2,4- | (d, J = 2.0 Hz, 1H), 7.85 (s, 1H), 7.59 (d, J = 3.4 Hz, 1H), 7.12- |
| | dimethylpent-4-en-2- | 6.91 (m, 4H), 6.59-6.56 (m, 1H), 4.87-4.85 (m, 1H), 4.78-4.70 |
| | yl)-1-methyl-1H- | (m, 1H), 4.32 (d, J = 9.0 Hz, 1H), 4.09-3.99 (m, 1H), 3.83 (s, 3H), |
| | pyrrolo[2,3-b]pyridine- | 2.91 (d, J = 13.2 Hz, 1H), 2.47 (d, J = 3.2 Hz, 1H), 1.74 (d, J = |
| | 5-carboxamide | 4.2 Hz, 3H), 1.45 (s, 3H); ESI MS (m/z) 381.7 (MH)⁺ |
| 122 | N-(1-(4- | ¹H-NMR (400 MHz, DMSO-d6) δ 8.98 (d, J = 2.0 Hz, 1H), 8.82 |
| | fluorophenoxy)-2,4- | (q, J = 0.9 Hz, 1H), 8.30 (d, J = 5.6 Hz, 1H), 8.10 (s, 1H), 7.62 |
| | dimethylpent-4-en-2- | (dd, J = 5.4, 0.7 Hz, 1H), 7.11-7.07 (m, 2H), 6.96-6.93 (m, 2H), |
| | yl)thieno[3,2-b] | 4.87 (s, 1H), 4.74 (s, 1H), 4.33 (d, J = 9.0 Hz, 1H), 4.07 (d, J = |
| | pyridine-6- | 9.0 Hz, 1H), 2.90 (d, J = 13.2 Hz, 1H), 1.76 (s, 3H), 1.45 (s, 3H); |
| | carboxamide | ESI MS (m/z) 384.55 (MH)⁺ |
| 123 | N-(1-(4-fluoro-3- | ¹H-NMR (400 MHz, DMSO-d6) δ 8.66 (d, J = 2.0 Hz, 1H), 8.33 |
| | methoxyphenyl)-1- | (d, J = 2.0 Hz, 1H), 7.63 (s, 1H), 7.58 (d, J = 3.4 Hz, 1H), 7.08 |
| | hydroxy-2- | (dd, J = 11.5, 8.3 Hz, 1H), 7.01 (dd, J = 8.7, 1.8 Hz, 1H), 6.86 |
| | methylpropan-2-yl)-1- | (ddd, J = 8.3, 4.5, 1.8 Hz, 1H), 6.55 (d, J = 3.4 Hz, 1H), 5.84 (d, |
| | methyl-1H- | J = 5.1 Hz, 1H),5.20 (d, J = 5.4 Hz, 1H), 3.85-3.80 (m, 3H), 3.63 |
| | pyrrolo[2,3-b]pyridine- | (s, 3H), 1.47 (s, 3H), 1.21 (s, 3H); ESI MS (m/z) 371.6 (MH)⁺ |
| | 5-carboxamide | |
| 124 | 2-methyl-2- | ¹H-NMR (400 MHz, DMSO-d6) δ 9.17 (d, J = 2.0 Hz, 1H), 9.11- |
| | (thieno[3,2-b]pyridine- | 9.10(m, 1H), 8.41 (d, J = 5.6 Hz, 1H), 7.99 (s, 1H), 7.67 (dd, J = |
| | 6-carboxamido)propyl | 5.6, 0.7 Hz, 1H), 7.52 (dd, J = 8.3, 2.0 Hz, 1H), 7.43 (qd, J = 4.3, |
| | 4-fluoro-3- | 2.1 Hz, 1H), 7.25 (dd, J = 11.2, 8.3 Hz, 1H), 4.56 (s, 2H), 3.84 (s, |
| | methoxybenzoate | 3H), 1.50 (s, 6H); ESI MS (m/z) 402.55 (MH)⁺ |
| 125 | 2-methyl-2-(1-methyl- | ¹H-NMR (400 MHz, DMSO-d6) δ 8.69 (d, J = 2.0 Hz, 1H), 8.40 |
| | 1H-pyrrolo[2,3-b] | (d, J = 2.1 Hz, 1H), 8.02 (s, 1H), 7.95 (dd, J = 8.8, 6.4 Hz, 1H), |
| | pyridine-5- | 7.58-7.53 (m, 1H), 7.20-7.10 (m, 2H), 6.55 (d, J = 3.4 Hz, 1H), |
| | carboxamido)propyl | 4.51 (s, 2H), 3.83 (s, 3H), 2.50 (s, 3H), 1.47 (s, 6H); ESI MS |
| | 4-fluoro-2- | (m/z) 383.6 (MH)⁺ |
| | methylbenzoate | |
| 126 | N-(2-ethyl-2-hydroxy- | ¹H-NMR (400 MHz, DMSO-d6) δ 9.05 (d, J = 2.2 Hz, 1H), 8.93 |
| | 1-phenylbutyl)thieno | (d, J = 1.0 Hz, 1H), 8.61 (d, J = 9.3 Hz, 1H), 8.31 (d, J = 5.6 Hz, |
| | [3,2-b]pyridine-6- | 1H), 7.63 (dd, J = 5.4, 0.7 Hz, 1H), 7.49-7.47 (m, 2H), 7.28 (t, J = |
| | carboxamide | 7.3 Hz, 2H), 7.24-7.20 (m, 1H), 5.08 (d, J = 9.3 Hz, 1H), 4.43 |
| | | (s, 1H), 1.65-1.52 (m, 2H), 1.27-1.09 (m, 2H), 0.83 (t, J = 7.5 Hz, |
| | | 3H), 0.76 (t, J = 7.5 Hz, 3H); ESI MS (m/z) 354.65 (M)⁺ |
| 127 | N-(1-(2-fluorophenyl)- | ¹H-NMR (400 MHz, DMSO-d6) δ 8.65 (d, J = 2.2 Hz, 1H), 8.31 |
| | 2,4-dimethylpent-4-en- | (d, J = 2.3 Hz, 1H), 7.61-7.58 (m, 2H), 7.25-7.20 (m, 1H), 7.17- |
| | 2-yl)-1-methyl-1H- | 7.11 (m, 2H), 7.03 (td, J = 7.5, 1.1 Hz, 1H), 6.55 (d, J = 3.4 Hz, |
| | pyrrolo[2,3-b]pyridine- | 1H), 4.84 (s, 1H), 4.72 (s, 1H), 3.83 (s, 3H), 3.60 (d, J = 13.4 Hz, |
| | 5-carboxamide | 1H), 3.34 (d, J = 13.4 Hz, 1H), 2.90 (d, J = 13.0 Hz, 1H), 2.21 (d, |
| | | J = 13.2 Hz, 1H), 1.74 (s, 3H), 1.11 (s, 3H); ESI MS (m/z) 366.0 |
| | | (MH)⁺ |
| 128 | N-(1-(4- | ¹H-NMR(400 MHz, DMSO-d6) δ 11.55 (s, 1H), 7.89 (s, 1H), |
| | fluorophenoxy)-2,4- | 7.79 (s, 1H), 7.40 (t, J = 2.9 Hz, 1H), 7.13-7.08 (m, 2H), 6.97- |
| | dimethylpent-4-en-2- | 6.94 (m, 2H), 6.43-6.42 (m, 1H), 4.90 (d, J = 1.0 Hz, 1H), 4.77 |
| | yl)-6-methyl-1H- | (d, J = 1.0 Hz, 1H), 4.37 (d, J = 9.0 Hz, 1H), 4.01 (d, J = 9.0 Hz, |
| | pyrrolo[2,3-b]pyridine- | 1H), 2.90 (d, J = 13.2 Hz, 1H), 2.51 (s, 3H), 2.42 (d, J = 13.2 Hz, |
| | 5-carboxamide | 1H), 1.82 (s, 3H), 1.37 (s, 3H), ESI MS (m/z) 382.1 (MH)⁺ |
| 129 | N-(1-(2-fluorophenyl)- | ¹H-NMR (400 MHz, DMSO-d6) δ 8.95 (d, J = 2.0 Hz, 1H), 8.77- |
| | 2,4-dimethylpent-4-en- | 8.76 (m, 1H), 8.30 (d, J = 5.6 Hz, 1H), 7.85 (s, 1H), 7.62 (dd, J = |
| | 2-yl)thieno[3,2-b] | 5.6, 0.7 Hz, 1H), 7.27-7.09 (m, 3H), 7.06 (td, J = 7.5, 1.2 Hz, 1H), |
| | pyridine-6- | 4.88-4.85 (m, 1H), 4.73-4.67 (m, 1H), 3.60 (d, J = 13.2 Hz, 1H), |
| | carboxamide | 3.34 (d, J = 13.4 Hz, 1H), 2.92 (d, J = 13.2 Hz, 1H), 2.32-2.21 |
| | | (m, 1H), 1.77-1.71 (m, 3H), 1.14 (d, J = 17.1 Hz, 3H); ESI MS |
| | | (m/z) 368.7 (MH)⁺ |
| 130 | N-(1-(4-fluoro-2- | ¹H-NMR (400 MHz, DMSO-d6) δ 8.70 (d, J = 2.2 Hz, 1H), 8.40 |
| | methylphenyl)-1- | (d, J = 2.0 Hz, 1H), 7.75 (s, 1H), 7.59 (d, J = 3.7 Hz, 1H), 7.45 |
| | hydroxy-2- | (dd, J = 8.6, 6.4 Hz, 1H), 7.00-6.92 (m, 2H), 6.56 (d, J = 3.7 Hz, |
| | methylpropan-2-yl)-1- | 1H), 5.73 (d, J = 4.9 Hz, 1H), 5.43 (d, J = 4.9 Hz, 1H), 3.84 (s, |
| | methyl-1H- | 3H), 2.35 (s, 3H), 1.35 (s, 3H), 1.28 (s, 3H); ESI MS (m/z) 355.65 |
| | pyrrolo[2,3-b]pyridine- | (MH)⁺ |
| | 5-carboxamide | |
| 131 | N-(2-ethyl-1-(4- | ¹H-NMR (400 MHz, DMSO-d6) δ 8.71 (d, J = 2.0 Hz, 1H), 8.45 |
| | fluorophenyl)-2- | (d, J = 2.0 Hz, 1H), 8.39 (d, J = 9.3 Hz, 1H), 7.61 (d, J = 3.4 Hz, |
| | hydroxybutyl)-1- | 1H), 7.49 (dd, J = 8.7, 5.8 Hz, 2H), 7.10 (t, J = 8.9 Hz, 2H), 6.59 |
| | methyl-1H- | (d, J = 3.4 Hz, 1H), 5.07 (d, J = 9.3 Hz, 1H), 4.47 (s, 1H), 3.84 |
| | pyrrolo[2,3-b]pyridine- | (s, 3H), 1.58 (qd, J = 14.2, 7.5 Hz, 2H), 1.25-1.05 (m, 2H), 0.82 |
| | 5-carboxamide | (t, J = 7.5 Hz, 3H), 0.75 (t, J = 7.5 Hz, 3H) ; ESI MS (m/z) 369.7 |
| | | (MH)⁺ |
| 132 | N-(2-ethyl-1-(4- | ¹H-NMR (400 MHz, DMSO-d6) δ 9.04 (d, J = 2.0 Hz, 1H), 8.92 |
| | fluorophenyl)-2- | (dd, J = 2.1, 0.6 Hz, 1H), 8.62 (d, J = 9.0 Hz, 1H), 8.31 (d, J = 5.6 |
| | hydroxybutyl)thieno | Hz, 1H), 7.63 (dd, J = 5.4, 0.7 Hz, 1H), 7.52 (dd, J = 8.8, 5.6 Hz, |
| | [3,2-b]pyridine-6- | 2H), 7.10 (t, J = 8.9 Hz, 2H), 5.09 (d, J = 9.3 Hz, 1H), 4.46 (s, |
| | carboxamide | 1H), 1.65-1.52 (m, 2H), 1.24 (q, J = 7.1 Hz, 1H), 1.09 (dd, J = |
| | | 14.1, 7.5 Hz, 1H), 0.83 (t, J = 7.3 Hz, 3H), 0.76 (t, J = 7.5 Hz, |
| | | 3H); ESI MS (m/z) 373.0 (MH)⁺ |
| 133 | N-(1-(5-fluoro-2- | ¹H-NMR (400 MHz, DMSO-d6) δ 9.02 (d, J = 2.0 Hz, 1H), 8.86 |
| | methylphenyl)-1- | (q, J = 0.9 Hz, 1H), 8.30 (d, J = 5.6 Hz, 1H), 8.01 (s, 1H), 7.63 |
| | hydroxy-2- | (dd, J = 5.5, 0.9 Hz, 1H), 7.19-7.13 (m, 2H), 6.96 (td, J = 8.4, 2.9 |
| | methylpropan-2- | Hz, 1H), 5.69 (d, J = 4.6 Hz, 1H), 5.53 (dd, J = 4.6, 1.7 Hz, 1H), |
| | yl)thieno[3,2-b] | 2.33 (s, 3H), 1.37 (s, 3H), 1.29 (s, 3H); ESI MS (m/z) 358.55 |
| | pyridine-6- | (MH)⁺ |
| | carboxamide | |
| 134 | N-(1-(2-fluorophenyl)- | ¹H-NMR (400 MHz, DMSO-d6) δ 8.95 (d, J = 1.8 Hz, 1H), 8.77 |
| | 2-methylpent-4-en-2- | (d, J = 1.5 Hz, 1H), 8.29 (d, J = 5.5 Hz, 1H), 7.85 (s, 1H), 7.62 |
| | yl)thieno[3,2-b] | (d, J = 5.5 Hz, 1H), 7.27-7.05 (m, 4H), 5.91-5.81 (m, 1H), 5.11- |
| | pyridine-6- | 5.07 (m, 2H), 3.45 (d, J = 13.4 Hz, 1H), 3.10-3.05 (m, 1H), 2.99 |
| | carboxamide | (d, J = 13.4 Hz, 1H), 2.34-3.29 (m, 1H), 1.13 (d, J = 22.6 Hz, 3H); |
| | | ESI MS (m/z) 354.75 (MH)⁺ |
| 135 | 3-bromo-N-(1-(5- | ¹H-NMR (400 MHz, DMSO-d6) δ 8.78 (d, J = 2.2 Hz, 1H), 8.32 |
| | fluoro-2- | (d, J = 2.0 Hz, 1H), 7.98 (s, 1H), 7.86 (s, 1H), 7.18-7.12 (m, 2H), |
| | methylphenyl)-1- | 6.96 (td, J = 8.4, 2.9 Hz, 1H), 5.75 (t, J = 2.4 Hz, 1H), 5.52-5.50 |
| | hydroxy-2- | (m, 1H), 3.84 (s, 3H), 2.33 (s, 3H), 1.37 (s, 3H), 1.30 (s, 3H); ESI |
| | methylpropan-2-yl)-1- | MS (m/z) 435.45 (MH)⁺ |
| | methyl-1H- | |
| | pyrrolo[2,3-b]pyridine- | |
| | 5-carboxamide | |
| 136 | N-(1-(4-fluorophenyl)- | ¹H-NMR (400 MHz, DMSO-d6) δ 9.05 (d, J = 2.0 Hz, 1H), 8.95- |
| | 2-hydroxy-2- | 8.93 (m, 1H), 8.66 (d, J = 9.3 Hz, 1H), 8.31 (d, J = 5.6 Hz, 1H), |
| | methylpropyl)thieno | 7.64 (dd, J = 5.4, 0.7 Hz, 1H), 7.51 (dd, J = 8.7, 5.7 Hz, 2H), 7.11 |
| | [3,2-b]pyridine-6- | (t, J = 8.9 Hz, 2H), 5.03 (d, J = 9.3 Hz, 1H), 4.71 (s, 1H), 1.20 (s, |
| | carboxamide | 3H), 1.04 (s, 3H) ; ESI MS (m/z) 344.75 (MH)⁺ |
| 137 | 3-chloro-N-(2,3- | ¹H-NMR (400 MHz, DMSO-d6) δ 8.86-8.84 (m, 2H), 8.47 (d, J = |
| | dihydro-1H-inden-2- | 2.2 Hz, 1H), 7.83 (s, 1H), 7.25-7.22 (m, 2H), 7.17-7.13 (m, |
| | yl)-1-methyl-1H- | 2H), 4.77-4.70 (m, 1H), 3.83 (s, 3H), 3.27 (dd, J = 15.9, 7.9 Hz, |
| | pyrrolo[2,3-b]pyridine- | 2H), 2.99 (dd, J = 15.9, 6.6 Hz, 2H); ESI MS (m/z) 325.50 (M)⁺ |
| | 5-carboxamide | |
| 138 | 3-chloro-N-(2-ethyl-2- | ¹H-NMR (400 MHz, DMSO-d6) δ 8.80 (d, J = 2.0 Hz, 1H), 8.57 |
| | hydroxy-1- | (d, J = 9.3 Hz, 1H), 8.46 (d, J = 2.2 Hz, 1H), 7.85 (s, 1H), 7.48- |
| | phenylbutyl)-1-methyl- | 7.46 (m, 2H), 7.30-7.18 (m, 3H), 5.09 (d, J = 9.3 Hz, 1H), 4.45 |
| | 1H-pyrrolo[2,3-b] | (s, 1H), 3.83 (s, 3H), 1.67-1.45 (m, 2H), 1.25-1.21 (m, 1H), 1.13- |
| | pyridine-5- | 1.06 (m, 1H), 0.82 (t, J = 7.3 Hz, 3H), 0.76 (t, J = 7.3 Hz, 3H); |
| | carboxamide | ESI MS (m/z) 385.65 (M)⁺ |
| 139 | N-(1-(2-fluorophenyl)- | ¹H-NMR (400 MHz, DMSO-d6) δ 11.85 (s, 1H), 8.60 (d, J = 2.2 |
| | 2,4-dimethylpent-4-en- | Hz, 1H), 8.30 (d, J = 1.5 Hz, 1H), 7.58 (s, 1H), 7.54-7.53 (m, 1H), |
| | 2-yl)-1H-pyrrolo[2,3-b] | 7.24-7.20 (m, 1H), 7.17-7.11 (m, 2H), 7.03 (t, J = 7.3 Hz, 1H), |
| | pyridine-5- | 6.53-6.52 (m, 1H), 4.84 (s, 1H), 4.72 (s, 1H), 3.61 (d, J = 13.0 |
| | carboxamide | Hz, 1H), 3.34 (d, J = 13.4 Hz, 1H), 2.90 (d, J = 13.0 Hz, 1H), 2.20 |
| | | (d, J = 13.2 Hz, 1H), 1.74 (s, 3H), 1.10 (s, 3H); ESI MS (m/z) |
| | | 352.0 (MH)⁺ |
| 140 | 3-chloro-N-(1-(3,5- | ¹H-NMR (400 MHz, DMSO-d6) δ 9.40 (s, 1H), 8.85 (d, J = 2.0 |
| | dichlorophenyl) | Hz, 1H), 8.50 (d, J = 2.0 Hz, 1H), 7.86 (s, 1H), 7.40 (t, J = 2.0 |
| | cyclopropyl)- | Hz, 1H), 7.23 (d, J = 1.7 Hz, 2H), 3.84 (s, 3H), 1.41-1.32 (m, 4H); |
| | 1-methyl-1H- | ESI MS (m/z) 393.85 (MH)⁺ |
| | pyrrolo[2,3-b]pyridine- | |
| | 5-carboxamide | |
| 141 | (S)-N-(1,1-bis(3,5- | ¹H-NMR(400 MHz, DMSO-d6) δ 8.52 (d, J = 2.0 Hz, 1H), 8.21 |
| | dichlorophenyl)-1- | (d, J = 2.0 Hz, 1H), 8.00 (d, J = 9.0 Hz, 1H), 7.63 (d, J = 2.0 Hz, |
| | hydroxypropan-2-yl)- | 2H), 7.59-7.57 (m, 3H), 7.50 (t, J = 1.8 Hz, 1H), 7.38 (t, J = 1.8 |
| | 1-methyl-1H- | Hz, 1H), 6.55 (d, J = 3.7 Hz, 1H), 6.53 (s, 1H), 5.26-5.20 (m, 1H), |
| | pyrrolo[2,3-b]pyridine- | 3.81 (s, 3H), 1.05 (d, J = 6.6 Hz, 3H); ESI MS (m/z) 523.7 (MH)⁺ |
| | 5-carboxamide | |
| 142 | 3-bromo-N-(1-(2- | ¹H-NMR (400 MHz, DMSO-d6) δ 8.75 (dd, J = 5.7, 2.1 Hz, 1H), |
| | fluorophenyl)-2,4- | 8.22 (d, J = 2.0 Hz, 1H), 7.86 (s, 1H), 7.78 (s, 1H), 7.27-7.20 (m, |
| | dimethylpent-4-en-2- | 1H), 7.17-7.11 (m, 2H), 7.03 (td, J = 7.4, 1.3 Hz, 1H), 4.86-4.83 |
| | yl)-1-methyl-1H- | (m, 1H), 4.72 (s, 1H), 3.83 (s, 3H), 3.61 (d, J = 13.2 Hz, 1H), 3.35 |
| | pyrrolo[2,3-b]pyridine- | (d, J = 13.4 Hz, 1H), 2.90 (d, J = 13.2 Hz, 1H), 2.22 (d, J = 13.2 |
| | 5-carboxamide | Hz, 1H), 1.72 (d, J = 9.8 Hz, 3H), 1.12 (s, 3H); ESI MS (m/z) |
| | | 445.85 (MH)⁺ |
| 143 | 3-chloro-N-(1-(3- | ¹H-NMR (400 MHz, DMSO-d6) δ 9.33 (s, 1H), 8.85 (d, J = 2.2 |
| | methoxyphenyl) | Hz, 1H), 8.50 (d, J = 2.0 Hz, 1H), 7.84 (s, 1H), 7.20-7.16 (m, 1H), |
| | rcyclopopyl)- | 6.80 (dt, J = 8.2, 1.0 Hz, 1H), 6.75-6.72 (m, 2H), 3.84 (s, 3H), |
| | 1-methyl-1H- | 3.70 (s, 3H), 1.31-1.25 (m, 4H), 0.94 (d, J = 6.4 Hz, OH), 0.84 (s, |
| | pyrrolo[2,3-b]pyridine- | 0H); ESI MS (m/z) 355.5 (MH)⁺ |
| | 5-carboxamide | |
| 144 | N-(1-(2-fluorophenyl)- | ¹H-NMR (400 MHz, DMSO-d6) δ 11.53 (s, 1H), 8.69 (d, J = 2.0 |
| | 2,4-dimethylpent-4-en- | Hz, 1H), 8.08 (s, 1H), 7.78 (t, J = 2.9 Hz, 1H), 7.66 (s, 1H), 7.22 |
| | 2-yl)-1H-pyrrolo[3,2-b] | (t, J = 7.7 Hz, 1H), 7.18-7.11 (m, 2H), 7.05-7.02 (m, 1H), 6.60 (s, |
| | pyridine-6- | 1H), 4.85 (s, 1H), 4.72 (d, J = 1.7 Hz, 1H), 3.61 (d, J = 12.5 Hz, |
| | carboxamide | 1H), 3.36 (s, 1H), 2.90 (d, J = 13.0 Hz, 1H), 2.21 (d, J = 13.4 Hz, |
| | | 1H), 1.74 (s, 3H), 1.11 (s, 3H); ESI MS (m/z) 351.60 (MH)⁺ |
| 145 | 2-(6,7-dihydro-5H- | ¹H-NMR (400 MHz, CHLOROFORM-D) δ 8.65 (d, J = 2.0 Hz, |
| | cyclopenta[b]pyridine- | 1H), 7.87 (t, J = 1.0 Hz, 1H), 7.83 (dt, J = 7.8, 1.2 Hz, 1H), 7.70 |
| | 3-carboxamido)-2- | (dq, J = 9.2, 1.3 Hz, 1H), 7.42 (td, J = 8.0, 5.5 Hz, 1H), 7.29-7.27 |
| | methylpropyl | (m, 1H), 6.41 (s, 1H), 4.56 (s, 2H), 3.03 (t, J = 7.8 Hz, 2H), 2.96 |
| | 3-fluorobenzoate | (t, J = 7.5 Hz, 2H), 2.19-2.12 (m, 2H), 1.58 (s, 6H); ESI MS |
| | | (m/z) 357.3 (MH)⁺ |
| 146 | 2-methyl-2-(1H- | ¹H-NMR (400 MHz, CHLOROFORM-D) δ 9.51 (s, 1H), 8.72 (d, |
| | pyrrolo[2,3-b]pyridine- | J = 2.0 Hz, 1H), 8.35 (d, J = 2.0 Hz, 1H), 7.85 (dt, J = 7.7, 1.3 |
| | 5-carboxamido)propyl | Hz, 1H), 7.72 (ddd, J = 9.2, 2.4, 1.6 Hz, 1H), 7.44-7.39 (m, 2H), |
| | 3-fluorobenzoate | 7.29-7.28 (m, 1.2 Hz, 1H), 6.60-6.58(m, 1H), 6.42 (s, 1H), 4.61 |
| | | (s, 2H), 1.55-1.61(s, 6H); ESI MS (m/z) 356.20 (MH)⁺ |
| 148 | 2-(6,7-dihydro-5H- | ¹H-NMR (400 MHz, DMSO-d6) δ 8.66 (d, J = 2.2 Hz, 1H), 7.99 |
| | cyclopenta[b]pyridine- | (s, 1H), 7.92 (t, J = 0.9 Hz, 1H), 7.85 (dd, J = 9.0, 6.4 Hz, 2H), |
| | 3-carboxamido)-2- | 7.81-7.74 (m, 2H), 4.55 (s, 2H), 2.95-2.86 (m, 4H), 2.10-2.02 (m, |
| | methylpropyl | 2H), 1.41 (s, 6H); ESI MS (m/z) 407.35 (MH)⁺ |
| | 2-(trifluoromethyl) | |
| | benzoate | |
| 149 | 2-(6,7-dihydro-5H- | ¹H-NMR (400 MHz, DMSO-d6) δ 8.67 (d, J = 2.2 Hz, 1H), 8.05 |
| | cyclopenta[b]pyridine- | (s, 1H), 7.93 (t, J = 1.0 Hz, 1H), 7.85 (dd, J = 7.7, 1.3 Hz, 1H), |
| | 3-carboxamido)-2- | 7.46 (td, J = 7.5, 1.3 Hz, 1H), 7.29 (t, J = 8.7 Hz, 2H), 4.47 (d, J = |
| | methylpropyl | 11.0 Hz, 2H), 2.93-2.89 (m, 4H), 2.10-2.02 (m, 2H), 1.44 (s, |
| | 2-methylbenzoate | 6H); ESI MS (m/z) 353.50 (MH)⁺ |
| 150 | 2-methyl-2-(5,6,7,8- | ¹H-NMR (400 MHz, DMSO-d6) δ 8.64 (d, J = 2.1 Hz, 1H), 7.97 |
| | tetrahydroquinoline-3- | (s, 1H), 7.87-7.83 (m, 2H), 7.80-7.74 (m, 3H), 4.55 (s, 2H), 2.81 |
| | carboxamido)propyl | (t, J = 6.4 Hz, 2H), 2.74 (t, J = 6.3 Hz, 2H), 1.84-1.78 (m, 2H), |
| | 2-(trifluoromethyl) | 1.76-1.72 (m, 2H), 1.41 (s, 6H); ESI MS (m/z) 421.2 (MH)⁺ |
| | benzoate | |
| 151 | 2-methyl-2-(5,6,7,8- | ¹H-NMR (400 MHz, DMSO-d6) δ 8.66 (d, J = 2.1 Hz, 1H), 8.03 |
| | tetrahydroquinoline-3- | (s, 1H), 7.86-7.84 (m, 1H), 7.81 (d, J = 2.1 Hz, 1H), 7.46 (td, J = |
| | carboxamido)propyl | 7.5, 1.4 Hz, 1H), 7.31-7.27 (m, 2H), 4.49 (s, 2H), 2.81 (t, J = 6.4 |
| | 2-methylbenzoate | Hz, 2H), 2.75 (t, J = 6.1 Hz, 2H), 2.48 (s, 3H), 1.84-1.78 (m, 2H), |
| | | 1.76-1.70 (m, 2H), 1.44 (s, 6H); ESI MS (m/z) 367.1 (MH)⁺ |
| 152 | 2-methyl-2-(5,6,7,8- | ¹H-NMR (400 MHz, DMSO-d6) δ 8.64 (d, J = 2.4 Hz, 1H), 8.05 |
| | tetrahydroquinoline-3- | (s, 1H), 7.82 (dt, J = 7.6, 1.4 Hz, 1H), 7.80-7.77 (m, 1H), 7.72- |
| | carboxamido)propyl | 7.69 (m, 1H), 7.58 (td, J = 7.9, 5.8 Hz, 1H), 7.53-7.51 (m, 1H), |
| | 3-fluorobenzoate | 4.50 (s, 2H), 2.81 (t, J = 6.4 Hz, 2H), 2.74 (t, J = 6.3 Hz, 2H), |
| | | 1.84-1.78 (m, 2H), 1.76-1.70 (m, 2H), 1.46 (s, 6H); ESI MS (m/z) |
| | | 371.0 (MH)⁺ |
| 153 | 2-(3-chloro-1-methyl- | ¹H-NMR (400 MHz, DMSO-d6) δ 8.75 (d, J = 2.0 Hz, 1H), 8.39 |
| | 1H-pyrrolo[2,3-b] | (d, J = 2.0 Hz, 1H), 8.19 (s, 1H), 7.83 (dt, J = 7.9, 1.5 Hz, 2H), |
| | pyridine-5- | 7.74-7.70 (m, 1H), 7.60-7.48 (m, 2H), 4.54 (s, 2H), 3.83 (s, 3H), |
| | carboxamido)-2- | 1.50 (s, 6H); ESI MS (m/z) 404.20 (MH)⁺ |
| | methylpropyl | |
| | 3-fluorobenzoate | |
| 155 | N-(1-(2- | ¹H-NMR (400 MHz, DMSO-d6) δ 8.66 (d, J = 2.0 Hz, 1H), 8.36 |
| | fluorophenoxy)-2,4- | (d, J = 2.0 Hz, 1H), 7.90 (s, 1H), 7.59 (d, J = 3.4 Hz, 1H), 7.19- |
| | dimethylpent-4-en-2- | 7.07 (m, 3H), 6.93-6.88 (m, 1H), 6.56 (d, J = 3.4 Hz, 1H), 4.87 |
| | yl)-1-methyl-1H- | (q, J = 1.4 Hz, 1H), 4.73 (d, J = 9.3 Hz, 1H), 4.45 (d, J = 9.0 Hz, |
| | pyrrolo[2,3-b]pyridine- | 1H), 4.17 (d, J = 9.0 Hz, 1H), 3.83 (s, 3H), 2.93 (d, J = 13.2 Hz, |
| | 5-carboxamide | 1H), 2.55-2.45(m, 1H) 1.76 (s, 3H), 1.46 (s, 3H); ESI MS (m/z) |
| | | 382.1 (MH)⁺ |
| 156 | 2-methyl-2-(1H- | ¹H-NMR (400 MHz, DMSO-d6) δ 11.55 (s, 1H), 8.76 (d, J = 2.0 |
| | pyrrolo[3,2-b]pyridine- | Hz, 1H), 8.14 (q, J = 1.0 Hz, 1H), 8.13 (s, 1H), 8.08 (s, 1H), 7.83 |
| | 6-carboxamido)propyl | (dt, J = 7.7, 1.3 Hz, 1H), 7.78 (t, J = 2.9 Hz, 1H), 7.71 (dd, J = |
| | 3-fluorobenzoate | 9.3, 2.4 Hz, 1H), 7.57 (td, J = 7.9, 5.8 Hz, 1H), 7.53-7.48 (m, 1H), |
| | | 4.53 (d, J = 5.9 Hz, 2H), 1.49 (s, 6H); ESI MS (m/z) 356.00 (MH)⁺ |
| | | ESI MS (m/z) 356.00 (MH)⁺ |
| 158 | 2-methyl-2-(1H- | ¹H-NMR (400 MHz, DMSO-d6) δ 11.85 (s, 1H), 8.65 (d, J = 2.0 |
| | pyrrolo[2,3-b]pyridine- | Hz, 1H), 8.37 (d, J = 1.7 Hz, 1H), 7.94 (s, 1H), 7.85 (dd, J = 6.2, |
| | 5-carboxamido)propyl | 2.8 Hz, 2H), 7.79-7.75 (m, 2H), 7.53-7.52 (m, 1H), 6.52 (q, J = |
| | 2-(trifluoromethyl) | 1.7 Hz, 1H), 4.59 (s, 2H), 1.44 (s, 6H);ESI MS (m/z) |
| | benzoate | 406.10 (MH)⁺ |
| 159 | N-(1-(2- | ¹H-NMR (400 MHz, DMSO-d6) δ 8.97 (d, J = 2.2 Hz, 1H), 8.82- |
| | fluorophenoxy)-2,4- | 8.79 (m, 1H), 8.30 (d, J = 5.4 Hz, 1H), 8.14 (s, 1H), 7.63-7.58 |
| | dimethylpent-4-en-2- | (m, 1H), 7.19-7.07 (m, 3H), 6.91 (tdd, J = 7.7, 4.6, 1.7 Hz, 1H), |
| | yl)thieno[3,2-b] | 4.88 (q, J = 1.4 Hz, 1H), 4.77-4.71 (m, 1H), 4.47 (d, J = 9.0 Hz, |
| | pyridine-6- | 1H), 4.17 (d, J = 9.3 Hz, 1H), 2.93 (d, J = 13.2 Hz, 1H), 2.46 (s, |
| | carboxamide | 1H), 1.76 (d, J = 6.4 Hz, 3H), 1.47 (s, 3H); ESI MS (m/z) |
| | | 385.4 (MH)⁺ |
| 160 | N-(1-(2- | ¹H-NMR (400 MHz, DMSO-d6) δ 8.63 (d, J = 2.1 Hz, 1H), 7.94 |
| | fluorophenoxy)-2,4- | (s, 1H), 7.89 (t, J = 1.1 Hz, 1H), 7.19-7.07 (m, 3H), 6.94-6.88 (m, |
| | dimethylpent-4-en-2- | 1H), 4.87-4.86(m, 1H), 4.71 (d, J = 2.1 Hz, 1H), 4.43 (d, J = 9.2 |
| | yl)-6,7-dihydro-5H- | Hz, 1H), 4.12 (d, J = 9.2 Hz, 1H), 2.93-2.87 (m, 5H), 2.43 (d, J = |
| | cyclopenta[b]pyridine- | 13.1 Hz, 1H), 2.10-2.03 (m, 2H), 1.73 (s, 3H), 1.43 (d, J = 6.7 |
| | 3-carboxamide | Hz, 3H); ESI MS (m/z) 369.30 (MH)⁺ |
| 161 | 2-methyl-2- | ¹H-NMR (400 MHz, DMSO-d6) δ 9.01 (d, J = 2.2 Hz, 1H), 8.85- |
| | (thieno[3,2-b]pyridine- | 8.83 (m, 1H), 8.29 (d, J = 5.4 Hz, 1H), 8.19 (s, 1H), 7.86-7.83 |
| | 6-carboxamido)propyl | (m, 2H), 7.80-7.73 (m, 2H), 7.62 (dd, J = 5.6, 0.7 Hz, 1H), 4.60 |
| | 2-(trifluoromethyl) | (s, 2H), 1.45 (s, 6H); ESI MS (m/z) 423.40 (MH)⁺ |
| | benzoate | |
| 162 | 2-methyl-2-(1H- | ¹H-NMR (400 MHz, DMSO-d6) δ 11.55 (s, 1H), 8.75 (d, J = 2.0 |
| | pyrrolo[3,2-b]pyridine- | Hz, 1H), 8.15-8.13 (m, 1H), 8.01 (s, 1H), 7.86-7.83 (m, 2H), |
| | 6-carboxamido)propyl | 7.79-7.73 (m, 3H), 6.60-6.59 (m, 1H), 4.59 (s, 2H), 1.44 (s, |
| | 2-(trifluoromethyl) | 6H); ESI MS (m/z) 406.15 (MH)⁺ |
| | benzoate | |
| 164 | 2-methyl-2- | ¹H-NMR (400 MHz, DMSO-d6) δ 9.02 (d, J = 2.0 Hz, 1H), 8.86- |
| | (thieno[3,2-b]pyridine- | 8.85 (m, 1H), 8.29 (d, J = 5.6 Hz, 1H), 8.25 (s, 1H), 7.86 (d, J = |
| | 6-carboxamido)propyl | 7.8 Hz, 1H), 7.62 (dd, J = 5.5, 0.6 Hz, 1H), 7.47-7.43 (m, 1H), |
| | 2-methylbenzoate | 7.28 (t, J = 8.3 Hz, 2H), 4.53 (s, 2H), 2.42-2.48 (3H), 1.52 (s, |
| | | 6H); ESI MS (m/z) 368.55 (MH)⁺ |
| 165 | N-(1-(4-fluoro-3- | ¹H-NMR (400 MHz, DMSO-d6) δ 8.61 (d, J = 2.0 Hz, 1H), 7.89 |
| | methoxyphenyl)-1- | (d, J = 2.0 Hz, 1H), 7.63 (s, 1H), 7.09 (dd, J = 11.6, 8.4 Hz, 1H), |
| | hydroxy-2- | 6.99 (dd, J = 8.6, 1.7 Hz, 1H), 6.83 (qd, J = 4.2, 1.8 Hz, 1H), 5.74 |
| | methylpropan-2-yl)- | (d, J = 5.1 Hz, 1H), 5.18 (d, J = 5.1 Hz, 1H), 3.67 (s, 3H), 2.91 |
| | 6,7-dihydro-5H- | (td, J = 7.5, 4.2 Hz, 4H), 2.11-2.03 (m, 2H), 1.44 (s, 3H), 1.17 (s, |
| | cyclopenta[b]pyridine- | 3H); ESI MS (m/z) 358.55 (MH)⁺ |
| | 3-carboxamide | |
| 166 | 2-methyl-2-(1H- | ¹H-NMR(400 MHz, DMSO-d6) δ 11.86 (s, 1H), 8.66 (d, J = 2.1 |
| | pyrrolo[2,3-b]pyridine- | Hz, 1H), 8.39 (d, J = 1.8 Hz, 1H), 8.00 (s, 1H), 7.87-7.85 (m, 1H), |
| | 5-carboxamido)propyl | 7.53 (t, J = 2.9 Hz, 1H), 7.45 (td, J = 7.5, 1.4 Hz, 1H), 7.28 (t, J = |
| | 2-methylbenzoate | 8.3 Hz, 2H), 6.52 (q, J = 1.8 Hz, 1H), 4.52 (s, 2H), 2.49 (q, J = |
| | | 1.8 Hz, 3H), 1.45 (d, J = 14.1 Hz, 6H); ESI MS (m/z) |
| | | 352.05 (MH)⁺ |
| 167 | 2-methyl-2-(1-methyl- | ¹H-NMR (400 MHz, DMSO-d6) δ 8.68 (d, J = 2.1 Hz, 1H), 8.39 |
| | 1H-pyrrolo[2,3-b] | (d, J = 2.1 Hz, 1H), 7.97 (s, 1H), 7.86-7.83 (m, 2H), 7.79-7.75 |
| | pyridine-5- | (m, 2H), 7.58 (d, J = 3.4 Hz, 1H), 6.54 (d, J = 3.4 Hz, 1H), 4.59 |
| | carboxamido)propyl | (s, 2H), 3.83 (s, 3H), 1.44 (s, 6H); ESI MS (m/z) 420.25 (MH)⁺ |
| | 2-(trifluoromethyl) | |
| | benzoate | |
| 168 | 2-methyl-2-(1-methyl- | ¹H-NMR (400 MHz, DMSO-d6) δ 8.68 (d, J = 2.0 Hz, 1H), 8.39 |
| | 1H-pyrrolo[2,3-b] | (d, J = 2.2 Hz, 1H), 8.04 (s, 1H), 7.83 (d, J = 7.6 Hz, 1H), 7.72- |
| | pyridine-5- | 7.70 (m, 1H), 7.60-7.50 (m, 3H), 6.54 (d, J = 3.4 Hz, 1H), 4.53 |
| | carboxamido)propyl | (s, 2H), 3.82 (s, 3H), 1.49 (s, 6H); ESI MS (m/z) 370.00 (MH)⁺ |
| | 3-fluorobenzoate | |
| 170 | N-(1-(4-fluorophenyl)- | ¹H-NMR (400 MHz, DMSO-d6) δ 11.81 (s, 1H), 8.46 (d, J = 9.5 |
| | 2-hydroxy-2- | Hz, 1H), 8.39 (dd, J = 4.6, 1.5 Hz, 1H), 7.78-7.75 (m, 1H), 7.52 |
| | methylpropyl)-1H- | (dd, J = 8.8, 5.6 Hz, 2H), 7.47-7.46 (m, 1H), 7.19-7.17 (m, 1H), |
| | pyrrolo[2,3-b]pyridine- | 7.12 (t, J = 8.9 Hz, 2H), 5.03 (d, J = 9.3 Hz, 1H), 4.73 (s, 1H), |
| | 5-carboxamide | 1.21 (s, 3H), 1.03 (s, 3H); ESI MS (m/z) 327.50 (MH)⁺ |
| 171 | N-(1-(2- | ¹H-NMR (400 MHz, DMSO-d6) δ 11.86 (s, 1H), 8.62 (d, J = 2.1 |
| | fluorophenoxy)-2,4- | Hz, 1H), 8.35 (d, J = 1.8 Hz, 1H), 7.88 (s, 1H), 7.53 (t, J = 2.9 |
| | dimethylpent-4-en-2- | Hz, 1H), 7.19-7.07 (m, 3H), 6.93-6.88 (m, 1H), 6.53 (q, J = 1.7 |
| | yl)-1H-pyrrolo[2,3-b] | Hz, 1H), 4.87 (d, J = 0.9 Hz, 1H), 4.74 (s, 1H), 4.45 (d, J = 9.2 |
| | pyridine-5- | Hz, 1H), 4.16 (d, J = 9.2 Hz, 1H), 1.76 (s, 3H), 1.46 (s, 3H); ESI |
| | carboxamide | MS (m/z) 367.6 (MH)⁺ |
| 172 | N-(1-(4-fluoro-2- | ¹H-NMR (400 MHz, DMSO-d6) δ 8.67 (d, J = 2.0 Hz, 1H), 7.94 |
| | methylphenyl)-1- | (d, J = 2.0 Hz, 1H), 7.75 (s, 1H), 7.44 (dd, J = 8.6, 6.4 Hz, 1H), |
| | hydroxy-2- | 7.00-6.92 (m, 2H), 5.60 (d, J = 4.9 Hz, 1H), 5.43 (d, J = 4.9 Hz, |
| | methylpropan-2-yl)- | 1H), 2.92 (t, J = 7.6 Hz, 4H), 2.34 (s, 3H), 2.11-2.04 (m, 2H), |
| | 6,7-dihydro-5H- | 1.33 (s, 3H), 1.24 (s, 3H); ESI MS (m/z) 343.1 (MH)⁺ |
| | cyclopenta[b]pyridine- | |
| | 3-carboxamide | |
| 173 | N-(1-(4-fluoro-2- | ¹H-NMR (400 MHz, DMSO-d6) δ 9.03 (d, J = 2.0 Hz, 1H), 8.86 |
| | methylphenyl)-1- | (d, J = 1.5 Hz, 1H), 8.30 (d, J = 5.6 Hz, 1H), 7.97 (d, J = 5.6 Hz, |
| | hydroxy-2- | 1H), 7.64-7.62 (m, 1H), 7.47 (dd, J = 8.6, 6.4 Hz, 1H), 7.01-6.94 |
| | methylpropan-2- | (m, 2H), 5.60 (d, J = 4.6 Hz, 1H), 5.51 (d, J = 4.6 Hz, 1H), 2.37 |
| | yl)thieno[3,2-b] | (s, 3H), 1.36 (s, 3H), 1.27 (s, 3H); ESI MS (m/z) 358.5 (MH)⁺ |
| | pyridine-6- | |
| | carboxamide | |
| 175 | N-(1-(2- | ¹H-NMR (400 MHz, DMSO-d6) δ 11.56 (s, 1H), 8.72 (d, J = 2.1 |
| | fluorophenoxy)-2,4- | Hz, 1H), 8.12 (d, J = 0.9 Hz, 1H), 7.96 (s, 1H), 7.78 (t, J = 3.1 |
| | dimethylpent-4-en-2- | Hz, 1H), 7.19-7.07 (m, 3H), 6.93-6.88 (m, 1H), 6.60 (t, J = 2.0 |
| | yl)-1H-pyrrolo[3,2-b] | Hz, 1H), 4.87 (d, J = 1.2 Hz, 1H), 4.74 (s, 1H), 4.46 (d, J = 9.2 |
| | pyridine-6- | Hz, 1H), 4.16 (d, J = 9.2 Hz, 1H), 2.93 (d, J = 13.1 Hz, 1H), 2.49- |
| | carboxamide | 2.45 (m, 1H), 1.75 (d, J = 10.4 Hz, 3H), 1.46 (s, 3H); ESI MS |
| | | (m/z) 367.6 (MH)⁺ |
| 176 | 3-bromo-N-(1-(2- | ¹H-NMR (400 MHz, DMSO-d6) δ 8.73 (dd, J = 5.4, 2.0 Hz, 1H), |
| | fluorophenoxy)-2,4- | 8.27 (d, J = 2.0 Hz, 1H), 8.08 (s, 1H), 7.85 (s, 1H), 7.19-7.13 (m, |
| | dimethylpent-4-en-2- | 2H), 7.11-7.07 (m, 1H), 6.90 (tdd, J = 7.7, 4.6, 1.6 Hz, 1H), 4.87 |
| | yl)-1-methyl-1H- | (q, J = 1.4 Hz, 1H), 4.74-4.67 (m, 1H), 4.48 (d, J = 9.3 Hz, 1H), |
| | pyrrolo[2,3-b]pyridine- | 4.17 (d, J = 9.3 Hz, 1H), 3.83 (s, 3H), 2.95 (d, J = 13.2 Hz, 1H), |
| | 5-carboxamide | 2.42 (d, J = 17.4 Hz, 1H), 1.76 (s, 3H), 1.47 (s, 3H), ); ESI MS |
| | | (m/z) 461.5 (MH)⁺ |
| 177 | N-(1-(2- | ¹H-NMR (400 MHz, DMSO-d6) δ 11.55 (s, 1H), 7.94 (s, 1H), |
| | fluorophenoxy)-2,4- | 7.79 (s, 1H), 7.40 (t, J = 2.9 Hz, 1H), 7.23-7.09 (m, 3H), 6.95- |
| | dimethylpent-4-en-2- | 6.89 (m, 1H), 6.42 (q, J = 1.7 Hz, 1H), 4.91 (d, J = 1.0 Hz, 1H), |
| | yl)-6-methyl-1H- | 4.79 (s, 1H), 4.51 (d, J = 9.0 Hz, 1H), 4.10 (d, J = 9.0 Hz, 1H), |
| | pyrrolo[2,3-b]pyridine- | 2.92 (d, J = 13.0 Hz, 1H), 2.52(s, 3H), 2.41 (d, J = 13.2 Hz, 1H), |
| | 5-carboxamide | 1.83 (s, 3H), 1.39 (s, 3H); ESI MS (m/z) 382.2 (MH)⁺ |
| 178 | 2-(3-bromo-1-methyl- | ¹H-NMR (400 MHz, DMSO-d6) δ 8.74 (d, J = 2.0 Hz, 1H), 8.29 |
| | 1H-pyrrolo[2,3-b] | (d, J = 2.0 Hz, 1H), 8.21 (s, 1H), 7.84 (d, J = 9.5 Hz, 2H), 7.72 |
| | pyridine-5- | (d, J = 9.5 Hz, 1H), 7.60-7.49 (m, 2H), 4.54 (s, 2H), 3.83 (s, 3H), |
| | carboxamido)-2- | 1.50 (s, 6H); ESI MS (m/z) 449.20 (MH)⁺ |
| | methylpropyl | |
| | 3-fluorobenzoate | |
| 179 | 3-chloro-N-(1-(2- | ¹H-NMR (400 MHz, DMSO-d6) δ 8.73 (d, J = 2.0 Hz, 1H), 8.37 |
| | fluorophenoxy)-2,4- | (d, J = 2.0 Hz, 1H), 8.06 (s, 1H), 7.82 (s, 1H), 7.20-7.07 (m, 3H), |
| | dimethylpent-4-en-2- | 6.95-6.87 (m, 1H), 4.87 (t, J = 1.2 Hz, 1H), 4.74 (s, 1H), 4.48 (d, |
| | yl)-1-methyl-1H- | J = 9.0 Hz, 1H), 4.17 (d, J = 9.0 Hz, 1H), 3.82 (s, 3H), 2.94 (d, J = |
| | pyrrolo[2,3-b]pyridine- | 13.2 Hz, 1H), 2.43 (d, J = 19.1 Hz, 1H), 1.73 (d, J = 16.9 Hz, |
| | 5-carboxamide | 3H), 1.47 (s, 3H); ESI MS (m/z) 415.55 (MH)⁺ |
| 180 | 2-(3-bromo-1-methyl- | ¹H-NMR (400 MHz, DMSO-d6) δ 8.76-8.74 (m, 1H), 8.30 (d, J = |
| | 1H-pyrrolo[2,3-b] | 2.2 Hz, 1H), 8.14 (s, 1H), 7.86-7.82 (m, 3H), 7.79-7.76 (m, |
| | pyridine-5- | 2H), 4.59 (s, 2H), 3.83 (s, 3H), 1.45 (s, 6H); ESI MS (m/z) |
| | carboxamido)-2- | 499.25 (MH)⁺ |
| | methylpropyl | |
| | 2-(trifluoromethyl) | |
| | benzoate | |
| 181 | N-(1-(4-fluorophenyl)- | ¹H-NMR (400 MHz, DMSO-d6) δ 8.64 (d, J = 2.2 Hz, 1H), 8.29 |
| | 2,4-dimethylpent-4-en- | (d, J = 2.0 Hz, 1H), 7.58 (d, J = 3.4 Hz, 1H), 7.51 (s, 1H), 7.15 |
| | 2-yl)-1-methyl-1H- | (dd, J = 8.6, 5.9 Hz, 2H), 7.04 (t, J = 8.9 Hz, 2H), 6.55 (d, J = 3.4 |
| | pyrrolo[2,3-b]pyridine- | Hz, 1H), 4.84 (s, 1H), 4.71 (s, 1H), 3.83 (s, 3H), 3.69 (d, J = 13.2 |
| | 5-carboxamide | Hz, 1H), 3.26 (d, J = 13.4 Hz, 1H), 2.65 (d, J = 13.0 Hz, 1H), 2.17 |
| | | (d, J = 13.2 Hz, 1H), 1.74 (s, 3H), 1.11 (s, 3H); ESI MS (m/z) |
| | | 366.05 (MH)⁺ |
| 182 | N-(1-(4-fluorophenyl)- | ¹H-NMR (400 MHz, DMSO-d6) δ 8.93 (d, J = 2.2 Hz, 1H), 8.75 |
| | 2,4-dimethylpent-4-en- | (q, J = 0.9 Hz, 1H), 8.29 (d, J = 5.6 Hz, 1H), 7.75 (s, 1H), 7.62 |
| | 2-yl)thieno[3,2-b] | (dd, J = 5.4, 0.7 Hz, 1H), 7.18-7.14 (m, 2H), 7.08-7.04 (m, 2H), |
| | pyridine-6- | 4.86-4.85 (m, 1H), 4.72 (s, 1H), 3.67 (d, J = 13.0 Hz, 1H), 3.25 |
| | carboxamide | (d, J = 13.4 Hz, 1H), 2.68 (d, J = 13.2 Hz, 1H), 2.20 (d, J = 13.4 |
| | | Hz, 1H), 1.75 (s, 3H), 1.12 (s, 3H); ESI MS (m/z) 368.65 (MH)⁺ |
| 185 | 2-(3-bromo-1-methyl- | ¹H-NMR (400 MHz, DMSO-d6) δ 8.75 (d, J = 2.0 Hz, 1H), 8.32 |
| | 1H-pyrrolo[2,3-b] | (d, J = 2.0 Hz, 1H), 8.20 (s, 1H), 7.88-7.82 (m, 2H), 7.45 (t, J = |
| | pyridine-5- | 7.5 Hz, 1H), 7.28 (t, J = 7.8 Hz, 2H), 4.53 (s, 2H), 3.82 (s, 3H), |
| | carboxamido)-2- | 2.53 (s, 3H), 1.48 (s, 6H); ESI MS (m/z) 443.95 (MH)⁺ |
| | methylpropyl | |
| | 2-methylbenzoate | |
| 186 | N-(1-(5-fluoro-2- | ¹H-NMR (400 MHz, DMSO-d6) δ 8.67 (d, J = 2.2 Hz, 1H), 7.94 |
| | methylphenyl)-1- | (t, J = 1.0 Hz, 1H), 7.79 (s, 1H), 7.16-7.12 (m, 2H), 6.96 (td, J = |
| | hydroxy-2- | 8.4, 2.9 Hz, 1H), 5.70 (d, J = 4.9 Hz, 1H), 5.45 (dd, J = 4.9, 1.7 |
| | methylpropan-2-yl)- | Hz, 1H), 2.92 (t, J = 7.6 Hz, 4H), 2.30 (s, 3H), 2.11-2.04 (m, 2H), |
| | 6,7-dihydro-5H- | 1.34 (s, 3H), 1.26 (s, 3H); ESI MS (m/z) 342.9 (MH)⁺ |
| | cyclopenta[b]pyridine- | |
| | 3-carboxamide | |
| 187 | N-(1-((2- | ¹H-NMR (400 MHz, CHLOROFORM-D) δ 8.71 (d, J = 2.1 Hz, |
| | fluorobenzyl)oxy)-2- | 1H), 8.25 (d, J = 2.1 Hz, 1H), 7.39 (td, J = 7.5, 1.8 Hz, 1H), 7.30- |
| | methylpropan-2-yl)-1- | 7.28 (m, 1H), 7.23 (d, J = 3.7 Hz, 1H), 7.13 (td, J = 7.5, 1.0 Hz, |
| | methyl-1H- | 1H), 7.08-7.03 (m, 1H), 6.52 (d, J = 3.7 Hz, 1H), 6.41 (s, 1H), |
| | pyrrolo[2,3-b]pyridine- | 4.66 (s, 2H), 3.91 (s, 3H), 3.59 (s, 2H), 1.51 (s, 6H); ESI MS |
| | 5-carboxamide | (m/z) 356.0 (MH)⁺ |
| 188 | N-(1-((2- | ¹H-NMR (400 MHz, CHLOROFORM-D) δ 8.67 (t, J = 1.1 Hz, |
| | cyanobenzyl)oxy)-2- | 1H), 7.87 (t, J = 1.0 Hz, 1H), 7.66 (dd, J = 7.8, 1.0 Hz, 1H), 7.57 |
| | methylpropan-2-yl)- | (td, J = 7.6, 1.3 Hz, 1H), 7.49 (dd, J = 7.8, 0.7 Hz, 1H), 7.40 (td, |
| | 6,7-dihydro-5H- | J = 7.6, 1.2 Hz, 1H), 6.38 (s, 1H), 4.72 (s, 2H), 3.65 (s, 2H), 3.03 |
| | cyclopenta[b]pyridine- | (t, J = 7.7 Hz, 2H), 2.96 (t, J = 7.5 Hz, 2H), 2.19-2.11 (m, 2H), |
| | 3-carboxamide | 1.51 (s, 6H); ESI MS (m/z) 350.3 (MH)⁺ |
| 189 | N-(1-((2- | ¹H-NMR (400 MHz, CHLOROFORM-D) δ 8.98 (d, J = 2.0 Hz, |
| | fluorobenzyl)oxy)-2- | 1H), 8.57 (q, J = 1.0 Hz, 1H), 7.90 (d, J = 5.4 Hz, 1H), 7.61 (dd, |
| | methylpropan-2- | J = 5.6, 0.7 Hz, 1H), 7.38 (td, J = 7.5, 1.6 Hz, 1H), 7.33-7.28 (m, |
| | yl)thieno[3,2-b] | 1H), 7.14 (td, J = 7.5, 1.1 Hz, 1H), 7.10-7.05 (m, 1H), 6.51 (s, |
| | pyridine-6- | 1H), 4.66 (s, 2H), 3.58 (s, 2H), 1.53 (d, J = 6.1 Hz, 6H); ESI MS |
| | carboxamide | (m/z) 359.8 (MH)⁺ |
| 190 | N-(1-((2- | ¹H-NMR (400 MHz, CHLOROFORM-D) δ 8.63 (d, J = 2.0 Hz, |
| | fluorobenzyl)oxy)-2- | 1H), 7.83 (t, J = 1.0 Hz, 1H), 7.37 (td, J = 7.5, 1.7 Hz, 1H), 7.32- |
| | methylpropan-2-yl)- | 7.27 (m, 1H), 7.13 (td, J = 7.5, 1.1 Hz, 1H), 7.08-7.03 (m, 1H), |
| | 6,7-dihydro-5H- | 6.37 (s, 1H), 4.64 (s, 2H), 3.54 (s, 2H), 3.04 (t, J = 7.7 Hz, 2H), |
| | cyclopenta[b]pyridine- | 2.96 (t, J = 7.6 Hz, 2H), 2.20-2.12 (m, 2H), 1.48 (s, 6H); ESI MS |
| | 3-carboxamide | (m/z) 342.7 (MH)⁺ |
| 191 | N-(1-((2- | ¹H-NMR (400 MHz, CHLOROFORM-D) δ 8.65 (d, J = 2.0 Hz, |
| | chlorobenzyl)oxy)-2- | 1H), 7.85 (t, J = 1.0 Hz, 1H), 7.43-7.41 (m, 1H), 7.38-7.36 (m, |
| | methylpropan-2-yl)- | 1H), 7.25-7.23 (m, 2H), 6.40 (s, 1H), 4.66 (s, 2H), 3.58 (s, 2H), |
| | 6,7-dihydro-5H- | 3.05 (t, J = 7.7 Hz, 2H), 2.96 (t, J = 7.5 Hz, 2H), 2.20-2.13 (m, |
| | cyclopenta[b]pyridine- | 2H), 1.51 (s, 6H). ESI MS (m/z) 359.0 (M)⁺ |
| | 3-carboxamide | |
| 192 | N-(1-((2- | ¹H-NMR (400 MHz, CHLOROFORM-D) δ 8.99 (d, J = 2.0 Hz, |
| | chlorobenzyl)oxy)-2- | 1H), 8.58 (q, J = 0.9 Hz, 1H), 7.89 (d, J = 5.6 Hz, 1H), 7.61 (dd, |
| | methylpropan-2- | J = 5.6, 0.7 Hz, 1H), 7.44-7.42 (m, 1H), 7.39-7.37 (m, 1H), 7.27 |
| | yl)thieno[3,2-b] | (d, J = 0.7 Hz, 1H), 7.25-7.25 (m, 1H), 6.55 (s, 1H), 4.68 (s, 2H), |
| | pyridine-6- | 3.61 (s, 2H), 1.53 (s, 6H); ESI MS (m/z) 374.4 (M)⁺ |
| | carboxamide | |
| 193 | N-(1-((2- | ¹H-NMR (400 MHz, CHLOROFORM-D) δ 9.03 (d, J = 2.0 Hz, |
| | cyanobenzyl)oxy)-2- | 1H), 8.64 (q, J = 0.9 Hz, 1H), 7.89 (d, J = 5.4 Hz, 1H), 7.67 (dd, |
| | methylpropan-2- | J = 7.8, 1.0 Hz, 1H), 7.60 (dd, J = 5.4, 0.7 Hz, 1H), 7.57 (dd, J = |
| | yl)thieno[3,2-b] | 7.7, 1.3 Hz, 1H), 7.50-7.48 (m, 1H), 7.41 (td, J = 7.6, 1.4 Hz, 1H), |
| | pyridine-6- | 6.57 (s, 1H), 4.74 (s, 2H), 3.68 (s, 2H), 1.55 (s, 6H); ESI MS |
| | carboxamide | (m/z) 365.5 (MH)⁺ |
| 194 | 3-bromo-N-(1-((2- | ¹H-NMR (400 MHz, CHLOROFORM-D) δ 8.76 (d, J = 2.0 Hz, |
| | fluorobenzyl)oxy)-2- | 1H), 8.17 (d, J = 2.0 Hz, 1H), 7.39 (td, J = 7.4, 1.9 Hz, 1H), 7.30- |
| | methylpropan-2-yl)-1- | 7.27 (m, 1H), 7.15-7.00 (m, 3H), 6.46 (s, 1H), 4.66 (s, 2H), 3.90 |
| | methyl-1H- | (s, 3H), 3.59 (s, 2H), 1.52 (s, 6H); m/z = 435 (M)⁺. |
| | pyrrolo[2,3-b]pyridine- | |
| | 5-carboxamide | |
| 195 | 3-bromo-N-(1-((2- | ¹H-NMR (400 MHz, CHLOROFORM-D) δ 8.78 (q, J = 2.3 Hz, |
| | chlorobenzyl)oxy)-2- | 1H), 8.18 (d, J = 2.0 Hz, 1H), 7.44 (dd, J = 7.1, 2.2 Hz, 1H), 7.38- |
| | methylpropan-2-yl)-1- | 7.34 (m, 1H), 7.30-7.27 (m, 2H), 7.25-7.21 (m, 1H), 6.49 (s, 1H), |
| | methyl-1H- | 4.67 (d, J = 5.6 Hz, 2H), 3.90-3.88 (m, 3H), 3.60 (d, J = 22.5 Hz, |
| | pyrrolo[2,3-b]pyridine- | 2H), 1.53 (s, 6H); m/z = 451.4 (MH)⁺. |
| | 5-carboxamide | |
| 196 | 3-bromo-N-(1-((2- | ¹H-NMR (400 MHz, CHLOROFORM-D) δ 8.78 (d, J = 2.0 Hz, |
| | cyanobenzyl)oxy)-2- | 1H), 8.22 (d, J = 2.2 Hz, 1H), 7.67 (dd, J = 7.7, 0.9 Hz, 1H), 7.58 |
| | methylpropan-2-yl)-1- | (td, J = 7.6, 1.4 Hz, 1H), 7.52-7.50 (m, 1H), 7.40 (td, J = 7.6, 1.5 |
| | methyl-1H- | Hz, 1H), 7.27 (s, 1H), 6.46 (s, 1H), 4.75 (s, 2H), 3.89 (d, J = 4.9 |
| | pyrrolo[2,3-b]pyridine- | Hz, 3H), 3.71 (s, 2H), 1.54 (s, 6H); m/z = 440.4 (MH)⁺. |
| | 5-carboxamide | |
| 197 | N-(1-((2-chloro-5- | ¹H-NMR (400 MHz, CHLOROFORM-D) δ 8.65 (t, J = 1.1 Hz, |
| | fluorobenzyl)oxy)-2- | 1H), 7.84 (t, J = 1.0 Hz, 1H), 7.30 (dd, J = 8.8, 5.1 Hz, 1H), 7.16 |
| | methylpropan-2-yl)- | (dd, J = 9.0, 3.2 Hz, 1H), 6.93 (td, J = 8.3, 3.1 Hz, 1H), 6.28 (s, |
| | 6,7-dihydro-5H- | 1H), 4.61 (s, 2H), 3.66 (s, 2H), 3.03 (t, J = 7.7 Hz, 2H), 2.96 (t, J = |
| | cyclopenta[b]pyridine- | 7.6 Hz, 2H), 2.19-2.12 (m, 2H), 1.51 (s, 6H): m/z = 377.0 |
| | 3-carboxamide | (MH)⁺. |
| 198 | N-(1-((2-chloro-5- | ¹H-NMR (400 MHz, CHLOROFORM-D) δ 8.99 (d, J = 2.2 Hz, |
| | fluorobenzyl)oxy)-2- | 1H), 8.60-8.57 (s, 1H), 7.89 (d, J = 5.6 Hz, 1H), 7.60 (dd, J = 5.5, |
| | methylpropan-2- | 0.9 Hz, 1H), 7.31 (dd, J = 8.7, 5.0 Hz, 1H), 7.17 (dd, J = 9.0, 2.9 |
| | yl)thieno[3,2-b] | Hz, 1H), 6.96-6.92 (m, 1H), 6.43 (s, 1H), 4.64 (s, 2H), 3.69 (s, |
| | pyridine-6- | 2H), 1.56 (s, 6H); m/z = 392.95 (MH)⁺. |
| | carboxamide | |
| 199 | N-(1-((2,4- | ¹H-NMR (400 MHz, CHLOROFORM-D) δ 8.61 (s, 1H), 7.81 (d, |
| | dimethylbenzyl)oxy)- | J = 0.9 Hz, 1H), 7.16 (d, J = 7.6 Hz, 1H), 6.98 (d, J = 11.0 Hz, |
| | 2-methylpropan-2-yl)- | 2H), 6.30 (s, 1H), 4.53 (s, 2H), 3.50 (s, 2H), 3.04 (t, J = 7.8 Hz, |
| | 6,7-dihydro-5H- | 2H), 2.96 (t, J = 7.5 Hz, 2H), 2.30 (s, 6H), 2.20-2.12 (m, 2H), |
| | cyclopenta[b]pyridine- | 1.45 (s, 6H); m/z = 353.3 (MH)⁺. |
| | 3-carboxamide | |
| 200 | N-(1-((2,4- | ¹H-NMR (400 MHz, CHLOROFORM-D) δ 8.96 (s, 1H), 8.53 (d, |
| | dimethylbenzyl)oxy)- | J = 1.2 Hz, 1H), 7.89 (d, J = 5.5 Hz, 1H), 7.61 (d, J = 5.5 Hz, 1H), |
| | 2-methylpropan-2- | 7.17 (d, J = 7.3 Hz, 1H), 7.00-6.98 (m, 2H), 6.44 (s, 1H), 4.55 (s, |
| | yl)thieno[3,2-b] | 2H), 3.53 (s, 2H), 2.31 (s, 3H), 2.30 (s, 3H), 1.51 (s, 6H); m/z = |
| | pyridine-6- | 369.3 (MH)⁺. |
| | carboxamide | |
| 201 | 3-bromo-N-(1-((2,4- | ¹H-NMR(400 MHz, CHLOROFORM-D) δ 8.73 (d, J = 2.0 Hz, |
| | dimethylbenzyl)oxy)- | 1H), 8.15 (d, J = 2.2 Hz, 1H), 7.27 (s, 1H), 7.18 (d, J = 7.3 Hz, |
| | 2-methylpropan-2-yl)- | 1H), 6.98 (d, J = 7.6 Hz, 2H), 6.40 (s, 1H), 4.55 (s, 2H), 3.89 (s, |
| | 1-methyl-1H- | 3H), 3.54 (s, 2H), 2.31 (s, 3H), 2.30 (s, 3H), 1.51 (s, 6H): m/z = |
| | pyrrolo[2,3-b]pyridine- | 446 (MH)⁺. |
| | 5-carboxamide | |
| 202 | N-(2-methyl-1- | ¹H-NMR (400 MHz, CHLOROFORM-D) δ 8.65 (t, J = 0.9 Hz, |
| | (pyridin-2- | 1H), 8.18 (dt, J = 4.1, 0.9 Hz, 1H), 7.98 (s, 1H), 7.89 (t, J = 0.9 |
| | yloxy)propan-2-yl)- | Hz, 1H), 7.62 (ddd, J = 8.7, 6.9, 1.7 Hz, 1H), 6.94 (ddd, J = 7.0, |
| | 6,7-dihydro-5H- | 5.2, 0.9 Hz, 1H), 6.81 (d, J = 8.3 Hz, 1H), 4.40 (s, 2H), 3.03 (t, J = |
| | cyclopenta[b]pyridine- | 7.6 Hz, 2H), 2.95 (t, J = 7.5 Hz, 2H), 2.19-2.12 (m, 2H), 1.60 |
| | 3-carboxamide | (s, 6H); m/z = 312.1 (MH)⁺. |
| 203 | 3-bromo-1-methyl-N- | ¹H-NMR (400 MHz, CHLOROFORM-D) δ 8.84 (dd, J = 5.8, 2.1 |
| | (2-methyl-1-(pyridin- | Hz, 1H), 8.30 (dq, J = 5.1, 0.9 Hz, 1H), 8.18-8.15 (m, 2H), 7.62 |
| | 2-yloxy)propan-2-yl)- | (ddd, J = 8.6, 6.8, 1.6 Hz, 1H), 6.95 (ddd, J = 7.0, 5.2, 0.9 Hz, |
| | 1H-pyrrolo[2,3-b] | 1H), 6.84-6.82 (m, 1H), 4.44 (s, 2H), 3.89 (d, J = 4.9 Hz, 3H), |
| | pyridine-5- | 1.65 (s, 6H); m/z = 404.8 (MH)⁺. |
| | carboxamide | |
| 204 | N-(1-((2- | ¹H-NMR (400 MHz, CHLOROFORM-D) δ 8.63 (d, J = 1.8 Hz, |
| | chloropyridin-4- | 1H), 8.17 (d, J = 5.8 Hz, 1H), 7.83 (t, J = 0.9 Hz, 1H), 6.87 (d, J = |
| | yl)oxy)-2- | 2.1 Hz, 1H), 6.76 (dd, J = 5.8, 2.1 Hz, 1H), 6.00 (s, 1H), 4.30 |
| | methylpropan-2-yl)- | (s, 2H), 3.04 (t, J = 7.6 Hz, 2H), 2.97 (t, J = 7.5 Hz, 2H), 2.20- |
| | 6,7-dihydro-5H- | 2.13 (m, 2H), 1.56 (t, J = 11.8 Hz, 6H); m/z = 345.9 (MH)⁺. |
| | cyclopenta[b]pyridine- | |
| | 3-carboxamide | |
| 205 | N-(1-((2- | ¹H-NMR(400 MHz, CHLOROFORM-D) δ 8.98 (d, J = 2.1 Hz, |
| | chloropyridin-4- | 1H), 8.60 (q, J = 0.8 Hz, 1H), 8.18 (d, J = 5.8 Hz, 1H), 7.92 (d, J = |
| | yl)oxy)-2- | 5.8 Hz, 1H), 7.61 (dd, J = 5.7, 0.8 Hz, 1H), 6.89 (d, J = 2.1 Hz, |
| | methylpropan-2- | 1H), 6.78 (q, J = 2.8 Hz, 1H), 6.13 (s, 1H), 4.33 (s, 2H), 1.62 (d, |
| | yl)thieno[3,2-b] | J = 8.9 Hz, 6H); m/z = 361.9 (MH)⁺. |
| | pyridine-6- | |
| | carboxamide | |
| 206 | 3-bromo-N-(1-((2- | ¹H-NMR(400 MHz, CHLOROFORM-D) δ 8.74 (d, J = 2.1 Hz, |
| | chloropyridin-4- | 1H), 8.18-8.17 (m, 2H), 7.30 (s, 1H), 6.89 (d, J = 2.1 Hz, 1H), |
| | yl)oxy)-2- | 6.79 (dd, J = 5.8, 2.1 Hz, 1H), 6.08 (s, 1H), 4.35 (s, 2H), 3.90 (s, |
| | methylpropan-2-yl)-1- | 3H), 1.60 (s, 6H); m/z = 438.8 (MH)⁺. |
| | methyl-1H- | |
| | pyrrolo[2,3-b]pyridine- | |
| | 5-carboxamide | |
| 207 | N-(1-((5-fluoropyridin- | ¹H-NMR (400 MHz, CHLOROFORM-D) δ 8.99 (d, J = 2.1 Hz, |
| | 2-yl)oxy)-2- | 1H), 8.60 (q, J = 0.9 Hz, 1H), 8.02 (d, J = 3.1 Hz, 1H), 7.89 (d, J = |
| | methylpropan-2- | 5.5 Hz, 1H), 7.61-7.57 (m, 2H), 7.41-7.36 (m, 1H), 6.81-6.78 |
| | yl)thieno[3,2-b] | (m, 1H), 4.41 (s, 2H), 1.63 (s, 6H); m/z = 345.9 (M)⁺. |
| | pyridine-6- | |
| | carboxamide | |
| 208 | 3-chloro-N-(1-((2- | ¹H-NMR (400 MHz, CHLOROFORM-D) δ 8.78 (d, J = 2.2 Hz, |
| | chlorobenzyl)oxy)-2- | 1H), 8.24 (d, J = 2.2 Hz, 1H), 7.44 (dd, J = 7.0, 2.3 Hz, 1H), 7.38- |
| | methylpropan-2-yl)-1- | 7.36 (m, 1H), 7.28-7.26 (m, 1H), 7.24 (dd, J = 4.0, 2.6 Hz, 1H), |
| | methyl-1H- | 7.22 (d, J = 2.9 Hz, 1H), 6.49 (s, 1H), 4.68 (s, 2H), 3.89 (s, 3H), |
| | pyrrolo[2,3-b]pyridine- | 3.62 (s, 2H), 1.55 (s, 6H); m/z = 407.8 (MH)⁺. |
| | 5-carboxamide | |
| 209 | 3-chloro-N-(1-((2- | ¹H-NMR (400 MHz, CHLOROFORM-D) δ 8.79 (d, J = 2.1 Hz, |
| | cyanobenzyl)oxy)-2- | 1H), 8.28 (d, J = 2.1 Hz, 1H), 7.66 (dd, J = 7.6, 1.2 Hz, 1H), 7.57 |
| | methylpropan-2-yl)-1- | (td, J = 7.6, 1.3 Hz, 1H), 7.51 (d, J = 7.3 Hz, 1H), 7.39 (td, J = |
| | methyl-1H- | 7.6, 1.3 Hz, 1H), 7.21 (s, 1H), 6.45 (s, 1H), 4.75 (s, 2H), 3.88 (s, |
| | pyrrolo[2,3-b]pyridine- | 3H), 3.71 (s, 2H), 1.55 (s, 6H); m/z = 397.3 (MH)⁺. |
| | 5-carboxamide | |
| 210 | 2-chloro-N-(2-methyl- | ¹H-NMR (400 MHz, CHLOROFORM-D) δ 7.78 (s, 1H), 7.30- |
| | 1-phenylpropan-2-yl)- | 7.28 (m, 2H), 7.24-7.20 (m, 3H), 5.93 (s, 1H), 3.16 (s, 2H), 2.96 |
| | 6,7-dihydro-5H- | (dt, J = 24.1, 7.6 Hz, 4H), 2.20-2.12 (m, 2H), 1.46 (s, 6H); ESI |
| | cyclopenta[b]pyridine- | MS (m/z) 329.0 (MH)⁺ |
| | 3-carboxamide | |
| 211 | 2-chloro-N-(2,4- | ¹H-NMR (400 MHz, CHLOROFORM-D) δ 7.80 (s, 1H), 7.29- |
| | dimethyl-1- | 7.27 (m, 2H), 7.25-7.18 (m, 3H), 5.89 (s, 1H), 3.38 (d, J = 13.2 |
| | phenylpentan-2-yl)- | Hz, 1H), 3.01-2.92 (m, 5H), 2.20-2.08 (m, 3H), 1.88 (t, J = 6.6 |
| | 6,7-dihydro-5H- | Hz, 1H), 1.60-1.57 (m, 1H), 1.36 (s, 3H), 0.99 (dd, J = 6.6, 3.9 |
| | cyclopenta[b]pyridine- | Hz, 6H); ESI MS (m/z) 371.1 (MH)⁺ |
| | 3-carboxamide | |
| 212 | N-(2-methyl-1- | ¹H-NMR (400 MHz, CHLOROFORM-D) δ 9.01 (d, J = 2.0 Hz, |
| | (pyridin-2- | 1H), 8.62 (q, J = 0.9 Hz, 1H), 8.33 (s, 1H), 8.22-8.21 (m, 1H), |
| | yloxy)propan-2- | 7.88 (d, J = 5.6 Hz, 1H), 7.66-7.60 (m, 2H), 6.97 (ddd, J = 7.1, |
| | yl)thieno[3,2-b] | 5.1, 1.0 Hz, 1H), 6.84 (dd, J = 7.6, 0.7 Hz, 1H), 4.44 (s, 2H), 1.65 |
| | pyridine-6- | (s, 6H); m/z = 327.9 (MH)⁺. |
| | carboxamide | |
| 213 | 3-chloro-N-(1-((2- | ¹H-NMR (400 MHz, CHLOROFORM-D) δ 8.77 (d, J = 2.2 Hz, |
| | fluorobenzyl)oxy)-2- | 1H), 8.24 (d, J = 2.0 Hz, 1H), 7.39 (td, J = 7.4, 1.6 Hz, 1H), 7.32- |
| | methylpropan-2-yl)-1- | 7.27 (m, 1H), 7.21 (s, 1H), 7.13 (td, J = 7.5, 1.1 Hz, 1H), 7.09- |
| | methyl-1H- | 7.04 (m, 1H), 6.45 (s, 1H), 4.66 (s, 2H), 3.89 (s, 3H), 3.59 (s, 2H), |
| | pyrrolo[2,3-b]pyridine- | 1.50 (d, J = 13.4 Hz, 6H); m/z = 390.3 (MH)⁺. |
| | 5-carboxamide | |
| 214 | 3-chloro-N-(1-((2- | ¹H-NMR (400 MHz, CHLOROFORM-D) δ 8.78 (d, J = 2.0 Hz, |
| | chloro-5- | 1H), 8.25 (d, J = 2.0 Hz, 1H), 7.30 (dd, J = 8.7, 5.0 Hz, 1H), 7.21 |
| | fluorobenzyl)oxy)-2- | (s, 1H), 7.18 (dd, J = 9.0, 3.2 Hz, 1H), 6.93 (td, J = 8.3, 3.1 Hz, |
| | methylpropan-2-yl)-1- | 1H), 6.37 (s, 1H), 4.64 (s, 2H), 3.89 (s, 3H), 3.70 (s, 2H), 1.54 (d, |
| | methyl-1H- | J = 12.0 Hz, 6H); m/z = 423.9 (MH)⁺. |
| | pyrrolo[2,3-b]pyridine- | |
| | 5-carboxamide | |
| 216 | 3-bromo-N-(1-((5- | ¹H-NMR (400 MHz, CHLOROFORM-D) δ 8.82-8.81 (m, 1H), |
| | fluoropyridin-2- | 8.14 (d, J = 2.1 Hz, 1H), 8.11 (d, J = 3.1 Hz, 1H), 7.59 (s, 1H), |
| | yl)oxy)-2- | 7.41-7.36 (m, 1H), 7.27 (s, 1H), 6.80 (dd, J = 9.0, 3.5 Hz, 1H), |
| | methylpropan-2-yl)-1- | 4.42 (s, 2H), 3.89 (s, 3H), 1.63 (s, 6H); m/z = 421.0 (MH)⁺. |
| | methyl-1H- | |
| | pyrrolo[2,3-b]pyridine- | |
| | 5-carboxamide | |
| 217 | 2-methoxy-N-(2- | ¹H-NMR (400 MHz, CHLOROFORM-D) δ 8.31 (s, 1H), 7.68 (s, |
| | methyl-1- | 1H), 7.25-7.22 (m, 2H), 7.21-7.20 (m, 1H), 7.19-7.15 (m, 2H), |
| | phenylpropan-2-yl)- | 3.92 (s, 3H), 3.14 (s, 2H), 2.95-2.88 (m, 4H), 2.18-2.09 (m, 2H), |
| | 6,7-dihydro-5H- | 1.43 (s, 6H); ESI MS (m/z) 325.35 (MH)⁺ |
| | cyclopenta[b]pyridine- | |
| | 3-carboxamide | |
| 218 | 3-chloro-N-(1-((2,4- | ¹H-NMR (400 MHz, CHLOROFORM-D) δ 8.74 (d, J = 2.1 Hz, |
| | dimethylbenzyl)oxy)- | 1H), 8.21 (d, J = 2.1 Hz, 1H), 7.21 (s, 1H), 7.18 (d, J = 7.6 Hz, |
| | 2-methylpropan-2-yl)- | 1H), 6.98 (d, J = 7.9 Hz, 2H), 6.39 (s, 1H), 4.55 (s, 2H), 3.88 (s, |
| | 1-methyl-1H- | 3H), 3.54 (s, 2H), 2.32 (s, 3H), 2.30 (s, 3H), 1.51 (t, J = 14.5 Hz, |
| | pyrrolo[2,3-b]pyridine- | 6H); ESI MS (m/z) 400.2 (MH)⁺ |
| | 5-carboxamide | |
| 219 | 3-chloro-1-methyl-N- | ¹H-NMR (400 MHz, CHLOROFORM-D) δ 8.84 (d, J = 1.8 Hz, |
| | (2-methyl-1-(pyridin- | 1H), 8.29-8.28 (m, 1H), 8.25 (d, J = 2.1 Hz, 1H), 8.14 (s, 1H), |
| | 2-yloxy)propan-2-yl)- | 7.63 (ddd, J = 8.8, 6.6, 1.6 Hz, 1H), 7.20 (s, 1H), 6.95 (ddd, J = |
| | 1H-pyrrolo[2,3-b] | 7.0, 5.2, 0.9 Hz, 1H), 6.84-6.82 (m, 1H), 4.44 (s, 2H), 3.89 (s, |
| | pyridine-5- | 3H), 1.65 (s, 6H); m/z = 358.9 (MH)⁺. |
| | carboxamide | |
| 220 | 3-chloro-N-(1-((2- | ¹H-NMR (400 MHz, CHLOROFORM-D) δ 8.74 (d, J = 2.1 Hz, |
| | chloropyridin-4- | 1H), 8.23 (d, J = 1.8 Hz, 1H), 8.16 (d, J = 5.8 Hz, 1H), 7.23 (s, |
| | yl)oxy)-2- | 1H), 6.89 (d, J = 2.4 Hz, 1H), 6.78 (dd, J = 5.8, 2.1 Hz, 1H), 6.10 |
| | methylpropan-2-yl)-1- | (s, 1H), 4.34 (s, 2H), 3.89 (s, 3H), 1.60 (s, 6H); m/z = 392.9 |
| | methyl-1H- | (MH)⁺. |
| | pyrrolo[2,3-b]pyridine- | |
| | 5-carboxamide | |
| 221 | 3-chloro-N-(1-((5- | ¹H-NMR (400 MHz, CHLOROFORM-D) δ 8.82 (d, J = 1.8 Hz, |
| | fluoropyridin-2- | 1H), 8.23 (d, J = 2.1 Hz, 1H), 8.09 (d, J = 3.1 Hz, 1H), 7.59 (s, |
| | yl)oxy)-2- | 1H), 7.41-7.36 (m, 1H), 7.22 (d, J = 2.1 Hz, 1H), 6.80 (dd, J = |
| | methylpropan-2-yl)-1- | 9.0, 3.5 Hz, 1H), 4.42 (s, 2H), 3.89 (s, 3H), 1.63 (s, 6H); m/z = |
| | methyl-1H- | 377.2 (MH)⁺. |
| | pyrrolo[2,3-b]pyridine- | |
| | 5-carboxamide | |
| 222 | 3-chloro-N-(1-((5- | ¹H-NMR (400 MHz, CHLOROFORM-D) δ 8.78 (d, J = 2.1 Hz, |
| | cyanopyridin-2- | 1H), 8.54 (dd, J = 2.4, 0.6 Hz, 1H), 8.19 (d, J = 2.1 Hz, 1H), 7.81 |
| | yl)oxy)-2- | (dd, J = 8.7, 2.3 Hz, 1H), 7.23 (s, 1H), 6.89-6.87 (m, 1H), 6.83 |
| | methylpropan-2-yl)-1- | (s, 1H), 4.61 (s, 2H), 3.89 (s, 3H), 1.62 (s, 6H); m/z = 383.9 |
| | methyl-1H- | (MH)⁺. |
| | pyrrolo[2,3-b]pyridine- | |
| | 5-carboxamide | |
| 223 | N-(1-((5-cyanopyridin- | ¹H-NMR (400 MHz, CHLOROFORM-D) δ 8.63 (s, 1H), 8.48 (d, |
| | 2-yl)oxy)-2- | J = 1.8 Hz, 1H), 7.87 (s, 1H), 7.80 (dd, J = 8.7, 2.3 Hz, 1H), 6.85 |
| | methylpropan-2-yl)- | (dd, J = 8.6, 0.6 Hz, 1H), 6.55 (s, 1H), 4.58 (s, 2H), 3.06 (t, J = |
| | 6,7-dihydro-5H- | 7.8 Hz, 2H), 3.00-2.95 (m, 2H), 2.21-2.13 (m, 2H), 1.58 (s, 6H); |
| | cyclopenta[b]pyridine- | m/z = 336.9 (MH)⁺. |
| | 3-carboxamide | |
| 224 | N-(1-((5-cyanopyridin- | ¹H-NMR (400 MHz, CHLOROFORM-D) δ 8.97 (d, J = 1.8 Hz, |
| | 2-yl)oxy)-2- | 1H), 8.60 (d, J = 2.1 Hz, 1H), 8.50 (d, J = 2.4 Hz, 1H), 7.92 (d, J = |
| | methylpropan-2- | 5.5 Hz, 1H), 7.81 (dd, J = 8.7, 2.3 Hz, 1H), 7.62 (d, J = 5.5 Hz, |
| | yl)thieno[3,2-b] | 1H), 6.87 (d, J = 8.6 Hz, 1H), 6.73 (s, 1H), 4.61 (s, 2H), 1.62- |
| | pyridine-6- | 1.64 (6H); m/z = 352.9 (MH)⁺. |
| | carboxamide | |
| 225 | 3-bromo-N-(1-((5- | ¹H-NMR (400 MHz, CHLOROFORM-D) δ 8.77 (d, J = 1.8 Hz, |
| | cyanopyridin-2- | 1H), 8.57 (t, J = 1.1 Hz, 1H), 8.13 (d, J = 2.1 Hz, 1H), 7.81 (dd, |
| | yl)oxy)-2- | J = 8.7, 2.3 Hz, 1H), 7.29 (s, 1H), 6.89-6.86 (m, 2H), 4.61 (s, 2H), |
| | methylpropan-2-yl)-1- | 3.90 (s, 3H), 1.63 (s, 6H); m/z = 429.6 (MH)⁺. |
| | methyl-1H- | |
| | pyrrolo[2,3-b]pyridine- | |
| | 5-carboxamide | |
| 226 | N-(1-((2-chloro-5- | ¹H-NMR (400 MHz, CHLOROFORM-D) δ 8.72 (d, J = 2.1 Hz, |
| | fluorobenzyl)oxy)-2- | 1H), 8.26 (dd, J = 11.3, 2.1 Hz, 1H), 7.30 (q, J = 4.6 Hz, 1H), |
| | methylpropan-2-yl)-1- | 7.24 (d, J = 3.7 Hz, 1H), 7.22-7.18 (m, 1H), 6.93 (td, J = 8.4, 3.1 |
| | methyl-1H- | Hz, 1H), 6.52 (d, J = 3.4 Hz, 1H), 6.33 (s, 1H), 4.68-4.60 (m, 2H), |
| | pyrrolo[2,3-b]pyridine- | 3.99-3.86 (m, 3H), 3.74-3.67 (m, 2H), 1.53 (d, J = 14.4 Hz, 6H); |
| | 5-carboxamide | m/z = 390.4 (MH)⁺. |
| 227 | N-(1-((2,5- | ¹H-NMR (400 MHz, CHLOROFORM-D) δ 8.69 (d, J = 1.8 Hz, |
| | dimethylbenzyl)oxy)- | 1H), 8.23 (d, J = 2.1 Hz, 1H), 7.23 (d, J = 3.7 Hz, 1H), 7.18 (d, J = |
| | 2-methylpropan-2-yl)- | 7.3 Hz, 1H), 6.98 (d, J = 7.6 Hz, 2H), 6.51 (d, J = 3.7 Hz, 1H), |
| | 1-methyl-1H- | 6.36 (s, 1H), 4.54 (s, 2H), 3.91 (s, 3H), 3.54 (s, 2H), 2.30 (s, 6H), |
| | pyrrolo[2,3-b]pyridine- | 1.50 (s, 6H); m/z = 366.5 MH)⁺. |
| | 5-carboxamide | |
| 228 | 1-methyl-N-(2-methyl- | ¹H-NMR (400 MHz, CHLOROFORM-D) δ 8.73 (d, J = 2.1 Hz, |
| | 1-(pyridin-2- | 1H), 8.28 (d, J = 2.1 Hz, 1H), 8.20-8.18 (m, 1H), 7.67 (s, 1H), |
| | yloxy)propan-2-yl)- | 7.61 (ddd, J = 8.7, 6.7, 1.7 Hz, 1H), 7.22 (d, J = 3.7 Hz, 1H), 6.93 |
| | 1H-pyrrolo[2,3-b] | (ddd, J = 7.0, 5.2, 0.9 Hz, 1H), 6.82 (dt, J = 8.4, 0.8 Hz, 1H), 6.50 |
| | pyridine-5- | (d, J = 3.7 Hz, 1H), 4.44 (s, 2H), 3.91 (s, 3H), 1.63 (s, 6H); m/z = |
| | carboxamide | 325.1 (MH)⁺. |
| 229 | N-(1-((2- | ¹H-NMR (400 MHz, CHLOROFORM-D) δ 8.69 (d, J = 2.1 Hz, |
| | chloropyridin-4- | 1H), 8.27 (d, J = 2.1 Hz, 1H), 8.17 (d, J = 5.8 Hz, 1H), 7.25 (s, |
| | yl)oxy)-2- | 1H), 6.89 (d, J = 2.1 Hz, 1H), 6.78 (q, J = 2.8 Hz, 1H), 6.53 (d, J = |
| | methylpropan-2-yl)-1- | 3.4 Hz, 1H), 6.06 (s, 1H), 5.30 (s, 1H), 4.33 (s, 2H), 3.92-3.89 |
| | methyl-1H- | (m, 3H), 1.59 (s, 6H); m/z = 359.0 (MH)⁺. |
| | pyrrolo[2,3-b]pyridine- | |
| | 5-carboxamide | |
| 230 | N-(1-((5-fluoropyridin- | ¹H-NMR (400 MHz, CHLOROFORM-D) δ 8.71 (d, J = 2.1 Hz, |
| | 2-yl)oxy)-2- | 1H), 8.27 (d, J = 2.1 Hz, 1H), 8.01 (d, J = 3.1 Hz, 1H), 7.39-7.34 |
| | methylpropan-2-yl)-1- | (m, 1H), 7.23 (t, J = 3.1 Hz, 1H), 7.14 (s, 1H), 6.78 (dd, J = 9.0, |
| | methyl-1H- | 3.5 Hz, 1H), 6.51 (d, J = 3.4 Hz, 1H), 4.42 (s, 2H), 3.91 (s, 3H), |
| | pyrrolo[2,3-b]pyridine- | 1.62 (s, 6H); m/z = 343.2 (MH)⁺. |
| | 5-carboxamide | |
| 231 | N-(1-((5-cyanopyridin- | ¹H-NMR (400 MHz, CHLOROFORM-D) δ 8.68 (d, J = 2.1 Hz, |
| | 2-yl)oxy)-2- | 1H), 8.49 (dd, J = 2.4, 0.6 Hz, 1H), 8.27 (d, J = 2.1 Hz, 1H), 7.79 |
| | methylpropan-2-yl)-1- | (dd, J = 8.6, 2.4 Hz, 1H), 7.24 (s, 1H), 6.85 (dd, J = 8.7, 0.8 Hz, |
| | methyl-1H- | 1H), 6.53 (t, J = 3.4 Hz, 2H), 4.61 (s, 2H), 3.91 (s, 3H), 1.67 (s, |
| | pyrrolo[2,3-b]pyridine- | 6H); m/z = 350.1 (MH)⁺. |
| | 5-carboxamide | |
| 232 | 3-bromo-N-(1-((2- | ¹H-NMR (400 MHz, CHLOROFORM-D) δ 8.77 (t, J = 2.3 Hz, |
| | chloro-5- | 1H), 8.18 (d, J = 1.8 Hz, 1H), 7.32-7.28 (m, 2H), 7.18 (dd, J = |
| | fluorobenzyl)oxy)-2- | 8.9, 3.1 Hz, 1H), 6.93 (td, J = 8.3, 3.2 Hz, 1H), 6.36 (s, 1H), 4.64 |
| | methylpropan-2-yl)-1- | (s, 2H), 3.90 (s, 3H), 3.71 (s, 2H), 1.56 (s, 6H); m/z = 469.7 (MH)⁺. |
| | methyl-1H- | |
| | pyrrolo[2,3-b]pyridine- | |
| | 5-carboxamide | |
| 233 | N-(1-((5-fluoropyridin- | ¹H-NMR (400 MHz, CHLOROFORM-D) δ 8.64 (t, J = 0.9 Hz, |
| | 2-yl)oxy)-2- | 1H), 8.00 (d, J = 3.1 Hz, 1H), 7.86 (t, J = 0.9 Hz, 1H), 7.40-7.29 |
| | methylpropan-2-yl)- | (m, 2H), 6.77 (dd, J = 9.2, 3.4 Hz, 1H), 4.38 (s, 2H), 3.04 (t, J = |
| | 6,7-dihydro-5H- | 7.8 Hz, 2H), 2.96 (t, J = 7.5 Hz, 2H), 2.20-2.12 (m, 2H), 1.59 (s, |
| | cyclopenta[b]pyridine- | 6H); m/z = 330.2 (MH)⁺. |
| | 3-carboxamide | |
| 234 | N-(1-((2- | ¹H-NMR (400 MHz, CHLOROFORM-D) δ 8.25 (s, 1H), 8.18 (s, |
| | cyanobenzyl)oxy)-2- | 1H), 7.64 (d, J = 7.6 Hz, 1H), 7.59-7.56 (m, 2H), 7.39-7.35 (m, |
| | methylpropan-2-yl)-2- | 1H), 4.77 (s, 2H), 3.95 (s, 3H), 3.71 (s, 2H), 2.90 (dt, J = 17.4, |
| | methoxy-6,7-dihydro- | 7.6 Hz, 4H), 2.17-2.09 (m, 2H), 1.50 (s, 6H); ESI MS (m/z) |
| | 5H- | 380.3 (MH)⁺ |
| | cyclopenta[b]pyridine- | |
| | 3-carboxamide | |
| 235 | 2-chloro-N-(1-((2- | ¹H-NMR (400 MHz, CHLOROFORM-D) δ 7.78 (s, 1H), 7.65 |
| | cyanobenzyl)oxy)-2- | (dd, J = 7.6, 0.7 Hz, 1H), 7.60-7.52 (m, 2H), 7.39 (td, J = 7.5, 1.4 |
| | methylpropan-2-yl)- | Hz, 1H), 6.53 (s, 1H), 4.73 (s, 2H), 3.65 (s, 2H), 3.01-2.88 (m, |
| | 6,7-dihydro-5H- | 4H), 2.19-2.12 (m, 2H), 1.51 (s, 6H); ESI MS (m/z) 384.3 (MH)₊ |
| | cyclopenta[b]pyridine- | |
| | 3-carboxamide | |
| 236 | N-(1-((2,4- | ¹H-NMR (400 MHz, CHLOROFORM-D) δ 8.25 (s, 2H), 7.21 (d, |
| | dimethylbenzyl)oxy)- | J = 8.1 Hz, 1H), 7.00-6.96 (m, 2H), 4.54 (s, 2H), 3.91 (s, 3H), |
| | 2-methylpropan-2-yl)- | 3.55 (s, 2H), 2.90 (dt, J = 17.4, 7.7 Hz, 4H), 2.30 (s, 3H), 2.28 (s, |
| | 2-methoxy-6,7- | 3H), 2.16-2.09 (m, 2H), 1.47 (s, 6H); ESI MS (m/z) 383.4 (MH)+ |
| | dihydro-5H- | |
| | cyclopenta[b]pyridine- | |
| | 3-carboxamide | |
| 237 | 2-chloro-N-(1-((2,4- | ¹H-NMR (400 MHz, CHLOROFORM-D) δ 7.75 (s, 1H), 7.17 (d, |
| | dimethylbenzyl)oxy)- | J = 8.1 Hz, 1H), 6.96 (d, J = 6.1 Hz, 2H), 6.47 (s, 1H), 4.51 (s, |
| | 2-methylpropan-2-yl)- | 2H), 3.50 (s, 2H), 2.96 (dt, J = 33.3, 7.6 Hz, 4H), 2.29 (s, 2H), |
| | 6,7-dihydro-5H- | 2.28 (s, 3H), 2.19-2.12 (m, 3H), 1.48 (s, 6H); ESI MS (m/z) |
| | cyclopenta[b]pyridine- | 387.1 (MH)⁺ |
| | 3-carboxamide | |
| 238 | N-(2,4-dimethyl-1- | ¹H-NMR (400 MHz, CHLOROFORM-D) δ 8.31 (s, 1H), 7.59 (s, |
| | phenylpentan-2-yl)-2- | 1H), 7.23-7.17 (m, 3H), 7.14-7.12 (m, 2H), 3.91 (s, 3H), 3.32 (d, |
| | methoxy-6,7-dihydro- | J = 13.2 Hz, 1H), 3.02 (d, J = 13.2 Hz, 1H), 2.92 (q, J = 7.8 Hz, |
| | 5H- | 4H), 2.18-2.10 (m, 2H), 2.00-1.95 (m, 1H), 1.85-1.81 (m, 1H), |
| | cyclopenta[b]pyridine- | 1.54 (dd, J = 14.3, 5.0 Hz, 1H), 1.37 (s, 3H), 0.95 (dd, J = 8.3, 6.6 |
| | 3-carboxamide | Hz, 6H); ESI MS (m/z) 367.35 (MH)⁺ |
| 239 | N-(1-(2- | ¹H-NMR (400 MHz, DMSO-d6) δ 8.12 (s, 1H), 7.84-7.78 (m, |
| | fluorophenyl)propan- | 1H), 7.58 (d, J = 3.4 Hz, 1H), 7.29-7.25 (m, 2H), 7.12-7.03 (m, |
| | 2-yl)-N-methoxy-1- | 2H), 6.50 (d, J = 3.4 Hz, 1H), 4.50 (s, 1H), 3.85-3.78 (m, 3H), |
| | methyl-1H- | 3.61 (s, 3H), 3.02 (dd, J = 13.4, 9.2 Hz, 1H), 2.84 (dd, J = 13.6, |
| | pyrrolo[2,3-b]pyridine- | 5.3 Hz, 1H), 1.43-1.29 (m, 3H); ESI MS (m/z) 342.3 (MH)⁺ |
| | 5-carboxamide | |
| 240 | 2-(2-methoxy-6,7- | ¹H-NMR (400 MHz, CHLOROFORM-D) δ 8.26 (s, 1H), 8.09 (s, |
| | dihydro-5H- | 1H), 7.84 (dt, J = 7.8, 1.3 Hz, 1H), 7.72 (dq, J = 9.3, 1.3 Hz, 1H), |
| | cyclopenta[b]pyridine- | 7.41 (td, J = 8.0, 5.5 Hz, 1H), 7.28- 7.24 (m, 1H), 4.55 (s, 2H), |
| | 3-carboxamido)-2- | 3.99 (s, 3H), 2.90 (dt, J = 18.7, 7.6 Hz, 4H), 2.17-2.09 (m, 2H), |
| | methylpropyl | 1.56 (s, 6H); ESI MS (m/z) 387.3 (MH)⁺ |
| | 3-fluorobenzoate | |
| 241 | 2-methoxy-N-(2- | ¹H-NMR (400 MHz, CHLOROFORM-D) δ 8.31 (s, 1H), 8.27 (s, |
| | methyl-1-(pyridin-2- | 1H), 8.16-8.14 (m, 1H), 7.59 (ddd, J = 8.7, 6.8, 1.8 Hz, 1H), 6.88 |
| | yloxy)propan-2-yl)- | (ddd, J = 7.1, 5.0, 0.9 Hz, 1H), 6.80 (d, J = 8.3 Hz, 1H), 4.40 (s, |
| | 6,7-dihydro-5H- | 2H), 3.95 (s, 3H), 2.89 (dt, J = 18.9, 7.6 Hz, 4H), 2.16-2.08 (m, |
| | cyclopenta[b]pyridine- | 2H), 1.58 (s, 6H); ESI MS (m/z) 342.05 (MH)⁺ |
| | 3-carboxamide | |
| 242 | 2-chloro-N-(2-methyl- | ¹H-NMR (400 MHz, CHLOROFORM-D) δ 8.07-8.04 (m, 1H), |
| | 1-(pyridin-2- | 7.74 (s, 1H), 7.58 (ddd, J = 8.7, 6.7, 1.6 Hz, 1H), 7.21 (s, 1H), |
| | yloxy)propan-2-yl)- | 6.87 (ddd, J = 7.1, 5.0, 0.9 Hz, 1H), 6.77 (dt, J = 8.3, 0.9 Hz, 1H), |
| | 6,7-dihydro-5H- | 4.39 (s, 2H), 2.99 (t, J = 7.7 Hz, 2H), 2.91 (t, J = 7.5 Hz, 2H), |
| | cyclopenta[b]pyridine- | 2.19-2.11 (m, 2H), 1.60 (s, 6H); ESI MS (m/z) 345.9 (MH)⁺ |
| | 3-carboxamide | |
| 243 | N-(1-((2- | ¹H-NMR (400 MHz, CHLOROFORM-D) δ 8.22 (s, 1H), 8.15 (d, |
| | chloropyridin-4- | J = 5.6 Hz, 1H), 8.05 (s, 1H), 6.88 (d, J = 2.2 Hz, 1H), 6.78 (dd, |
| | yl)oxy)-2- | J = 5.9, 2.2 Hz, 1H), 4.29 (s, 2H), 4.04 (s, 3H), 2.90 (dt, J = 21.7, |
| | methylpropan-2-yl)-2- | 7.5 Hz, 4H), 2.16-2.09 (m, 2H), 1.54 (s, 6H); ESI MS (m/z) |
| | methoxy-6,7-dihydro- | 375.9 (MH)⁺ |
| | 5H- | |
| | cyclopenta[b]pyridine- | |
| | 3-carboxamide | |
| 244 | 2-chloro-N-(1-((2- | ¹H-NMR (400 MHz, CHLOROFORM-D) δ 8.18 (d, J = 5.9 Hz, |
| | chloropyridin-4- | 1H), 7.77 (s, 1H), 6.88 (d, J = 2.2 Hz, 1H), 6.78 (dd, J = 5.7, 2.3 |
| | yl)oxy)-2- | Hz, 1H), 6.34 (s, 1H), 4.28 (s, 2H), 3.00 (t, J = 7.7 Hz, 2H), 2.92 |
| | methylpropan-2-yl)- | (t, J = 7.5 Hz, 2H), 2.20-2.12 (m, 2H), 1.56 (s, 6H); ESI MS (m/z) |
| | 6,7-dihydro-5H- | 379.9 (MH)⁺ |
| | cyclopenta[b]pyridine- | |
| | 3-carboxamide | |
| 245 | N-(1- | ¹H-NMR (400 MHz, CHLOROFORM-D) δ 8.29 (s, 1H), 7.94 (s, |
| | benzylcyclopropyl)-2- | 1H), 7.28 (dd, J = 8.3, 1.8 Hz, 2H), 7.24-7.19 (m, 3H), 3.84 (s, |
| | methoxy-6,7-dihydro- | 3H), 3.00 (s, 2H), 2.90 (q, J = 8.2 Hz, 4H), 2.17-2.09 (m, 2H), |
| | 5H- | 0.89 (dd, J = 15.6, 10.4 Hz, 4H); ESI MS (m/z) 323.0 (MH)⁺ |
| | cyclopenta[b]pyridine- | |
| | 3-carboxamide | |
| 246 | N-(1- | ¹H-NMR (400 MHz, CHLOROFORM-D) δ 7.74 (s, 1H), 7.31- |
| | benzylcyclopropyl)-2- | 7.28 (m, 2H), 7.25-7.21 (m, 2H), 6.42 (s, 1H), 3.04 (s, 2H), 2.98 |
| | chloro-6,7-dihydro- | (t, J = 7.8 Hz, 2H), 2.92 (t, J = 7.5 Hz, 2H), 2.19-2.11 (m, 2H), |
| | 5H- | 0.99-0.92 (m, 4H); ESI MS (m/z) 326.9 (MH)⁺ |
| | cyclopenta[b]pyridine- | |
| | 3-carboxamide | |
| 247 | 2-methoxy-N-((1- | ¹H-NMR (400 MHz, CHLOROFORM-D) δ 8.28 (s, 1H), 8.05 (s, |
| | phenylcyclopropyl) | 1H), 7.39 (dt, J = 8.0, 1.6 Hz, 2H), 7.35-7.31 (m, 2H), 7.25-7.22 |
| | methyl)-6,7-dihydro-5H- | (m, 1H), 3.89 (s, 3H), 3.64 (d, J = 5.2 Hz, 2H), 2.93-2.86 (m, 4H), |
| | cyclopenta[b]pyridine- | 2.16-2.08 (m, 2H), 0.97-0.89 (m, 4H); ESI MS (m/z) 323.05 (MH)⁺ |
| | 3-carboxamide | |
| 248 | 2-chloro-N-((1- | ¹H-NMR (400 MHz, CHLOROFORM-D) δ 7.80 (d, J = 0.9 Hz, |
| | phenylcyclopropyl) | 1H), 7.36-7.28 (m, 4H), 7.24-7.20 (m, 1H), 6.47 (s, 1H), 3.66 (d, |
| | methyl)-6,7-dihydro-5H- | J = 5.5 Hz, 2H), 2.99 (t, J = 7.8 Hz, 2H), 2.91 (t, J = 7.5 Hz, 2H), |
| | cyclopenta[b]pyridine- | 2.19-2.11 (m, 2H), 1.00-0.94 (m, 4H); ESI MS (m/z) 326.95 (MH)⁺ |
| | 3-carboxamide | |
| 249 | 2-methoxy-N-(4- | ¹H-NMR (400 MHz, CHLOROFORM-D) δ 8.32 (s, 1H), 7.79 (d, |
| | phenylbutan-2-yl)-6,7- | J = 7.9 Hz, 1H), 7.28 (dd, J = 7.0, 1.2 Hz, 1H), 7.25 (d, J = 1.8 |
| | dihydro-5H- | Hz, 1H), 7.20-7.15 (m, 3H), 4.25 (dt, J = 14.7, 6.7 Hz, 1H), 2.96- |
| | cyclopenta[b]pyridine- | 2.88 (m, 4H), 2.73-2.69 (m, 2H), 2.18-2.10 (m, 2H), 1.90-1.84 |
| | 3-carboxamide | (m, 2H), 1.27 (t, J = 6.7 Hz, 3H); ESI MS (m/z) 325.3 (MH)⁺ |
| 250 | 2-chloro-N-(4- | ¹H-NMR (400 MHz, CHLOROFORM-D) δ 7.84 (s, 1H), 7.30- |
| | phenylbutan-2-yl)-6,7- | 7.28 (m, 2H), 7.22-7.16 (m, 3H), 6.20 (d, J = 8.3 Hz, 1H), 4.31- |
| | dihydro-5H- | 4.24 (m, 1H), 3.02 (t, J = 7.8 Hz, 2H), 2.94 (t, J = 7.3 Hz, 2H), |
| | cyclopenta[b]pyridine- | 2.77-2.70 (m, 2H), 2.21-2.14 (m, 2H), 1.91-1.85 (m, 2H), 1.30 |
| | 3-carboxamide | (d, J = 6.7 Hz, 3H); ESI MS (m/z) 328.9 (MH)⁺ |
| 251 | 2-chloro-N-(1- | ¹H-NMR (400 MHz, CHLOROFORM-D) δ 7.89 (s, 1H), 7.35- |
| | phenylcyclopropyl)- | 7.28 (m, 4H), 7.23-7.19 (m, 1H), 7.07 (s, 1H), 3.01 (t, J = 7.8 Hz, |
| | 6,7-dihydro-5H- | 2H), 2.94 (t, J = 7.3 Hz, 2H), 2.17 (tt, J = 11.6, 3.8 Hz, 2H), 1.41- |
| | cyclopenta[b]pyridine- | 1.36 (m, 4H); ESI MS (m/z) 312.95 (MH)+ |
| | 3-carboxamide | |
| 252 | 2-methoxy-N-(1- | ¹H-NMR (400 MHz, CHLOROFORM-D) δ 8.53 (s, 1H), 8.31 (s, |
| | phenylcyclopropyl)- | 1H), 7.28 (td, J = 6.0, 2.5 Hz, 4H), 7.19-7.14 (m, 1H), 4.09 (s, |
| | 6,7-dihydro-5H- | 3H), 2.94 (t, J = 7.6 Hz, 2H), 2.88 (t, J = 7.5 Hz, 2H), 2.17-2.10 |
| | cyclopenta[b]pyridine- | (m, 2H), 1.37 (s, 4H); ESI MS (m/z) 309.1 (MH)⁺ |
| | 3-carboxamide | |
| 253 | N-(1-(2- | ¹H-NMR (400 MHz, DMSO-d6) δ 7.30-7.23 (m, 3H), 7.12-7.05 |
| | fluorophenyl)propan- | (m, 2H), 4.38 (d, J = 26.0 Hz, 1H), 3.59 (s, 3H), 3.02-2.80 (m, |
| | 2-yl)-N-methoxy-6,7- | 6H), 2.08-2.01 (m, 2H), 1.36-1.29 (m, 3H); ESI MS (m/z) |
| | dihydro-5H- | 329.10 (MH)⁺ |
| | cyclopenta[b]pyridine- | |
| | 3-carboxamide | |
| 254 | 2-chloro-N-(1-(2- | ¹H-NMR (400 MHz, DMSO-d6) δ 7.33-7.10 (m, 5H), 3.75-3.59 |
| | fluorophenyl)propan- | (bs, 4H), 2.93-2.85 (m, 4H), 2.13-2.05 (m, 2H), 1.35 (d, J = 6.7 |
| | 2-yl)-N-methoxy-6,7- | Hz, 3H); ESI MS (m/z) 363.0 (MH)⁺ |
| | dihydro-5H- | |
| | cyclopenta[b]pyridine- | |
| | 3-carboxamide | |
| 255 | N-(1-(2- | ¹H-NMR (400 MHz, DMSO-d6) δ 11.86 (s, 1H), 8.05 (s, 1H), |
| | fluorophenyl)propan- | 7.78 (s, 1H), 7.53 (t, J = 2.9 Hz, 1H), 7.27 (dd, J = 12.2, 7.3 Hz, |
| | 2-yl)-N-methoxy-1H- | 2H), 7.12-7.02 (m, 2H), 6.48 (q, J = 1.8 Hz, 1H), 4.53-4.41(m, |
| | pyrrolo[2,3-b]pyridine- | 1H), 3.60 (s, 3H), 3.02 (dd, J = 13.8, 8.9 Hz, 1H), 2.85-2.82 (m, |
| | 5-carboxamide | 1H), 1.36 (d, J = 6.7 Hz, 3H); ESI MS (m/z) = 327.95 (MH)⁺ |
| 256 | 2-chloro-N-(1-(4- | ¹H-NMR (400 MHz, CHLOROFORM-D) δ 7.80 (s, 1H), 7.19- |
| | fluorophenyl)-2,4- | 7.15 (m, 2H), 6.98-6.93 (m, 2H), 6.03 (s, 1H), 4.94 (q, J = 1.2 Hz, |
| | dimethylpent-4-en-2- | 1H), 4.76 (s, 1H), 3.47 (d, J = 13.7 Hz, 1H), 3.02-2.92 (m, 6H), |
| | yl)-6,7-dihydro-5H- | 2.34 (d, J = 13.4 Hz, 1H), 2.20-2.13 (m, 2H), 1.83 (s, 3H), 1.33 |
| | cyclopenta[b]pyridine- | (s, 3H); ESI MS (m/z) = 387.40 (MH)⁺ |
| | 3-carboxamide | |
| 257 | 2-chloro-N-(1-(2- | ¹H-NMR (400 MHz, CHLOROFORM-D) δ 7.73 (s, 1H), 7.08- |
| | fluorophenoxy)-2,4- | 6.98 (m, 3H), 6.93-6.87 (m, 1H), 6.47 (s, 1H), 4.97 (t, J = 1.8 Hz, |
| | dimethylpent-4-en-2- | 1H), 4.84 (s, 1H), 4.33 (d, J = 9.0 Hz, 1H), 4.18 (d, J = 9.0 Hz, |
| | yl)-6,7-dihydro-5H- | 1H), 3.00 (t, J = 7.8 Hz, 2H), 2.92 (t, J = 7.5 Hz, 2H), 2.87 (d, J = |
| | cyclopenta[b]pyridine- | 13.4 Hz, 1H), 2.61 (d, J = 13.4 Hz, 1H), 2.20-2.12 (m, 2H), |
| | 3-carboxamide | 1.86 (s, 3H), 1.64 (s, 3H); ESI MS (m/z) = 403.25 (MH)⁺ |
| 258 | 2-(2-chloro-6,7- | ¹H-NMR (400 MHz, CHLOROFORM-D) δ 7.86-7.81 (m, 2H), |
| | dihydro-5H- | 7.71 (m, 1H), 7.42 (m, 1H), 7.29-7.27 (m, 1H), 6.44 (s, 1H), 4.56 |
| | cyclopenta[b]pyridine- | (d, J = 7.6 Hz, 2H), 2.96 (dt, J = 29.9, 7.6 Hz, 4H), 2.20-2.12 (m, |
| | 3-carboxamido)-2- | 2H), 1.60 (s, 6H); ESI MS (m/z) = 391.20 (MH)⁺ |
| | methylpropyl | |
| | 3-fluorobenzoate | |
| 259 | 2-methoxy-N-(4,4,4- | ¹H-NMR (400 MHz, CHLOROFORM-D) δ 8.29 (s, 1H), 7.78 (s, |
| | trifluoro-2-methyl-1- | 1H), 7.25-7.21 (m, 3H), 7.18-7.14 (m, 2H), 3.93 (s, 3H), 3.52 (d, |
| | phenylbutan-2-yl)-6,7- | J = 13.2 Hz, 1H), 3.35-3.22 (m, 1H), 3.02-2.89 (m, 5H), 2.63- |
| | dihydro-5H- | 2.51 (m, 1H), 2.19-2.11 (m, 2H), 1.42 (s, 3H); ESI MS (m/z) = |
| | cyclopenta[b]pyridine- | 393.45 (MH)⁺ |
| | 3-carboxamide | |
| 260 | 2-chloro-N-(2-methyl- | ¹H-NMR(400 MHz, CHLOROFORM-D) δ 7.72 (s, 1H), 7.29- |
| | 1-phenylpropan-2-yl)- | 7.26 (m, 2H), 7.24-7.18 (m, 3H), 6.08 (s, 1H), 3.15 (s, 2H), 2.87 |
| | 5,6,7,8- | (t, J = 6.4 Hz, 2H), 2.76 (t, J = 6.3 Hz, 2H), 1.90-1.77 (m, 4H), |
| | tetrahydroquinoline-3- | 1.45 (s, 6H); ESI MS (m/z) = 343.35 (MH)⁺ |
| | carboxamide | |
| 261 | 2-chloro-N-(2,4- | ¹H-NMR (400 MHz, CHLOROFORM-D) δ 7.74 (s, 1H), 7.28 (t, |
| | dimethyl-1- | J = 1.7 Hz, 1H), 7.26-7.17 (m, 4H), 6.02 (s, 1H), 3.38 (d, J = 13.1 |
| | phenylpentan-2-yl)- | Hz, 1H), 2.96 (d, J = 13.1 Hz, 1H), 2.88 (t, J = 6.4 Hz, 2H), 2.76 |
| | 5,6,7,8- | (t, J = 6.3 Hz, 2H), 2.10 (dd, J = 14.2, 6.0 Hz, 1H), 1.90-1.79 (m, |
| | tetrahydroquinoline-3- | 5H), 1.58 (dd, J = 14.4, 5.5 Hz, 1H), 1.36 (s, 3H), 0.99 (dd, J = |
| | carboxamide | 6.7, 4.9 Hz, 6H); ESI MS (m/z) = 385.40 (MH)⁺ |
| 262 | 2-chloro-N-(1-((2,4- | ¹H-NMR (400 MHz, CHLOROFORM-D) δ 7.71 (s, 1H), 7.17 (d, |
| | dimethylbenzyl)oxy)- | J = 8.3 Hz, 1H), 7.00-6.96 (m, 2H), 6.62 (s, 1H), 4.52 (s, 2H), |
| | 2-methylpropan-2-yl)- | 3.50 (s, 2H), 2.88 (t, J = 6.4 Hz, 2H), 2.74 (t, J = 6.3 Hz, 2H), |
| | 5,6,7,8- | 2.29 (d, J = 4.6 Hz, 6H), 1.90-1.77 (m, 4H), 1.47 (s, 6H); ESI MS |
| | tetrahydroquinoline-3- | (m/z) = 401.40 (MH)⁺ |
| | carboxamide | |
| 263 | N-(2,4-dimethyl-1- | ¹H-NMR (400 MHz, CHLOROFORM-D) δ 9.35 (s, 1H), 8.48 (d, |
| | phenylpentan-2-yl)-2- | J = 1.7 Hz, 1H), 8.09 (d, J = 2.0 Hz, 1H), 7.23-7.16 (m, 5H), 6.24 |
| | methyl-1H- | (s, 1H), 5.59 (s, 1H), 3.45 (d, J = 13.2 Hz, 1H), 2.96 (d, J = 13.2 |
| | pyrrolo[2,3-b]pyridine- | Hz, 1H), 2.51 (d, J = 0.7 Hz, 3H), 2.15 (q, J = 7.1 Hz, 1H), 1.92- |
| | 5-carboxamide | 1.86 (m, 1H), 1.61-1.57 (m, 1H), 1.39 (d, J = 6.6 Hz, 3H), 1.00 |
| | | (d, J = 6.6 Hz, 6H); ESI MS (m/z) = 350.05 (MH)⁺. |
| 264 | N-(2,4-dimethyl-1- | ¹H-NMR (400 MHz, CHLOROFORM-D) δ 8.51 (d, J = 2.2 Hz, |
| | phenylpentan-2-yl)- | 1H), 8.10 (d, J = 2.0 Hz, 1H), 7.25-7.15 (m, 5H), 6.27 (d, J = 1.0 |
| | 1,2-dimethyl-1H- | Hz, 1H), 5.59 (s, 1H), 3.80 (d, J = 5.4 Hz, 3H), 3.43 (d, J = 13.2 |
| | pyrrolo[2,3-b]pyridine- | Hz, 1H), 2.98 (d, J = 13.2 Hz, 1H), 2.47 (d, J = 1.0 Hz, 3H), 2.12 |
| | 5-carboxamide | (q, J = 7.1 Hz, 1H), 1.92-1.85 (m, 1H), 1.61-1.57 (m, 1H), 1.38 |
| | | (s, 3H), 1.00 (d, J = 6.6 Hz, 6H); ESI MS (m/z) = 364.45 (MH)⁺ |
| 265 | N-(1- | ¹H-NMR (400 MHz, CHLOROFORM-D) δ 8.48 (d, J = 2.2 Hz, |
| | benzylcyclopropyl)- | 1H), 8.08 (d, J = 2.0 Hz, 1H), 7.30-7.27 (m, 2H), 7.24-7.20 (m, |
| | 1,2-dimethyl-1H- | 3H), 6.24 (d, J = 1.2 Hz, 2H), 3.78 (d, J = 7.1 Hz, 3H), 3.03 (s, |
| | pyrrolo[2,3-b]pyridine- | 2H), 2.45 (d, J = 0.7 Hz, 3H), 0.98-0.91 (m, 4H); ESI MS (m/z) = |
| | 5-carboxamide | 320.00 (MH)⁺. |
| 266 | 2-chloro-N-(1-((2- | ¹H-NMR (400 MHz, CHLOROFORM-D) δ 7.73 (s, 1H), 7.47- |
| | chlorobenzyl)oxy)-2- | 7.45 (m, 1H), 7.34 (m, 1H), 7.25 (d, J = 1.7 Hz, 1H), 7.24 (q, J = |
| | methylpropan-2-yl)- | 1.3 Hz, 1H), 6.68 (s, 1H), 4.65 (s, 2H), 3.61 (s, 2H), 2.88 (t, J = |
| | 5,6,7,8- | 6.5 Hz, 2H), 2.75 (t, J = 6.2 Hz, 2H), 1.90-1.84 (m, 2H), 1.82- |
| | tetrahydroquinoline-3- | 1.77 (m, 2H), 1.51 (s, 6H); ESI MS (m/z) = 409.15 (MH)⁺ |
| | carboxamide | |
| 267 | 2-chloro-N-(1-((2- | ¹H-NMR (400 MHz, CHLOROFORM-D) δ 7.73 (s, 1H), 7.39 |
| | fluorobenzyl)oxy)-2- | (td, J = 7.5, 1.7 Hz, 1H), 7.30-7.27 (m, 1H), 7.11 (td, J = 7.5, 1.1 |
| | methylpropan-2-yl)- | Hz, 1H), 7.05-7.00 (m, 1H), 6.67 (s, 1H), 4.63 (s, 2H), 3.56 (s, |
| | 5,6,7,8- | 2H), 2.88 (t, J = 6.5 Hz, 2H), 2.74 (t, J = 6.2 Hz, 2H), 1.90-1.76 |
| | tetrahydroquinoline-3- | (m, 4H), 1.49 (s, 6H); ESI MS (m/z) = 391.40 (MH)⁺ |
| | carboxamide | |
| 268 | 2-chloro-N-(1-((2- | ¹H-NMR (400 MHz, CHLOROFORM-D) δ 7.73 (s, 1H), 7.67- |
| | cyanobenzyl)oxy)-2- | 7.64 (m, 1H), 7.60-7.52 (m, 2H), 7.39 (td, J = 7.5, 1.6 Hz, 1H), |
| | methylpropan-2-yl)- | 6.65 (s, 1H), 4.74 (s, 2H), 3.66 (s, 2H), 2.88 (t, J = 6.5 Hz, 2H), |
| | 5,6,7,8- | 2.75 (t, J = 6.2 Hz, 2H), 1.90-1.76 (m, 4H), 1.51 (s, 6H); ESI MS |
| | tetrahydroquinoline-3- | (m/z) = 398.50 (MH)⁺ |
| | carboxamide | |
| 269 | 2-chloro-N-(1-(4- | ¹H-NMR (400 MHz, CHLOROFORM-D) δ 7.76 (s, 1H), 7.18- |
| | fluorophenyl)-2,4- | 7.14 (m, 2H), 6.95 (t, J = 8.6 Hz, 2H), 6.14 (s, 1H), 4.94-4.93 (m, |
| | dimethylpent-4-en-2- | 1H), 4.76 (d, J = 3.4 Hz, 1H), 3.48 (d, J = 14.2 Hz, 1H), 2.99-2.95 |
| | yl)-5,6,7,8- | (m, 2H), 2.90-2.87 (m, 2H), 2.77 (t, J = 6.4 Hz, 2H), 2.34 (d, J = |
| | tetrahydroquinoline-3- | 12.5 Hz, 1H), 1.85 (dd, J = 29.7, 12.3 Hz, 7H), 1.33 (s, 3H); ESI |
| | carboxamide | MS (m/z) = 401.00 (MH)⁺ |
| 270 | 2-chloro-N-(1-(2- | ¹H-NMR (400 MHz, CHLOROFORM-D) δ 7.67 (s, 1H), 7.08- |
| | fluorophenoxy)-2,4- | 6.98 (m, 3H), 6.93-6.87 (m, 1H), 6.60 (s, 1H), 4.97 (dd, J = 2.1, |
| | dimethylpent-4-en-2- | 1.6 Hz, 1H), 4.84 (d, J = 1.2 Hz, 1H), 4.33 (d, J = 9.0 Hz, 1H), |
| | yl)-5,6,7,8- | 4.18 (d, J = 9.0 Hz, 1H), 2.88 (m, 3H), 2.75 (t, J = 6.1 Hz, 2H), |
| | tetrahydroquinoline-3- | 2.63-2.59 (m, 1H), 1.91-1.77 (m, 7H), 1.58 (s, 3H); ESI MS |
| | carboxamide | (m/z) = 417.35 (MH)⁺ |
| 271 | N-(1-((2- | ¹H-NMR (400 MHz, CHLOROFORM-D) δ 8.27 (s, 2H), 7.52- |
| | chlorobenzyl)oxy)-2- | 7.50 (m, 1H), 7.34 (dd, J = 7.6, 1.7 Hz, 1H), 7.22 (m, 2H), 4.69 |
| | methylpropan-2-yl)-2- | (s, 2H), 3.93 (s, 3H), 3.66 (s, 2H), 2.94-2.86 (m, 4H), 2.16-2.08 |
| | methoxy-6,7-dihydro- | (m, 2H), 1.51 (s, 6H); ESI MS (m/z) = 389.20 (MH)⁺ |
| | 5H- | |
| | cyclopenta[b]pyridine- | |
| | 3-carboxamide | |
| 272 | N-(1-((2- | ¹H-NMR (400 MHz, CHLOROFORM-D) δ 8.26 (s, 2H), 7.44 |
| | fluorobenzyl)oxy)-2- | (m, 1H), 7.29-7.27 (m, 1H), 7.11 (m, 1H), 7.05-7.01 (m, 1H), |
| | methylpropan-2-yl)-2- | 4.66 (s, 2H), 3.94 (s, 3H), 3.60 (s, 2H), 2.95-2.86 (m, 4H), 2.16- |
| | methoxy-6,7-dihydro- | 2.08 (m, 2H), 1.48 (s, 6H); ESI MS (m/z) = 373.55 (MH)⁺ |
| | 5H- | |
| | cyclopenta[b]pyridine- | |
| | 3-carboxamide | |
| 273 | N-(1-(4-fluorophenyl)- | ¹H-NMR (400 MHz, CHLOROFORM-D) δ 8.31 (s, 1H), 7.59 (s, |
| | 2,4-dimethylpent-4-en- | 1H), 7.11-7.08 (m, 2H), 6.93-6.88 (m, 2H), 4.90 (m, 1H), 4.73 |
| | 2-yl)-2-methoxy-6,7- | (m, 1H), 3.91 (s, 3H), 3.47 (d, J = 13.4 Hz, 1H), 3.00-2.89 (m, |
| | dihydro-5H- | 6H), 2.31-2.27 (m, 1H), 2.14 (m, 2H), 1.78 (s, 3H), 1.27 (s, 3H); |
| | cyclopenta[b]pyridine- | ESI MS (m/z) = 383.20 (MH)⁺ |
| | 3-carboxamide | |
| 274 | N-(1-(2- | ¹H-NMR (400 MHz, CHLOROFORM-D) δ 8.24 (m, 2H), 7.07- |
| | fluorophenoxy)-2,4- | 6.98 (m, 3H), 6.90-6.84 (m, 1H), 4.94 (q, J = 1.2 Hz, 1H), 4.83 |
| | dimethylpent-4-en-2- | (d, J = 1.5 Hz, 1H), 4.33 (d, J = 8.8 Hz, 1H), 4.18 (d, J = 9.0 Hz, |
| | yl)-2-methoxy-6,7- | 1H), 4.02 (s, 3H), 2.95-2.85 (m, 5H), 2.65 (d, J = 13.7 Hz, 1H), |
| | dihydro-5H- | 2.16-2.09 (m, 2H), 1.82 (s, 3H), 1.61 (s, 3H); ESI MS (m/z) = |
| | cyclopenta[b]pyridine- | 399.35 (MH)⁺ |
| | 3-carboxamide | |
| 275 | N-(1-((2-chloro-5- | ¹H-NMR (400 MHz, CHLOROFORM-D) δ 8.26 (s, 1H), 8.20 (s, |
| | fluorobenzyl)oxy)-2- | 1H), 7.29-7.27 (m, 1H), 7.25-7.24 (m, 1H), 6.93-6.88 (m, 1H), |
| | methylpropan-2-yl)-2- | 4.63 (s, 2H), 3.96 (s, 3H), 3.71 (s, 2H), 2.90 (m, 4H), 2.16-2.09 |
| | methoxy-6,7-dihydro- | (m, 2H), 1.49 (s, 6H); ESI MS (m/z) = 407.00 (M)⁺ |
| | 5H- | |
| | cyclopenta[b]pyridine- | |
| | 3-carboxamide | |
| 276 | N-(1-(4-fluorophenyl)- | ¹H-NMR (400 MHz, CHLOROFORM-D) δ 8.52 (d, J = 2.1 Hz, |
| | 2-methylpropan-2-yl)- | 1H), 8.09 (d, J = 2.1 Hz, 1H), 7.13 (ddd, J = 11.8, 5.3, 3.1 Hz, |
| | 1,2-dimethyl-1H- | 2H), 6.95-6.90 (m, 2H), 6.26 (d, J = 0.9 Hz, 1H), 5.66 (s, 1H), |
| | pyrrolo[2,3-b]pyridine- | 3.80 (s, 3H), 3.18 (s, 2H), 2.47 (d, J = 0.9 Hz, 3H), 1.46 (s, 6H); |
| | 5-carboxamide | ESI MS (m/z) = 340.25 (MH)⁺ |
| 277 | 1,2-dimethyl-N-(2- | ¹H-NMR (400 MHz, CHLOROFORM-D) δ 8.45 (d, J = 2.1 Hz, |
| | phenylpropyl)-1H- | 1H), 8.10 (d, J = 1.8 Hz, 1H), 7.37-7.33 (m, 2H), 7.30-7.27 (m, |
| | pyrrolo[2,3-b]pyridine- | 2H), 7.25-7.22 (m, 1H), 6.24 (d, J = 0.9 Hz, 1H), 5.96 (s, 1H), |
| | 5-carboxamide | 3.94-3.86 (m, 1H), 3.77 (s, 3H), 3.46-3.39 (m, 1H), 3.15-3.06 (m, |
| | | 1H), 2.45 (s, 3H), 1.36 (d, J = 7.0 Hz, 3H); ESI MS (m/z) = 308.5 |
| | | (MH)⁺ |
| 278 | 1,2-dimethyl-N-((1- | ¹H-NMR (400 MHz, CHLOROFORM-D) δ 8.52 (d, J = 2.1 Hz, |
| | phenylcyclopropyl) | 1H), 8.13 (d, J = 1.8 Hz, 1H), 7.39-7.31 (m, 4H), 7.25-7.21 (m, |
| | methyl)-1H-pyrrolo | 1H), 6.25 (d, J = 1.2 Hz, 1H), 6.13 (s, 1H), 3.78 (s, 3H), 3.67 (d, |
| | [2,3-b]pyridine-5- | J = 5.5 Hz, 2H), 2.46 (d, J = 0.6 Hz, 3H), 1.02-0.92 (m, 4H); ESI |
| | carboxamide | MS (m/z) 320.5 (MH)⁺ |
| 279 | 2-chloro-N-methoxy- | ¹H-NMR (400 MHz, CHLOROFORM-D) δ 7.29 (broad, 4H), |
| | N-(1-phenylpropan-2- | 7.07 (1H), 5.53 (s, 1H), 4.08 (s, 3H), 3.65 (d, J = 7.1 Hz, 1H), |
| | yl)-6,7-dihydro-5H- | 3.39 (s, 1H), 2.98-2.90 (m, 2H), 2.72-2.63 (m, 2H), 2.51-2.47 (m, |
| | cyclopenta[b]pyridine- | 1H), 2.17-2.04 (m, 2H), 1.52 (d, J = 6.4 Hz, 3H); ESI MS (m/z) = |
| | 3-carboxamide | 345.30 (MH)⁺ |
| 280 | 4-methyl-N-(1- | ¹H-NMR (400 MHz, CHLOROFORM-D) δ 8.24 (d, J = 2.0 Hz, |
| | phenylcyclopropyl)- | 1H), 7.39 (d, J = 2.0 Hz, 1H), 7.28 (m, 2H), 7.27 (m, 2H), 7.17 |
| | 3,4-dihydro-2H- | (m, 1H), 6.86 (s, 1H), 4.22 (t, J = 4.5 Hz, 2H), 3.52 (t, J = 4.5 Hz, |
| | pyrido[3,2-b] | 2H), 3.25 (s, 3H), 1.38-1.31 (m, 4H); ESI MS (m/z) = 309.95 |
| | [1,4]oxazine-7- | (M)⁺ |
| | carboxamide | |
| 281 | N-(1-(4- | ¹H-NMR (400 MHz, CHLOROFORM-D) δ 8.21 (d, J = 2.0 Hz, |
| | fluorophenyl) | 1H), 7.36 (d, J = 2.0 Hz, 1H), 7.32-7.27 (m, 2H), 6.98-6.92 (m, |
| | cyclopropyl)- | 2H), 6.89 (s, 1H), 4.22 (t, J = 4.5 Hz, 2H), 3.52 (t, J = 4.5 Hz, |
| | 4-methyl-3,4- | 2H), 3.22 (s, 3H), 1.34-1.25 (m, 4H); ESI MS (m/z) = 327.90 |
| | dihydro-2H- | (M)⁺ |
| | pyrido[3,2-b] | |
| | [1,4]oxazine-7- | |
| | carboxamide | |
| 282 | 4-methyl-N-((1- | ¹H-NMR (400 MHz, CHLOROFORM-D) δ 7.99 (d, J = 2.0 Hz, |
| | phenylcyclopropyl) | 1H), 7.28-7.27 (m, 1H), 7.25-7.20 (m, 4H), 7.17-7.13 (m, 1H), |
| | methyl)-3,4-dihydro- | 5.92 (s, 1H), 4.16-4.14 (m, 2H), 3.53 (d, J = 5.6 Hz, 2H), 3.45- |
| | 2H-pyrido[3,2-b] | 3.42 (m, 2H), 3.12 (s, 3H), 0.91-0.88 (m, 2H), 0.85-0.82 (m, 2H); |
| | [1,4]oxazine-7- | ESI MS (m/z) = 324.10 (MH)⁺ |
| | carboxamide | |
| 283 | N-(1- | ¹H-NMR (400 MHz, CHLOROFORM-D) δ 7.98 (d, J = 2.2 Hz, |
| | benzylcyclopropyl)-4- | 1H), 7.28 (m, 2H), 7.26-7.21 (m, 2H), 7.18-7.16 (m, 2H), 6.07 (s, |
| | methyl-3,4-dihydro- | 1H), 4.21 (t, 2H), 3.50 (t, 2H), 3.19 (s, 3H), 2.97 (s, 2H), 0.93- |
| | 2H-pyrido[3,2-b] | 0.90 (m, 2H), 0.88-0.85 (m, 2H); ESI MS (m/z) = 324.15 (MH)⁺. |
| | [1,4]oxazine-7- | |
| | carboxamide | |
| 284 | 2-chloro-N-(2-methyl- | ¹H-NMR (400 MHz, CHLOROFORM-D) δ 8.08 (m, 1H), 7.68 |
| | 1-(pyridin-2- | (s, 1H), 7.59 (m, 1H), 7.23 (s, 1H), 6.89 (m, 1H), 6.78 (d, J = 8.3 |
| | yloxy)propan-2-yl)- | Hz, 1H), 4.41 (s, 2H), 2.87 (t, J = 6.5 Hz, 2H), 2.75 (d, J = 6.0 |
| | 5,6,7,8- | Hz, 2H), 1.92-1.75 (m, 4H), 1.58 (s, 6H); ESI MS (m/z) = 360.15 |
| | tetrahydroquinoline-3- | (MH)⁺ |
| | carboxamide | |
| 285 | 2-chloro-N-(1-((2- | ¹H-NMR (400 MHz, CHLOROFORM-D) δ 8.20 (d, 1H), 7.73 (s, |
| | chloropyridin-4- | 1H), 6.88 (d, J = 2.2 Hz, 1H), 6.79 (dd, J = 5.9, 2.4 Hz, 1H), 6.49 |
| | yl)oxy)-2- | (s, 1H), 4.29 (s, 2H), 2.89 (t, J = 6.5 Hz, 2H), 2.75 (t, J = 6.2 Hz, |
| | methylpropan-2-yl)- | 2H), 1.92-1.76 (m, 4H), 1.56 (s, 6H); ESI MS (m/z) = 394.15 |
| | 5,6,7,8- | (MH)⁺ |
| | tetrahydroquinoline-3- | |
| | carboxamide | |
| 286 | 2-chloro-N-(4,4,4- | ¹H-NMR (400 MHz, CHLOROFORM-D) δ 7.35-7.27 (m, 4H), |
| | trifluoro-2-methyl-1- | 7.15-7.13 (m, 3H), 4.83 (s, 2H), 3.33 (d, J = 13.4 Hz, 2H), 3.17- |
| | phenylbutan-2-yl)-6,7- | 3.05 (m, 2H), 2.87 (d, J = 13.4 Hz, 2H), 2.50-2.38 (m, 2H), 1.32 |
| | dihydro-5H- | (s, 3H); ESI MS (m/z) = 397.05 (MH)⁺ |
| | cyclopenta[b]pyridine- | |
| | 3-carboxamide | |
| 287 | 2-chloro-N-(1-((2- | ¹H-NMR (400 MHz, CHLOROFORM-D) δ 7.75 (s, 1H), 7.28 |
| | chloro-5- | (dd, J = 8.7, 5.0 Hz, 1H), 7.20 (dd, J = 9.2, 3.1 Hz, 1H), 6.94-6.90 |
| | fluorobenzyl)oxy)-2- | (m, 1H), 6.62 (s, 1H), 4.60 (s, 2H), 3.68 (s, 2H), 2.89 (t, J = 6.4 |
| | methylpropan-2-yl)- | Hz, 2H), 2.75 (t, J = 6.2 Hz, 2H), 1.90-1.77 (m, 4H), 1.51 (s, 6H); |
| | 5,6,7,8- | ESI MS (m/z) = 424.95 (M)⁺ |
| | tetrahydroquinoline-3- | |
| | carboxamide | |
| 288 | 2-methoxy-N-(2- | ¹H-NMR (400 MHz, CHLOROFORM-D) δ 8.27 (s, 1H), 8.17 |
| | methyl-1-(pyridin-2- | (dt, J = 5.2, 0.9 Hz, 1H), 8.12 (s, 1H), 7.64-7.60 (m, 1H), 6.90 |
| | yloxy)propan-2-yl)- | (m, 1H), 6.82 (d, J = 8.3 Hz, 1H), 4.43 (s, 2H), 3.93 (s, 3H), 2.78 |
| | 5,6,7,8- | (t, J = 6.4 Hz, 2H), 2.70 (t, J = 6.1 Hz, 2H), 1.87-1.74 (m, 4H), |
| | tetrahydroquinoline-3- | 1.58 (s, 6H); ESI MS (m/z) = 356.25 (MH)⁺ |
| | carboxamide | |
| 289 | 2-methoxy-N-(2- | ¹H-NMR (400 MHz, CHLOROFORM-D) δ 8.19 (s, 1H), 7.67 (s, |
| | methyl-1- | 1H), 7.24-7.20 (m, 3H), 7.18-7.15 (m, 2H), 3.91 (s, 3H), 3.14 (s, |
| | phenylpropan-2-yl)- | 2H), 2.80 (t, J = 6.4 Hz, 2H), 2.74 (t, J = 6.3 Hz, 2H), 1.89-1.77 |
| | 5,6,7,8- | (m, 5H), 1.43 (s, 6H); ESI MS (m/z) m/z = 339.60 (MH)⁺ |
| | tetrahydroquinoline-3- | |
| | carboxamide | |
| 290 | N-(2,4-dimethyl-1- | ¹H-NMR (400 MHz, CHLOROFORM-D) δ 8.18 (s, 1H), 7.61 (s, |
| | phenylpentan-2-yl)-2- | 1H), 7.24-7.15 (m, 3H), 7.14-7.12 (m, 2H), 3.89 (s, 3H), 3.32 (d, |
| | methoxy-5,6,7,8- | J = 13.2 Hz, 1H), 3.02 (d, J = 13.2 Hz, 1H), 2.77 (m, 4H), 1.96 |
| | tetrahydroquinoline-3- | (m, 1H), 1.89-1.77 (m, 5H), 1.54 (m, 1H), 1.37 (s, 3H), 0.95 (dd, |
| | carboxamide | J = 7.5, 6.7 Hz, 6H); ESI MS (m/z) m/z = 381.25 (MH)⁺ |
| 291 | 2-methoxy-N-(1- | ¹H-NMR (400 MHz, CHLOROFORM-D) δ 8.50 (s, 1H), 8.19 (s, |
| | phenylcyclopropyl)- | 1H), 7.30-7.27 (m, 4H), 7.19-7.14 (m, 1H), 4.07 (s, 3H), 2.82 (t, |
| | 5,6,7,8- | J = 6.3 Hz, 2H), 2.71 (t, J = 6.1 Hz, 2H), 1.88-1.83 (m, 2H), 1.81- |
| | tetrahydroquinoline-3- | 1.75 (m, 2H), 1.37 (s, 4H); ESI MS (m/z) 323.50 (MH)⁺ |
| | carboxamide | |
| 292 | 2-methoxy-N-((1- | ¹H-NMR (400 MHz, CHLOROFORM-D) δ 8.14 (s, 1H), 8.04 (s, |
| | phenylcyclopropyl)met | 1H), 7.40-7.37 (m, 2H), 7.35-7.31 (m, 2H), 7.26-7.22 (m, 1H), |
| | hyl)-5,6,7,8- | 3.86 (s, 3H), 3.67-3.63 (m, 2H), 2.77 (t, J = 6.4 Hz, 2H), 2.71 (t, |
| | tetrahydroquinoline-3- | J = 6.2 Hz, 2H), 1.87-1.74 (m, 4H), 0.96-0.89 (m, 4H); ESI MS |
| | carboxamide | (m/z) 337.15 (MH)⁺ |
| 293 | 2-methoxy-N-(4- | ¹H-NMR (400 MHz, CHLOROFORM-D) δ 8.19 (s, 1H), 7.79- |
| | phenylbutan-2-yl)- | 7.76 (m, 1H), 7.29-7.27 (m, 1H), 7.25 (d, J = 1.8 Hz, 1H), 7.20- |
| | 5,6,7,8- | 7.15 (m, 3H), 4.28-4.21 (m, 1H), 4.05 (s, 3H), 2.82 (t, J = 6.4 Hz, |
| | tetrahydroquinoline-3- | 2H), 2.75-2.68 (m, 4H), 1.89-1.84 (m, 4H), 1.82-1.76 (m, 2H), |
| | carboxamide | 1.28-1.26 (m, 3H); ESI MS (m/z) = 339.15 (MH)⁺ |
| 294 | N-(1-(4-fluorophenyl)- | ¹H-NMR (400 MHz, CHLOROFORM-D) δ 8.19 (s, 1H), 7.61 (s, |
| | 2-methylpropan-2-yl)- | 1H), 7.11-7.08 (m, 2H), 6.95-6.89 (m, 2H), 3.93 (s, 3H), 3.13 (s, |
| | 2-methoxy-5,6,7,8- | 2H), 2.82 (t, J = 6.4 Hz, 2H), 2.74 (t, J = 6.1 Hz, 2H), 1.90-1.79 |
| | tetrahydroquinoline-3- | (m, 4H), 1.40-1.38 (m, 6H); ESI MS (m/z) = 357.55 (MH)⁺ |
| | carboxamide | |
| 295 | 2-chloro-N-(4- | ¹H-NMR(400 MHz, CHLOROFORM-D) δ 7.80 (s, 1H), 7.30- |
| | phenylbutan-2-yl)- | 7.27 (m, 2H), 7.21-7.16 (m, 3H), 6.34 (d, J = 7.9 Hz, 1H), 4.27 |
| | 5,6,7,8- | (m, 1H), 2.91 (t, J = 6.4 Hz, 2H), 2.79-2.68 (m, 4H), 1.91-1.79 |
| | tetrahydroquinoline-3- | (m, 6H), 1.34-1.29 (m, 3H); ESI MS (m/z) = 343.05 (MH)⁺ |
| | carboxamide | |
| 296 | 2-chloro-N-(1-(4- | ¹H-NMR (400 MHz, CHLOROFORM-D) δ 7.75 (s, 1H), 7.15 |
| | fluorophenyl)-2- | (td, J = 6.0, 2.4 Hz, 2H), 6.98-6.93 (m, 2H), 6.07 (s, 1H), 3.15 (s, |
| | methylpropan-2-yl)- | 2H), 2.89 (t, J = 6.4 Hz, 2H), 2.77 (t, J = 6.3 Hz, 2H), 1.91-1.78 |
| | 5,6,7,8- | (m, 4H), 1.41 (s, 6H); ESI MS (m/z) = 361.15 (MH)⁺ |
| | tetrahydroquinoline-3- | |
| | carboxamide | |
| 297 | N-(1-(4-fluorophenyl)- | ¹H-NMR (400 MHz, CHLOROFORM-D) δ 8.75 (s, 1H), 7.97 (s, |
| | 2,4-dimethylpent-4-en- | 1H), 7.11 (dd, J = 8.6, 5.6 Hz, 2H), 6.92 (t, J = 8.7 Hz, 2H), 6.22 |
| | 2-yl)-5,6,7,8- | (s, 1H), 4.93 (s, 1H), 4.74 (s, 1H), 3.51 (d, J = 13.7 Hz, 1H), 3.09- |
| | tetrahydroquinoline-3- | 3.04 (m, 3H), 2.94 (d, J = 13.7 Hz, 1H), 2.87 (t, J = 6.1 Hz, 2H), |
| | carboxamide | 2.29 (d, J = 13.7 Hz, 1H), 1.96-1.83 (m, 4H), 1.82 (s, 3H), 1.34 |
| | | (s, 3H); ESI MS (m/z) = 367.15 (MH)⁺ |
| 298 | N-(1-(2- | ¹H-NMR (400 MHz, DMSO-d6) δ 8.62 (d, J = 2.1 Hz, 1H), 7.91 |
| | fluorophenoxy)-2,4- | (s, 1H), 7.77 (d, J = 2.1 Hz, 1H), 7.19-7.07 (m, 3H), 6.93-6.88 |
| | dimethylpent-4-en-2- | (m, 1H), 4.86 (q, J = 1.3 Hz, 1H), 4.71 (d, J = 1.8 Hz, 1H), 4.43 |
| | yl)-5,6,7,8- | (d, J = 9.2 Hz, 1H), 4.12 (d, J = 9.2 Hz, 1H), 2.89 (d, J = 13.4 Hz, |
| | tetrahydroquinoline-3- | 1H), 2.82 (t, J = 6.4 Hz, 2H), 2.75 (t, J = 6.1 Hz, 2H), 2.43 (d, J = |
| | carboxamide | 13.1 Hz, 1H), 1.84-1.78 (m, 2H), 1.76-1.69 (m, 5H), 1.42 (s, |
| | | 3H); ESI MS (m/z) = 382.1 (MH)⁺ |
| 299 | N-(1-(4- | ¹H-NMR (400 MHz, CHLOROFORM-D) δ 8.50 (s, 1H), 8.15 (s, |
| | fluorophenyl) | 1H), 7.32-7.27 (m, 2H), 6.98-6.93 (m, 2H), 4.05 (s, 3H), 2.80 (t, |
| | cyclopropyl)- | J = 6.4 Hz, 2H), 2.70 (t, J = 6.2 Hz, 2H), 1.89-1.75 (m, 4H), 1.35- |
| | 2-methoxy- | 1.26 (m, 4H); ESI MS (m/z) = 341.25 (MH)⁺ |
| | 5,6,7,8- | |
| | tetrahydroquinoline-3- | |
| | carboxamide | |
| 300 | N-(4,4,4-trifluoro-2- | ESI MS (m/z) 362.50 (MH)⁺ |
| | methyl-1-phenylbutan- | |
| | 2-yl)-6,7-dihydro-5H- | |
| | cyclopenta[b]pyridine- | |
| | 3-carboxamide | |
| 301 | 2-chloro-N-(4,4,4- | ESI MS (m/z) 410.40 (MH)⁺ |
| | trifluoro-2-methyl-1- | |
| | phenylbutan-2-yl)- | |
| | 5,6,7,8- | |
| | tetrahydroquinoline-3- | |
| | carboxamide | |
| 302 | 2-methoxy-N-(4,4,4- | ESI MS (m/z) 406.50 (MH)⁺ |
| | trifluoro-2-methyl-1- | |
| | phenylbutan-2-yl)- | |
| | 5,6,7,8- | |
| | tetrahydroquinoline-3- | |
| | carboxamide | |
| 303 | 1-methyl-N-(4,4,4- | ESI MS (m/z) 475.6 (MH)⁺ |
| | trifluoro-2-methyl-1- | |
| | phenylbutan-2-yl)-1H- | |
| | pyrrolo[2,3-b]pyridine- | |
| | 5-carboxamide | |
| 304 | 3-chloro-1-methyl-N- | ESI MS (m/z) 409.65 (MH)⁺ |
| | (4,4,4-trifluoro-2- | |
| | methyl-1-phenylbutan- | |
| | 2-yl)-1H-pyrrolo[2,3-b] | |
| | pyridine-5- | |
| | carboxamide | |
| 305 | 3-chloro-6-methoxy-1- | ESI MS (m/z) 439.55 (MH)⁺ |
| | methyl-N-(4,4,4- | |
| | trifluoro-2-methyl-1- | |
| | phenylbutan-2-yl)-1H- | |
| | pyrrolo[2,3-b]pyridine- | |
| | 5-carboxamide | |
| 306 | 6,6-difluoro-N-(4,4,4- | ESI MS (m/z) 412.7 (MH)⁺ |
| | trifluoro-2-methyl-1- | |
| | phenylbutan-2-yl)- | |
| | 5,6,7,8- | |
| | tetrahydroquinoline-3- | |
| | carboxamide | |
| 307 | N-(2,4-dimethyl-1- | ESI MS (m/z) 387.0 (MH)⁺ |
| | phenylpentan-2-yl)- | |
| | 6,6-difluoro-5,6,7,8- | |
| | tetrahydroquinoline-3- | |
| | carboxamide | |
| 308 | 4-chloro-N-(4,4,4- | ESI MS (m/z) 410.5 (MH)⁺ |
| | trifluoro-2-methyl-1- | |
| | phenylbutan-2-yl)- | |
| | 5,6,7,8- | |
| | tetrahydroquinoline-3- | |
| | carboxamide | |
| 309 | 4-chloro-N-(2,4- | ESI MS (m/z) 470.9 (MH)⁺ |
| | dimethyl-1- | |
| | phenylpentan-2-yl)- | |
| | 6,7-dihydro-5H- | |
| | cyclopenta[b]pyridine- | |
| | 3-carboxamide | |
| 310 | 4-chloro-N-(2,4- | ESI MS (m/z) 468.5 (MH)⁺ |
| | dimethyl-1- | |
| | phenylpent-4-en-2-yl)- | |
| | 6,7-dihydro-5H- | |
| | cyclopenta[b]pyridine- | |
| | 3-carboxamide | |
| 311 | 4-chloro-N-(2,4- | ESI MS (m/z) 384.7 (MH)⁺ |
| | dimethyl-1- | |
| | phenylpentan-2-yl)- | |
| | 5,6,7,8- | |
| | tetrahydroquinoline-3- | |
| | carboxamide | |
| 312 | N-(2,4-dimethyl-1- | ESI MS (m/z) 381.0 (MH)⁺ |
| | phenylpentan-2-yl)-4- | |
| | methoxy-5,6,7,8- | |
| | tetrahydroquinoline-3- | |
| | carboxamide | |
| 313 | N-(2,4-dimethyl-1- | ESI MS (m/z) 366.5 (MH)⁺ |
| | phenylpentan-2-yl)-4- | |
| | methoxy-6,7-dihydro- | |
| | 5H- | |
| | cyclopenta[b]pyridine- | |
| | 3-carboxamide | |
| 314 | N-(2,4-dimethyl-1- | ESI MS (m/z) 364.7 (MH)⁺ |
| | phenylpent-4-en-2-yl)- | |
| | 4-methoxy-6,7- | |
| | dihydro-5H- | |
| | cyclopenta[b]pyridine- | |
| | 3-carboxamide | |
| 315 | 4-chloro-N-(1-(2- | ESI MS (m/z) 346.6 (MH)⁺ |
| | fluorophenyl)-2- | |
| | methylpropan-2-yl)- | |
| | 6,7-dihydro-5H- | |
| | cyclopenta[b]pyridine- | |
| | 3-carboxamide | |
| 316 | 4-chloro-N-(1-(4- | ESI MS (m/z) 346.5 (MH)⁺ |
| | fluorophenyl)-2- | |
| | methylpropan-2-yl)- | |
| | 6,7-dihydro-5H- | |
| | cyclopenta[b]pyridine- | |
| | 3-carboxamide | |
| 317 | N-(1-(2-fluorophenyl)- | ESI MS (m/z) 342.5 (MH)⁺ |
| | 2-methylpropan-2-yl)- | |
| | 4-methoxy-6,7- | |
| | dihydro-5H- | |
| | cyclopenta[b]pyridine- | |
| | 3-carboxamide | |
| 318 | N-(1-(4-fluorophenyl)- | ESI MS (m/z) 342.6 (MH)⁺ |
| | 2-methylpropan-2-yl)- | |
| | 4-methoxy-6,7- | |
| | dihydro-5H- | |
| | cyclopenta[b]pyridine- | |
| | 3-carboxamide | |

### BIOLOGY EXAMPLES

As described herein the compounds of the general Formula (I) show fungicidal activities which are exerted with respect to numerous phytopathogenic fungi which attack on important agricultural crops. The compounds of the present invention were assessed for their activity as described in the following tests:

### Example 1: Pyricularia Oryzae (Rice Blast

The compounds were dissolved in 0.3% dimethyl sulfoxide and then added to potato dextrose agar medium just prior to dispensing it into petri dishes. 5 mL medium, with the compound in the desired test concentration, was dispensed into 60 mm sterile petri-plates. After solidification, each plate was seeded with a 5 mm size mycelial disc taken from the periphery of an actively growing virulent culture plate. The plates were incubated in growth chambers at 25° C. temperature and 60% relative humidity for seven days and the radial growth was measured and compared to the one of the untreated control. Compounds 1 2 3 7 8 11 12 13 20 21 23 24 25 29 30 34 36 38 39 40 42 43 44 46 47 48 50 51 53 56 58 59 63 64 65 69 71 79 81 83 85 98 99 100 108 109 119 124 131 133 141 143 161 176 180 185 189 214 219 220 239 254 at 300 ppm gave more than or equal to 70% control in these tests when compared to the untreated check which showed extensive pathogen growth.

### Example 2: Botrytis cinerea (Gray Mold

The compounds were dissolved in 0.3% dimethyl sulfoxide and then added to potato dextrose agar medium just prior to dispensing it into petri dishes. 5 mL medium, with the compound in the desired concentration, was dispensed into 60 mm sterile petri-plates. After solidification, each plate was seeded with a 5 mm size mycelial disc taken from the periphery of an actively growing virulent culture plate. The plates were incubated in growth chambers at 22° C. temperature and 90% relative humidity for seven days and the radial growth was measured and compared to the one of the untreated control. Compounds 40 50 55 63 67 72 73 75 78 82 89 98 100 101 105 106 109 118 144 148 149 159 160 187 189 190 194 252 254 at 300 ppm gave more than or equal to 70% control in these tests when compared to the untreated check which showed extensive pathogen growth.

### Example 3: Alternaria solani (Early Blight of Tomato/Potato

The compounds were dissolved in 0.3% dimethyl sulfoxide and then added to potato dextrose agar medium just prior to dispensing it into petri dishes. 5 mL medium, with the compound in the desired concentration, was dispensed into 60 mm sterile petri-plates. After solidification, each plate was seeded with a 5 mm size mycelial disc taken from the periphery of an actively growing virulent culture plate. The plates were incubated in growth chambers at 25° C. temperature and 60% relative humidity for seven days and the radial growth was measured and compared to the one of the untreated control. Compounds 1 2 5 6 7 10 11 12 13 14 28 47 50 85 99 109 129 144 at 300 ppm gave more than or equal to 70% control in these tests when compared to the untreated check which showed extensive pathogen growth.

### Example 4: Colletotrichum Capsici (Anthracnose

The compounds were dissolved in 0.3% dimethyl sulfoxide and then added to potato dextrose agar medium just prior to dispensing it into petri dishes. 5 mL medium, with the compound in the desired concentration, was dispensed into 60 mm sterile petri-plates. After solidification, each plate was seeded with a 5 mm size mycelial disc taken from the periphery of an actively growing virulent culture plate. The plates were incubated in growth chambers at 25° C. temperature and 60% relative humidity for seven days and the radial growth was measured and compared to the one of the untreated control. Compounds 1 2 3 6 8 10 11 12 13 20 21 23 25 28 29 34 36 39 44 45 50 62 73 75 80 81 90 99 101 102 103 116 121 122 127 129 144 155 160 175 181 187 190 211 230 247 252 at 300 ppm gave more than or equal to 70% control in these tests when compared to the untreated check which showed extensive pathogen growth.

### Example 5: Corynespora Cassiicola (Lest Spot of Tomato

The compounds were dissolved in 0.3% dimethyl sulfoxide and then added to potato dextrose agar medium just prior to dispensing it into petri dishes. 5 mL medium, with the compound in the desired concentration, was dispensed into 60 mm sterile petri-plates. After solidification, each plate was seeded with a 5 mm size mycelial disc taken from the periphery of an actively growing virulent culture plate. The plates were incubated in growth chambers at 25° C. temperature and 70% relative humidity for seven days and the radial growth was measured and compared to the one of the untreated control. Compounds 2 6 43 107 109 121 125 127 129 at 300 ppm gave more than or equal to 70% control in these tests when compared to the untreated check which showed extensive pathogen growth.

### Example 6: Fusarium culmorum (Foot Rot/Head Blight of Cereals

The compounds were dissolved in 0.3% dimethyl sulfoxide and then added to Potato Dextrose Agar medium just prior to dispensing it into petri dishes. 5 ml medium, with compound in the desired concentration, was dispensed into 60 mm sterile petri-plates. After solidification, each plate was seeded with a 5 mm size mycelial disc taken from the periphery of actively growing virulent culture plates. The plates were incubated in growth chambers at 25° C. temperature and 60% relative humidity for seven days and the radial growth was measured. Compounds 2 and 13 at 300 ppm gave more than or equal to 70% control in these tests when compared to the untreated check which showed extensive pathogen growth.

### Example 7: Phytophthora infestans (Late Blight of Potato & Tomato

The compounds were dissolved in 0.3% DMSO and then added to Rye Agar medium just prior to dispensing it into petri dishes. 5 mL medium, with a compound in the desired concentration, was dispensed into 60 mm sterile petri-plates. After solidification, each plate was seeded with a 5 mm size mycelial disc taken form the periphery of an actively growing virulent culture plate. The plates were incubated in growth chambers at 18° C. temperature and 95% relative humidity for seven days and the radial growth was measured. Compounds 10 13 27 28 30 39 41 73 79 86 91 92 95 98 105 109 111 114 119 123 144 146 156 175 196 201 202 207 212 215 216 223 229 230 233 246 248 249 250 252 at 300 ppm gave more than or equal to 70% control in these tests when compared to the untreated check which showed extensive disease development.

### Example 8: Parastagonospora Nodorum (PARANO

Potato dextrose (PDB) liquid medium (Difco) containing a PARANO (10⁵ spores/ml) spore suspension was prepared. For the inhibition assay, each test compound was solved in dimethyl sulfoxide. 100 μl of the test compound in media-solution was added to a 96-well microtiter plate, consequently, the same volume (100 μl) of spore suspension was added to the well making the final test concentrations and the plate was incubated at 18° C. for 5-6 days. The growth inhibition was evaluated by measuring the OD at 600. Percent inhibition was calculated with the below formula:

$$I = ( C - B ) - ( T - B ) / ( C - B ) * 100$$

Where T=treatment, C=control, and B=blank
Compounds 5 6 7 8 10 11 13 16 17 18 23 25 26 27 28 34 36 40 42 43 44 45 47 48 50 56 60 63 67 73 75 78 79 80 81 82 85 86 90 95 99 100 101 102 103 105 106 108 109 118 119 124 135 144 149 150 151 153 155 159 160 173 175 176 179 181 187 190 200 205 210 211 217 223 227 230 232 239 243 244 245 247 249 252 253 254 at 300 ppm gave more than or equal to 70% control in these tests when compared to the untreated check which showed extensive pathogen growth.

### Example 9: Venturia Inequalis (VENTIN

Potato dextrose (PDB) liquid medium containing a VENTIN (10⁵ spores/ml) spore suspension was prepared. For the inhibition assay, each test compound was solved in dimethyl sulfoxide. 100 μl of the test compound in media-solution was added to a 96-well microtiter plate, consequently, the same volume (100 μl) of spore suspension was added to the well making the final test concentrations and the treated plate was incubated at 18° C. for 15-18 days. The growth inhibition was evaluated by measuring the OD at 600. Percent inhibition was calculated with the below formula:

$$I = ( C - B ) - ( T - B ) / ( C - B ) * 100$$

Where T=treatment, Control, and B=blank
Compounds 39 41 44 47 49 52 53 62 63 64 67 68 69 73 75 79 80 81 83 102 107 127 132 144 146 155 160 161 167 at 300 ppm gave more than or equal to 70% control in these tests when compared to the untreated check which showed extensive pathogen growth.

### Example 10: Botrytis Cinerea Liq. (BOTRCI

Yeast bacterial peptone and sodium acetate (YBA) liquid medium containing a BOTRCI (10⁴ spores/ml) spore suspension was prepared. For the inhibition assay, each test compound was solved in dimethyl sulfoxide. 100 μl of the test compound in media-solution was added to a 96-well microtiter plate, consequently, the same volume (100 μl) of spore suspension was added to the well making the final test concentrations and the plate was incubated at 22° C. for 5-6 days. The growth inhibition was evaluated by measuring the OD at 405 nm. Percent inhibition was calculated with the below formula:

$$I = ( C - B ) - ( T - B ) / ( C - B ) * 100$$

Where T=treatment, C=control, and B=blank

Compounds 6 8 13 14 20 50 55 63 67 72 73 75 78 82 89 98 100 101 105 106 109 118 144 148 149 187 189 190 194 202 219 221 223 230 232 239 242 244 247 252 254 at 300 ppm gave more than or equal to 70% control in these tests when compared to the untreated check which showed extensive pathogen growth.

### Biological Test Examples on Plants

The compounds were further selected for glasshouse testing. The methods followed to check the efficacy of compounds on the respective pathogens were as following:

### Example A: Pyricularia Oryzae Test on Rice

The respective compound was dissolved in 2% dimethyl sulfoxide/acetone and then mixed with water containing an emulsifier to the calibrated spray volume of 30 mL. The spray solution was poured into a spray bottle for further application.

To test the preventive activity of a compound, healthy young rice seedlings/plants, raised in the greenhouse, were sprayed with the active compound preparation at the stated application rate inside the spray cabinets using hollow cone nozzles. One day after treatment, the plants were inoculated with a spore suspension containing 1.4×10⁶ Pyricularia oryzae inoculum. The inoculated plants were then kept in a greenhouse chamber at 24° C. temperature and 95% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 26 116 119 137 at 500 ppm gave more than or equal to 70% control in these tests when compared to the untreated check which showed extensive disease development.

### Example B: Fusarium culmorum Test on Wheat

The respective compound was dissolved in 2% dimethyl sulfoxide/acetone and then mixed with water containing an emulsifier to a calibrated spray volume of 30 mL. Each spray solution was poured into a spray bottle for further application.

To test the preventive activity of a compound, healthy young wheat plants, raised in the greenhouse, were sprayed with the active compound preparation at the stated application rate inside the spray cabinets using hollow cone nozzles. One day after the treatment, the plants were inoculated with a spore suspension containing 2×10⁶ Fusarium culmorum inoculum. The inoculated plants were then kept in a greenhouse chamber at 24° C. temperature and 80-90% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 44 50 62 63 64 67 81 92 99 101 102 103 121 143 155 at 500 ppm gave more than or equal to 70% control in these tests when compared to the untreated check which showed extensive disease development.

### Example C: Botrytis cinerea Test on Tomato

The respective compound was dissolved in 2% dimethyl sulfoxide/acetone and then mixed with water containing an emulsifier to the calibrated spray volume of 30 mL. Each spray solution was poured into a spray bottle for further application.

To test the preventive activity of a compound, healthy young bean/chili plants, raised in the greenhouse, were sprayed with the active compound preparation at the stated application rate inside the spray cabinets using hollow cone nozzles. One day after treatment, the plants were inoculated with a spore suspension containing 1.2×10⁶ Botrytis cinerea inoculum. The inoculated plants were then kept in a greenhouse chamber at 18-20° C. temperature and 90-100% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 1 2 19 30 35 38 41 44 46 54 55 57 61 62 63 66 67 73 74 75 76 81 83 84 86 92 95 96 97 98 100 102 106 113 120 122 124 125 128 144 150 151 153 at 500 ppm gave more than or equal to 70% control in these tests when compared to the untreated check which showed extensive disease development.

### Example D: Alternaria solani Test in Tomato

The respective compound was dissolved in 2% dimethyl sulfoxide/acetone and then mixed with water containing an emulsifier to the calibrated spray volume of 30 mL. Each spray solution was poured into a spray bottle for further application.

To test the preventive activity of a compound, healthy young tomato plants, raised in the greenhouse, were sprayed with the active compound preparation at the stated application rate inside the spray cabinets using hollow cone nozzles. One day after treatment, the plants were inoculated with a spore suspension containing 0.24×10⁶ Alternaria solani inoculum. The inoculated plants were then kept in a greenhouse chamber at 22-24° C. temperature and 90-95% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 2 3 15 19 21 26 30 51 59 73 80 118 127 128 131 132 151 153 at 500 ppm gave more than or equal to 70% control in these tests when compared to the untreated check which showed extensive disease development.

### Example E: Corynespora cassiicola Test on Tomato

The respective compound was dissolved in 2% dimethyl sulfoxide/acetone and then mixed with water containing an emulsifier to the calibrated spray volume of 30 mL. Each spray solution was poured into a spray bottle for further application.

To test the preventive activity of a compound, healthy young tomato plants, raised in the greenhouse, were sprayed with the active compound preparation at the stated application rate inside the spray cabinets using hollow cone nozzles. One day after treatment, the plants were inoculated with a spore suspension containing 2.6×10⁶ Corynespora cassiicola inoculum. The inoculated plants were then kept in a greenhouse chamber at 22-24° C. temperature and 90-95% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compound 64 at 500 ppm gave more than or equal to 70% control in these tests when compared to the untreated check which showed extensive disease development.

### Example F: Phytophthora infestans Test on Tomato

The respective compound was dissolved in 2% dimethyl sulfoxide/acetone and then mixed with water containing an emulsifier to the calibrated spray volume of 30 mL. Each spray solution was poured into a spray bottle for further application.

To test the preventive activity of a compound, healthy young tomato plants, raised in the greenhouse, were sprayed with the active compound preparation at the stated application rate inside the spray cabinets using hollow cone nozzles. One day after treatment, the plants were inoculated with a sporangial suspension containing 0.24×10⁶ Phytophthora infestans inoculum. After inoculation, the plants were kept in darkness at 15° C. during 24 h, and then transferred to a greenhouse chamber at 18° C. temperature and 95-100% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 3 10 38 39 40 41 42 47 48 52 53 62 63 64 67 68 69 71 73 75 79 80 81 83 85 143 144 145 at 500 ppm gave more than or equal to 70% control in these tests when compared to the untreated check which showed extensive disease development.

### Example G: Phakopsora pachyrhizi Test on Soybean

The respective compound was dissolved in 2% dimethyl sulfoxide/acetone and then mixed with water containing emulsifier to the calibrated spray volume of 30 mL. Each spray solution was poured into a spray bottle for further application.

To test the preventive activity of a compound, healthy young soybean plants, raised in the greenhouse, were sprayed with the active compound preparation at the stated application rate inside the spray cabinets using hollow cone nozzles. One day after treatment, the plants were inoculated with a conidial suspension containing 2×10⁵ Phakopsora pachyrhizi inoculum. The inoculated plants were then kept in a greenhouse chamber at 22-24° C. temperature and 80-90% relative humidity for disease expression.

A visual assessment of the compound's performance was carried out by rating the disease severity (0 100% scale) on treated plants 3, 7, 10 and 15 days after application. Efficacy (% control) of the compound was calculated by comparing the disease rating in the treatment with the one of the untreated control. The treated plants were also assessed for plant damage by recording symptoms like necrosis, chlorosis and stunting. Compounds 1 2 5 10 19 21 22 23 24 25 26 28 29 34 35 38 39 40 41 42 43 44 45 46 47 48 49 50 51 52 53 54 56 57 58 59 62 63 64 65 66 67 68 71 75 76 83 84 85 86 87 88 89 93 94 95 96 97 99 101 102 103 104 106 107 119 125 127 129 131 132 133 134 137 138 140 141 142 143 144 146 150 151 155 at 500 ppm gave more than or equal to 70% control in these tests when compared to the untreated check which showed extensive disease development.

Having described the invention with reference to certain preferred embodiments, other embodiments will become apparent to one skilled in the art from the consideration of the specification. It will be apparent to those skilled in the art that many modifications, both to materials and methods, may be practiced without departing from the scope of the invention.

1. A compound of formula (I); 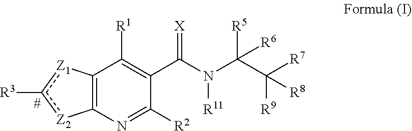
wherein,
“═” indicates presence or absence of double bond;
X is O or S;
Z¹ is C(R⁴)₁₋₂, CHR⁴—*CHR⁴, O—*CHR⁴, O, S, or N(R¹⁰)₀₋₁; wherein the carbon atom marked with “*” indicates the point of attachment to the carbon atom marked with “#”;
Z² is C(R⁴)₁₋₂ or NR¹⁰;
provided that
a) when Z¹ is S and Z² is CR⁴ then R⁵ and R⁶ will not form a ring;
b) when Z¹ is NH, Z² is NH and R³ is oxo then R⁵ and R⁶ will not form a ring;
c) when Z¹ is N, and Z² is N(CH₃) then R⁵ and R⁶ will not form a ring;
R¹ is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy may optionally be substituted with one or more groups of R^{1a};
R^{1a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R² is selected from the group consisting of hydrogen, halogen, cyano, hydroxy, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio, C₃-C₁₀-cycloalkyl and phenoxy may optionally be substituted with one or more groups of R^{2a};
R^{2a} is selected from the group consisting of halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R³ is selected from the group consisting of hydrogen, halogen, oxo, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl may optionally be substituted with one or more groups of R^{3a};
R^{3a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁴ is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylthio and C₃-C₁₀-cycloalkyl may optionally be substituted with one or more groups of R^{4a};
R^{4a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy and C₂-C₆-alkynyloxy;
R⁵ and R⁶ are each independently selected from the group consisting of hydrogen, cyano, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylcarbonyl, C₁-C₆-alkylthio, C₃-C₁₀-cycloalkyl, C₃-C₁₀-cycloalkyl-C₁-C₆-alkyl and C₆-C₁₀-aryl; wherein C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₂-C₆-alkenyloxy, C₂-C₆-alkynyloxy, C₁-C₆-alkylcarbonyl, C₁-C₆-alkylthio, C₃-C₁₀-cycloalkyl, C₃-C₁₀-cycloalkyl-C₁-C₆-alkyl and C₆-C₁₀-aryl may optionally be substituted with one or more groups of R^{5a}; or
R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₁₀-cycloalkyl and C₁-C₆-alkoxy-C₁-C₆-alkyl;
R⁷ and R⁸ are each independently selected from the group consisting of hydrogen, cyano, halogen, hydroxy, C₁-C₆-alkyl, C₁-C₆-haloalkyl, C₁-C₆-alkoxy, C₂-C₆-alkenyloxy, C₂-C₆-alkynyloxy, C₁-C₆-alkylcarbonyl and C₃-C₁₀-cycloalkyl; wherein C₁-C₆-alkyl, C₁-C₆-haloalkyl, C₁-C₆-alkoxy, C₂-C₆-alkenyloxy, C₂-C₆-alkynyloxy, C₁-C₆-alkylcarbonyl and C₃-C₁₀-cycloalkyl may optionally be substituted with one or more groups of R^{7a}; or
R⁷ and R⁸ together with the atoms to which they are attached may form a 3 to 6 membered non-aromatic or aromatic carbocyclic ring, or non-aromatic or aromatic heterocyclic ring; wherein one or more heteroatoms of said ring are selected from N, O or S(a)₀₋₂ and one or more C atom/s of the ring may optionally be replaced by C(═O) or C(═S); the ring may optionally be substituted with one or more identical or different groups of R^{7a}; provided that when R⁷ and R⁸ forms an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₁₀-cycloalkyl and C₁-C₆-alkoxy-C₁-C₆-alkyl;
R⁹ is selected from the group consisting of hydrogen, halogen, C₃-C₁₀-cycloalkyl, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contains 1, 2 or 3 heteroatoms selected from N, O and S; and wherein in each case one or two CH₂ groups of the carbocycle or heterocycle may be replaced by a group independently selected from C(═O) and C(═S); wherein C₃-C₁₀-cycloalkyl, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy may optionally be substituted with one or more groups of R^{9a}; or
R⁵ and R⁹ together with the atoms to which they are attached may form a 3 to 12 membered non-aromatic carbocyclic ring, or non-aromatic heterocyclic ring; wherein one or more heteroatoms of said ring are selected from N, O or S(═O)₀₋₂ and one or more C atom/s of the ring may optionally be replaced by C(═O), C(═S); the ring may optionally be substituted with one or more identical or different groups of R^{9a};
R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₁₀-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy;
R′ is selected from the group consisting of C₁-C₆-alkyl, C₂-C₆-alkenyl, C₂-C₆-alkynyl, wherein each group of R′ may optionally be substituted with groups selected from halogen, cyano, C₁-C₆-alkoxy, C₃-C₁₀-cycloalkyl, C₃-C₁₀-cycloalkyloxy, C₆-C₁₀-aryl, C₇-C₁₄-aralkyl and C₆-C₁₀-aryloxy;
R″ is selected from hydrogen, C₁-C₆-alkyl, C₂-C₆-alkenyl, C₂-C₆-alkynyl, C₃-C₁₀-cycloalkyl and C₃-C₁₀-cycloalkenyl;
R¹⁰ is selected from the group consisting of hydrogen, C₁-C₆-alkyl, C₁-C₆-haloalkyl, C₃-C₁₀-cycloalkyl and C₃-C₁₀-cycloalkyl-C₁-C₆-alkyl;
R¹¹ is selected from the group consisting of hydrogen, C₁-C₆-alkyl and C₁-C₆-alkoxy;
each group of R¹ to R¹¹, R^{1a} to R^{5a}, R^{7a} or R^{9a} may optionally be substituted with one or more groups selected from cyano, halogen, C₁-C₆-alkyl, C₂-C₆-alkenyl, C₂-C₆-alkynyl, C₃-C₁₀-cycloalkyl, C₃-C₁₀-cycloalkenyl and C₆-C₁₀-aryl;
and N-oxides, metal complexes, stereoisomers, polymorphs, or the agriculturally acceptable salts thereof,
with the proviso that, following compounds are excluded from the definition of compounds of formula (I),
2-methyl-N-(2,4,4-trimethylpentan-2-yl)-6,7-dihydro-5H-cyclopenta[b]pyridine-3-carboxamide,
N-(tert-butyl)-2-methyl-6,7-dihydro-5H-cyclopenta[b]pyridine-3-carboxamide
N-(tert-butyl)-2-methyl-5,6,7,8-tetrahydroquinoline-3-carboxamide,
2-methyl-N-(2,4,4-trimethylpentan-2-yl)-5,6,7,8-tetrahydroquinoline-3-carboxamide,
N-(1-(2-chlorophenoxy)-2-cyanopropan-2-yl)-4-methyl-3,4-dihydro-2H-pyrido[3,2-b][1,4]oxazine-7-carboxamide,
N-(2-cyano-1-(5-cyano-2-(trifluoromethyl)phenoxy)propan-2-yl)-4-methyl-3,4-dihydro-2H-pyrido[3,2-b][1,4]oxazine-7-carboxamide,
N-(2-(1,2,4-oxadiazol-3-yl)propan-2-yl)-3-methyl-3H-imidazo[4,5-b]pyridine-6-carboxamide,
N-(2-cyclohexylpropan-2-yl)-3-methyl-3H-imidazo[4,5-b]pyridine-6-carboxamide,
3-methyl-N-(1,1,1-trifluoro-2-methylbutan-2-yl)-3H-imidazo[4,5-b]pyridine-6-carboxamide,
N-(2-cyclopropyl-1,1,1-trifluoropropan-2-yl)-3-methyl-3H-imidazo[4,5-b]pyridine-6-carboxamide,
3-methyl-N-(4,4,4-trifluoro-2-methylbutan-2-yl)-3H-imidazo[4,5-b]pyridine-6-carboxamide,
N-(4-(azepan-1-yl)-2-methylbutan-2-yl)-3-methyl-3H-imidazo[4,5-b]pyridine-6-carboxamide,
N-(2-cyclohexyl-1-hydroxypropan-2-yl)-3-methyl-3H-imidazo[4,5-b]pyridine-6-carboxamide,
N-(2-cyclopropyl-1-hydroxypropan-2-yl)-3-methyl-3H-imidazo[4,5-b]pyridine-6-carboxamide,
N-(1-hydroxy-2-methylbutan-2-yl)-3-methyl-3H-imidazo[4,5-b]pyridine-6-carboxamide,
N-(tert-butyl)-1H-imidazo[4,5-b]pyridine-6-carboxamide,
N-(1-(azepan-1-yl)-2-methylpropan-2-yl)-N-methyl-3H-imidazo[4,5-b]pyridine-6-carboxamide,
N-(1-(azepan-1-yl)-2-methylpropan-2-yl)-N-methyl-2-oxo-2,3-dihydro-1H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(2-methyl-4-(4-methylpiperazin-1-yl)butan-2-yl)-2-oxo-2,3-dihydro-1H-imidazo[4,5-b]pyridine-6-carboxamide,
N-(2-(3,5-dimethylisoxazol-4-yl)propan-2-yl)-3-methyl-3H-imidazo[4,5-b]pyridine-6-carboxamide,
N-(2-(4,5-dimethylthiazol-2-yl)propan-2-yl)-2-oxo-2,3-dihydro-1H-imidazo[4,5-b]pyridine-6-carboxamide,
N-(1-(3-methoxyphenyl)-2-methylpropan-2-yl)-3-methyl-3H-imidazo[4,5-b]pyridine-6-carboxamide,
N-(2-(tetrahydro-2H-pyran-4-yl)propan-2-yl)thieno[3,2-b]pyridine-6-carboxamide,
N-(2-methyl-1-morpholinopropan-2-yl)thieno[3,2-b]pyridine-6-carboxamide and
N-(2-(1-methyl-1H-1,2,3-triazol-4-yl)propan-2-yl)thieno[3,2-b]pyridine-6-carboxamide.

2. The compound of formula (I) according to claim 1, wherein X is O;
R¹ is selected from the group consisting of hydrogen, halogen, cyano, hydroxy, C₁-C₆-alkyl, C₁-C₆-haloalkyl and C₁-C₆-alkoxy;
R² is selected from the group consisting of hydrogen, halogen, cyano, hydroxy, C₁-C₆-alkyl, C₁-C₆-haloalkyl, C₁-C₆-alkoxy, C₁-C₆-alkylthio and C₃-C₆-cycloalkyl;
R³ is selected from the group consisting of hydrogen, halogen, oxo, cyano, C₁-C₆-alkyl, C₁-C₆-haloalkyl, C₁-C₆-alkoxy, C₁-C₆-alkylthio and C₃-C₆-cycloalkyl;
R⁴ is selected from the group consisting of hydrogen, halogen, cyano, C₁-C₆-alkyl, C₁-C₆-haloalkyl, C₁-C₆-alkoxy, C₁-C₆-alkylthio and C₃-C₆-cycloalkyl;
R⁵ and R⁶ are each independently selected from the group consisting of hydrogen, cyano, halogen, C₁-C₆-alkyl, C₂-C₆-alkenyl, C₁-C₆-alkoxy, C₁-C₆-alkylcarbonyl, C₁-C₆-alkylthio, C₃-C₆-cycloalkyl, C₃-C₆-cycloalkyl-C₁-C₆-alkyl; wherein each group of R⁵ and R⁶ may optionally be substituted with one or more groups of R^{5a}; or
R⁵ and R⁶ together with the atoms to which they are attached may form a 3 to 6 membered non-aromatic carbocyclic ring, or non-aromatic heterocyclic ring; wherein one or more heteroatoms of said ring are selected from N, O or S(a)₀₋₂ and one or more C atom/s of the ring may optionally be replaced by C(═O), C(═S); the ring may optionally be substituted with one or more identical or different groups of R^{5a};
R^{5a} is selected from the group consisting of halogen, cyano, nitro, hydroxyl, thiol, amino and C₁-C₆-alkyl;
R⁷ and R⁸ are each independently selected from the group consisting of hydrogen, cyano, halogen, hydroxy, C₁-C₆-alkyl, C₁-C₆-haloalkyl and C₁-C₆-alkoxy, wherein C₁-C₆-alkyl, C₁-C₆-haloalkyl and C₁-C₆-alkoxy may optionally be substituted with one or more groups of R^{7a}; or
R⁷ and R⁸ together with the atoms to which they are attached may form a 3 to 6 membered non-aromatic or aromatic carbocyclic ring, or non-aromatic or aromatic heterocyclic ring; wherein one or more heteroatoms of said ring are selected from N, O or S(a)₀₋₂ and one or more C atom/s of the ring may optionally be replaced by C(═O) or C(═S); the ring may optionally be substituted with one or more identical or different groups of R^{7a}; provided that when R⁷ and R⁸ forms an aromatic ring then R⁹ is absent;
R^{7a} is selected from the group consisting of halogen, cyano, nitro, hydroxyl, thiol, amino, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl and C₁-C₄-alkoxy-C₁-C₄-alkyl;
R⁹ is selected from the group consisting of hydrogen, halogen, C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy; wherein the heterocyclyl or heterocycloxy contains 1, 2 or 3 heteroatoms selected from N, O and S; and wherein in each case one or two CH₂ groups of the carbocycle or heterocycle may be replaced by a group independently selected from C(═O) and C(═S); wherein C₆-C₁₀-aryl, C₇-C₁₄-aralkyl, C₆-C₁₀-aryloxy, benzyloxy, —O—C(O)—C₆-C₁₀-aryl, O—C(O)—C₂-C₁₀-heterocyclyl, C₂-C₁₀-heterocyclyl and C₂-C₁₀-heterocycloxy may optionally be substituted with one or more groups of R; or
R⁵ and R⁹ together with the atoms to which they are attached may form a 3 to 12 membered non-aromatic carbocyclic ring, or non-aromatic heterocyclic ring; wherein one or more heteroatoms of said ring are selected from N, O or S(a)₀₋₂ and one or more C atom/s of the ring may optionally be replaced by C(═O), C(═S); the ring may optionally be substituted with one or more identical or different groups of R^{9a};
R^{9a} is selected from the group consisting of hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₂-C₆-alkenyloxy, C₂-C₆-alkynyloxy, C₃-C₆-cycloalkyl, C(R″)═N—OR′, C₃-C₁₀-cycloalkyloxy, C₃-C₁₀-cycloalkenyl, C₃-C₁₀-cycloalkenyloxy, C₆-C₁₀-aryl, C₇-C₁₄-aralkyl, C₆-C₁₀-aryloxy, C₂-C₁₀-heterocyclyl and C₂-C₁₀-heterocycloxy;
R′ is selected from the group consisting of C₁-C₆-alkyl, C₂-C₆-alkenyl, C₂-C₆-alkynyl, wherein each group of R′ may optionally be substituted with groups selected from halogen, cyano, C₁-C₆-alkoxy, C₃-C₆-cycloalkyl, C₃-C₆-cycloalkyloxy, C₆-C₁₀-aryl, C₇-C₁₄-aralkyl and C₆-C₁₀-aryloxy;
R″ is selected from hydrogen, C₁-C₆-alkyl, C₂-C₆-alkenyl, C₂-C₆-alkynyl, C₃-C₆-cycloalkyl and C₃-C₆-cycloalkenyl;
R¹⁰ is selected from the group consisting of hydrogen, C₁-C₄-alkyl, C₁-C₄-haloalkyl, C₃-C₅-cycloalkyl and C₃-C₅-cycloalkyl-C₁-C₃-alkyl; and
R¹¹ is selected from the group consisting of hydrogen, C₁-C₄-alkyl and C₁-C₄-alkoxy.

3. The compound of formula (I) according to claim 1, wherein Z¹ is CH(R⁴); and Z² is NR¹⁰.

4. The compound of formula (I) according to claim 1, wherein Z¹ is S and Z² is CHR⁴.

5. The compound of formula (I) according to claim 1, wherein Z¹ is CHR⁴—*CHR⁴; wherein the carbon atom marked with “*” indicates the point of attachment to the carbon atom marked with “#”; and Z² is CHR⁴.

6. The compound of formula (I) according to claim 1, wherein Z¹ is CHR⁴ and Z² is CHR⁴.

7. The compound of formula (I) according to claim 1, wherein Z¹ is O—*CHR⁴; wherein the carbon atom marked with “*” indicates the point of attachment to the carbon atom marked with “#”; and Z² is NR¹⁰.

8. The compound of formula (I) according to claim 1, wherein Z¹ is NR¹⁰; and Z² is CHR⁴.

9. The compound of formula (I) according to claim 1, wherein Z¹ is O; Z² is NR¹⁰ and R³ is oxo.

10. The compound of formula (I) according to claim 1, wherein said compound of formula (I) is selected from 1-methyl-N-(2-methyl-1-phenylpropan-2-yl)-1H-pyrrolo[2,3-b]pyridine-5-carboxamide, N-(2,4-dimethyl-1-phenylpentan-2-yl)-1-methyl-1H-pyrrolo[2,3-b]pyridine-5-carboxamide, N-(3-(2-fluorophenyl)-2-methylbutan-2-yl)-1-methyl-1H-pyrrolo[2,3-b]pyridine-5-carboxamide, N-(2,3-dimethyl-3-phenylbutan-2-yl)-1-methyl-1H-pyrrolo[2,3-b]pyridine-5-carboxamide, 1-methyl-N-(2-methyl-1-phenylbutan-2-yl)-1H-pyrrolo[2,3-b]pyridine-5-carboxamide, N-(3-(2-chlorophenyl)-2-methylbutan-2-yl)-1-methyl-1H-pyrrolo[2,3-b]pyridine-5-carboxamide, 1-methyl-N-(2-methyl-3-phenylbutan-2-yl)-1H-pyrrolo[2,3-b]pyridine-5-carboxamide, N-(1-benzylcyclopentyl)-1-methyl-1H-pyrrolo[2,3-b]pyridine-5-carboxamide, N-(1-benzylcyclohexyl)-1-methyl-1H-pyrrolo[2,3-b]pyridine-5-carboxamide, N-(1-(3-chlorobenzyl)cyclohexyl)-1-methyl-1H-pyrrolo[2,3-b]pyridine-5-carboxamide, N-(1-(2-fluorophenyl)-2-methylpropan-2-yl)-1-methyl-1H-pyrrolo[2,3-b]pyridine-5-carboxamide, 1-methyl-N-(2-methyl-1-(3-(trifluoromethyl)phenyl)propan-2-yl)-1H-pyrrolo[2,3-b]pyridine-5-carboxamide, N-(1-(3,5-difluorophenyl)-2-methylpropan-2-yl)-1-methyl-1H-pyrrolo[2,3-b]pyridine-5-carboxamide, N-(1-(4-(tert-butyl)phenyl)-2-methylpropan-2-yl)-1-methyl-1H-pyrrolo[2,3-b]pyridine-5-carboxamide, N-(1-(2-chlorophenyl)-2-methylpropan-2-yl)-1-methyl-1H-pyrrolo[2,3-b]pyridine-5-carboxamide, N-(1-(4-isopropylphenyl)-2-methylpropan-2-yl)-1-methyl-1H-pyrrolo[2,3-b]pyridine-5-carboxamide, 1-methyl-N-(2-methyl-1-(4-(trifluoromethoxy)phenyl)propan-2-yl)-1H-pyrrolo[2,3-b]pyridine-5-carboxamide, N-(1-(2,4-difluorophenyl)-2-methylpropan-2-yl)-1-methyl-1H-pyrrolo[2,3-b]pyridine-5-carboxamide, N-(1-(3-fluorophenyl)-2-methylpropan-2-yl)-1-methyl-1H-pyrrolo[2,3-b]pyridine-5-carboxamide, N-(1-(3-chlorophenyl)-2-methylpropan-2-yl)-1-methyl-1H-pyrrolo[2,3-b]pyridine-5-carboxamide, N-(1-(3,4-difluorophenyl)-2-methylpropan-2-yl)-1-methyl-1H-pyrrolo[2,3-b]pyridine-5-carboxamide, N-(1-(3,4-dichlorophenyl)-2-methylpropan-2-yl)-1-methyl-1H-pyrrolo[2,3-b]pyridine-5-carboxamide, N-(4,4,4-trifluoro-2-methyl-1-phenylbutan-2-yl)thieno[3,2-b]pyridine-6-carboxamide, N-(1-(2-bromophenyl)-2-methylpropan-2-yl)-1-methyl-1H-pyrrolo[2,3-b]pyridine-5-carboxamide, 1-methyl-N-(2-methyl-1-(m-tolyl)propan-2-yl)-1H-pyrrolo[2,3-b]pyridine-5-carboxamide, N-(2,4-dimethyl-1-phenylpentan-2-yl)-1H-pyrrolo[2,3-b]pyridine-5-carboxamide, N-(2,4-dimethyl-1-phenylpentan-2-yl)thieno[3,2-b]pyridine-6-carboxamide, N-(2-methyl-1-phenylpropan-2-yl)thieno[3,2-b]pyridine-6-carboxamide, 1,6-dimethyl-N-(2-methyl-1-phenylpropan-2-yl)-1H-pyrrolo[2,3-b]pyridine-5-carboxamide, N-(2,4-dimethyl-1-phenylpentan-2-yl)-1,6-dimethyl-1H-pyrrolo[2,3-b]pyridine-5-carboxamide, N-(3-(2-fluorophenyl)-2-methylbutan-2-yl)-1,6-dimethyl-1H-pyrrolo[2,3-b]pyridine-5-carboxamide, N-(1-(2-fluorophenyl)-2,4-dimethylpentan-2-yl)-1-methyl-1H-pyrrolo[2,3-b]pyridine-5-carboxamide, N-(2,3-dimethyl-3-phenylbutan-2-yl)-1,6-dimethyl-1H-pyrrolo[2,3-b]pyridine-5-carboxamide, N-(1-(2-fluorophenyl)-2-methylpropan-2-yl)-1,6-dimethyl-1H-pyrrolo[2,3-b]pyridine-5-carboxamide, N-(1-benzylcyclopropyl)-1-methyl-1H-pyrrolo[2,3-b]pyridine-5-carboxamide, N-(3-(2-chlorophenyl)-2-methylbutan-2-yl)-1,6-dimethyl-1H-pyrrolo[2,3-b]pyridine-5-carboxamide, N-(1-(4-fluorophenyl)-2-methylpropan-2-yl)-1H-pyrrolo[2,3-b]pyridine-5-carboxamide, N-(1-(4-chloro-3-fluorophenyl)-2-methylpropan-2-yl)-1-methyl-1H-pyrrolo[2,3-b]pyridine-5-carboxamide, N-(1-(4-chlorophenyl)-2-methylpropan-2-yl)-1-methyl-1H-pyrrolo[2,3-b]pyridine-5-carboxamide, N-(2,4-dimethyl-1-phenylpentan-2-yl)-5,6,7,8-tetrahydroquinoline-3-carboxamide, N-(2-methyl-1-phenylpropan-2-yl)-5,6,7,8-tetrahydroquinoline-3-carboxamide, 3-bromo-1-methyl-N-(2-methyl-1-phenylpropan-2-yl)-1H-pyrrolo[2,3-b]pyridine-5-carboxamide, N-(1-(4-chloro-2-fluorophenyl)-2-methylpropan-2-yl)-1-methyl-1H-pyrrolo[2,3-b]pyridine-5-carboxamide, N-(1-(3-bromophenyl)-2-methylpropan-2-yl)-1-methyl-1H-pyrrolo[2,3-b]pyridine-5-carboxamide, N-(1-(2-methoxyphenyl)-2-methylpropan-2-yl)-1-methyl-1H-pyrrolo[2,3-b]pyridine-5-carboxamide, 1-methyl-N-(2-methyl-1-(p-tolyl)propan-2-yl)-1H-pyrrolo[2,3-b]pyridine-5-carboxamide, 3-bromo-N-(2,4-dimethyl-1-phenylpentan-2-yl)-1-methyl-1H-pyrrolo[2,3-b]pyridine-5-carboxamide, 3-chloro-N-(2,4-dimethyl-1-phenylpentan-2-yl)-1-methyl-1H-pyrrolo[2,3-b]pyridine-5-carboxamide, 3-chloro-1-methyl-N-(2-methyl-1-phenylpropan-2-yl)-1H-pyrrolo[2,3-b]pyridine-5-carboxamide, 3-cyclopropyl-1-methyl-N-(2-methyl-1-phenylpropan-2-yl)-1H-pyrrolo[2,3-b]pyridine-5-carboxamide, 3-cyclopropyl-N-(2,4-dimethyl-1-phenylpentan-2-yl)-1-methyl-1H-pyrrolo[2,3-b]pyridine-5-carboxamide, 3-chloro-N,1-dimethyl-N-(2-methyl-1-phenylpropan-2-yl)-1H-pyrrolo[2,3-b]pyridine-5-carboxamide, 3-cyclopropyl-N,1-dimethyl-N-(2-methyl-1-phenylpropan-2-yl)-1H-pyrrolo[2,3-b]pyridine-5-carboxamide, 3-bromo-N-(1-(2-fluorophenyl)-2-methylpropan-2-yl)-1-methyl-1H-pyrrolo[2,3-b]pyridine-5-carboxamide, 3-chloro-N-(1-(2-fluorophenyl)-2-methylpropan-2-yl)-1-methyl-1H-pyrrolo[2,3-b]pyridine-5-carboxamide, 3-cyclopropyl-N-(1-(2-fluorophenyl)-2-methylpropan-2-yl)-1-methyl-1H-pyrrolo[2,3-b]pyridine-5-carboxamide, N-(1-(4-bromophenyl)-2-methylpropan-2-yl)-1-methyl-1H-pyrrolo[2,3-b]pyridine-5-carboxamide, N-(2,4-dimethyl-1-phenylpentan-2-yl)-6,7-dihydro-5H-cyclopenta[b]pyridine-3-carboxamide, 3-bromo-N-(3-(2-fluorophenyl)-2-methylbutan-2-yl)-1-methyl-1H-pyrrolo[2,3-b]pyridine-5-carboxamide, 3-chloro-N-(3-(2-fluorophenyl)-2-methylbutan-2-yl)-1-methyl-1H-pyrrolo[2,3-b]pyridine-5-carboxamide, 3-cyclopropyl-N-(3-(2-fluorophenyl)-2-methylbutan-2-yl)-1-methyl-1H-pyrrolo[2,3-b]pyridine-5-carboxamide, N-(1-(4-chlorophenyl)-2-methylpropan-2-yl)-3-fluoro-1-methyl-1H-pyrrolo[2,3-b]pyridine-5-carboxamide, N-(2-methyl-1-phenylpropan-2-yl)-6,7-dihydro-5H-cyclopenta[b]pyridine-3-carboxamide, 3-fluoro-N-(1-(2-fluorophenyl)-2-methylpropan-2-yl)-1-methyl-1H-pyrrolo[2,3-b]pyridine-5-carboxamide, N-(1-(4-chloro-3-fluorophenyl)-2-methylpropan-2-yl)-3-fluoro-1-methyl-1H-pyrrolo[2,3-b]pyridine-5-carboxamide, 3-fluoro-1-methyl-N-(2-methyl-1-(p-tolyl)propan-2-yl)-1H-pyrrolo[2,3-b]pyridine-5-carboxamide, N-(3-(2-chlorophenyl)-2-methylbutan-2-yl)-3-fluoro-1-methyl-1H-pyrrolo[2,3-b]pyridine-5-carboxamide, 3-fluoro-N-(1-(2-methoxyphenyl)-2-methylpropan-2-yl)-1-methyl-1H-pyrrolo[2,3-b]pyridine-5-carboxamide, N-(2,4-dimethyl-1-phenylpentan-2-yl)-1H-pyrrolo[3,2-b]pyridine-6-carboxamide, N-(1-(4-fluorophenyl)-2-methylpropan-2-yl)-1-methyl-1H-pyrrolo[2,3-b]pyridine-5-carboxamide, 1-methyl-N-(4-phenylbutan-2-yl)-1H-pyrrolo[2,3-b]pyridine-5-carboxamide, N-(2,3-dihydro-1H-inden-2-yl)-1-methyl-1H-pyrrolo[2,3-b]pyridine-5-carboxamide, 1-methyl-N-(2-methyl-1-(naphthalen-2-yl)propan-2-yl)-1H-pyrrolo[2,3-b]pyridine-5-carboxamide, 1-methyl-N-(2-methyl-2,3-dihydro-1H-inden-2-yl)-1H-pyrrolo[2,3-b]pyridine-5-carboxamide, 1-methyl-N-((1-phenylcyclopropyl)methyl)-1H-pyrrolo[2,3-b]pyridine-5-carboxamide, N-(2,3-dimethyl-4-phenylbutan-2-yl)-1-methyl-1H-pyrrolo[2,3-b]pyridine-5-carboxamide, N-(2,4-dimethyl-1-phenylpentan-2-yl)-4-methyl-3,4-dihydro-2H-pyrido[3,2-b][1,4]oxazine-7-carboxamide, 4-methyl-N-(2-methyl-1-phenylpropan-2-yl)-3,4-dihydro-2H-pyrido[3,2-b][1,4]oxazine-7-carboxamide, 3-chloro-N-(1-(2-chlorophenyl)-2-methylpropan-2-yl)-1-methyl-1H-pyrrolo[2,3-b]pyridine-5-carboxamide, 3-bromo-N-(1-(2-chlorophenyl)-2-methylpropan-2-yl)-1-methyl-1H-pyrrolo[2,3-b]pyridine-5-carboxamide, N-(1-(2-chlorophenyl)-2-methylpropan-2-yl)-3-cyclopropyl-1-methyl-1H-pyrrolo[2,3-b]pyridine-5-carboxamide, 1-methyl-N-(2-methyl-1-phenylpropan-2-yl)-1H-pyrrolo[3,2-b]pyridine-6-carboxamide, 3-chloro-1-methyl-N-(2-methyl-3-phenylbutan-2-yl)-1H-pyrrolo[2,3-b]pyridine-5-carboxamide, 6-methoxy-N-(2-methyl-1-phenylpropan-2-yl)-1H-pyrrolo[2,3-b]pyridine-5-carboxamide, N-(2,4-dimethyl-1-phenylpentan-2-yl)-6-methoxy-1H-pyrrolo[2,3-b]pyridine-5-carboxamide, 1-methyl-N-(2-methyl-1-(4-(methylthio)phenyl)propyl)-1H-pyrrolo[2,3-b]pyridine-5-carboxamide, 6-methoxy-1-methyl-N-(2-methyl-1-phenylpropan-2-yl)-1H-pyrrolo[2,3-b]pyridine-5-carboxamide, N-(2,4-dimethyl-1-phenylpentan-2-yl)-6-methoxy-1-methyl-1H-pyrrolo[2,3-b]pyridine-5-carboxamide, 6-methyl-N-(2-methyl-1-phenylpropan-2-yl)-1H-pyrrolo[2,3-b]pyridine-5-carboxamide, N-(1-(4-fluorophenyl)-2-methylpropan-2-yl)-6-methyl-1H-pyrrolo[2,3-b]pyridine-5-carboxamide, N-(2-methyl-1-phenylpropan-2-yl)-1H-pyrrolo[3,2-b]pyridine-6-carboxamide, N-(2,4-dimethyl-1-phenylpentan-2-yl)-6-methyl-1H-pyrrolo[2,3-b]pyridine-5-carboxamide, N-(1-benzylcyclopropyl)-6-methyl-1H-pyrrolo[2,3-b]pyridine-5-carboxamide, N-(2,4-dimethyl-1-phenylpentan-2-yl)-2-oxo-2,3-dihydrooxazolo[4,5-b]pyridine-6-carboxamide, 1,2-dimethyl-N-(2-methyl-1-phenylpropan-2-yl)-1H-pyrrolo[2,3-b]pyridine-5-carboxamide, N-(2,3-dimethyl-3-phenylbutan-2-yl)-1,2-dimethyl-1H-pyrrolo[2,3-b]pyridine-5-carboxamide, N-(1-(4-chlorophenyl)-2-methylpropan-2-yl)-1,2-dimethyl-1H-pyrrolo[2,3-b]pyridine-5-carboxamide, 1,2-dimethyl-N-(2-methyl-1-(p-tolyl)propan-2-yl)-1H-pyrrolo[2,3-b]pyridine-5-carboxamide, N-(1-(2-fluorophenyl)-2-methylpropan-2-yl)-1,2-dimethyl-1H-pyrrolo[2,3-b]pyridine-5-carboxamide, N-(3-hydroxy-3-methyl-1-(m-tolyl)butan-2-yl)-1H-pyrrolo[2,3-b]pyridine-5-carboxamide, N-(3-hydroxy-2-methyl-4-phenylbutan-2-yl)thieno[3,2-b]pyridine-6-carboxamide, N-(2-cyanopropan-2-yl)thieno[3,2-b]pyridine-6-carboxamide, N-(tert-butyl)thieno[3,2-b]pyridine-6-carboxamide, N-(1-(trifluoromethyl)cyclopropyl)thieno[3,2-b]pyridine-6-carboxamide, N-(1,1,1-trifluoro-2-methylpropan-2-yl)thieno[3,2-b]pyridine-6-carboxamide, N-(1,1,1-trifluoro-2-methylbutan-2-yl)thieno[3,2-b]pyridine-6-carboxamide, N-(1-hydroxy-2-methylpropan-2-yl)thieno[3,2-b]pyridine-6-carboxamide, N-(3-hydroxy-3-methyl-1-(m-tolyl)butan-2-yl)thieno[3,2-b]pyridine-6-carboxamide, N-(3-hydroxy-2-methyl-4-phenylbutan-2-yl)-1-methyl-1H-pyrrolo[2,3-b]pyridine-5-carboxamide, N-(1-hydroxy-2-methyl-1-phenylpropan-2-yl)-1-methyl-1H-pyrrolo[2,3-b]pyridine-5-carboxamide, N-(1-hydroxy-2-methyl-1-phenylpropan-2-yl)thieno[3,2-b]pyridine-6-carboxamide, N-(1-(4-fluoro-3-methoxyphenyl)-1-hydroxy-2-methylpropan-2-yl)thieno[3,2-b]pyridine-6-carboxamide, N-(2-ethyl-2-hydroxy-1-phenylbutyl)-1-methyl-1H-pyrrolo[2,3-b]pyridine-5-carboxamide, N-(1-(4-fluorophenoxy)-2,4-dimethylpent-4-en-2-yl)-1-methyl-1H-pyrrolo[2,3-b]pyridine-5-carboxamide, N-(1-(4-fluorophenoxy)-2,4-dimethylpent-4-en-2-yl)thieno[3,2-b]pyridine-6-carboxamide, N-(1-(4-fluoro-3-methoxyphenyl)-1-hydroxy-2-methylpropan-2-yl)-1-methyl-1H-pyrrolo[2,3-b]pyridine-5-carboxamide, 2-methyl-2-(thieno[3,2-b]pyridine-6-carboxamido)propyl 4-fluoro-3-methoxybenzoate, 2-methyl-2-(1-methyl-1H-pyrrolo[2,3-b]pyridine-5-carboxamido)propyl 4-fluoro-2-methylbenzoate, N-(2-ethyl-2-hydroxy-1-phenylbutyl)thieno[3,2-b]pyridine-6-carboxamide, N-(1-(2-fluorophenyl)-2,4-dimethylpent-4-en-2-yl)-1-methyl-1H-pyrrolo[2,3-b]pyridine-5-carboxamide, N-(1-(4-fluorophenoxy)-2,4-dimethylpent-4-en-2-yl)-6-methyl-1H-pyrrolo[2,3-b]pyridine-5-carboxamide, N-(1-(2-fluorophenyl)-2,4-dimethylpent-4-en-2-yl)thieno[3,2-b]pyridine-6-carboxamide, N-(1-(4-fluoro-2-methylphenyl)-1-hydroxy-2-methylpropan-2-yl)-1-methyl-1H-pyrrolo[2,3-b]pyridine-5-carboxamide, N-(2-ethyl-1-(4-fluorophenyl)-2-hydroxybutyl)-1-methyl-1H-pyrrolo[2,3-b]pyridine-5-carboxamide, N-(2-ethyl-1-(4-fluorophenyl)-2-hydroxybutyl)thieno[3,2-b]pyridine-6-carboxamide, N-(1-(5-fluoro-2-methylphenyl)-1-hydroxy-2-methylpropan-2-yl)thieno[3,2-b]pyridine-6-carboxamide, N-(1-(2-fluorophenyl)-2-methylpent-4-en-2-yl)thieno[3,2-b]pyridine-6-carboxamide, 3-bromo-N-(1-(5-fluoro-2-methylphenyl)-1-hydroxy-2-methylpropan-2-yl)-1-methyl-1H-pyrrolo[2,3-b]pyridine-5-carboxamide, N-(1-(4-fluorophenyl)-2-hydroxy-2-methylpropyl)thieno[3,2-b]pyridine-6-carboxamide, 3-chloro-N-(2,3-dihydro-1H-inden-2-yl)-1-methyl-1H-pyrrolo[2,3-b]pyridine-5-carboxamide, 3-chloro-N-(2-ethyl-2-hydroxy-1-phenylbutyl)-1-methyl-1H-pyrrolo[2,3-b]pyridine-5-carboxamide, N-(1-(2-fluorophenyl)-2,4-dimethylpent-4-en-2-yl)-1H-pyrrolo[2,3-b]pyridine-5-carboxamide, 3-chloro-N-(1-(3,5-dichlorophenyl)cyclopropyl)-1-methyl-1H-pyrrolo[2,3-b]pyridine-5-carboxamide, (S)—N-(1,1-bis(3,5-dichlorophenyl)-1-hydroxypropan-2-yl)-1-methyl-1H-pyrrolo[2,3-b]pyridine-5-carboxamide, 3-bromo-N-(1-(2-fluorophenyl)-2,4-dimethylpent-4-en-2-yl)-1-methyl-1H-pyrrolo[2,3-b]pyridine-5-carboxamide, 3-chloro-N-(1-(3-methoxyphenyl)cyclopropyl)-1-methyl-1H-pyrrolo[2,3-b]pyridine-5-carboxamide, N-(1-(2-fluorophenyl)-2,4-dimethylpent-4-en-2-yl)-1H-pyrrolo[3,2-b]pyridine-6-carboxamide, 2-(6,7-dihydro-5H-cyclopenta[b]pyridine-3-carboxamido)-2-methylpropyl 3-fluorobenzoate, 2-methyl-2-(1H-pyrrolo[2,3-b]pyridine-5-carboxamido)propyl 3-fluorobenzoate, 2-(6,7-dihydro-5H-cyclopenta[b]pyridine-3-carboxamido)-2-methylpropyl 2-(trifluoromethyl)benzoate, 2-(6,7-dihydro-5H-cyclopenta[b]pyridine-3-carboxamido)-2-methylpropyl 2-methylbenzoate, 2-methyl-2-(5,6,7,8-tetrahydroquinoline-3-carboxamido)propyl 2-(trifluoromethyl)benzoate, 2-methyl-2-(5,6,7,8-tetrahydroquinoline-3-carboxamido)propyl 2-methylbenzoate, 2-methyl-2-(5,6,7,8-tetrahydroquinoline-3-carboxamido)propyl 3-fluorobenzoate, 2-(3-chloro-1-methyl-1H-pyrrolo[2,3-b]pyridine-5-carboxamido)-2-methylpropyl 3-fluorobenzoate, N-(1-(2-fluorophenoxy)-2,4-dimethylpent-4-en-2-yl)-1-methyl-1H-pyrrolo[2,3-b]pyridine-5-carboxamide, 2-methyl-2-(1H-pyrrolo[3,2-b]pyridine-6-carboxamido)propyl 3-fluorobenzoate, 2-methyl-2-(1H-pyrrolo[2,3-b]pyridine-5-carboxamido)propyl 2-(trifluoromethyl)benzoate, N-(1-(2-fluorophenoxy)-2,4-dimethylpent-4-en-2-yl)thieno[3,2-b]pyridine-6-carboxamide, N-(1-(2-fluorophenoxy)-2,4-dimethylpent-4-en-2-yl)-6,7-dihydro-5H-cyclopenta[b]pyridine-3-carboxamide, 2-methyl-2-(thieno[3,2-b]pyridine-6-carboxamido)propyl 2-(trifluoromethyl)benzoate, 2-methyl-2-(1H-pyrrolo[3,2-b]pyridine-6-carboxamido)propyl 2-(trifluoromethyl)benzoate, 2-methyl-2-(thieno[3,2-b]pyridine-6-carboxamido)propyl 2-methylbenzoate, N-(1-(4-fluoro-3-methoxyphenyl)-1-hydroxy-2-methylpropan-2-yl)-6,7-dihydro-5H-cyclopenta[b]pyridine-3-carboxamide, 2-methyl-2-(1H-pyrrolo[2,3-b]pyridine-5-carboxamido)propyl 2-methylbenzoate, 2-methyl-2-(1-methyl-1H-pyrrolo[2,3-b]pyridine-5-carboxamido)propyl 2-(trifluoromethyl)benzoate, 2-methyl-2-(1-methyl-1H-pyrrolo[2,3-b]pyridine-5-carboxamido)propyl 3-fluorobenzoate, N-(1-(4-fluorophenyl)-2-hydroxy-2-methylpropyl)-1H-pyrrolo[2,3-b]pyridine-5-carboxamide, N-(1-(2-fluorophenoxy)-2,4-dimethylpent-4-en-2-yl)-1H-pyrrolo[2,3-b]pyridine-5-carboxamide, N-(1-(4-fluoro-2-methylphenyl)-1-hydroxy-2-methylpropan-2-yl)-6,7-dihydro-5H-cyclopenta[b]pyridine-3-carboxamide, N-(1-(4-fluoro-2-methylphenyl)-1-hydroxy-2-methylpropan-2-yl)thieno[3,2-b]pyridine-6-carboxamide, N-(1-(2-fluorophenoxy)-2,4-dimethylpent-4-en-2-yl)-1H-pyrrolo[3,2-b]pyridine-6-carboxamide, 3-bromo-N-(1-(2-fluorophenoxy)-2,4-dimethylpent-4-en-2-yl)-1-methyl-1H-pyrrolo[2,3-b]pyridine-5-carboxamide, N-(1-(2-fluorophenoxy)-2,4-dimethylpent-4-en-2-yl)-6-methyl-1H-pyrrolo[2,3-b]pyridine-5-carboxamide, 2-(3-bromo-1-methyl-1H-pyrrolo[2,3-b]pyridine-5-carboxamido)-2-methylpropyl 3-fluorobenzoate, 3-chloro-N-(1-(2-fluorophenoxy)-2,4-dimethylpent-4-en-2-yl)-1-methyl-1H-pyrrolo[2,3-b]pyridine-5-carboxamide, 2-(3-bromo-1-methyl-1H-pyrrolo[2,3-b]pyridine-5-carboxamido)-2-methylpropyl 2-(trifluoromethyl)benzoate, N-(1-(4-fluorophenyl)-2,4-dimethylpent-4-en-2-yl)-1-methyl-1H-pyrrolo[2,3-b]pyridine-5-carboxamide, N-(1-(4-fluorophenyl)-2,4-dimethylpent-4-en-2-yl)thieno[3,2-b]pyridine-6-carboxamide, 2-(3-bromo-1-methyl-1H-pyrrolo[2,3-b]pyridine-5-carboxamido)-2-methylpropyl 2-methylbenzoate, N-(1-(5-fluoro-2-methylphenyl)-1-hydroxy-2-methylpropan-2-yl)-6,7-dihydro-5H-cyclopenta[b]pyridine-3-carboxamide, N-(1-((2-fluorobenzyl)oxy)-2-methylpropan-2-yl)-1-methyl-1H-pyrrolo[2,3-b]pyridine-5-carboxamide, N-(1-((2-cyanobenzyl)oxy)-2-methylpropan-2-yl)-6,7-dihydro-5H-cyclopenta[b]pyridine-3-carboxamide, N-(1-((2-fluorobenzyl)oxy)-2-methylpropan-2-yl)thieno[3,2-b]pyridine-6-carboxamide, N-(1-((2-fluorobenzyl)oxy)-2-methylpropan-2-yl)-6,7-dihydro-5H-cyclopenta[b]pyridine-3-carboxamide, N-(1-((2-chlorobenzyl)oxy)-2-methylpropan-2-yl)-6,7-dihydro-5H-cyclopenta[b]pyridine-3-carboxamide, N-(1-((2-chlorobenzyl)oxy)-2-methylpropan-2-yl)thieno[3,2-b]pyridine-6-carboxamide, N-(1-((2-cyanobenzyl)oxy)-2-methylpropan-2-yl)thieno[3,2-b]pyridine-6-carboxamide, 3-bromo-N-(1-((2-fluorobenzyl)oxy)-2-methylpropan-2-yl)-1-methyl-1H-pyrrolo[2,3-b]pyridine-5-carboxamide, 3-bromo-N-(1-((2-chlorobenzyl)oxy)-2-methylpropan-2-yl)-1-methyl-1H-pyrrolo[2,3-b]pyridine-5-carboxamide, 3-bromo-N-(1-((2-cyanobenzyl)oxy)-2-methylpropan-2-yl)-1-methyl-1H-pyrrolo[2,3-b]pyridine-5-carboxamide, N-(1-((2-chloro-5-fluorobenzyl)oxy)-2-methylpropan-2-yl)-6,7-dihydro-5H-cyclopenta[b]pyridine-3-carboxamide, N-(1-((2-chloro-5-fluorobenzyl)oxy)-2-methylpropan-2-yl)thieno[3,2-b]pyridine-6-carboxamide, N-(1-((2,4-dimethylbenzyl)oxy)-2-methylpropan-2-yl)-6,7-dihydro-5H-cyclopenta[b]pyridine-3-carboxamide, N-(1-((2,4-dimethylbenzyl)oxy)-2-methylpropan-2-yl)thieno[3,2-b]pyridine-6-carboxamide, 3-bromo-N-(1-((2,4-dimethylbenzyl)oxy)-2-methylpropan-2-yl)-1-methyl-1H-pyrrolo[2,3-b]pyridine-5-carboxamide, N-(2-methyl-1-(pyridin-2-yloxy)propan-2-yl)-6,7-dihydro-5H-cyclopenta[b]pyridine-3-carboxamide, 3-bromo-1-methyl-N-(2-methyl-1-(pyridin-2-yloxy)propan-2-yl)-1H-pyrrolo[2,3-b]pyridine-5-carboxamide, N-(1-((2-chloropyridin-4-yl)oxy)-2-methylpropan-2-yl)-6,7-dihydro-5H-cyclopenta[b]pyridine-3-carboxamide, N-(1-((2-chloropyridin-4-yl)oxy)-2-methylpropan-2-yl)thieno[3,2-b]pyridine-6-carboxamide, 3-bromo-N-(1-((2-chloropyridin-4-yl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)thieno[3,2-b]pyridine-6-carboxamide, 3-chloro-N-(1-((2-chlorobenzyl)oxy)-2-methylpropan-2-yl)-1-methyl-1H-pyrrolo[2,3-b]pyridine-5-carboxamide, 3-chloro-N-(1-((2-cyanobenzyl)oxy)-2-methylpropan-2-yl)-1-methyl-1H-pyrrolo[2,3-b]pyridine-5-carboxamide, 2-chloro-N-(2-methyl-1-phenylpropan-2-yl)-6,7-dihydro-5H-cyclopenta[b]pyridine-3-carboxamide, 2-chloro-N-(2,4-dimethyl-1-phenylpentan-2-yl)-6,7-dihydro-5H-cyclopenta[b]pyridine-3-carboxamide, N-(2-methyl-1-(pyridin-2-yloxy)propan-2-yl)thieno[3,2-b]pyridine-6-carboxamide, 3-chloro-N-(1-((2-fluorobenzyl)oxy)-2-methylpropan-2-yl)-1-methyl-1H-pyrrolo[2,3-b]pyridine-5-carboxamide, 3-chloro-N-(1-((2-chloro-5-fluorobenzyl)oxy)-2-methylpropan-2-yl)-1-methyl-1H-pyrrolo[2,3-b]pyridine-5-carboxamide, 3-bromo-N-(1-((5-fluoropyridin-2-yl)oxy)-2-methylpropan-2-yl)-1-methyl-1H-pyrrolo[2,3-b]pyridine-5-carboxamide, 2-methoxy-N-(2-methyl-1-phenylpropan-2-yl)-6,7-dihydro-5H-cyclopenta[b]pyridine-3-carboxamide, 3-chloro-N-(1-((2,4-dimethylbenzyl)oxy)-2-methylpropan-2-yl)-1-methyl-1H-pyrrolo[2,3-b]pyridine-5-carboxamide, 3-chloro-1-methyl-N-(2-methyl-1-(pyridin-2-yloxy)propan-2-yl)-1H-pyrrolo[2,3-b]pyridine-5-carboxamide, 3-chloro-N-(1-((2-chloropyridin-4-yl)oxy)-2-methylpropan-2-yl)-1-methyl-1H-pyrrolo[2,3-b]pyridine-5-carboxamide, 3-chloro-N-(1-((5-fluoropyridin-2-yl)oxy)-2-methylpropan-2-yl)-1-methyl-1H-pyrrolo[2,3-b]pyridine-5-carboxamide, 3-chloro-N-(1-((5-cyanopyridin-2-yl)oxy)-2-methylpropan-2-yl)-1-methyl-1H-pyrrolo[2,3-b]pyridine-5-carboxamide, N-(1-((5-cyanopyridin-2-yl)oxy)-2-methylpropan-2-yl)-6,7-dihydro-5H-cyclopenta[b]pyridine-3-carboxamide, N-(1-((5-cyanopyridin-2-yl)oxy)-2-methylpropan-2-yl)thieno[3,2-b]pyridine-6-carboxamide, 3-bromo-N-(1-((5-cyanopyridin-2-yl)oxy)-2-methylpropan-2-yl)-1-methyl-1H-pyrrolo[2,3-b]pyridine-5-carboxamide, N-(1-((2-chloro-5-fluorobenzyl)oxy)-2-methylpropan-2-yl)-1-methyl-1H-pyrrolo[2,3-b]pyridine-5-carboxamide, N-(1-((2,5-dimethylbenzyl)oxy)-2-methylpropan-2-yl)-1-methyl-1H-pyrrolo[2,3-b]pyridine-5-carboxamide, 1-methyl-N-(2-methyl-1-(pyridin-2-yloxy)propan-2-yl)-1H-pyrrolo[2,3-b]pyridine-5-carboxamide, N-(1-((2-chloropyridin-4-yl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)-1-methyl-1H-pyrrolo[2,3-b]pyridine-5-carboxamide, N-(1-((5-cyanopyridin-2-yl)oxy)-2-methylpropan-2-yl)-1-methyl-1H-pyrrolo[2,3-b]pyridine-5-carboxamide, 3-bromo-N-(1-((2-chloro-5-fluorobenzyl)oxy)-2-methylpropan-2-yl)-1-methyl-1H-pyrrolo[2,3-b]pyridine-5-carboxamide, N-(1-((5-fluoropyridin-2-yl)oxy)-2-methylpropan-2-yl)-6,7-dihydro-5H-cyclopenta[b]pyridine-3-carboxamide, N-(1-((2-cyanobenzyl)oxy)-2-methylpropan-2-yl)-2-methoxy-6,7-dihydro-5H-cyclopenta[b]pyridine-3-carboxamide, 2-chloro-N-(1-((2-cyanobenzyl)oxy)-2-methylpropan-2-yl)-6,7-dihydro-5H-cyclopenta[b]pyridine-3-carboxamide, N-(1-((2,4-dimethylbenzyl)oxy)-2-methylpropan-2-yl)-2-methoxy-6,7-dihydro-5H-cyclopenta[b]pyridine-3-carboxamide, 2-chloro-N-(1-((2,4-dimethylbenzyl)oxy)-2-methylpropan-2-yl)-6,7-dihydro-5H-cyclopenta[b]pyridine-3-carboxamide, N-(2,4-dimethyl-1-phenylpentan-2-yl)-2-methoxy-6,7-dihydro-5H-cyclopenta[b]pyridine-3-carboxamide, N-(1-(2-fluorophenyl)propan-2-yl)-N-methoxy-1-methyl-1H-pyrrolo[2,3-b]pyridine-5-carboxamide, 2-(2-methoxy-6,7-dihydro-5H-cyclopenta[b]pyridine-3-carboxamido)-2-methylpropyl 3-fluorobenzoate, 2-methoxy-N-(2-methyl-1-(pyridin-2-yloxy)propan-2-yl)-6,7-dihydro-5H-cyclopenta[b]pyridine-3-carboxamide, 2-chloro-N-(2-methyl-1-(pyridin-2-yloxy)propan-2-yl)-6,7-dihydro-5H-cyclopenta[b]pyridine-3-carboxamide, N-(1-((2-chloropyridin-4-yl)oxy)-2-methylpropan-2-yl)-2-methoxy-6,7-dihydro-5H-cyclopenta[b]pyridine-3-carboxamide, 2-chloro-N-(1-((2-chloropyridin-4-yl)oxy)-2-methylpropan-2-yl)-6,7-dihydro-5H-cyclopenta[b]pyridine-3-carboxamide, N-(1-benzylcyclopropyl)-2-methoxy-6,7-dihydro-5H-cyclopenta[b]pyridine-3-carboxamide, N-(1-benzylcyclopropyl)-2-chloro-6,7-dihydro-5H-cyclopenta[b]pyridine-3-carboxamide, 2-methoxy-N-((1-phenylcyclopropyl)methyl)-6,7-dihydro-5H-cyclopenta[b]pyridine-3-carboxamide, 2-chloro-N-((1-phenylcyclopropyl)methyl)-6,7-dihydro-5H-cyclopenta[b]pyridine-3-carboxamide, 2-methoxy-N-(4-phenylbutan-2-yl)-6,7-dihydro-5H-cyclopenta[b]pyridine-3-carboxamide, 2-chloro-N-(4-phenylbutan-2-yl)-6,7-dihydro-5H-cyclopenta[b]pyridine-3-carboxamide, 2-chloro-N-(1-phenylcyclopropyl)-6,7-dihydro-5H-cyclopenta[b]pyridine-3-carboxamide, 2-methoxy-N-(1-phenylcyclopropyl)-6,7-dihydro-5H-cyclopenta[b]pyridine-3-carboxamide, N-(1-(2-fluorophenyl)propan-2-yl)-N-methoxy-6,7-dihydro-5H-cyclopenta[b]pyridine-3-carboxamide, 2-chloro-N-(1-(2-fluorophenyl)propan-2-yl)-N-methoxy-6,7-dihydro-5H-cyclopenta[b]pyridine-3-carboxamide, N-(1-(2-fluorophenyl)propan-2-yl)-N-methoxy-1H-pyrrolo[2,3-b]pyridine-5-carboxamide, 2-chloro-N-(1-(4-fluorophenyl)-2,4-dimethylpent-4-en-2-yl)-6,7-dihydro-5H-cyclopenta[b]pyridine-3-carboxamide, 2-chloro-N-(1-(2-fluorophenoxy)-2,4-dimethylpent-4-en-2-yl)-6,7-dihydro-5H-cyclopenta[b]pyridine-3-carboxamide, 2-(2-chloro-6,7-dihydro-5H-cyclopenta[b]pyridine-3-carboxamido)-2-methylpropyl 3-fluorobenzoate, 2-methoxy-N-(4,4,4-trifluoro-2-methyl-1-phenylbutan-2-yl)-6,7-dihydro-5H-cyclopenta[b]pyridine-3-carboxamide, 2-chloro-N-(2-methyl-1-phenylpropan-2-yl)-5,6,7,8-tetrahydroquinoline-3-carboxamide, 2-chloro-N-(2,4-dimethyl-1-phenylpentan-2-yl)-5,6,7,8-tetrahydroquinoline-3-carboxamide, 2-chloro-N-(1-((2,4-dimethylbenzyl)oxy)-2-methylpropan-2-yl)-5,6,7,8-tetrahydroquinoline-3-carboxamide, N-(2,4-dimethyl-1-phenylpentan-2-yl)-2-methyl-1H-pyrrolo[2,3-b]pyridine-5-carboxamide, N-(2,4-dimethyl-1-phenylpentan-2-yl)-1,2-dimethyl-1H-pyrrolo[2,3-b]pyridine-5-carboxamide, N-(1-benzylcyclopropyl)-1,2-dimethyl-1H-pyrrolo[2,3-b]pyridine-5-carboxamide, 2-chloro-N-(1-((2-chlorobenzyl)oxy)-2-methylpropan-2-yl)-5,6,7,8-tetrahydroquinoline-3-carboxamide, 2-chloro-N-(1-((2-fluorobenzyl)oxy)-2-methylpropan-2-yl)-5,6,7,8-tetrahydroquinoline-3-carboxamide, 2-chloro-N-(1-((2-cyanobenzyl)oxy)-2-methylpropan-2-yl)-5,6,7,8-tetrahydroquinoline-3-carboxamide, 2-chloro-N-(1-(4-fluorophenyl)-2,4-dimethylpent-4-en-2-yl)-5,6,7,8-tetrahydroquinoline-3-carboxamide, 2-chloro-N-(1-(2-fluorophenoxy)-2,4-dimethylpent-4-en-2-yl)-5,6,7,8-tetrahydroquinoline-3-carboxamide, N-(1-((2-chlorobenzyl)oxy)-2-methylpropan-2-yl)-2-methoxy-6,7-dihydro-5H-cyclopenta[b]pyridine-3-carboxamide, N-(1-((2-fluorobenzyl)oxy)-2-methylpropan-2-yl)-2-methoxy-6,7-dihydro-5H-cyclopenta[b]pyridine-3-carboxamide, N-(1-(4-fluorophenyl)-2,4-dimethylpent-4-en-2-yl)-2-methoxy-6,7-dihydro-5H-cyclopenta[b]pyridine-3-carboxamide, N-(1-(2-fluorophenoxy)-2,4-dimethylpent-4-en-2-yl)-2-methoxy-6,7-dihydro-5H-cyclopenta[b]pyridine-3-carboxamide, N-(1-((2-chloro-5-fluorobenzyl)oxy)-2-methylpropan-2-yl)-2-methoxy-6,7-dihydro-5H-cyclopenta[b]pyridine-3-carboxamide, N-(1-(4-fluorophenyl)-2-methylpropan-2-yl)-1,2-dimethyl-1H-pyrrolo[2,3-b]pyridine-5-carboxamide, 1,2-dimethyl-N-(2-phenylpropyl)-1H-pyrrolo[2,3-b]pyridine-5-carboxamide, 1,2-dimethyl-N-((1-phenylcyclopropyl)methyl)-1H-pyrrolo[2,3-b]pyridine-5-carboxamide, 2-chloro-N-methoxy-N-(1-phenylpropan-2-yl)-6,7-dihydro-5H-cyclopenta[b]pyridine-3-carboxamide, 4-methyl-N-(1-phenylcyclopropyl)-3,4-dihydro-2H-pyrido[3,2-b][1,4]oxazine-7-carboxamide, N-(1-(4-fluorophenyl)cyclopropyl)-4-methyl-3,4-dihydro-2H-pyrido[3,2-b][1,4]oxazine-7-carboxamide, 4-methyl-N-((1-phenylcyclopropyl)methyl)-3,4-dihydro-2H-pyrido[3,2-b][1,4]oxazine-7-carboxamide, N-(1-benzylcyclopropyl)-4-methyl-3,4-dihydro-2H-pyrido[3,2-b][1,4]oxazine-7-carboxamide, 2-chloro-N-(2-methyl-1-(pyridin-2-yloxy)propan-2-yl)-5,6,7,8-tetrahydroquinoline-3-carboxamide, 2-chloro-N-(1-((2-chloropyridin-4-yl)oxy)-2-methylpropan-2-yl)-5,6,7,8-tetrahydroquinoline-3-carboxamide, 2-chloro-N-(4,4,4-trifluoro-2-methyl-1-phenylbutan-2-yl)-6,7-dihydro-5H-cyclopenta[b]pyridine-3-carboxamide, 2-chloro-N-(1-((2-chloro-5-fluorobenzyl)oxy)-2-methylpropan-2-yl)-5,6,7,8-tetrahydroquinoline-3-carboxamide, 2-methoxy-N-(2-methyl-1-(pyridin-2-yloxy)propan-2-yl)-5,6,7,8-tetrahydroquinoline-3-carboxamide, 2-methoxy-N-(2-methyl-1-phenylpropan-2-yl)-5,6,7,8-tetrahydroquinoline-3-carboxamide, N-(2,4-dimethyl-1-phenylpentan-2-yl)-2-methoxy-5,6,7,8-tetrahydroquinoline-3-carboxamide, 2-methoxy-N-(1-phenylcyclopropyl)-5,6,7,8-tetrahydroquinoline-3-carboxamide, 2-methoxy-N-((1-phenylcyclopropyl)methyl)-5,6,7,8-tetrahydroquinoline-3-carboxamide, 2-methoxy-N-(4-phenylbutan-2-yl)-5,6,7,8-tetrahydroquinoline-3-carboxamide, N-(1-(4-fluorophenyl)-2-methylpropan-2-yl)-2-methoxy-5,6,7,8-tetrahydroquinoline-3-carboxamide, 2-chloro-N-(4-phenylbutan-2-yl)-5,6,7,8-tetrahydroquinoline-3-carboxamide, 2-chloro-N-(1-(4-fluorophenyl)-2-methylpropan-2-yl)-5,6,7,8-tetrahydroquinoline-3-carboxamide, N-(1-(4-fluorophenyl)-2,4-dimethylpent-4-en-2-yl)-5,6,7,8-tetrahydroquinoline-3-carboxamide, N-(1-(2-fluorophenoxy)-2,4-dimethylpent-4-en-2-yl)-5,6,7,8-tetrahydroquinoline-3-carboxamide, N-(1-(4-fluorophenyl)cyclopropyl)-2-methoxy-5,6,7,8-tetrahydroquinoline-3-carboxamide, N-(4,4,4-trifluoro-2-methyl-1-phenylbutan-2-yl)-6,7-dihydro-5H-cyclopenta[b]pyridine-3-carboxamide, 2-chloro-N-(4,4,4-trifluoro-2-methyl-1-phenylbutan-2-yl)-5,6,7,8-tetrahydroquinoline-3-carboxamide, 2-methoxy-N-(4,4,4-trifluoro-2-methyl-1-phenylbutan-2-yl)-5,6,7,8-tetrahydroquinoline-3-carboxamide, 1-methyl-N-(4,4,4-trifluoro-2-methyl-1-phenylbutan-2-yl)-1H-pyrrolo[2,3-b]pyridine-5-carboxamide, 3-chloro-1-methyl-N-(4,4,4-trifluoro-2-methyl-1-phenylbutan-2-yl)-1H-pyrrolo[2,3-b]pyridine-5-carboxamide, 3-chloro-6-methoxy-1-methyl-N-(4,4,4-trifluoro-2-methyl-1-phenylbutan-2-yl)-1H-pyrrolo[2,3-b]pyridine-5-carboxamide, 6,6-difluoro-N-(4,4,4-trifluoro-2-methyl-1-phenylbutan-2-yl)-5,6,7,8-tetrahydroquinoline-3-carboxamide, N-(2,4-dimethyl-1-phenylpentan-2-yl)-6,6-difluoro-5,6,7,8-tetrahydroquinoline-3-carboxamide, 4-chloro-N-(4,4,4-trifluoro-2-methyl-1-phenylbutan-2-yl)-5,6,7,8-tetrahydroquinoline-3-carboxamide, 4-chloro-N-(2,4-dimethyl-1-phenylpentan-2-yl)-6,7-dihydro-5H-cyclopenta[b]pyridine-3-carboxamide, 4-chloro-N-(2,4-dimethyl-1-phenylpent-4-en-2-yl)-6,7-dihydro-5H-cyclopenta[b]pyridine-3-carboxamide, 4-chloro-N-(2,4-dimethyl-1-phenylpentan-2-yl)-5,6,7,8-tetrahydroquinoline-3-carboxamide, N-(2,4-dimethyl-1-phenylpentan-2-yl)-4-methoxy-5,6,7,8-tetrahydroquinoline-3-carboxamide, N-(2,4-dimethyl-1-phenylpentan-2-yl)-4-methoxy-6,7-dihydro-5H-cyclopenta[b]pyridine-3-carboxamide, N-(2,4-dimethyl-1-phenylpent-4-en-2-yl)-4-methoxy-6,7-dihydro-5H-cyclopenta[b]pyridine-3-carboxamide, 4-chloro-N-(1-(2-fluorophenyl)-2-methylpropan-2-yl)-6,7-dihydro-5H-cyclopenta[b]pyridine-3-carboxamide, 4-chloro-N-(1-(4-fluorophenyl)-2-methylpropan-2-yl)-6,7-dihydro-5H-cyclopenta[b]pyridine-3-carboxamide, or N-(1-(2-fluorophenyl)-2-methylpropan-2-yl)-4-methoxy-6,7-dihydro-5H-cyclopenta[b]pyridine-3-carboxamide, N-(1-(4-fluorophenyl)-2-methylpropan-2-yl)-4-methoxy-6,7-dihydro-5H-cyclopenta[b]pyridine-3-carboxamide.

11. A combination comprising the compound of Formula (I) according to claim 1 and at least one further pesticidally active substance selected from the group consisting of fungicides, insecticides, nematicides, acaricides, biopesticides, herbicides, safeners, plant growth regulators, antibiotics, fertilizers and nutrients.

12. An agrochemical composition comprising the compound of Formula (I) according to claim 1 and at least one agrochemically acceptable auxiliary selected from the group consisting of inert carrier, surfactants, additives, solid diluents and liquid diluents.

13. The composition according to claim 12, wherein said composition may further comprise at least one additional active ingredient selected from the group consisting of fungicides, insecticides, nematicides, acaricides, biopesticides, herbicides, safeners, plant growth regulators, antibiotics, fertilizers and nutrients.

14. The composition according to claim 13, wherein said composition is applied to a seed and the compound of the Formula (I) in said composition is in an amount in a range of 0.1 g a.i. to 10 kg a.i. per 100 kg of seeds.

15. A method for controlling or preventing phytopathogenic microorganisms, wherein the method comprises treating the fungi or the materials, plants, plant parts, locus thereof, soil or seeds to be protected against fungal attack, with an effective amount of at least one compound of formula (I) according to claim 1.

16. A method for controlling or preventing an infestation of plants by phytopathogenic microorganisms in agricultural crops and/or horticultural crops wherein an effective amount of at least one compound of formula (I) according to claim 1 is applied to the plants, to parts thereof or locus thereof.

17. The method according to claim 16, wherein the phytopathogenic microorganisms are selected from wheat leaf blotch (Zymoseptoria tritici), wheat brown rust (Puccinia triticina), wheat stripe rust (Puccinia striiformis), scab of apple (Venturia inaequalis), powdery mildew of grapevine (Uncinula necator), barley scald (Rhynchosporium secalis), blast of rice (Pyricularia oryzae), rust of soybean (Phakopsora pachyrhizi), glume blotch of wheat (Leptosphaeria nodorum), powdery mildew of wheat (Blumeria graminis f. sp. tritici), powdery mildew of barley (Blumeria graminis f. sp. hordei), powdery mildew of cucurbits (Erysiphe cichoracearum), anthracnose of cucurbits (Colletotrichum lagenarium), leaf spot of beet and soybean (Cercospora beticola, Cercospora sojina), early blight of tomato (Alternaria solani), Gray mold (Botrytis cinerea), target spot (Corynespora cassiicola), Late blight of potato & tomato (Phytophthora infestans), rhizoctonia aerial, foliage, and web blight (Rhizoctonia solani), Septoria nodorum blotch on wheat (Parastagonospora nodorum), root rot, scab or head blight on cereals (e.g. wheat or barley) (Fusarium culmorum) or spot blotch of barley (Cochliobolus sativus).
